(19) United States
(12) Patent Application Publication
(10) Pub. No.: US 2014/0341950 A1
(43) Pub. Date: Nov. 20, 2014

(54) RECOMBINANT AVIAN PARAMYXOVIRUS VACCINE AND METHOD FOR MAKING AND USING THEREOF

(71) Applicants: Michel Bublot, Chaponost (FR); Teshome Mebatsion, Watkinsville, GA (US); Joyce Pritchard, Gainesville, GA (US); Egbert Mundt, Dessau (DE)

(72) Inventors: Michel Bublot, Chaponost (FR); Teshome Mebatsion, Watkinsville, GA (US); Joyce Pritchard, Gainesville, GA (US); Egbert Mundt, Dessau (DE)

(73) Assignees: UNIVERSITY OF GEORGIA RESEARCH FOUNDATION, INC., Athens, GA (US); MERIAL LIMITED, Duluth, GA (US)

(21) Appl. No.: 13/894,582

(22) Filed: May 15, 2013

(51) Int. Cl. A61K 39/155

(52) U.S. Cl. CPC A61K 39/155
USPC 424/211.1; 536/23.1; 435/320.1

(57) ABSTRACT

The present invention encompasses engineered APMV compositions or vaccines. The vaccine or composition may be a recombinant APMV composition or vaccine. The present invention encompasses methods for modifying the genome of APMV to produce recombinant APMV; modified APMV prepared by such methods; DNA and protein sequences; and methods for infecting cells and host animals with such recombinant APMV.

### INCORPORATION BY REFERENCE

This application claims benefit of U.S. provisional application Ser. No. 61/235,912 filed Aug. 21, 2009.

### FIELD OF THE INVENTION

The invention relates to avian paramyxoviruses (APMV) and APMV sequences. The invention relates to viral vectors for the insertion and expression of foreign genes for use as safe immunization vehicles to protect against a variety of pathogens. It relates to vector vaccine in a reverse genetics system for the production of live attenuated vaccines. It also relates to polynucleotides which can be used for the production of subunits in an in vitro expression vector or as sequences to be integrated into a viral or plasmid type in vivo expression vector.

The present invention relates to unmodified and modified APMV virus, to methods of making and using the same, and to certain DNA and protein sequences. More in particular, the invention relates to APMV virus in which the naturally occurring genome of the virus has been altered (“APMV mutants” or “recombinant APMV”) and to methods of making and using such APMV mutants or recombinant APMV.

### BACKGROUND OF THE INVENTION

Viral vector vaccines represent one of the most rapidly growing areas in vaccine development. Many vaccines in clinical development for major global infectious diseases, HIV, tuberculosis and malaria, are based on viral vectors. Viral vector vaccines for animals are already on the market, e.g. avipox vector vaccines for companion animals and poultry, avian herpesviruses vectored vaccines for poultry, and vaccinia virus vectored vaccines for wildlife. But other livestock vector vaccines are in development. The advantage of viral vector vaccines is that they can be administered safely due to the use of a vector backbone which is strongly attenuated and does not cause disease in the animal itself. The disadvantage of currently used viral vectors is the existence of maternally derived or antibodies acquired due to a past infection. These antibodies will neutralize the vector virus and thus diminish the success of the vector vaccine. One major impetus for the development of vector vaccines was the occurrence of highly pathogenic influenza virus H5N1 occurring first in Asia and later in Europe and Africa. Several vector vaccine candidates have been developed including fowl poxvirus (Taylor et al, 1988), vaccinia virus (Chambers et al., 1988), Rous sarcoma virus (Hunt et al, 1988), adenoviruses (Tang et al., 2002, Gao et al, 2006), Venezuelan equine encephalitis virus (Schultz-Cherry et al, 2000), Newcastle disease virus (U.S. Pat. No. 6,719,979, Veits et al., 2006, Swayne et al, 2002, Park et al, 2006), herpesvirus of infectious laryngotracheitis (Veits et al. 2003), herpesvirus of turkey (Darteil et al., 1995), and adenovirus based vector vaccines (Hoelscher et al, 2008, Toro et al, 2007). The efficacy of these vector vaccines have been tested in naive birds, but so far no reports have been published on the efficacy of these vector vaccines in birds with a preexisting immunity to the viral vector and/or to the protein coded by the insert.

The virus family Paramyxoviridae includes both human (measles, mumps, parainfluenza and respiratory syncytial virus) and animal pathogens (Newcastle disease virus and rinderpest virus) that cause significant impact on public health as well as the global economy (Lamb et al., 2007). Members of this virus family are defined by having a monopartite, negative sense, single-stranded RNA genome. The Paramyxoviridae family consists of two subfamilies namely Paramyxovirinae and Pneumovirinae. Owing to recent reclassification, the subfamily Paramyxovirinae includes five genera, i.e Morbillivirus, Henipavirus, Rubulavirus, Respirovirus and Avulavirus while Pneumovirinae includes Pneumovirus and Metapneumovirus (Mayo, 2002). Avian paramyxoviruses (APMV) are classified in the genus Avulavirus and comprise nine antigenically distinct serotypes that have been defined using hemagglutination inhibition (HI) tests (Alexander, 1988). Of the nine serotypes, isolates belonging to the APMV-1 subtype can cause a devastating disease in commercial poultry and are classified as velogenic Newcastle disease virus (NDV). Milder forms of NDV are designated as mesogenic and lentogenic isolates, wherein the latter form is mostly asymptomatic in domestic poultry. Isolates belonging to the APMV-2, 3, 6 and 7 have also been associated with disease in domestic poultry. Specifically, infections by isolates of APMV-2 and 3 can cause mild respiratory disease and problems in egg quality and quantity (Bankowski et al., 1981; Redmann et al., 1991; Tumova et al., 1979; Zhang et al., 2007). Isolates of APMV-6 and 7 have been known to infect turkeys, ducks and migratory birds and can induce respiratory disease that may be complicated by secondary infection (Saif et al., 1997; Shortridge et al., 1980). On the other hand, isolates of APMV-4, 5, 8 and 9 have been isolated from ducks, waterfowl and other wild birds but the birds rarely show clinical signs after viral infection (Alexander et al., 1983; Capua et al., 2004; Gough et al., 1984; Maldonado et al., 1995; Shortridge et al., 1980).

The complete genomic sequences of several NDV isolates have been established and used to elucidate the various determinants of NDV virulence (de Leeuw et al., 1999; Krishnamurthy et al., 1998; Zou et al., 2005). In the recent two years several APMV sequences other than APMV1 have been published, such as GenBank accession number EU338414 for APMV-2, EU403085 for APMV-3, FJ177514 for APMV-4, EU622637 for APMV-6, FJ231524 for APMV-7, FJ215863, FJ215864 and FJ619036 for APMV-8, EU910942 for APMV-9. Besides the sequence information, not much is known about virulence factors. Isolates of APMV 2-9 have been mostly isolated from migratory birds. Interestingly, there are very few reports of experimental infection of chickens with such isolates (Saif et al., 1997). Since these APMV circulate widely in wild birds and in certain cases have been isolated from commercial flocks (Zhang et al., 2007) that sometimes cause disease in them (Saif et al., 1997; Shihmanter et al., 1998; Shihmanter et al., 1998), knowledge about their virulence in poultry is needed.

Most of the APMV isolates cause a relatively mild disease that may be exacerbated in the presence of concomitant bacterial or viral infections which might lead to economic impact. In particular, APMV-2 was first isolated as a secondary pathogen in 1956 from chickens affected by acute laryngotracheitis in Southern California (Bankowski et al., 1960). Since then numerous strains of this serotype have been isolated from several avian species signifying that APMV-2 is widely disseminated worldwide (Andral et al., 1984; Bradshaw et al., 1979; Fleury et al., 1979; Goodman et al., 1988; Lang et al., 1975; Lipkind et al., 1982; Lipkind et al., 1979; Zhang et al., 2006). Bankowski et. al. reported that natural as well as artificial exposure of laying turkey hens to APMV-2 caused a pronounced decline in hatchability and poultry yield (Bankowski et al., 1981). Initial examples of APMV-4 isolation were from hunter-killed feral ducks on the Mississippi flyway in the United States (Webster et al., 1976) and from chickens, ducks and geese in Hong Kong during influenza surveillance programs of poultry (Alexander et al., 1979). Apart from an isolate from a ringed teal suffering from hemorrhagic enteritis (Gough et al., 1984), all other isolates were seemingly non-pathogenic in poultry and found to have wide distribution among waterfowl throughout the world (Stanislawek et al., 2002; Tumova et al., 1989; Yamane et al., 1982). Gough et al. reported that no clinical signs and very low HI titers (1:8 or less) were obtained after the intranasal inoculation of one-week old ducklings and two-week old chickens with the isolate from a ringed teal (Gough et al., 1984). Similarly, the first isolates of APMV-6 were also from domestic poultry in Hong Kong as a result of an influenza surveillance program and were reported to be non-pathogenic in chickens based on low HI titers from experimentally infected chickens (Shortridge et al., 1980). However, there have been reports of APMV-6 infection of turkeys leading to mild respiratory disease and egg production problems (Alexander, 2003).

APMV-8 (Goose/Delaware/1053/76) was first isolated in the USA from a hunter-killed Canada goose (Branta canadensis) (Rosenberger et al., 1974). A serological survey (from 1990 to 1992) of wildfowl in southern Spain showed a notable prevalence of APMV-8 antibodies in up to 43% of the tested sera (Maldonado et al., 1995). Another serological study to determine the status of live, healthy mallard ducks in New Zealand for APMV infection revealed the presence of APMV-8 antibodies in 56% of the tested sera (Stanislawek et al., 2002). Warke et al (2008) described that between 16% to 31% of investigated chicken sera might have had APMV-8 antibodies. But due to existing high titers against APMV 1 the probability of a false positive HI test is possible since the sera do not react very specifically in the HI assay. With the exception of a few waterfowl isolates of APMV-8 isolated while the populations were being surveyed for avian influenza viruses (Stallknecht et al., 1991), there has been a dearth of information about the prevalence and pathogenicity of this virus.

The development of reverse genetics systems for the negative stranded RNA genome of NDV has made it possible to insert foreign gene sequences into the genome, thus making it possible to create recombinant NDV vectors for vaccination and gene therapy (Krishnamurthy et al., 2000; Peeters et al., 1999; Roemer-Oberdoerfer et al., 1999). Recombinant NDV vectors expressing foreign viral proteins such as the HA protein of the H1 subtype of influenza A virus (Nakaya et al., 2001), VP2 protein of infectious bursal disease virus (IBDV) (Huang et al., 2004), avian influenza virus hemagglutinin of subtype H5 (Veits et al., 2006; Ge et al., 2007) and subtype H7 (Park et al., 2006) have been reported. However the efficacy of most of such vaccines has been demonstrated only in SPF birds. NDV causes a devastating disease in poultry leading to serious economic losses in the poultry industry. Commercial chickens therefore are routinely vaccinated against NDV in most countries of the world. Due to this, chickens from immunized parent flocks have a high level of maternally derived antibodies. Conventional live NDV vaccines provide protection even in the presence of these antibodies. However recombinant NDV vaccines (with foreign gene insertions) are generally more attenuated as compared to live NDV vaccines and their efficacy may be impaired in presence of NDV maternal antibodies. Therefore, there is a need for a vector vaccine platform which can provide the basis for safe vaccines for the expression of heterologous antigens. Ideally, the recombinant vaccine can induce a strong humoral immune response, can be applicable by mass administration, and is inexpensive.

### SUMMARY OF THE INVENTION

The present invention relates to a vaccine or composition comprising (i) a recombinant APMV and (ii) a pharmaceutically or veterinarily acceptable carrier. The present invention encompasses methods for modifying the genome of APMV to produce recombinant APMV virus or APMV rival vector; modified APMV prepared by such methods; DNA and protein sequences; and methods for infecting cells and host animals with such recombinant APMV to provoke the amplification of exogenous DNA and proteins encoded by the exogenous DNA, including antigenic proteins, by said cells and host animals.

One aspect of the invention relates to APMV virus, DNA and protein sequences involved in making modified or recombinant virus. One embodiment of the invention relates to the genomic and protein sequence of APMV-2, 4, 6, or 8.

Another aspect of the invention relates to a modified recombinant APMV virus, which viruses have enhanced safety, strong humoral immune response, and a method of making such recombinant viruses.

Another aspect of the invention relates to a recombinant APMV virus vaccine or composition having an increased level of safety compared to known APMV or other recombinant vaccines.

In another aspect, the present invention provides unmodified and modified APMV viral vector for expressing a gene product in a host.

Another aspect of the invention is directed to a recombinant APMV virus modified by the insertion therein of DNA from any source into the intergenic region or the nonessential region of the APMV genome. Synthetically modified APMV virus recombinants carrying heterologous genes coding for and expressing an antigen, are used according to the invention to create novel compositions or vaccines.

Another aspect of the invention relates to an APMV viral vector which provides a reverse genetics system, wherein the vector can be used as a backbone for recombinant vaccines or compositions in different host animals.

In one aspect, the present invention relates to a pharmaceutical composition or vaccine for inducing an immunological response in a host animal inoculated with the composition or vaccine, the composition or vaccine including a pharmaceutical acceptable carrier and a modified APMV recombinant virus or viral vector. In yet another aspect of the invention, the recombinant APMV virus or viral vector includes, within a non-essential region of the virus genome, a heterologous DNA which encodes an antigenic protein derived from a pathogen wherein the composition or vaccine when administered to a host, is capable of inducing an immunological response specific to the protein encoded by the pathogen.

Another aspect of the invention relates to a method for inducing an immunological response in an animal to an antigen, which method comprises inoculating the animal with a vaccine or a pharmaceutical composition containing modified recombinant APMV virus or viral vector which comprises and expresses the antigenic determinant of a pathogen for said animal. Yet another aspect of the invention relates to a method for inducing an immunological response in an animal to an antigen in a prime-boost regime.

Another aspect of the invention relates to a method of expressing a gene product in a cell culture in vitro by introducing into the cell a modified recombinant APMV virus, wherein the gene may be an antigenic protein derived from a pathogen.

### BRIEF DESCRIPTION OF THE DRAWINGS

The following detailed description, given by way of example, and which is not intended to limit the invention to specific embodiments described, may be understood in conjunction with the accompanying figures, in which:

FIG. 1 is a table showing virus isolation from several organs of chickens after experimental infection with APMV-2, 4, 6 in embryonated chicken eggs.

FIG. 2 is a table showing the histology results of several organs of chickens after experimental infection with APMV-2, 4, 6.

FIG. 3 depicts HI antibody titers in SPF chickens experimentally inoculated with APMV-2, 4 or 6. Chicken sera samples collected at day 2, 4, 7, 14 and 28 post infection were subject to the HI test to analyze the presence of HI antibodies.

FIG. 4 shows HI antibody titers in SPF chickens and ducks experimentally infected with APMV-8. Chickens and ducks were oro-nasal infected with a dose of 10⁶ EID₅₀ of APMV-8. Sera samples were collected at day 2, 4, 7, 14, and 28 p. i. and analyzed by the HI test with the APMV-8 antigen. The HI serum titers (in log₂) are shown on the left axis

FIG. 5 shows the development of HI antibody titers in SPF chickens during a prime/boost vaccination scheme with APMV-8. One-day old SPF chickens were infected at day 1 (prime) and day 14 (boost) with a dose of 10⁶ EID₅₀ of APMV-8. Serum samples were obtained at day 7 and 14 p.i. after the first infection and day 7 and 14 p.i. after the second infection. The sera were subjected to the HI test. The HI serum titers (in log 2) are shown on the left axis.

FIG. 6 shows the analysis of RT-PCR products by agarose gel electrophoresis. Tracheal tissues were taken at day 2 p.i. from non-infected ducks (C1-C5) and APMV-8 infected ducks (I1-I5). The tissues were homogenized and RNA was prepared for RT-PCR. A water control (W) was prepared in parallel. The reaction products were separated on a 1.5% agarose gel. The size of the fragment was controlled by using the 100 bp ladder (New England Biolabs). The sizes of the DNA fragments are shown at the right.

FIG. 7 is a table showing virus isolation from chicken and ducks experimentally infected with APMV-8. The virus isolation from chicken tissue was performed in embryonated chicken eggs and the detection of viral RNA from duck tissue by RT-PCR.

FIG. 8 is a table showing the results of the histological examination of several organs after infection of chicken and Pekin ducks with APMV-8.

FIG. 9 shows the development of HI antibody titers in SPF chickens after infection with different doses of APMV-8. One-day old SPF chickens were infected at day 1 with different infectious doses (ID) of APMV-8 or mock-infected with virus transport medium (VTM). Blood was taken at day 7 and 14 p.i. and the obtained serum samples were analyzed by the HI test. The HI serum titers (in log 2) are shown on the left axis. The geometric mean titer (GMT) of the serum samples are shown in the lowest row.

FIG. 10 shows the development of HI antibody titers in Pekin ducks after infection with different doses of APMV-8. One-day old SPF Pekin ducks were infected at day 1 with different infectious doses (ID) of APMV-8 or mock-infected with virus transport medium (VTM). Blood was taken at day 7 and 14 p.i. and the obtained serum samples were analyzed by the HI test. The HI serum titers (in log 2) are shown on the left axis. The geometric mean titer (GMT) of the serum samples are shown in the lowest row.

FIG. 11 is a table showing the SEQ ID NOs of the corresponding DNA and protein sequences.

FIG. 12 depicts the full length genome sequence of APMV-8 strain (APMV-8: SCWDS ID: MA-7, isolated from a mallard) and a genetic map of the full length APMV-8 genome.

FIG. 12 depicts the DNA sequence (SEQ ID NO:2) encoding APMV-8 Nucleoprotein (NP) and the NP protein sequence (SEQ ID NO:3).

FIG. 13 depicts the DNA sequence (SEQ ID NO:4) encoding APMV-8 Phospho Protein (P) and the P protein sequence (SEQ ID NO:5).

FIG. 14 depicts the DNA sequence (SEQ ID NO:6) encoding APMV-8 Matrixprotein (M) and the M protein sequence (SEQ ID NO:7).

FIG. 15 depicts the DNA sequence (SEQ ID NO:8) encoding APMV-8 Fusionprotein (F) and the F protein sequence (SEQ ID NO:9).

FIG. 16 depicts the DNA sequence (SEQ ID NO:10) encoding APMV-8 Hemagglutinin/neuraminidase (HN) and the FIN protein sequence (SEQ ID NO:11).

FIG. 17 depicts the DNA sequence (SEQ ID NO:12) encoding APMV-8 Polymerase (L) and the L protein sequence (SEQ ID NO:13). This APMV-8 L(1) protein is translated from the ATG codon located at positions 8273-8275 of SEQ ID NO:1.

FIG. 18 depicts the protein sequence (2) of APMV-8 Polymerase (L) (SEQ ID NO:14). This APMV-8 L(2) protein is translated from the ATG codon located at positions 8297-8299 of SEQ ID NO:1. SEQ ID NO:14 does not contain the first 8 amino acids of SEQ ID NO:13.

FIG. 19A is a flow diagram of the APMV-8 reverse genetics system. FIG. 19B depicts the result of replication of APMV-8 virus in MDCK cells.

FIG. 20 depicts the HI test result of commercial broiler chickens 2 weeks after APMV-8 vaccination.

FIG. 21 depicts the HI test result of commercial broiler chickens 4 weeks after APMV-8 vaccination.

FIG. 22 shows the HI test results after the in ovo vaccination at day 18 (study 1).

FIG. 23 depicts the HI test results after the in ovo vaccination at day 19 (study 2).

FIG. 24 shows the HI test results after the in ovo vaccination at day 18 (study 3).

FIG. 25 depicts 5′-full length genome (5′-FLG) and 3′-full length genome (3′-FLG) sequences, including the franking sequences.

FIG. 26 depicts plasmid maps of pcNDA-NP, pcNDA-P, pcDNA-L, and pcDNA3-T7.

FIG. 27 depicts plasmid maps of pUC18-MG-APMV-8 and pCITE4A-EGFP.

FIG. 28 depicts plasmid map of pUC57-FL-APMV-8.

FIG. 29 depicts minigenome APMV-8 sequence.

FIG. 30 shows the NP protein sequence alignment and the sequence identity at the DNA and protein levels.

FIG. 31 shows the P protein sequence alignment and the sequence identity at the DNA and protein levels.

FIG. 32 shows the M protein sequence alignment and the sequence identity at the DNA and protein levels.

FIG. 33 shows the F protein sequence alignment and the sequence identity at the DNA and protein levels.

FIG. 34 shows the HN protein sequence alignment and the sequence identity at the DNA and protein levels.

FIG. 35 shows the L protein sequence alignment and the sequence identity at the DNA and protein levels.

### DETAILED DESCRIPTION OF THE INVENTION

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Unless otherwise noted, technical terms are used according to conventional usage. Definitions of common terms in molecular biology may be found in Benjamin Lewin, Genes V. published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

It is noted in this disclosure and the appended claims and/or paragraphs, the term “Avian Paramyxoviruses” or “APMV” is used interchangeably, and refers to and includes APMV-1, APMV-2, APMV-3, APMV-4, APMV-5, APMV-6, APMV-7, APMV-8, and APMV-9.

The term “animal” is used herein to include all mammals, birds and fish. The animal as used herein may be selected from the group consisting of equine (e.g., horse), canine (e.g., dogs, wolves, foxes, coyotes, jackals), feline (e.g., lions, tigers, domestic cats, wild cats, other big cats, and other felines including cheetahs and lynx), bovine (e.g., cattle), porcine (e.g., pig), ovine (e.g., sheep, goats, lamas, bisons), avian (e.g., chicken, duck, goose, turkey, quail, pheasant, parrot, finches, hawk, crow, ostrich, emu and cassowary), primate (e.g., prosimian, tarsier, monkey, gibbon, ape), humans, and fish. The term “animal” also includes an individual animal in all stages of development, including embryonic and fetal stages.

The terms “polypeptide” and “protein” are used interchangeably herein to refer to a polymer of consecutive amino acid residues.

The terms “nucleic acid”, “nucleotide”, and “polynucleotide” are used interchangeably and refer to RNA, DNA, cDNA (complementary DNA), or cRNA (complementary RNA) and derivatives thereof, such as those containing modified backbones. It should be appreciated that the invention provides polynucleotides comprising sequences complementary to those described herein. The “polynucleotide” contemplated in the present invention includes both the forward strand (5′ to 3′) and reverse complementary strand (3′ to 5′). Polynucleotides according to the invention can be prepared in different ways (e.g. by chemical synthesis, by gene cloning etc.) and can take various forms (e.g. linear or branched, single or double stranded, or a hybrid thereof, primers, probes etc.).

The term “genomic DNA” or “genome” is used interchangeably and refers to the heritable genetic information of a host organism. The genomic DNA comprises the DNA of the nucleus (also referred to as chromosomal DNA) but also the DNA of the plastids (e.g., chloroplasts) and other cellular organelles (e.g., mitochondria). The genomic DNA or genome contemplated in the present invention also refers to the RNA of a virus. The RNA may be a positive strand or a negative strand RNA. The term “genomic DNA” contemplated in the present invention includes the genomic DNA containing sequences complementary to those described herein. The term “genomic DNA” also refers to messenger RNA (mRNA), complementary DNA (cDNA), and complementary RNA (cRNA). The term “genomic RA (nucleic acid)” as used herein includes RNA, mRNA, cRNA, DNA and cDNA.

The term “gene” is used broadly to refer to any segment of polynucleotide associated with a biological function. Thus, genes or polynucleotides include introns and exons as in genomic sequence, or just the coding sequences as in cDNAs, such as an open reading frame (ORF), starting from the start codon (methionine codon) and ending with a termination signal (stop codon). Genes and polynucleotides can also include regions that regulate their expression, such as transcription initiation, translation and transcription termination. Thus, also included are promoters and ribosome binding regions (in general these regulatory elements lie approximately between 60 and 250 nucleotides upstream of the start codon of the coding sequence or gene; Doree S M et al.; Pandher K et al.; Chung J Y et al.), transcription terminators (in general the terminator is located within approximately 50 nucleotides downstream of the stop codon of the coding sequence or gene; Ward C K et al.). Gene or polynucleotide also refers to a nucleic acid fragment that expresses mRNA or functional RNA, or encodes a specific protein, and which includes regulatory sequences.

The term “heterologous DNA” as used herein refers to the DNA derived from a different organism, such as a different cell type or a different species from the recipient. The term also refers a DNA or fragment thereof on the same genome of the host DNA wherein the heterologous DNA is inserted into a region of the genome which is different from its original location.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. Alternately, the immunogen or antigen may comprise a toxin or antitoxin.

The term “immunogenic protein or peptide” as used herein includes polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques well known in the art. See, e.g., Epitope Mapping Protocols in Methods in Molecular Biology, Vol. 66 (Glenn E. Morris, Ed., 1996). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., 1984; Geysen et al., 1986. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

The term “immunogenic protein or peptide” further contemplates deletions, additions and substitutions to the sequence, so long as the polypeptide functions to produce an immunological response as defined herein. The term “conservative variation” denotes the replacement of an amino acid residue by another biologically similar residue, or the replacement of a nucleotide in a nucleic acid sequence such that the encoded amino acid residue does not change or is another biologically similar residue. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine, threonine, tyrosine. Phenylalanine, tryptophan, and tyrosine are sometimes classified as aromatic amino acids. Examples of conservative variations include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like; or a similar conservative replacement of an amino acid with a structurally related amino acid that will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide. All of the polypeptides produced by these modifications are included herein. The term “conservative variation” also includes the use of a substituted amino acid in place of an unsubstituted parent amino acid provided that antibodies raised to the substituted polypeptide also immunoreact with the unsubstituted polypeptide.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof. Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

One embodiment of the invention provides the genomic DNA sequence and encoded protein sequences of APMV-8. The complementary genomic DNA (cDNA) sequence of APMV-8 strain of the present invention has a polynucleotide sequence as set forth in SEQ ID NO:1. The APMV-8 genomic cDNA sequence (SEQ ID NO:1) has 48% sequence identity to APMV-1 genomic DNA (SEQ ID NO:15), 61% sequence identity to APMV-2 genomic DNA (SEQ ID NO:16), 47.2% sequence identity to APMV-3 genomic DNA (SEQ ID NO:17), 47.6% sequence identity to APMV-4 genomic DNA (SEQ ID NO:18), 52% sequence identity to APMV-6 genomic DNA (SEQ ID NO:19), 53% sequence identity to APMV-7 genomic DNA (SEQ ID NO:20), 99.1% sequence identity to APMV-8 genomic DNA (SEQ ID NO:37), 96.5% sequence identity to APMV-8 genomic DNA (SEQ ID NO:38), 96.4% sequence identity to APMV-8 genomic DNA (SEQ ID NO:39), 48% sequence identity to APMV-9 genomic DNA (SEQ ID NO:40). In another embodiment, the invention provides a polynucleotide having a sequence as set forth in SEQ ID NO:1, 2, 4, 6, 8, 10 or 12, and variant or fragment thereof. The invention further comprises a complementary strand to a polynucleotide described herein. In yet another embodiment, the invention provides a polypeptide having a sequence as set forth in SEQ ID NO:3, 5, 7, 9, 11, 13 or 14, and variant or fragment thereof.

Moreover, homologs of polynucleotides or polypeptides from APMV, for example APMV-8, APMV-2, APMV-4, APMV-6 strains are intended to be within the scope of the present invention. As used herein, the term “homologs” includes orthologs, analogs and paralogs. The term “analogs” refers to two polynucleotides or polypeptides that have the same or similar function, but that have evolved separately in unrelated organisms. The term “orthologs” refers to two polynucleotides or polypeptides from different species, but that have evolved from a common ancestral gene by speciation. Normally, orthologs encode polypeptides having the same or similar functions. The term “paralogs” refers to two polynucleotides or polypeptides that are related by duplication within a genome. Paralogs usually have different functions, but these functions may be related. Analogs, orthologs, and paralogs of a wild-type APMV polypeptide can differ from the wild-type APMV polypeptide by post-translational modifications, by amino acid sequence differences, or by both. In particular, homologs of the invention will generally exhibit at least 80-85%, 85-90%, 90-95%, or 95%, 96%, 97%, 98%, 99% sequence identity with all or part of the polynucleotide or polypeptide sequences of APMV-8, and will exhibit a similar function.

In another aspect, the present invention provides a genomic cDNA of APMV-8 having the sequence as set forth in SEQ ID NO:1. In yet another embodiment, the polynucleotide is a reverse complementary strand of the polynucleotide having the sequence as set forth in SEQ ID NO:1. In yet another embodiment, the polynucleotide or a reverse complementary strand of a polynucleotide of the present invention has at least 70%, at least 75%, at least 80%, at least 85%, at least 90%, at least 95%, 96%, 97%, 98% or 99% sequence identity to a polypeptide having a sequence as set forth in SEQ ID NO:1.

In one embodiment, the present invention provides a fragment of polynucleotide encoding an AMPV-8 polypeptide, such as a polynucleotide encoding a polypeptide having a sequence as set forth in SEQ ID NO: 3, 5, 7, 9, 11, 13 or 14. In yet another aspect, the present invention provides a polynucleotide encoding a polypeptide having at least 70%, at least 75%, at least 80%, at least 85%, at least 90%, at least 95%, 96%, 97%, 98% or 99% sequence identity to a polypeptide having a sequence as set forth in SEQ ID NO: 3, 5, 7, 9, 11, 13 or 14, or a conservative variant, an allelic variant, a homolog or an immunogenic fragment comprising at least eight or at east ten consecutive amino acids of one of these polypeptides, or a combination of these polypeptides.

In another aspect, the present invention provides a polynucleotide having a nucleotide sequence as set forth in SEQ ID NO:1, 2, 4, 6, 8, 10, or 12, or a variant thereof. In yet another embodiment, the polynucleotide is a reverse complementary strand of the polynucleotide having the sequence as set forth in SEQ ID NO:1. In yet another aspect, the present invention provides a polynucleotide or a reverse complementary strand of a polynucleotide having at least 70%, at least 75%, at least 80%, at least 85%, at least 90%, at least 95%, 96%, 97%, 98% or 99% sequence identity to one of a polynucleotide having a sequence as set forth in SEQ ID NO:1, 2, 4, 6, 8, 10, or 12, or a variant thereof.

In another aspect, the present invention provides a polypeptide having at least 70%, at least 75%, at least 80%, at least 85%, at least 90%, at least 95%, 96%, 97%, 98% or 99% sequence identity to a polypeptide having a sequence as set forth in SEQ ID NO:3, 5, 7, 9, 11, 13 or 14. In yet another aspect, the present invention provides fragments and variants of the APMV polypeptides identified above (SEQ ID NO: 3, 5, 7, 9, 11, 13 or 14) which may readily be prepared by one of skill in the art using well-known molecular biology techniques.

Variants are homologous polypeptides having an amino acid sequence at least 75%, 80%, 85%, 90%, 95%, 96%, 97%, 98%, or 99% identity to the amino acid sequence as set forth in SEQ ID NO: 3, 5, 7, 9, 11, 13 or 14.

Variants include allelic variants. The term “allelic variant” refers to a polynucleotide or a polypeptide containing polymorphisms that lead to changes in the amino acid sequences of a protein and that exist within a natural population (e.g., a virus species or variety). Such natural allelic variations can typically result in 1-5% variance in a polynucleotide or a polypeptide. Allelic variants can be identified by sequencing the nucleic acid sequence of interest in a number of different species, which can be readily carried out by using hybridization probes to identify the same genetic locus in those species. Any and all such nucleic acid variations and resulting amino acid polymorphisms or variations that are the result of natural allelic variation and that do not alter the functional activity of gene of interest, are intended to be within the scope of the invention.

The term “identity” with respect to sequences can refer to, for example, the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman). The sequence identity or sequence similarity of two amino acid sequences, or the sequence identity between two nucleotide sequences can be determined using Vector NTI software package (Invitrogen, 1600 Faraday Ave., Carlsbad, Calif.). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

In one aspect, the present invention relates to a pharmaceutical composition or vaccine for inducing an immunological response in a host animal inoculated with the vaccine or composition, the vaccine or composition including a pharmaceutical acceptable carrier and a modified APMV recombinant virus or viral vector. In yet another aspect of the invention, the recombinant APMV virus or viral vector includes, within a non-essential region of the virus genome, a heterologous DNA sequence which encodes an antigenic protein derived from a pathogen wherein the composition or vaccine when administered to a host, is capable of inducing an immunological response specific to the protein encoded by the pathogen.

A “vector” refers to a recombinant DNA or RNA plasmid, bacteriophage, or virus that comprises a heterologous polynucleotide to be delivered to a target cell, either in vitro or in vivo. The heterologous polynucleotide may comprise a sequence of interest for purposes of prevention or therapy, and may optionally be in the form of an expression cassette. As used herein, a vector needs not be capable of replication in the ultimate target cell or subject. The term includes vectors for cloning as well as viral vectors.

The term “engineered” or “recombinant” means a polynucleotide of semisynthetic, or synthetic origin that either does not occur in nature or is linked to another polynucleotide in an arrangement not found in nature.

The term “nonessential region” refers to a region of a virus genome which is not essential for replication and propagation of the virus in tissue culture and whose deletion or inactivation may reduce virulence in a variety of animal systems. Any nonessential region or portion thereof can be deleted from the APMV genome or a foreign sequence can be inserted in it, and the viability and stability of the recombinant APMV resulting from the deletion or insertion can be used to ascertain whether a deleted region or portion thereof is indeed nonessential. In one embodiment, the nonessential region of the APMV genome is any region on the APMV-2, 4, 6, or 8 genome that does not encode Polymerase (L). In yet another embodiment, the nonessential region comprises an open reading frame encoding a nonessential protein. In this aspect, the open reading frame is selected from the group consisting of nucleoprotein (NP), phosphoprotein (P), matrixprotein (M), fusionprotein (F), and hemagglutinin/neuraminidase (HN). In one embodiment, the nonessential region is located upstream of the NP gene. In another embodiment, the nonessential region is located downstream of the L gene. In yet another embodiment, the nonessential region is a non-coding or intergenic region. In this aspect, the non-coding or intergenic region may be a region between NP and P genes, between P and M genes, between M and F genes, or between F and FIN genes on the APMV-2, 4, 6, or 8 genome. In yet another embodiment, the nonessential region may be in the region of nucleotide positions 1-140, 1526-1692, 2910-3085, 4195-4498, 6130-6382, 8116-8272, 8116-8289, or 15013-15342 of SEQ ID NO:1.

In another aspect, the invention includes APMV chimeras in which one part or the full gene or several parts or full genes of the APMV vector are replaced by similar genes from other viruses, in particular those belonging to the paramyxoviridae family.

In one embodiment of the invention, the vaccine or pharmaceutical composition comprises an antigen selected from the group of avian pathogens including, but not limited to, Salmonella typhimurium, Salmonella enteritidis, Infectious Bronchitis virus (IBV), Newcastle Disease virus (NDV), egg drop syndrome virus (EDS), or Infectious Bursal Disease virus (IBDV), Infectious Laryngotracheitis virus (ILTV), avian adenoviruses, Marek's disease virus (MDV), fowlpox virus, duck enteritis virus (DEV), duck parvoviruses, avian influenza virus, APMV, such as APMV-1, and the like, and combinations thereof.

In another embodiment, the vaccine or pharmaceutical composition comprises an antigen selected from a feline pathogen such as, but not limited to, feline herpesvirus (FHV), feline calicivirus (FCV), feline leukemia virus (FeLV), feline immunodeficiency virus (Fly), feline parvovirus (FPV), feline infectious peritonitis virus (FIPV), rabies virus, and the like, and combinations thereof.

In yet another embodiment, the vaccine or pharmaceutical composition of the present invention comprises an antigen selected from a canine pathogen including, but not limited to, rabies virus, canine herpesvirus (CHV), canine parvovirus (CPV), canine distemper virus (CDV), canine parainfluenza 2 (CPI2), canine coronavirus, Leptospira canicola, Leptospira icterohaemorragiae, Leptospira grippotyphosa, Borrelia burgdorferi, Bordetella bronchiseptica and the like, and combinations thereof.

In yet another embodiment, the vaccine or pharmaceutical composition comprises an antigen selected from an equine pathogen, such as equine herpesvirus (type 1 or type 4), equine influenza virus, tetanus, west nile virus, equine arterivirus and the like or combinations thereof.

In yet another embodiment, the vaccine or pharmaceutical composition comprises an antigen selected from a bovine, ovine or caprine pathogen, such as rabies virus, bovine rotavirus, bovine parainfluenza virus type 3 (bPIV-3), bovine coronavirus, bovine viral diarrhea virus (BVDV), foot and mouth disease virus (FMDV), Rinderpest virus (RPV), Peste des Petits Ruminants virus (PPRV), malignant catarrhal fever viruses, bovine respiratory syncytial virus (BRSV), Infectious Bovine Rhinotracheitis virus (IBR), Escherichia coli, Pasteurella multocida, Pasteurella haemolytica and the like, and combinations thereof.

In still another embodiment, the vaccine or pharmaceutical composition comprises an antigen selected from a porcine pathogen such as, but not limited to, swine influenza virus (SIV), porcine circovirus type 2 (PCV-2), porcine reproductive respiratory syndrome virus (PRRS), pseudorabies virus (PRV), porcine parvovirus (PPV), FMDV, Mycoplasma hyopneumoniae, Erysipelothrix rhusiopathiae, Pasteurella multocida, Bordetella bronchiseptica, Escherichia coli and the like, and combinations thereof.

Construction of recombinant virus is well known in the art as described in, e.g., U.S. Pat. Nos. 4,769,330, 4,722,848, 4,603, 112, 5,174,993, and 5,756,103, 6,719,979. Specifically, a recombinant APMV virus may be constructed in two steps. First, the gene of interest to be inserted into the virus, such as an open reading frame of an antigen from an APMV-1 (NDV) or avian influenza virus or other organism, is placed into an E. coli plasmid construct into which the cDNA homologous to a section of cDNA of the APMV is inserted. Separately, the cDNA gene sequence to be inserted is preceded by a promoter region (gene start region) and followed by a gene end region which is specific for the APMV vector. The gene start/foreign antigen/gene end DNA fragment is flanked by cDNA fragment homologous of APMV-8 cDNA containing unique restriction enzyme cleavage sites. The resulting plasmid construct is then amplified by growing in E. coli bacteria and isolated. Next the recombinant plasmid is used in a restriction enzyme digest to cut out the gene start/foreign antigen/gene end DNA fragment that is flanked by cDNA homologous of APMV-8 cDNA and this fragment is ligated into the appropriately cleaved full length construct of APMV-8.

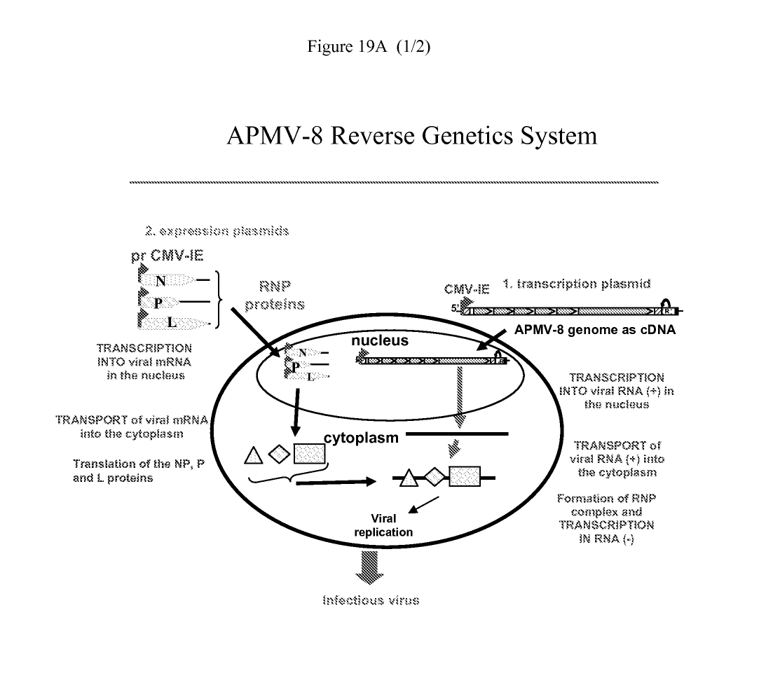

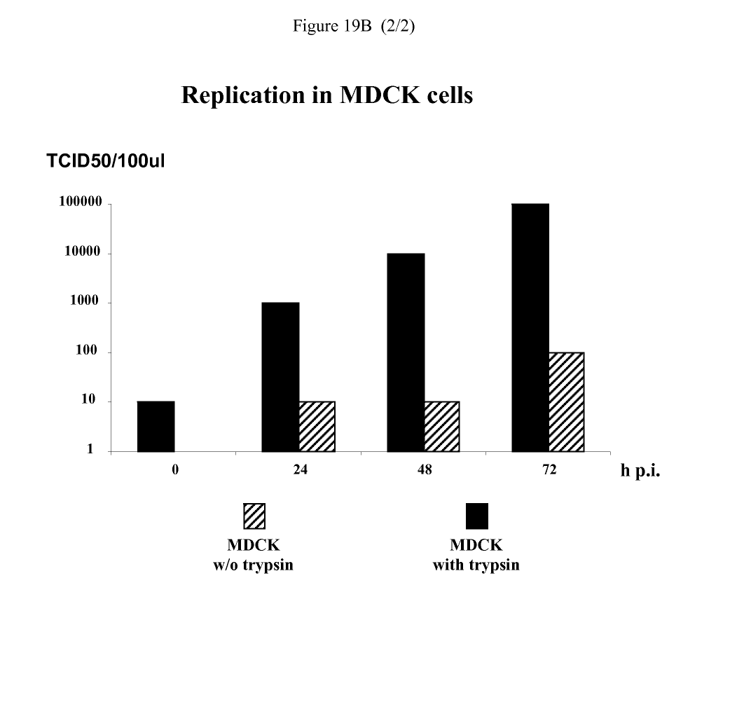

The full length construct containing the gene of interest is transfected into cells along with plasmids containing polynucleotides for expression of the APMV nucleoprotein (NP), the APMV phosphoprotein (P) and the APMV RNA polymerase (L) as well as the T7 RNA polymerase. All APMV cDNA constructs are under the control of the T7 polymerase promoter. The rescue of infectious virus is performed as described in Römer-Oberdörfer et al., 1999 and as shown in FIG. 19A. The expression of the T7 RNA polymerase in transfected cells can be obtained by different means including transfection of plasmid DNA containing an expression cassette of the T7 RNA polymerase, recombinant virus (such as fowlpox or canarypox virus) expressing the T7 RNA polymerase or in cells that express the T7 RNA polymerase. In another aspect, the polynucleotides for expression of the APMV nucleoprotein (NP), the APMV phosphoprotein (P) and the APMV RNA polymerase (L) and the full length viral cRNA are under the control of the immediate early promoter of the human cytomegalovirus. The rescue of the virus is performed as described in Inoue K, et al., 2003.

Successful expression of the inserted cDNA of interest (foreign cDNA or heterologous cDNA) by the modified infectious virus requires two conditions. First, the insertion must be introduced into a region of the genome of the virus in order that the modified virus remains viable. The second condition for expression of inserted cDNA is the presence of a regulatory sequences allowing expression of the gene in the viral background (for instance: gene start, gene stop, promoter, enhancer, polyadenylation signals, intergenic and untranslated regions).

In general, it is advantageous to employ a strong promoter functional in eukaryotic cells. In one embodiment, the promoter used for the transcription of viral mRNA by the viral RNA polymerase is the “gene start sequence”. The “gene start sequence” is the binding site for the L protein to bind and to transcribe the downstream located viral RNA into viral mRNA.

In one embodiment, the invention provides for the administration of a therapeutically effective amount of an APMV vaccine for the delivery and expression of an antigen, epitope or immunogen in a target cell. Determination of the therapeutically effective amount is routine experimentation for one of ordinary skill in the art. In one embodiment, the APMV vaccine formulation comprises an expression vector comprising a polynucleotide that encodes an antigen, epitope or immunogen and a pharmaceutically or veterinarily acceptable carrier, vehicle or excipient. In another embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro), or facilitating transfection and/or improve preservation of the vector (or protein). Doses and dose volumes are herein discussed in the general description and can also be determined by the skilled artisan from this disclosure read in conjunction with the knowledge in the art, without any undue experimentation.

In another embodiment, pharmaceutically or veterinarily acceptable carrier, excipient, or vehicle may be a water-in-oil emulsion. Examples of suitable water-in-oil emulsions include oil-based water-in-oil vaccinal emulsions which are stable and fluid at 4° C. containing: from 6 to 50 v/v % of an antigen-containing aqueous phase, from 12 to 25 v/v %, from 50 to 94 v/v % of an oil phase containing in total or in part a non-metabolizable oil (e.g., mineral oil such as paraffin oil) and/or metabolizable oil (e.g., vegetable oil, or fatty acid, polyol or alcohol esters), from 0.2 to 20 p/v % of surfactants, from 3 to 8 p/v %, the latter being in total or in part, or in a mixture either polyglycerol esters, said polyglycerol esters being polyglycerol (poly)ricinoleates, or polyoxyethylene ricin oils or else hydrogenated polyoxyethylene ricin oils. Examples of surfactants that may be used in a water-in-oil emulsion include ethoxylated sorbitan esters (e.g., polyoxyethylene (20) sorbitan monooleate (TWEEN 80®), available from AppliChem, Inc., Cheshire, Conn.) and sorbitan esters (e.g., sorbitan monooleate (SPAN 80®), available from Sigma Aldrich, St. Louis, Mo.). In addition, with respect to a water-in-oil emulsion, see also U.S. Pat. No. 6,919,084. In some embodiments, the antigen-containing aqueous phase comprises a saline solution comprising one or more buffering agents. An example of a suitable buffering solution is phosphate buffered saline. In one embodiment, the water-in-oil emulsion may be a water/oil/water (W/O/W) triple emulsion (see, e.g., U.S. Pat. No. 6,358,500). Examples of other suitable emulsions are described in U.S. Pat. No. 7,371,395.

The pharmaceutical compositions and vaccines according to the invention may comprise or consist essentially of one or more adjuvants. Suitable adjuvants for use in the practice of the present invention are (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al., 1996; WO98/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, e.g., DDA (5) cytokines, (6) aluminum hydroxide or aluminum phosphate, (7) saponin or (8) other adjuvants discussed in any document cited and incorporated by reference into the instant application, or (9) any combinations or mixtures thereof.

The oil in water emulsion (3), which is especially appropriate for viral vectors, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil is used in combination with emulsifiers to form an emulsion. The emulsifiers may be nonionic surfactants, such as: esters of on the one hand sorbitan, mannide (e.g. anhydromannitol oleate), glycerol, polyglycerol or propylene glycol and on the other hand oleic, isostearic, ricinoleic or hydroxystearic acids, said esters being optionally ethoxylated, or polyoxypropylene-polyoxyethylene copolymer blocks, such as Pluronic, e.g., L121. Among the type (1) adjuvant polymers, preference is given to polymers of crosslinked acrylic or methacrylic acid, especially crosslinked by polyalkenyl ethers of sugars or polyalcohols. These compounds are known under the name carbomer (Pharmeuropa, vol. 8, no. 2, June 1996). One skilled in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name Carbopol (BF Goodrich, Ohio, USA) are especially suitable. They are crosslinked by allyl saccharose or by allyl pentaerythritol. Among them, reference is made to Carbopol 974P, 934P and 971P.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., 1960.

With regard to structure, the acrylic or methacrylic acid polymers and EMA are preferably formed by basic units having the following formula:

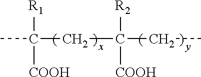

in which:
R1 and R2, which can be the same or different, represent H or CH3
x=0 or 1, preferably x=1
y=1 or 2, with x+y=2.

For EMA, x=0 and y=2 and for carbomers x=y=1.

These polymers are soluble in water or physiological salt solution (20 g/l NaCl) and the pH can be adjusted to 7.3 to 7.4, e.g., by soda (NaOH), to provide the adjuvant solution in which the expression vector(s) can be incorporated. The polymer concentration in the final immunological or vaccine composition can range between 0.01 and 1.5% w/v, between 0.05 and 1% w/v, and between 0.1 and 0.4% w/v.

Another aspect of the invention relates to a method for inducing an immunological response in an animal to an antigen, which method comprises inoculating the animal with a vaccine or a pharmaceutical composition including modified recombinant APMV virus which comprises and encodes the antigen of a pathogen for said animal. Yet another aspect of the invention relates to a method for inducing an immunological response in an animal to an antigen in a prime-boost administration regimen, which is comprised of at least one primary administration and at least one booster administration using at least one common polypeptide, antigen, epitope or immunogen. The immunological composition or vaccine used in primary administration may be same, may be different in nature from those used as a booster. In one aspect of the prime-boost protocol of the invention, a composition or vaccine comprising the recombinant APMV virus (viral vector) of the present invention is administered followed by the administration of an inactivated viral vaccine or composition comprising an antigen, or a vaccine or composition comprising a subunit (protein, antigen), or a DNA plasmid vaccine or composition that contains or expresses an antigen. Likewise, a prime-boost protocol may comprise the administration of an inactivated viral vaccine or composition comprising an antigen, or a vaccine or composition comprising a subunit (protein, antigen), or a DNA plasmid vaccine or composition that contains or expresses an antigen, followed by the administration of a composition or vaccine comprising the recombinant APMV virus (viral vector) of the present invention. It is further noted that both the primary and the secondary administrations may comprise the composition or vaccine comprising the recombinant APMV virus (viral vector) of the present invention.

The primary administration may comprise one or more administrations of the same viral vector-based immunological compositions of vaccines. Similarly, the booster administration may comprise one or more administrations of the same viral vector-based or immunological composition of vaccine. The administration route of the prime and the boost may be the same or different. Similarly, the origin of the protective gene present in the prime and the boost may be the same or different (e.g. different strain).

The various administrations are preferably carried out 1 to 6 weeks apart, and more particularly about 3 weeks apart. According to a preferred mode, an annual booster, preferably using the viral vector-based immunological composition of vaccine, is also envisaged. The animals are preferably at least one day old at the time of the first administration.

A variety of administration routes may be used such as subcutaneously or intramuscularly, intradermally, transdermally, spray, drinking water, eye drop, intranasal, oral, oral baits, in ovo or a combination (e.g. oculonasal, oronasal).

The invention will now be further described by way of the following non-limiting examples.

### EXAMPLES

### Example 1

### APMV-2, APMV-4, and APMV-6

### A. Viruses and Birds

One-day-old SPF (specific pathogen free) chickens (Merial, Gainesville, Ga.) were housed in positive pressure Horsfall-Baur isolation units. Feed and water were provided ad libitum and the birds were examined twice daily. The viruses (APMV-2, 4, and 6) used for experimental studies were isolated from wild birds and classified by the National Veterinary Service Laboratory (NVSL, Ames Iowa, USA). Viruses were propagated in 9-day-old embryonated SPF chicken eggs (SunRise Farms, Catskill, N.Y., USA) by inoculation via the allantoic route. The allantoic fluid was harvested at day 3 after inoculation, aliquoted, and stored at −80° C. The APMV subtype was confirmed by the HI test using standard sera (NVSL, Ames, Iowa, USA). The 50% egg infectious dose (EID₅₀) for each isolate was determined by inoculating 10-fold serial dilutions of allantoic fluid in embryonated SPF eggs. The titer was calculated following the method as described by Reed and Muench (Reed, L J et al., 1938, Am. J. Epidemiol. 27:493-497).

### B. Experimental Infection

Twenty-five one-day-old SPF chickens per group were infected with 10⁶ EID₅₀ per chicken by the ocular-nasal route. Chickens of the control group were mock inoculated with PBS (phosphate buffered saline). At days 2, 4, 7, 14, and 28 p. i. (post infection) five birds from each group were bled via the wing vein to collect serum samples, euthanized with CO₂, and necropsied. Tissue specimens of the trachea, lung, pancreas and gut were collected. For each organ, a fresh pair of sterile scissors and forceps was used. Half of the tissue sample was placed in a Lysing Matrix D tube (MP Biomedicals, Solon, Ohio) containing viral transport medium (1× minimal essential medium, 7.5% sodium bicarbonate, 15 mM HEPES, 1% fetal bovine serum, 4,000 U/ml penicillin, 400 μg/ml gentamycin, 8 μg/ml amphotericin B, 4,000 μg/ml streptomycin, 1000 μg/ml kanamycin sulfate). The second half of the tissue sample was fixed in 10% buffered formalin and embedded in paraffin wax. Sections of the paraffin-embedded tissues were stained with Mayer's hematoxylin and eosin (H&E).

The results showed that mild diarrhea was observed at day four and seven p. i. in birds infected with APMV-2 or APMV 4. During necropsy, birds infected with APMV-2 showed slightly enlarged pancreas at day two and four after infection. No other gross lesions were observed in any group.

### C. Serology

The hemagglutination (HA) and hemagglutination-inhibition (HI) tests were used to detect virus in allantoic fluid and analyze the presence of HI antibodies in collected serum samples, respectively. The tests were conducted by standard procedures using 0.8% chicken red blood cells resuspended in PBS. The HI test was performed by the diluted-serum constant-antigen method. Eight HA units of viral antigen was used for each serum dilution.

The HI antibody titers were investigated at day 2, 4, 7, 14, and 28 p.i. Positive HI titers (≧1:16) were observed in serum samples of APMV-2 infected birds at day 7 (1/5), day 14 (5/5), and day 28 p.i. (5/5). Interestingly, only one chicken infected with APMV-4 developed an HI titer which has been considered as positive during the course of the experiment at day 14 p.i. Similarly for APMV-6, two chickens out of five developed a HI titer of 1:16, only on day 28 p.i. Mock inoculated birds remained negative for HI antibodies to all three APMV used in this experiment. In addition, to exclude cross contamination all sera were tested for HI antibodies against the other two antigens and remained negative.

### D. Virus Isolation

The tissue samples collected in the Lysing Matrix D tubes (MP Biomedicals, Solon, Ohio, USA) were homogenized twice using the Fastprep®-24 (MP Biomedicals, Solon, Ohio) at a setting of 4.0 M/S for 20 seconds. Following incubation for 15 min at room temperature, the homogenized samples were centrifuged for 20 min at 2000 g at 4° C. Sterility was tested after inoculation of 50 μl of the obtained supernatant in 2 ml tryptose phosphate broth (TPB) (DIFCO, Becton Dickenson, Sparks, Md., USA) supplemented with 10% hydrolactalbumin by incubation at 37° C. in an orbital shaker overnight. Non-sterile samples were filtered with 0.45 μm syringe filters (Whatman Inc., Florham Park, N.J., USA). Samples were stored at −80° C. Virus isolation was performed by inoculation of 0.1 ml into the allantoic cavity of 9-day-old embryonated SPF chicken eggs. After incubation for three days at 37.5° C., the allantoic fluid was harvested and tested for the presence of haemagglutinating activity by HA.

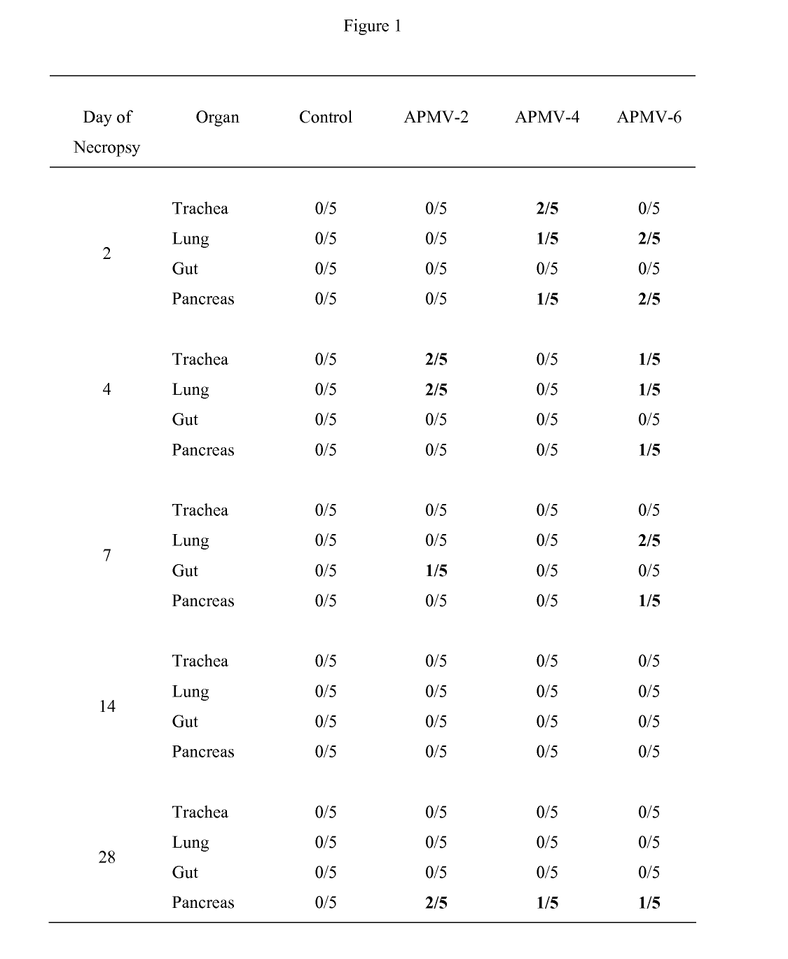

To analyze sites of virus replication, several organs (trachea, lung, gut, pancreas) were analyzed for infectious virus by virus isolation in embryonated eggs (FIG. 1). Overall, replicating virus was only detected in a few chickens. Briefly, APMV-4 was recovered on day 2 from trachea, lung and pancreas while APMV-6 was isolated from lung and pancreas. At day 4, APMV-2 was isolated from trachea and lung while APMV-6 was isolated from all tested organs except the gut. At day 7, APMV-2 was isolated from a single gut sample, APMV-4 was isolated from the pancreas while APMV-6 was isolated from lung and pancreas samples. Surprisingly, at day 14 p.i. no virus was isolated whereas at day 28 p.i. APMV-2, 4 and 6 could be isolated from the pancreas. No virus was isolated from mock inoculated birds. The identity of the back isolated virus was confirmed by HI test using standard sera as provided by NVSL.

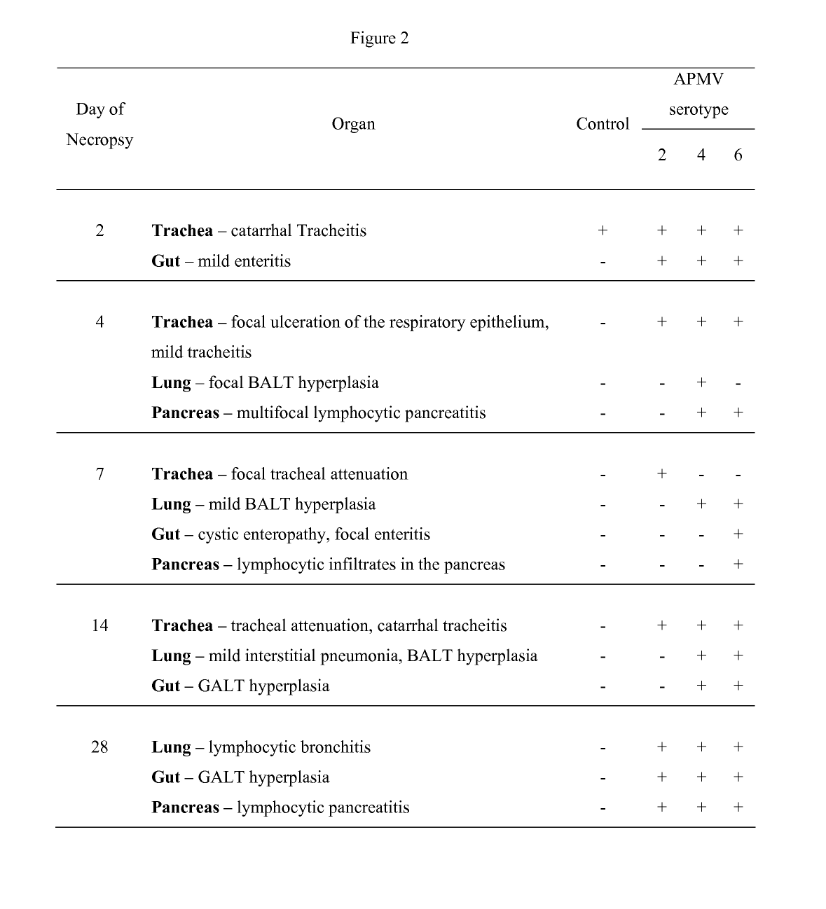

To evaluate the pathological potential of the investigated viruses, microscopical lesions in the obtained organs were analyzed (FIG. 2). At day 2 p.i., a catarrhal tracheitis in addition to ciliary loss on the respiratory epithelium and mild enteritis was observed in all infected chickens. At day 4 p.i., APMV-2 infected chickens showed an increased number of hypertrophic mucous glands in the trachea and focal ulcerations of the respiratory epithelium. APMV-4 infected birds showed changes highly suggestive of a respiratory infection such as mild tracheitis, mild to moderate multifocal lymphocytic pancreatitis and also a focal BALT (Bronchus-Associated Lymphoid Tissue) hyperplasia at day 4 p.i. The investigation on organs of APMV-6 infected chickens revealed tracheal changes such as catarrhal and ulcerative tracheitis and a focal pancreatitis that are consistent with a viral stimulation. At day 7 p.i., APMV-2 infected chickens showed focal tracheal attenuation or replacement of respiratory epithelium as indicative of healing. Birds infected with APMV-4 showed mild BALT hyperplasia while APMV-6 infected birds showed cystic enteropathy, focal enteritis and lymphocytic infiltrates in the pancreas. In addition to mild lymphocytic enteritis and mild GALT hyperplasia, APMV-2 infected birds also showed healing changes such as tracheal attenuation at day 14 p.i. Organ samples from chicken infected with APMV-4 or 6 showed changes suggestive of viral infection such as mild interstitial pneumonia, catarrhal tracheitis and BALT or GALT hyperplasia at day 14 p.i. All investigated samples obtained from infected chicken showed lesions such as GALT hyperplasia, lymphocytic pancreatitis and lymphocytic bronchitis at day 28 p.i. At day 2, birds of the control group showed a mild catarrhal tracheitis which could be attributed to environmental factors.

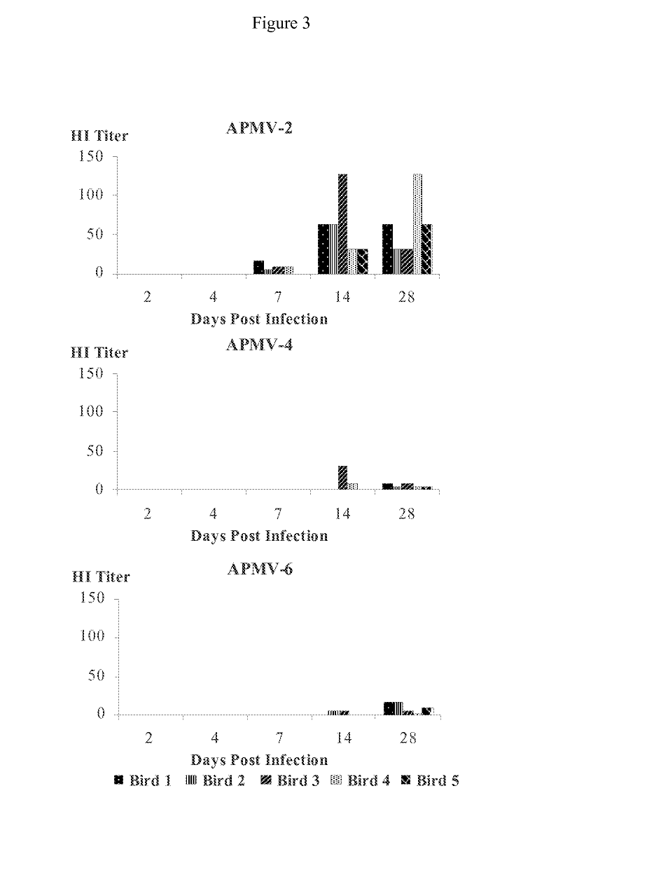

FIG. 3 shows low HI titers (up to 1:32 at day 14) from SPF chickens infected with APMV-4 or APMV-6 indicating that only APMV-2 infection elicited an HI response that could be characterized as seropositive. Nevertheless, all three viruses were recovered from the infected birds' trachea, lungs, gut and pancreas up to day 7 p.i. and from the pancreas up to day 28 p.i. Infection with APMV-2, 4, or 6 showed characteristic histopathological lesions (summarized in FIG. 2) in all the infected birds, indicative of stimulation with a viral antigen. The viral isolation and histological profiles of the infected birds clearly depicted the tropism of the viruses in the infected birds (trachea and lungs from day 2 to day 7 and gut, lung and pancreas from day 7 onwards). All isolates were detected in the pancreas up to 28 days p.i. but virus isolation was not possible at day 14 p.i. This indicated that the investigated APMV can probably persist and later become reactivated. Thus virus carriers can be present in infected flocks. Only APMV-2 induced HI antibodies while HI antibodies of chickens infected with APMV-4 and 6 were not detected.

### Example 2

### APMV-8

### A. Viruses and Birds

One-day-old SPF chickens (Merial, Gainesville, Ga., USA) and Pekin ducks (Metzer Farms, Gonzales, Calif., USA) were housed in positive pressure Horsfall Baur isolation units. Feed and water were provided ad libitum, and the birds were examined twice daily. The APMV-8 virus (APMV-8: SCWDS ID: MA-7) used for experimental studies was isolated from a mallard and classified by the National Veterinary Service Laboratory (NVSL, Ames Iowa, USA). The virus was propagated in 9-day-old embryonated SPF chicken eggs by inoculation via the allantoic route. Allantoic fluids were harvested at day 3 after inoculation, pooled, aliquoted and stored at −80° C. The APMV-8 subtype was confirmed by HI testing using standard sera as provided by the National Veterinary Service Laboratory (Ames, Iowa, USA). The EID₅₀ was determined by inoculating 10-fold serial dilutions of allantoic fluid in embryonated SPF eggs. The titer was calculated following the method as described by Reed and Muench (Reed & Muench, 1938).

### B. Experimental Infection

Twenty five one-day-old SPF chickens or Pekin ducks per group were infected via the oculonasal route with 10⁶ EID₅₀ per bird diluted in PBS. Birds of the control chicken or duck group were mock inoculated with PBS. Five birds from each group were bled via the brachial vein to collect serum samples, humanely euthanized with CO₂, and necropsy was performed at two, four, seven, fourteen and twenty eight days post infection (d p.i). Tissue specimens of the trachea, lung, pancreas, and gut (duodenum) were collected. For each organ, a fresh pair of sterile scissors and forceps was used. Half of the tissue sample was placed in a Lysing Matrix D tube (MP Biomedicals, Solon, Ohio, USA) containing viral transport medium (VTM, 1× minimal essential medium, 7.5% sodium bicarbonate, 15 mM HEPES, 1% fetal bovine serum, 4,000 U/ml penicillin, 400 μg/ml gentamycin, 8 μg/ml amphotericin B, 4,000 μg/ml streptomycin, 1000 μg/ml kanamycin sulfate). The second half of the tissue sample was fixed in 10% buffered formalin and routinely processed, embedded, sectioned and stained with hematoxylin and eosin (H&E).

### C. Virus Isolation

The tissue samples collected in the Lysing Matrix D tubes were homogenized twice using the FastPrep-24 (MP Biomedicals) at a setting of 4.0 M/S for 20 seconds. The homogenized samples were incubated for 15 min at room temperature and then centrifuged for 20 min at 2000×g at 4° C. 50 μl of the obtained supernatant was inoculated in 2 ml sterile TFB supplemented with 10% hydrolactalbumin followed by incubation at 37° C. in an orbital shaker overnight to test for sterility. Non-sterile samples were filtered with 0.45 μm syringe filters (Whatman Inc.). Samples were stored at −80° C. Virus isolation was performed by inoculation of 0.1 ml into the allantoic cavity of 9-day-old embryonated SPF chicken eggs. After incubation for three days at 37.5° C., the allantoic fluid was harvested and tested for the presence of hemagluttinating activity by HA.

### D. Serology

The hemagglutination (HA) and hemagglutination-inhibition (HI) tests were used to detect virus in allantoic fluid and to analyze the presence of HI antibodies in collected serum samples, respectively. The tests were conducted by standard procedures using 0.8% chicken red blood cells resuspended in PBS. The HI test was performed by the diluted-serum constant-antigen method. Eight HA units of viral antigen were used for each serum dilution. The geometric mean titer was determined as described previously (Brugh, 1978).

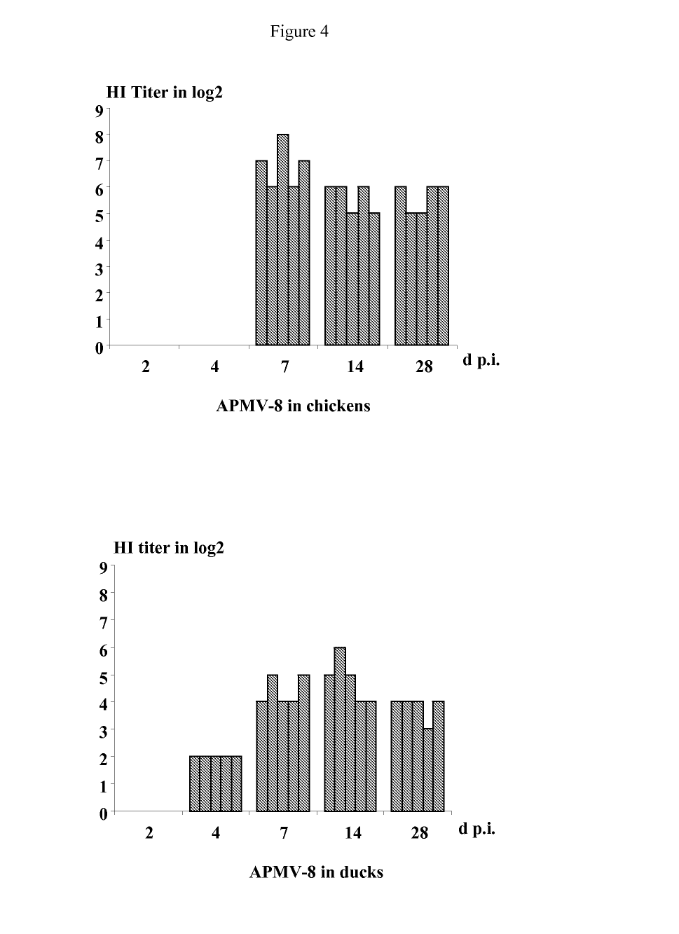

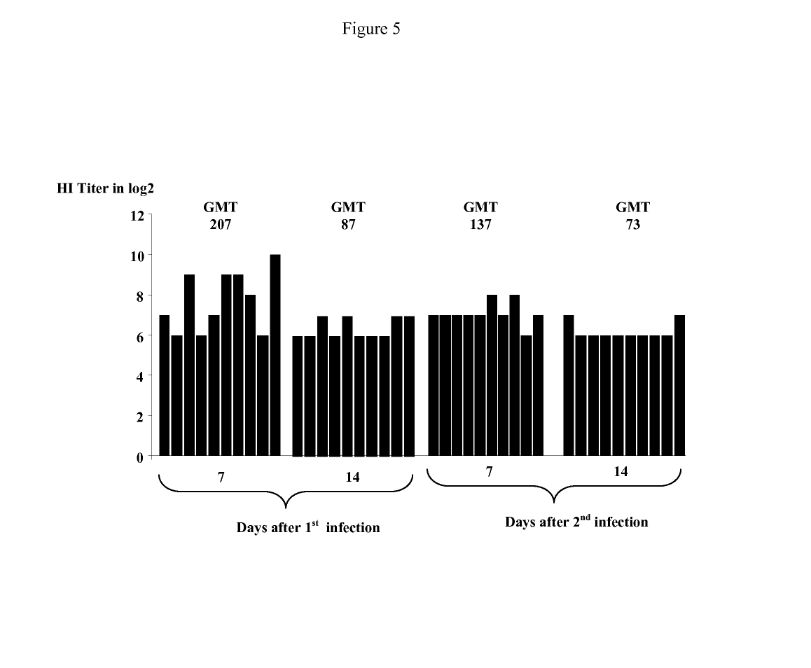

FIG. 4 shows HI antibody titers in SPF chickens and ducks infected with APMV-8. Chickens and ducks were oro-nasal infected with a dose of 10⁶ EID₅₀ of APMV-8. Sera samples were collected at day 2, 4, 7, 14, and 28 p.i. and analyzed by the HI test with the APMV-8 antigen. The HI serum titers (in log₂) are shown on the left axis.

### E. Pathogenicity Indices of APMV-8 in Chicken

To assess the virulence of APMV-8, the intracerebral pathogenicity index (ICPI) was determined following the World Organization for Animal Health (OIE, 2008) procedures for Newcastle disease virus. The mean dead time (MDT) in chicken embryos was determined as described previously (Swayne et al., 1998) using a serial dilution of APMV-8 from 10⁻¹ through 10⁻⁸.

The determination of the mean dead time (MDT) in embryonated eggs as well as the assessment of the intracerebral pathogenicity index (ICPI) is an important measurement for the pathogencity of the virus. With respect to the MDT in embryonated eggs, none of the embryos of the inoculated eggs died after the 7 day period and thus the APMV-8 isolate can be classified as lentogenic. The presence of virus was confirmed by HA test using the allantoic fluid of eggs inoculated with the 10⁻⁶ dilution. All the chickens intracerebrally inoculated showed no clinical signs over the observation time resulting in an ICPI value of zero which results in a lentogenic phenotype.

### F. APMV-8 Booster Vaccination

Ten one-day-old SPF chickens per group were infected via the oculo-nasal route with 10⁶ EID₅₀ per bird. Birds of the control chicken group were mock inoculated with PBS. The birds were infected again with the same dose 14 days after the first infection. The presence of infectious virus was monitored at day 2, 4, 7 and 14 after the first infection and day 2 and 4 after the second infection by virus isolation from tracheal swabs using 9-day-old embryonated SPF eggs. Antibody response was monitored by HI titer of serum samples collected at 0, 7, and 14 days after each vaccination.

In order to investigate if a second immunization would allow developing a more sustainable HI titer, a prime/boost scheme experiment was performed (FIG. 5). On day 7 after the first infection, all ten birds showed an HI ranging from 64 to 1024 (GMT 207). The titer declined at 14 d p.i. after the first infection (GMT 84) but increased after the boost infection at day 14 after the initial infection. On day 7 after the boost vaccination, the GMT increased to 137 and decreased again to a GMT of 73 at day 14 after the second infection. Infectious virus could be isolated from tracheal swabs at day 2 (5/10 birds) and day 4 (4/10) birds after the first infection. After the second infection no virus was isolated from the swabs taken at day 2 and 4 p.i.

### G. Detection of Viral RNA by RT-PCR

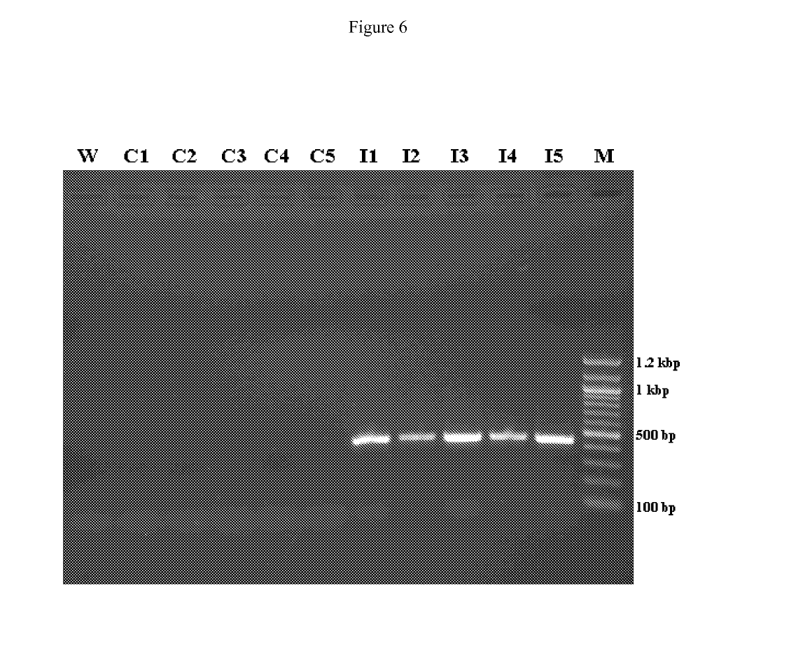

The detection of viral RNA from tissue samples was performed after homogenization of tissue samples followed by isolation of RNA by using the High Pure RNA isolation kit (Roche, Mannheim, Germany). A primer pair (8NPf1, 8NPr, see table 1) was used in an RT-PCR using the Supercript III One Step RT-PCR kit with Platinum Taq (Invitrogen, Carlsbad, Calif., USA) following the instructions of the manufacturer. The obtained reaction products were analyzed on a 1% agarose gel (FIG. 6). Tracheal tissues were taken at day 2 p.i. from non-infected ducks (C1-C5) and APMV-8 infected ducks (I1-I5). The tissues were homogenized and RNA was prepared for RT-PCR. A water control (W) was prepared in parallel. The reaction products were separated on a 1.5% agarose gel. The size of the fragment was controlled by using the 100 bp ladder (New England Biolabs, Boston, Mass., USA). The sizes of the DNA fragments are shown at the right.

TABLE 1
Oligonucleotides used for RT-PCR for the
detection of viral RNA in tissue
samples

| | | | | SEQ |
|---|---|---|---|---|
| Name | Sequence | orientation | Location^{A} | ID NO |
| APMV- | TTTTTTTTTTTTT | sense | 1-14 | 21 |
| | TTTTTACCAAACAR | | | |
| PolyT | RGAA | | | |
| 8NPfl | CAGGAGACCTGAT | sense | 200-223 | 22 |
| | GTTGCCTCAAC | | | |
| 8NPr | GCAGGCGATCTAT | antisense | 618-642 | 23 |
| | AGTCTCTGATAG | | | |

### H. Determination of the Minimal Infectious Dose in Chicken and Ducks

In order to determine which viral titer would be sufficient in chickens to detect a seroconversion, one-day-old SPF chickens were infected with different doses of APMV-8. The birds were housed as described above. Ten chickens per group were infected each with 10¹, 10², 10³, 10⁴, 10⁵, or 10⁶ EID₅₀. The virus was diluted in VTM. One group was inoculated with VTM and served as control. The birds were bled at day 7 and 14 after infection via the brachial vein. Based on the results obtained during the chicken experiment three-day-old Pekin ducks were infected with different amount of virus. The infection dose was chosen 10³, 10⁴, 10⁵, or 10⁶ EID₅₀ per duck. One group was mock-infected with VTM. The birds were bled at day 7 and 14 after infection via the leg vein. The serum sample was analyzed for the presence of virus specific antibodies by HI as described above.

### I. Determination of the Pathogenicity in Ducks and Chickens

During the experiments, no clinical signs were observed in chickens and ducks. During necropsy, three infected chickens showed slightly enlarged pancreas and inflamed duodenum at day two and four p.i. No other gross lesions were observed in any group.

The serological response was examined at 2, 4, 7, 14, and 28 d p.i. by investigation of the HI titers in the serum (FIG. 4). Serum samples of APMV-8 infected chickens showed positive HI titers (≧16) starting with 7 d p.i. (5/5, GMT: 111), 14 d p.i. (5/5, GMT: 48), and 28 d p.i. (5/5, GMT: 48). Serum samples of APMV-8 infected ducks also showed positive HI titers (≧16) at 7 (5/5, GMT 21), 14 (5/5, GMT 28), and 28 (4/5, GMT 14) d.p.i. The HI titers ranged from 32 to 256 for chickens, while for ducks the range was from 16 to 64. Sham inoculated birds remained negative for HI antibodies to APMV-8 at all time points investigated in both species.

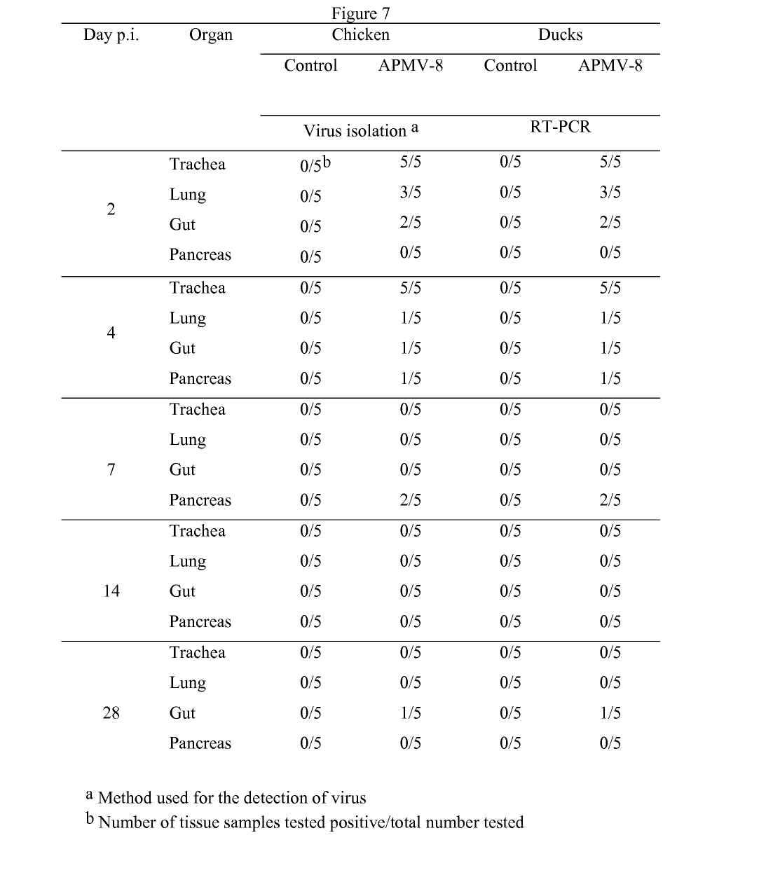

To determine the sites of virus replication in chickens and ducks, several organs (trachea, lung, duodenum, and pancreas) were analyzed for infectious virus by virus isolation in embryonated eggs (FIG. 7). In chickens, APMV-8 was recovered at 2 d p.i. from trachea, lung, and duodenum. At 4 d p.i., APMV-8 was isolated from all the analyzed organs; while at 7 d p.i., APMV-8 was isolated only from the pancreas. At 14 and 28 d p.i. no virus was isolated from any organ. No virus was isolated from sham inoculated birds. The identity of the back isolated virus was confirmed by HI test using standard sera as provided by NVSL. No virus was isolated from any of the collected duck tissues at any time point even after two subpassages in 9-day-old embryonated SPF chicken eggs. Therefore, RT-PCR primers were designed based on the APMV-8 sequence information available to detect the presence of viral RNA in the collected tissue samples (FIG. 7). At 2 d p.i viral RNA was detected in the trachea (FIG. 6), gut and pancreas while at 4 d p.i viral RNA was detected in all the organs analyzed. At 7, 14, and 28 d p.i, viral RNA was detected only in the trachea and lung. RT-PCR using RNA obtained from organs of mock inoculated birds did not result in the amplification of an RT-PCR fragment which indicates the absence of APMV-8 in these birds.

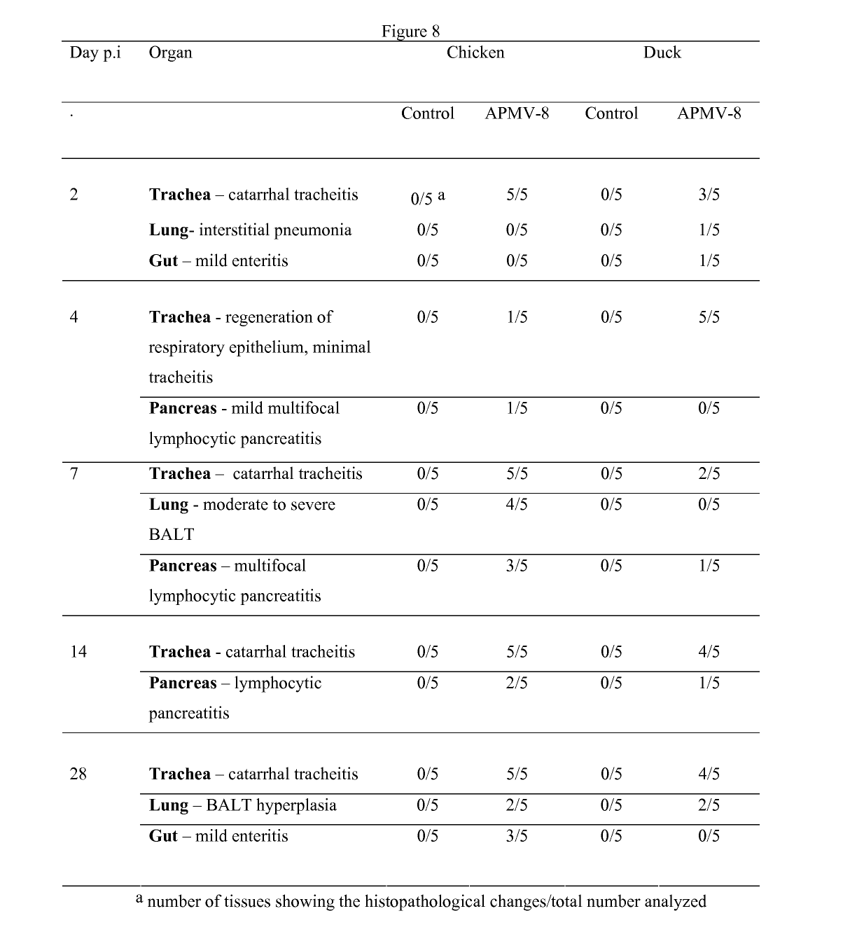

To evaluate the pathological potential of the investigated virus, the organs were analyzed for the presence of microscopic lesions (FIG. 8). At 2 d p.i., a mild multifocal proliferative tracheitis was observed in all infected chickens. The remaining organs showed no difference to the control group. APMV-8 infected chickens showed focal attenuation or regeneration of respiratory epithelium in the trachea at 4 d p.i. as indicative of healing. Additionally, the birds also showed mild multifocal lymphocytic pancreatitis which indicated a viral infection. Infected chicken showed changes in the lung at 7 d p.i. such as moderate to severe BALT, tracheal changes such as catarrhal tracheitis and multifocal lymphocytic pancreatitis. These findings are consistent with an antigenic stimulation. At 14 d p.i, tracheal changes consistent with healing and pancreatic changes such as lymphocytic pancreatitis suggestive of viral infection were observed in infected chickens. At 28 d p.i., only a mild catarrhal tracheitis and mild enteritis were observed in some of the infected chickens. In infected ducks, multifocal mild lymphocytic tracheitis, lung changes (interstitial pneumonia) and intestinal changes (lymphocytic enteritis) were observed at 2 d p.i while tracheal changes consistent with respiratory infection were seen at 4 d.p.i. At 7 d p.i., infected ducks showed lymphocytic tracheitis and pancreatitis consistent with viral infection whereas the observed catarrhal tracheitis was a suggestive of healing at 14 d p.i. In addition, infected ducks showed a lymphocytic pancreatitis at 14 d p.i. Later, at 28 d p.i., in the lung of infected ducks a BALT hyperplasia and also mild multifocal heterophilic tracheitis were noticed. Both pathological microscopic lesions were indicative of a viral infection. In the non infected controls no changes in the examined organs were observed.

### J. Determination of the Minimal Dose Needed for Induction of an Immune Response

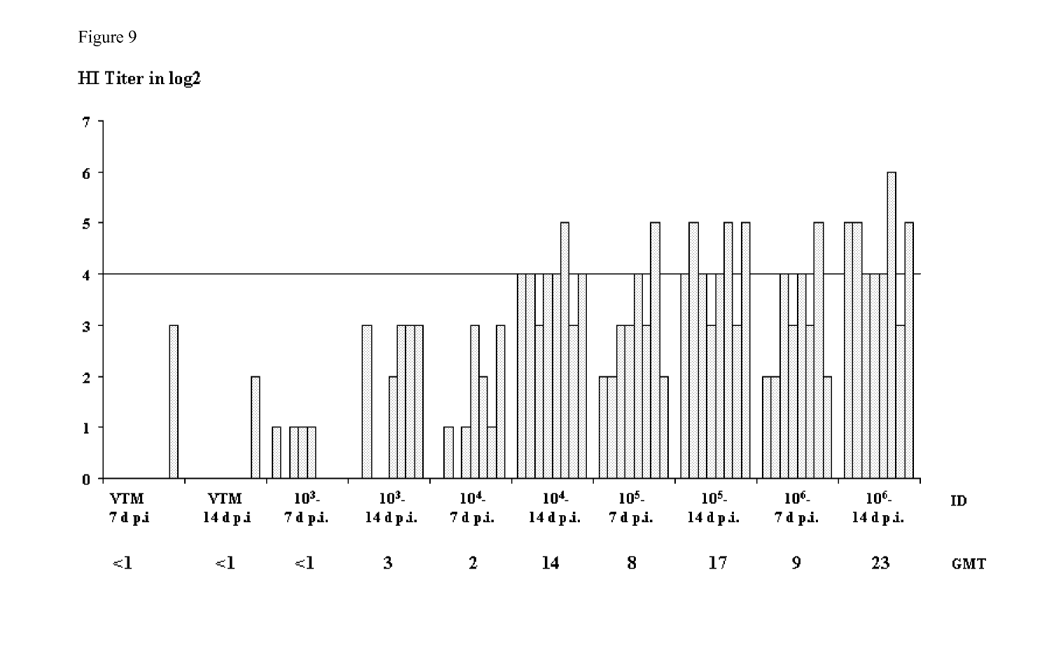

In order to examine the minimal infectious dose which is necessary to induce a seroconversion in chicken, tenfold dilutions of APMV-8 were used to infect ten one-day-old SPF chickens (FIG. 9). For the HI test, 4 HA units were used which results in a threshold of 16 to be considered as positive. The serum samples taken at day 14 p.i. showed that an EID₅₀ of 10³ was sufficient to induce an immune response in 4/10 chickens which was considered as positive (GMT 11). At day 14 after infection with an EID₅₀ of 10⁴ nine of ten birds showed a titer ≧16 (GMT 34). The infection with an EID₅₀ of 10⁵ and 10⁶ induced an HI titer of ≧16 at day 14 p.i. in all birds with a GMT of 73 and 137, respectively.

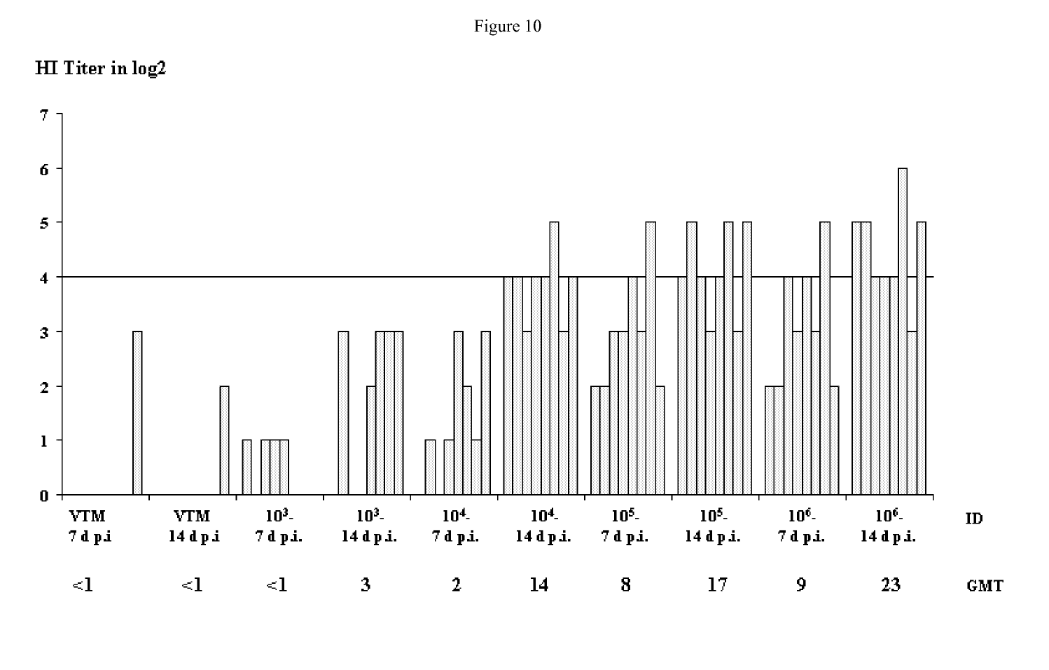

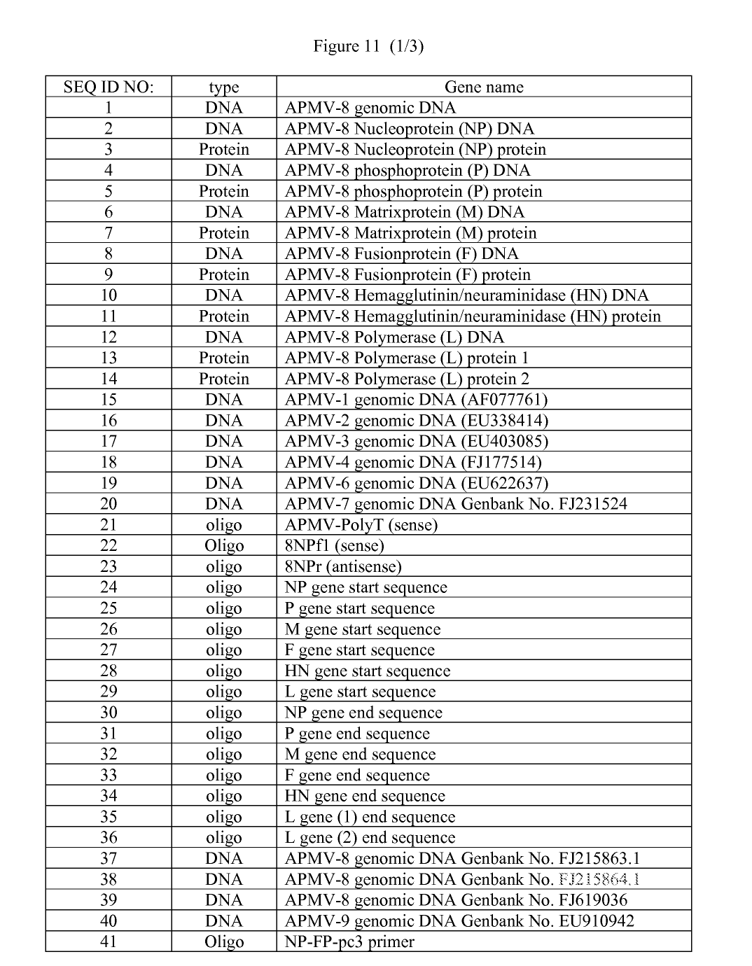

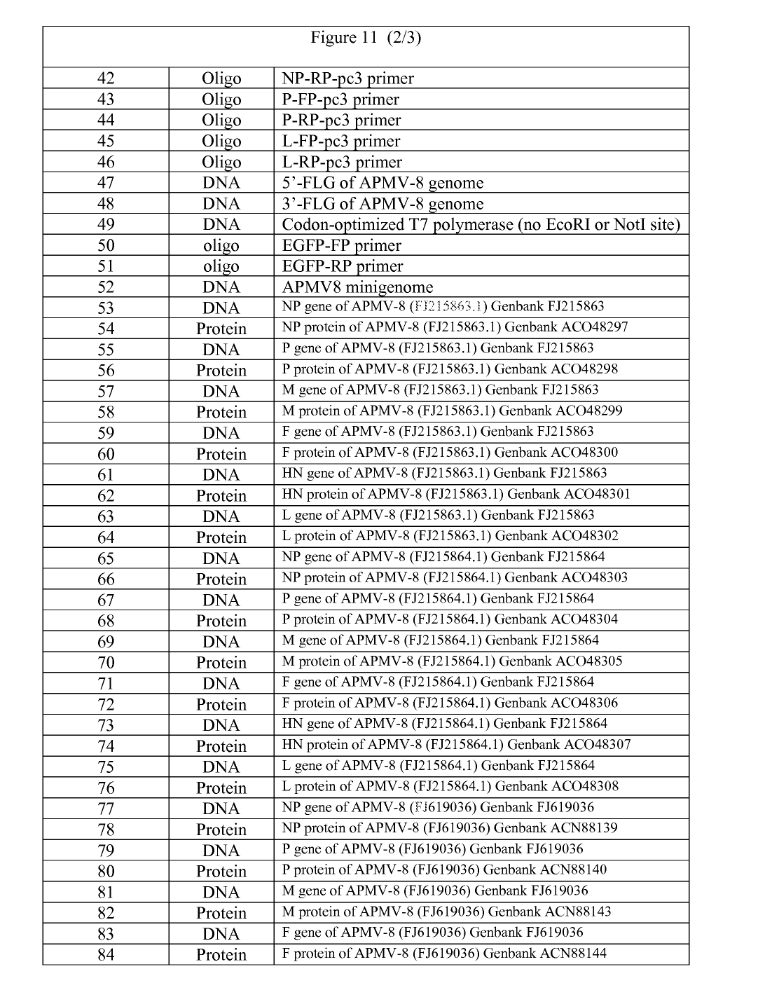

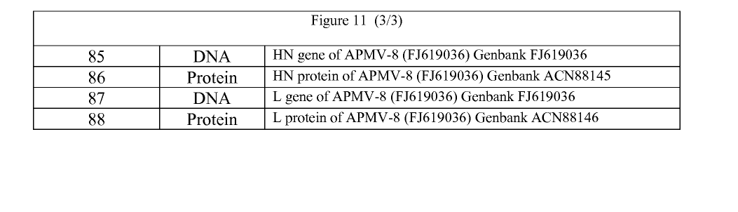

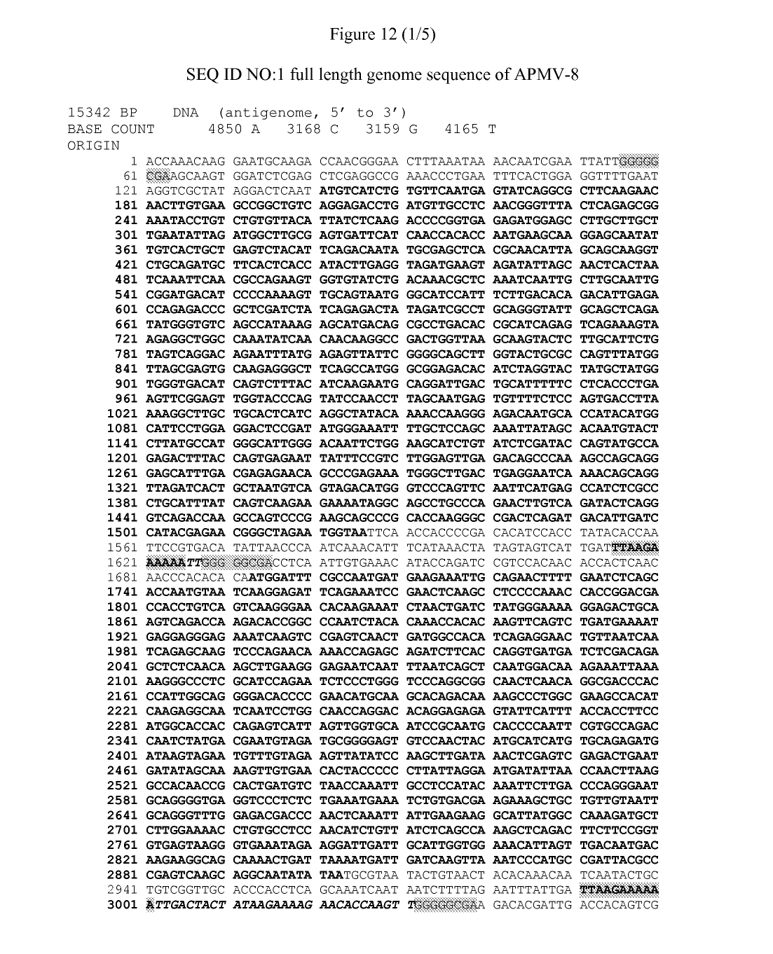

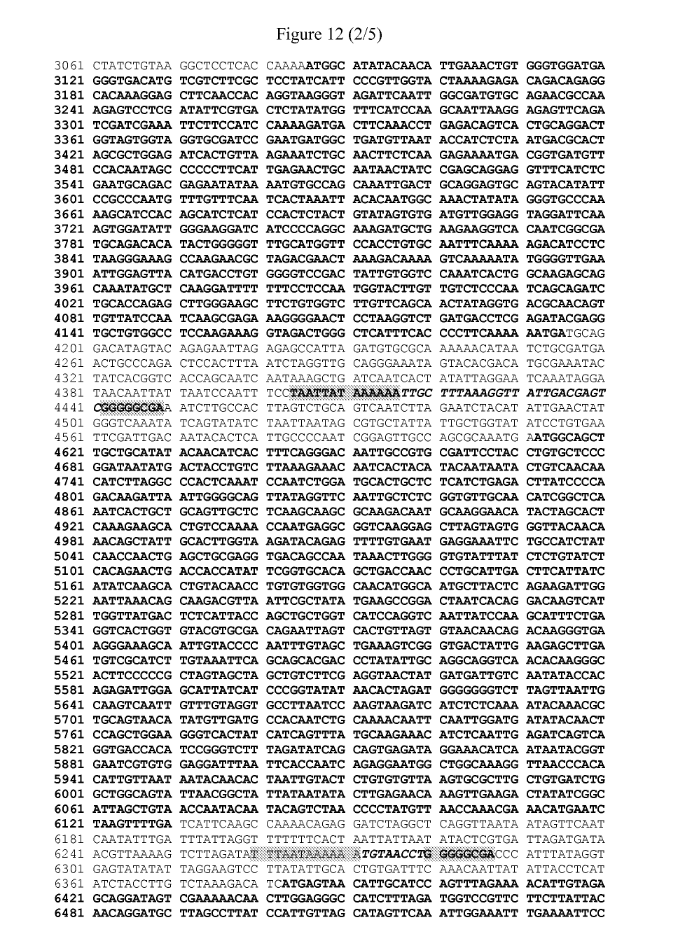

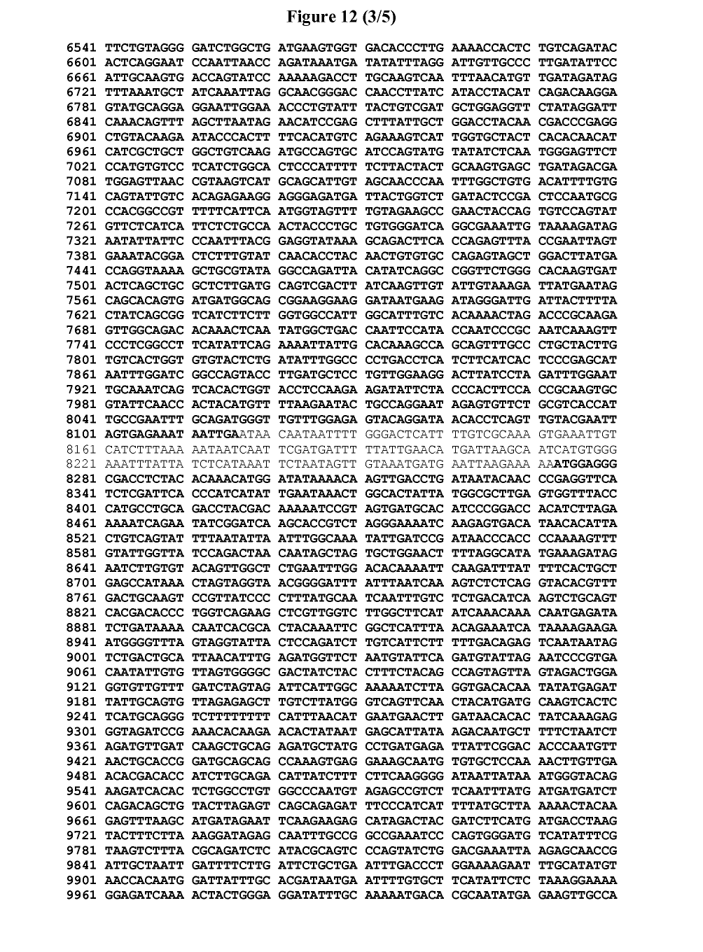

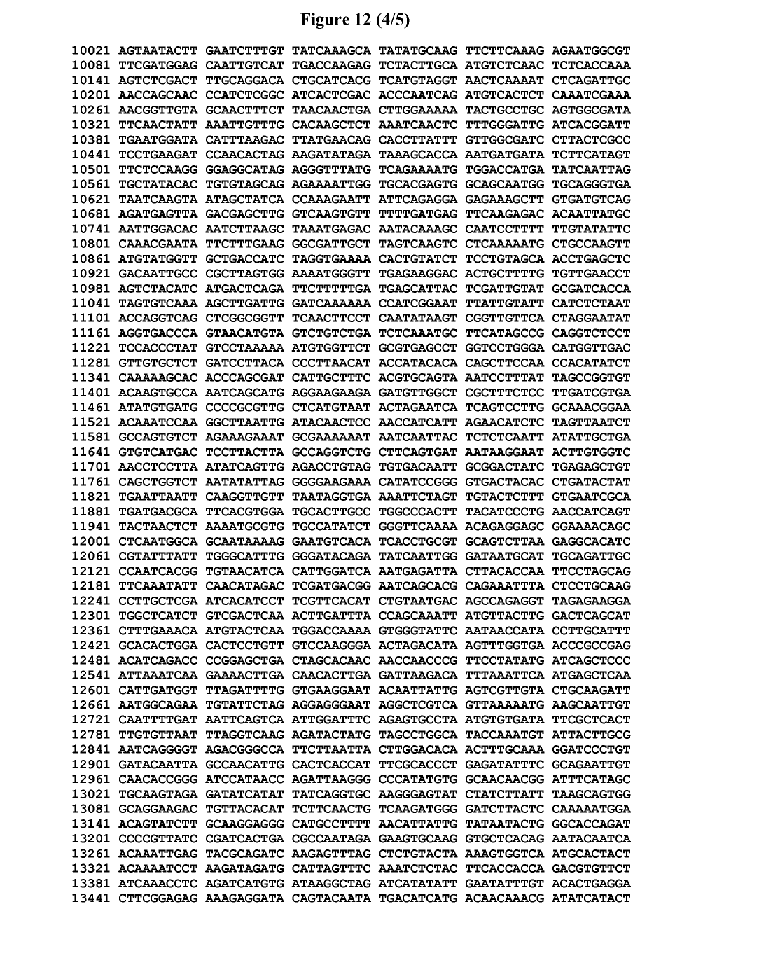

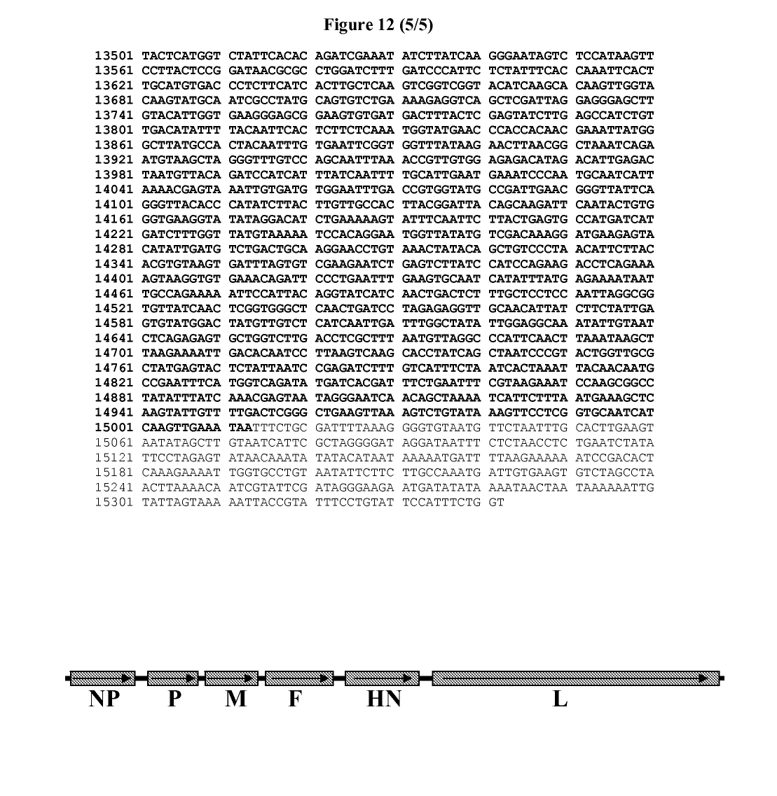

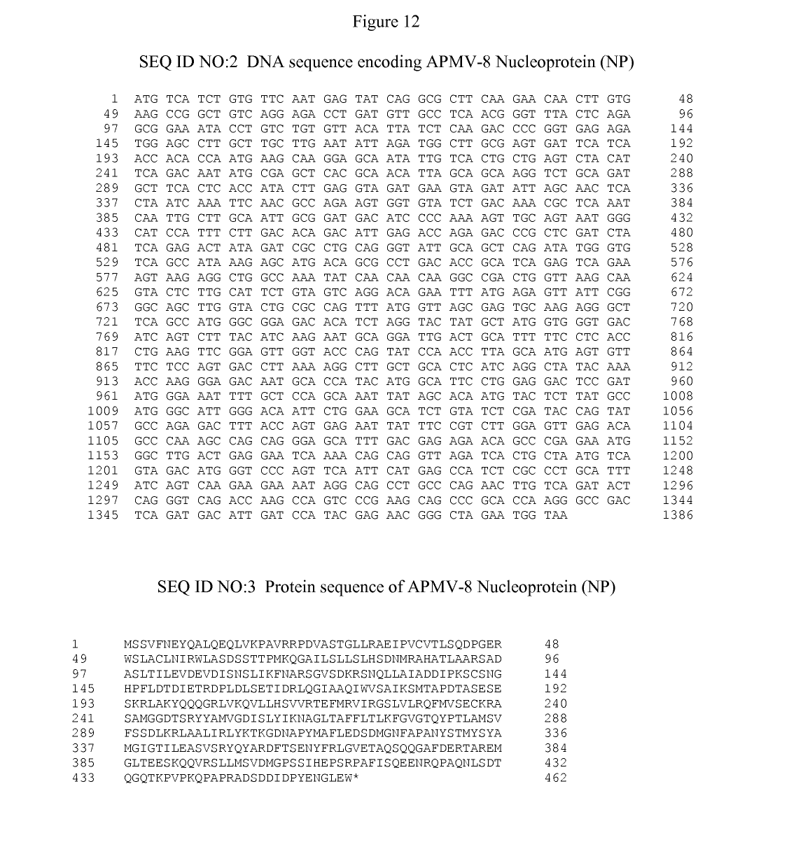

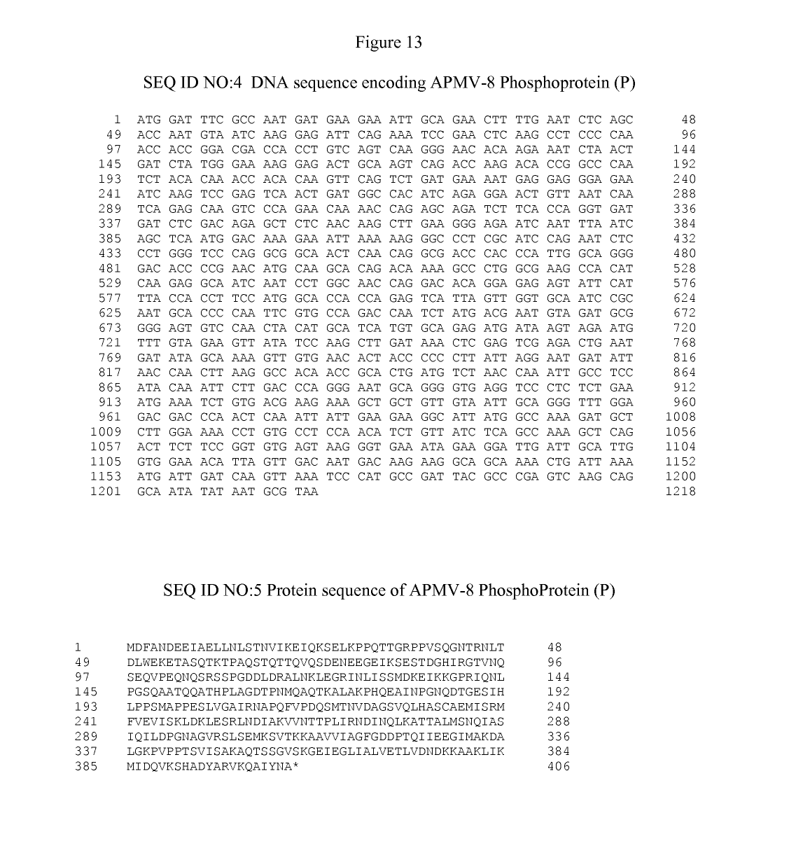

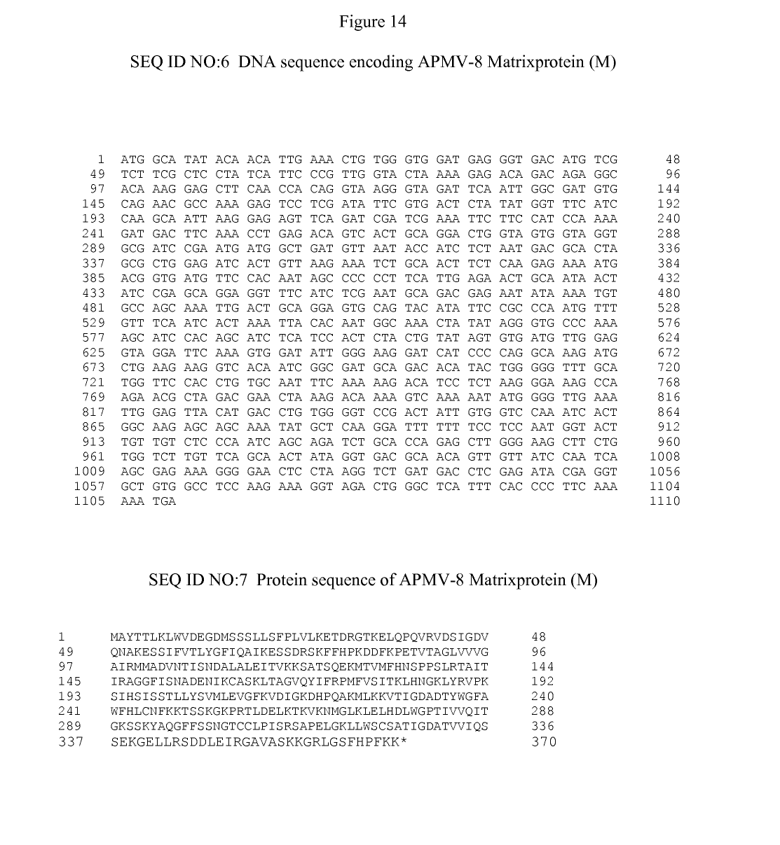

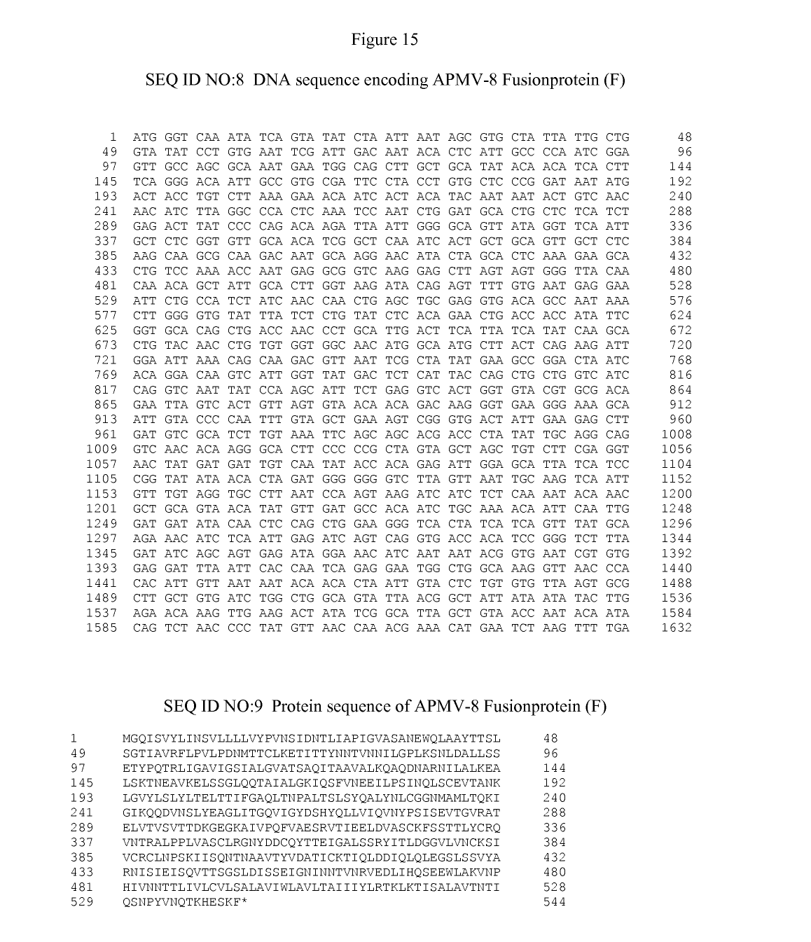

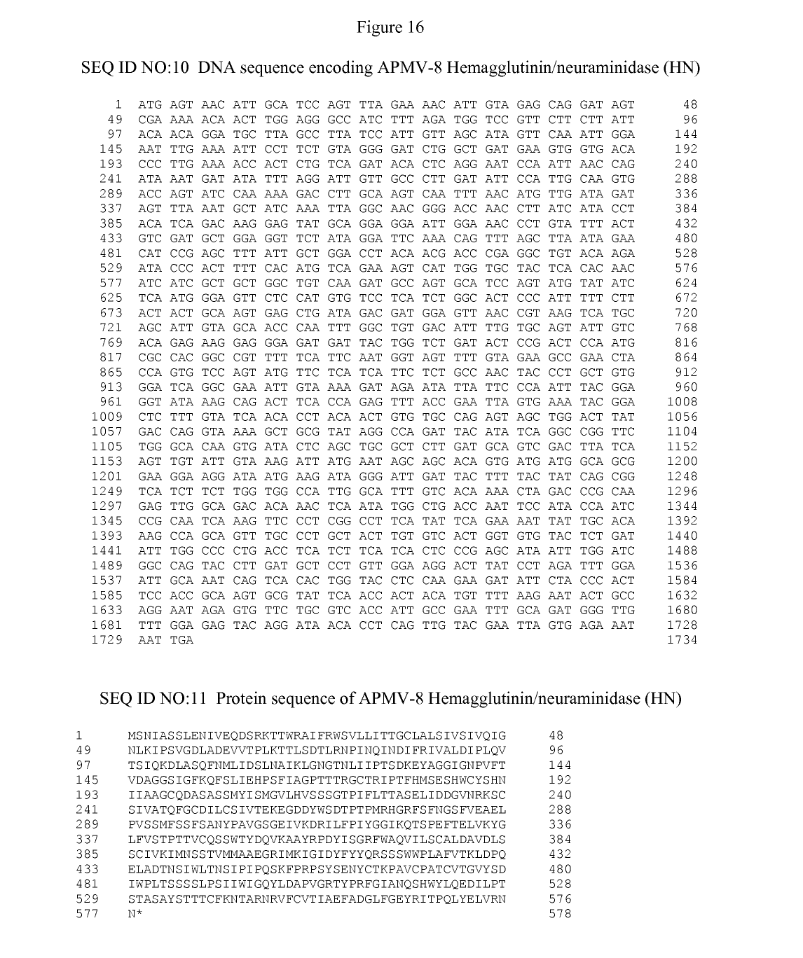

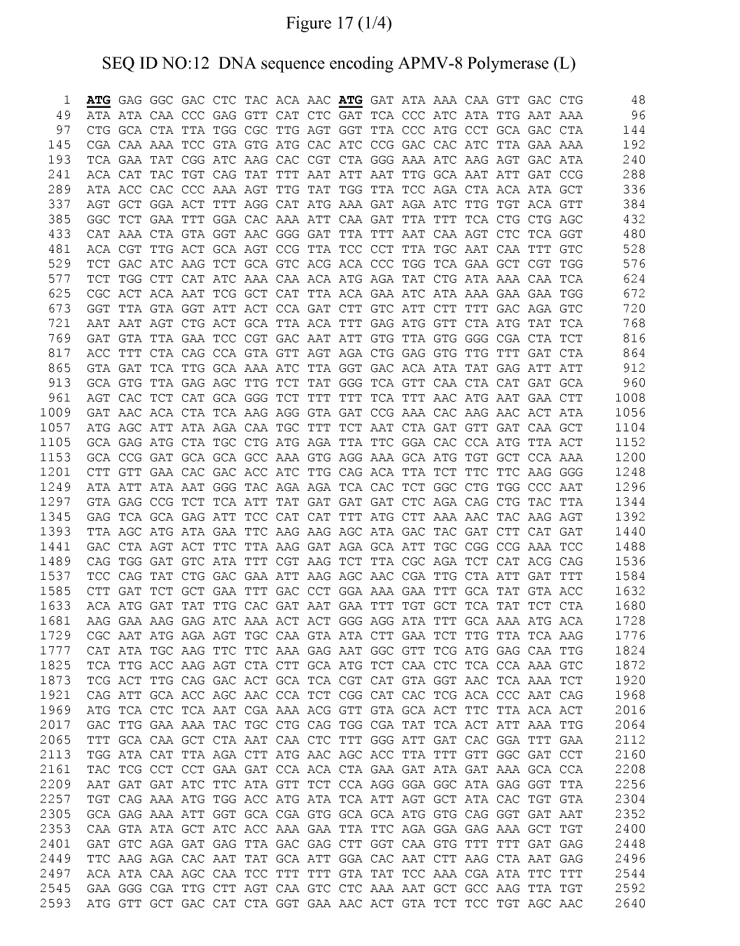

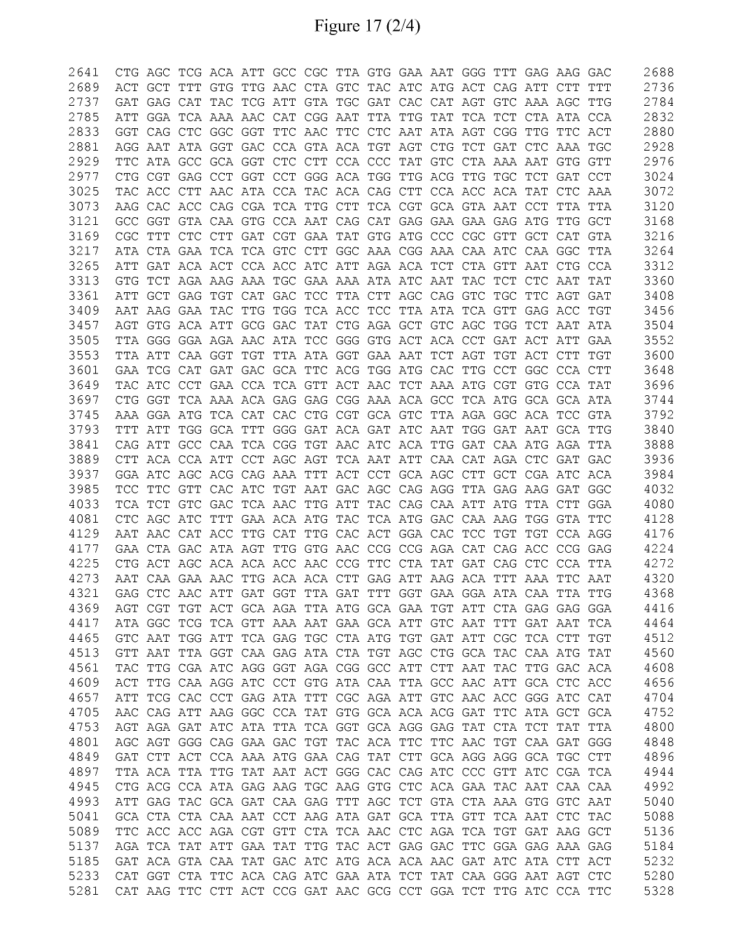

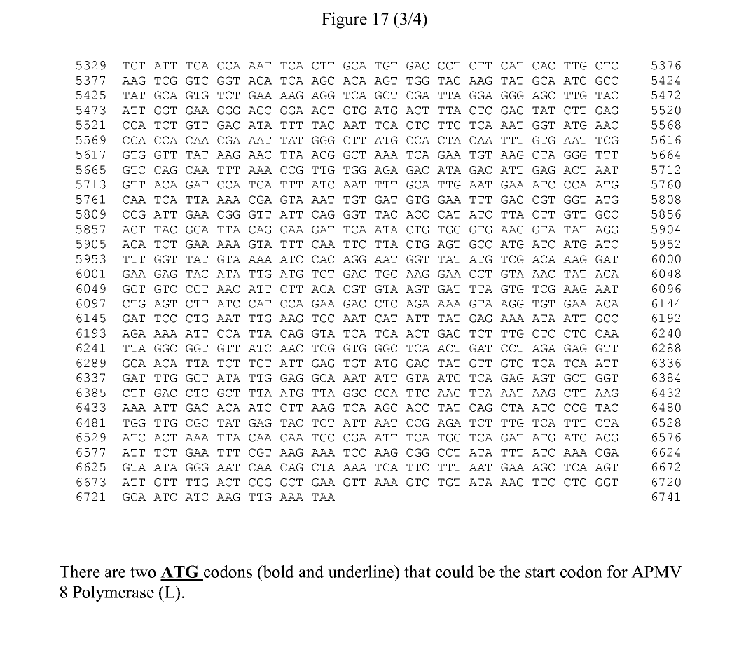

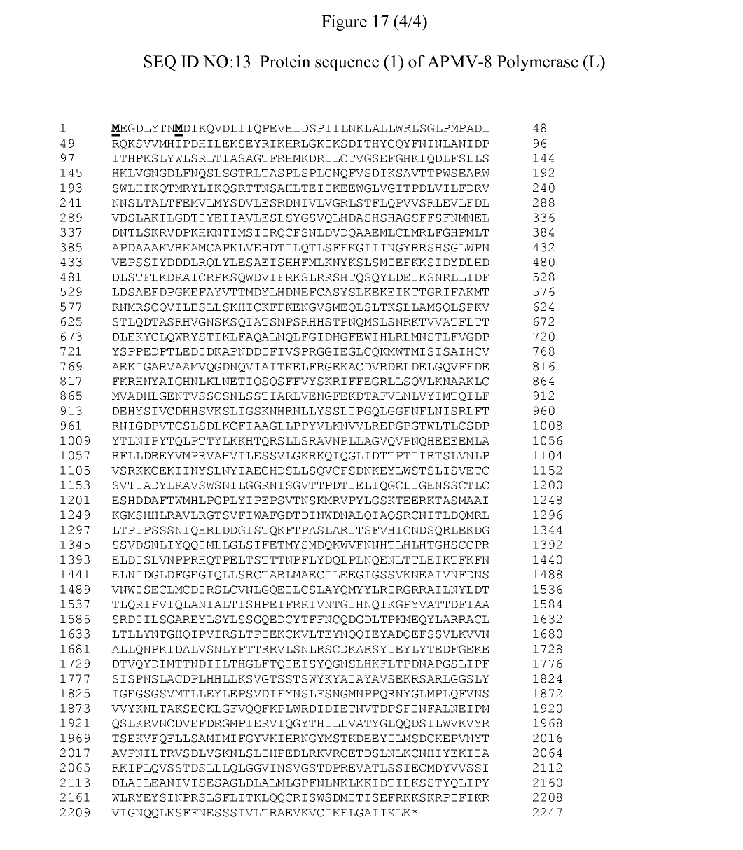

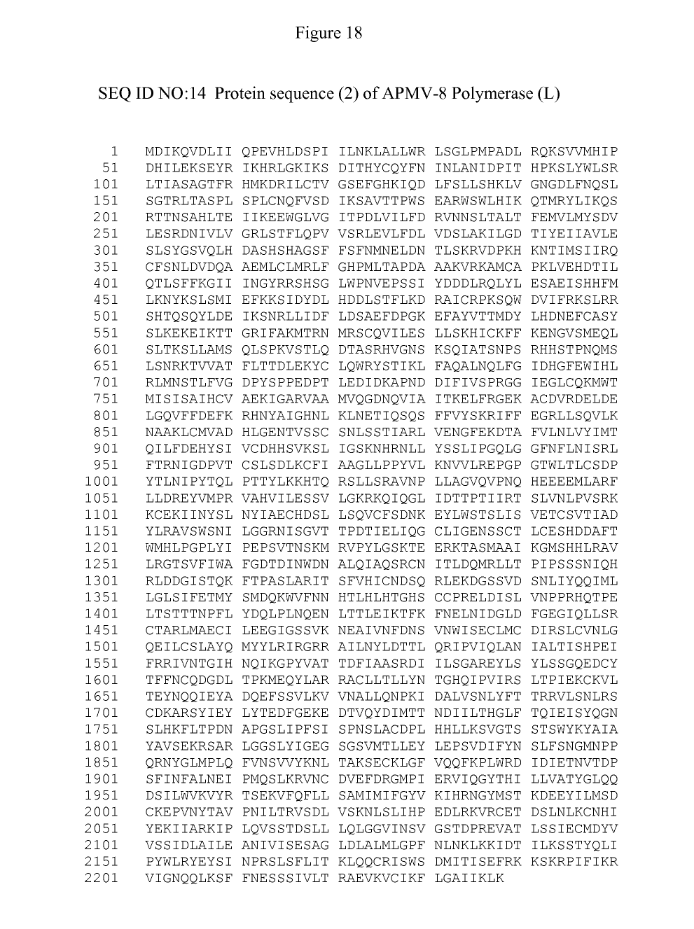

Based on this result, 8 ducks each were infected with APMV-8 starting with a dose EID₅₀ of 10³ per bird up to a dose of EID₅₀ of 10⁶ per bird (FIG. 10). Six out of eight ducks developed significant titers (≧16) 14 days after infection with a GMT of 14 after infection with an EID₅₀ of 10⁴/bird. At day 14 p.i. 6/8 ducks infected with an EID₅₀ of 10⁵/bird and 7/8 ducks infected with an EID₅₀ of 10⁶/birds developed significant titers (≧16) with a GMT of 17 and 23, respectively.

### Example 3

### Determination of the Full Length Sequence of APMV-8

For the determination of the full length sequence of APMV-8, viral RNA sequence information is initially needed. To this end, the 3′-end of the viral genome was cloned by using a primer (APMV-polyT, see table 1) which contained a degenerated sequence based on available 3′-sequences of APMV1 (Genbank accession No. AF077761), APMV-2 (Genbank accession No. EU338414), and APMV-6 (Genbank accession No. EF569970). Viral RNA was purified from allantoic fluid using the High Pure RNA isolation kit (Roche, Mannheim, Germany). The sequence was amplified using the 5′ RACE System for Rapid Amplification of cDNA Ends Version 2.0 (Invitrogen) following the manufacturer's instructions. Several fragments were obtained, gel eluted and cloned into the Topo TA cloning vector (Invitrogen) and positive selected clones were sequenced. The obtained nucleotide sequences were analyzed in an nblast search against the NCBI database which resulted in no similarities. A tblastx search against the NCBI database showed similarities to the nucleoprotein of 83% similarity of an Avian paramyxovirus 2 (APMV-2/Chicken/California/Yucaipa/56, Genbank accession No. EU338414) and of 56% similarity of an Avian paramyxovirus 6 strain (APMV-6/Goose/FarEast/4440/2003, Genbank accession No. EF569970). Using this primer, the primer walking method was employed using the 5′ RACE System for Rapid Amplification of cDNA Ends Version 2.0 (Invitrogen). The 5′-RACE produced an approximately 800 bp fragment. Using this technique, new sequence information was obtained based on sequence information from the previous sequence which has been used for the delineation of new oligonucleotides. The 5′-end of the viral genome was also determined by the 5′-RACE method. The 3′-end of the viral genome was obtained after ligation of the RNA with T4 RNA ligase1 (New England Biolabs). The ligation reaction was purified again with High Pure RNA isolation kit (Roche) and an RT-PCR was performed using Superscript III One Step RT-PCR kit with Platinum Taq (Invitrogen), The obtained cDNA fragment was cloned into the pCR2.1 vector (Invitrogen) and sequenced. Three plasmids from each cloned fragment were sequenced in both directions, thus resulting in sequence 6×-coverage of per nucleotide.

The full length genome sequence of the analyzed APMV-8 strain is 15342 nucleotides, this is in accordance to the rule of six (Calain, P. & Roux, L., 1993) for Paramyxovirinae. Six open reading frames (ORF) have been detected and are encoding for proteins. The order of the proteins was determined as 3′-NP-P-M-F-HN-L-5′ (the genome sequence SEQ ID NO:1 is in the 5′ to 3′ orientation antigenomic) using similarities of the protein sequence to proteins of other avian paramyxoviruses. The putative start and stop codons of the ORF's and the theoretical molecular weight (Swiss Institute of Bioinformatics ExPASy website) of the proteins are shown in table 2.

TABLE 2
Parameter of the proteins encoded by the APMV-8 sequence

| | | | Theoretical |
|---|---|---|---|
| Protein | Start codon | Stop codon | MW (kD) |
| Nucleoprotein | 141-143 | 1524-1526 | 51.2 |
| (NP) | | | |
| Phospho protein (P) | 1693-1695 | 2908-2910 | 43.5 |
| Matrixprotein (M) | 3076-3078 | 4193-4195 | 40.6 |
| Fusioprotein (F) | 4499-4501 | 6128-6130 | 58.5 |
| Hemagglutinin/ | 6383-6385 | 8114-8116 | 63.5 |
| neuraminidase (HN) | | | |
| Polymerase (L) | 8273-8275 or | 15011-15013 | 254.6 |
| | 8297-8299 | | 253.6 |

The putative genomic leader and trailer sequences were determined by determination of the putative gene start sequence of the NP gene (leader) and putative gene end sequence of the L protein (trailer). The leader sequence is located from nucleotide 1 to nucleotide 55. The putative gene start sequence (nt 56-63) of the NP gene ends the leader sequence. The trailer sequence is localized behind the last gene end sequence in the viral genome. Due to the presence of two putative gene end sequences for the RNA polymerase gene (nt 15161-15171 or 15288-15297) two putative trailer sequences have been identified (nt 15172-15342 or nt 15289-15342). The location of the putative gene start sequence (poly G containing sequences) and gene end sequences (signal sequence for a polyadenylation) and the intergenic sequences were summarized in table 3.

TABLE 3
Sequence and location of putative gene start,
intergenic, and gene end sequence of APMV-8

| Gene | Gene start | Gene end | intergenic |
|---|---|---|---|
| Nucleoprotein | 56-63 | 1615-1625 | 1626-1627 |
| Phosphoprotein | 1628-1635 | 2991-3001 | 3002-3031 |
| Matrixprotein | 3032-3039 | 4404-4416 | 4417-4441 |
| Fusionprotein | 4442-4449 | 6260-6271 | 6272-6278 |
| Hemagglutinin/neuraminidase | 6279-6287 | 8261-8273 | 8274-8275 |
| RNA polymerase | 8275-8283 | 15161-15171 | |
| | | or | |
| | | 15288-15297 | |

TABLE 4
SEQ ID NO v. DNA and protein sequences.

| SEQ ID NO | Gene name | Type |
|---|---|---|
| 1 | APMV-8 genome sequence | DNA or RNA |
| 2 | APMV-8 Nucleoprotien (NP) | DNA or RNA |
| 3 | APMV-8 Nucleoprotien (NP) | Protein |
| 4 | APMV-8 phospho protein (P) | DNA or RNA |
| 5 | APMV-8 phospho protein (P) | Protein |
| 6 | APMV-8 Matrixprotein (M) | DNA or RNA |
| 7 | APMV-8 Matrixprotein (M) | Protein |
| 8 | APMV-8 Fusioprotein (F) | DNA or RNA |
| 9 | APMV-8 Fusioprotein (F) | Protein |
| 10 | APMV-8 Hemagglutinin/ | DNA or RNA |
| | neuraminidase (HN) | |
| 11 | APMV-8 Hemagglutinin/ | Protein |
| | neuraminidase (HN) | |
| 12 | APMV-8 Polymerase (L) DNA | DNA or RNA |
| 13 | APMV-8 Polymerase (L) protein 1 | Protein |
| 14 | APMV-8 Polymerase (L) protein 2 | Protein |

The putative gene start sequences for APMV-8 were conserved containing a poly (C)₅ sequence followed by a 3′-GCU-5′ sequence. The only exception is the putative gene start sequence for the viral RNA polymerase (3′-CUCCCGCU-5′). The putative gene end sequences were also conserved and contain a poly (U)₆ sequence at the genomic viral 5′ sequence (Table 5).

TABLE 5
Gene Start and Gene End
sequences of APMV-8

| | | Sequences | |
|---|---|---|---|
| | Gene | (5-3′antigenomic | SEQ |
| | Name | orientation) | ID NO |
| Gene start | NP gene | CCCCCGCUUCUGUCA | 24 |
| | P gene | CCCCCGCUGGAGUUA | 25 |
| | M gene | CCCCCGCUUCUGUGC | 26 |
| | F gene | CCCCCGCUUUAGAAC | 27 |
| | HN gene | CCCCCGCUGGGUAAA | 28 |
| | L | CUCCCGCUGGAGAUG | 29 |
| Gene end | NP gene | AACUAAAUUCUUUUUU | 30 |
| | P gene | UAACUAAUUCUUUUUU | 31 |
| | M gene | AGGAUUAAUAUUUUUU | 32 |
| | F gene | CUAUAAAUUAUUUUUU | 33 |
| | HN gene | UACUUAAUUCUUUUUU | 34 |
| | L gene | ACUAAAAUUCUUUUUU | 35 |
| | (1) | | |
| | L gene | UUAUUGAUUUUUUUUU | 36 |
| | (2) | | |

These sequences were predicted based on sequences which were described for other paramyxoviruses of the genus Avulavirus (Chang et al., 2001, Nayak et al, 2008, Jeon et al., 2008). There are two possible start codons for the ORF of the RNA polymerase. The first start codon (nt 8273-8275) is localized in the gene end—intergenic region—gene start region between the FIN ORF and the viral RNA polymerase ORF. This makes this start codon unlikely but not impossible. The second start codon (8297-8299) is down stream of the gene end—intergenic region—gene start region and may act as initiation codon for the start of the translation of the RNA polymerase of APMV-8.

The genome of APMV-8 is 15342 nt long. This is larger than APMV-1 (SQ ID NO:15, 15186 nt, de Leeuw & Peeters, 1999), APMV-2 (SEQ ID NO:16, 14,904 nt, Subbiah et al., 2008), and APMV-4 (SEQ ID NO:18, 15054 nt, Nayak et al., 2008), and smaller than APMV-3 (SEQ ID NO:17, 16,272 nt, Kumar et al., 2008) and APMV-9 (SEQ ID NO:20, 15,438 nt, Samuel et al., 2009). The length of 55 nt of the leader sequence seemed to be conserved between all APMV (Krishnamurthy & Samal, 1998, de Leeuw & Peeters, 1999, Subbiah et al., 2008, Nayak et al., 2008, Kumar et al., 2008, Samuel et al., 2009) whereas the trailer sequence seemed to be variable in length. The gene start and gene end sequences of the viral genes were also highly conserved for APMV-8 (as shown in Table 5). This has been also described for sequences of APMV-2 (Subbiah et al, 2008), APMV-3 (Kumar et al, 2008), APMV-4 (Jeon et al., 2008, Nayak et al, 2008), APMV-6 (Chang et al, 2001), and recently for APMV-9 (Samuel et al., 2009). The number of the nucleotides of the full length sequence is a multiple of six which is agreement with the role of six for the genomes of paramyxoviruses (Kolakofsky et al., 1998).

The sequence identity between APMV-1, 2, 3, 4, 6, 8, and 9 genome sequences are shown in Table 6.

TABLE 6
sequence identity percentage between the genome of
APMV-1, 2, 3, 4, 6, 7, 8, and 9

| | | APMV | | | | | | | | | | |
|---|---|---|---|---|---|---|---|---|---|---|---|---|
| | | 1 | 2 | 3 | 4 | 6 | 7 | 8 | 8 | 8 | 8 | 9 |
| | | SEQ ID NO | | | | | | | | | | |
| | | 15 | 16 | 17 | 18 | 19 | 20 | 1 | 37 | 38 | 39 | 40 |
| 8 | 1 | 48 | 61 | 47.2 | 47.6 | 52 | 53 | 100 | 99.1 | 96.5 | 96.4 | 48 |

The percent sequence identity between two nucleic acid or polypeptide sequences is determined using Vector NTI 11.0 (PC) software package (Invitrogen, 1600 Faraday Ave., Carlsbad, Calif.). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. The percent identity was calculated based on the shorter sequence.

### Example 4

### Vaccination of One-Day-Old Broiler Chickens with APMV-8 Strain

Twenty one-day-old broiler chickens were separated into two groups according to table 7 shown below.

At day 1, the one-day-old chickens were bled to determine the antibody status directed against Newcastle disease virus (NDV) and APMV-8 using the hemagglutination inhibition assay (HI test). The test was performed using allantoic fluid from either NDV strain Lasota or APMV-8 infected SPF eggs. Four HA units of NDV strain Lasota or APMV-8 and 1% chicken red blood cells were used for the HI test. The resulting HI titers show that the serum of the chickens contained HI antibodies directed against NDV but no detectable antibodies against APMV-8 virus.

TABLE 7
Infection/vaccination of one-day-old broiler chickens

| | Group 1 | Group 2 (control) |
|---|---|---|
| | (ten one-day-old | (ten one-day-old |
| Treatment | chickens) | chickens) |
| Day 1: vaccination | Yes | No |
| with APMV-8 strain | | |
| Day 14: HI test | Yes | Yes |
| Day 14: second | 5 chickens (group 1-1): | 5 chickens (group 2-1): |
| vaccination with | 2nd vaccination/ | 2nd vaccination |
| APMV-8 strain | 1^{st} vaccination. | 5 chickens (group 2-2): |
| (boost) | 5 chickens (group 1-2): | not vaccinated |
| | no 2^{nd} vaccination/ | |
| | 1^{st} vaccination | |
| Day 28: HI test | Yes | Yes |

At day 1, group 1 of 10 chickens were infected with via the nasal route with 10⁶ EID₅₀ of APMV-8 strain, group 2 of 10 chickens were not infected serving as control.

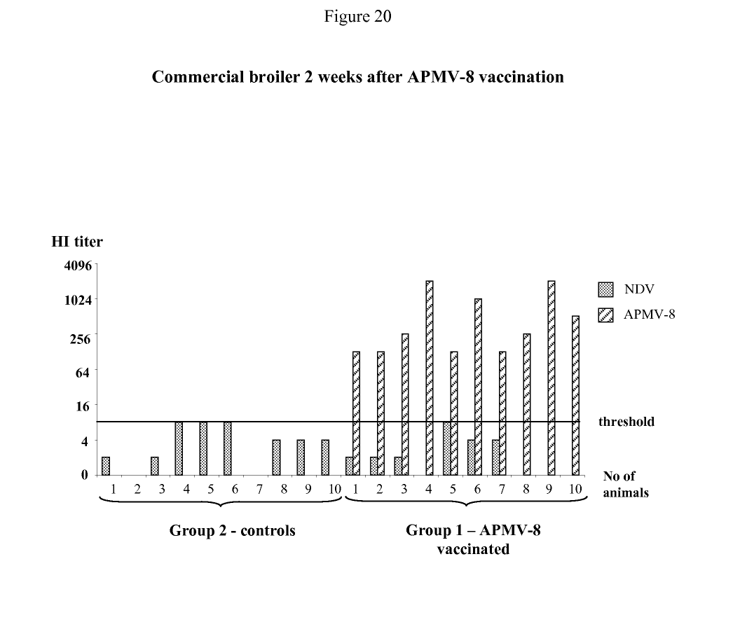

Fourteen days after infection (day 14), the chickens were bled and the obtained serum samples were analyzed again for the presence of HI antibodies directed against NDV and APMV-8 (FIG. 20). The result showed that the APMV-8 vaccinated chickens showed HI titers using APMV-8 as antigen. The APMV-8 specific HI titers were between 128 and 2048. The HI titers against NDV declined to a HI titer below 16, thus they are not considered as NDV positive. The result showed that chickens maternal derived antibodies directed against NDV did not omit the infection with APMV-8, thus interference of such antibodies with APMV-8 vaccination is unlikely.

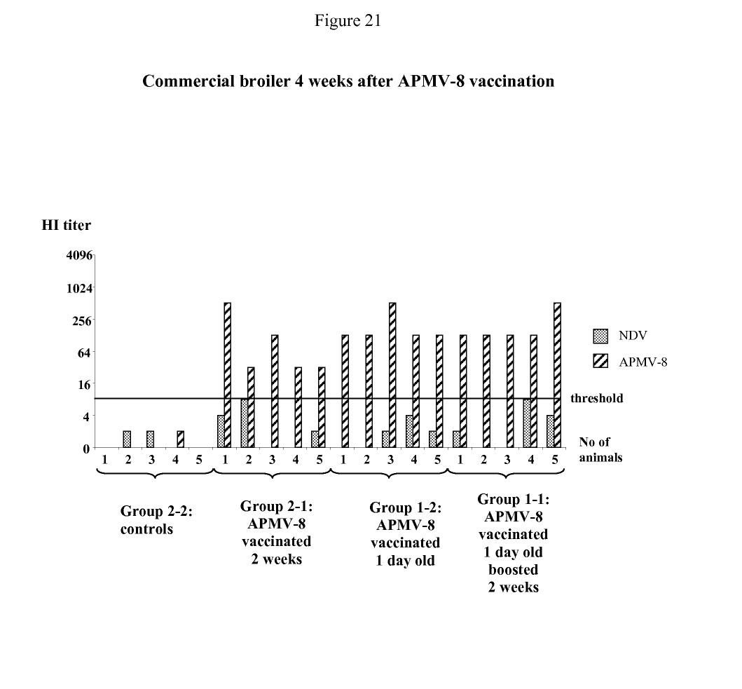

Fourteen days after infection (day 14), the chickens in group 1 and group 2 were split. Five chickens in each of group 1 (group 1-1) and group 2 (group 2-1) were again infected with 10⁶ EID₅₀ of APMV-8 strain (table 7), the remaining five chickens in each group (group 1-2 and group 2-2) were not infected. This experiment is designed to investigate where a later infection of chickens would have an effect on the infection and whether a second infection (boost vaccination) would increase the antibody titer. Fourteen days later (day 28), all chickens were bled again and the serum was investigated for the presence of APMV-8 and NDV antibodies. The serum titers (FIG. 21) showed that a first vaccination at day 14 (group 2-1) did induce APMV-8 specific antibody titers ranging from 32 to 512. In chickens vaccinated only at day 1 (group 1-2), the antibody titers declined to titers in a range from 128 to 512. In chickens which have been vaccinated at day 1 and day 14 (group 1-1), the APMV-8 specific antibody titer did not increase, suggesting that the virus used for the second infection was neutralized by APMV-8 specific antibodies induced by the first infection. The serum of the non-vaccinated controls (group 2-2) did not contain APMV-8 specific HI antibodies. At day 28, the NDV antibodies declined further, only 11 chickens out of the 20 chickens showed any HI titers with the NDV antigen whereas at day 14 fourteen chickens showed low antibody titers against NDV.

### Example 5

### In Ovo Vaccination of Embryonated SPF Eggs

This study was performed to test if an in ovo vaccination with APMV-8 would result in an antibody response in chickens and if in ovo vaccination would interfere with hatchability and livability.

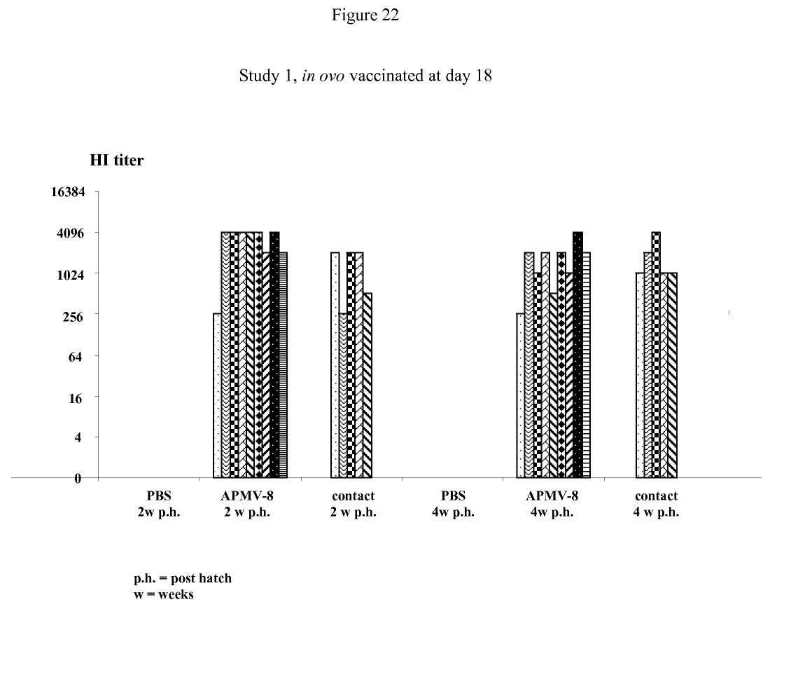

In study 1, 108 SPF eggs were in ovo vaccinated with APMV-8 virus strain using the INOVOJECT (Pfizer Animal Health, NY, USA) at day 18 of incubation. The virus was diluted in 0.9% sterile NaCl saline. The back titration of the diluted virus revealed a titer of 10^{5.5} EID₅₀/100 μl. As control, 108 eggs were inoculated with 0.9% sterile NaCl saline. The volume for the inoculation was 100 μl per egg. Eighty chickens hatched from the control group and forty-five chickens hatched from the APMV-8 vaccinated group. Ten chickens from each group were transferred to one Horsefall-Bauer unit. In addition, the chickens of the APMV-8 vaccinated group and five chickens of the control group were co-mingled in a Horsefall-Bauer unit to test transmission of APMV-8 after vaccination. Water and feed were provided ad libitum. Fourteen and twenty-eight days after hatch, blood samples were taken and tested for the presence of HI antibodies directed against APMV-8 using 4 HA units and 1% of chicken red blood cells. The results (FIG. 22) showed that in ovo vaccination at day 18 of incubation resulted in an immune response as indicated by the presence of HI titers in the tested serum samples. Fourteen days after hatch, an HI titer from 256 to 4048 was observed in the in ovo vaccinated group. In the serum of the contact chickens, an HI titer from 256 to 2048 was observed 14 days after contact with chickens from the vaccinated group, indicating shedding of the virus used for vaccination. The control group did not show any HI titer specific for APMV-8. Fourteen days later, the chickens were bled again and showed titers from 256 to 4096 in the APMV-8 vaccinated group and 256 to 1024 in the APMV-8 contact group. The control group showed no presence of APMV-8 HI antibodies.

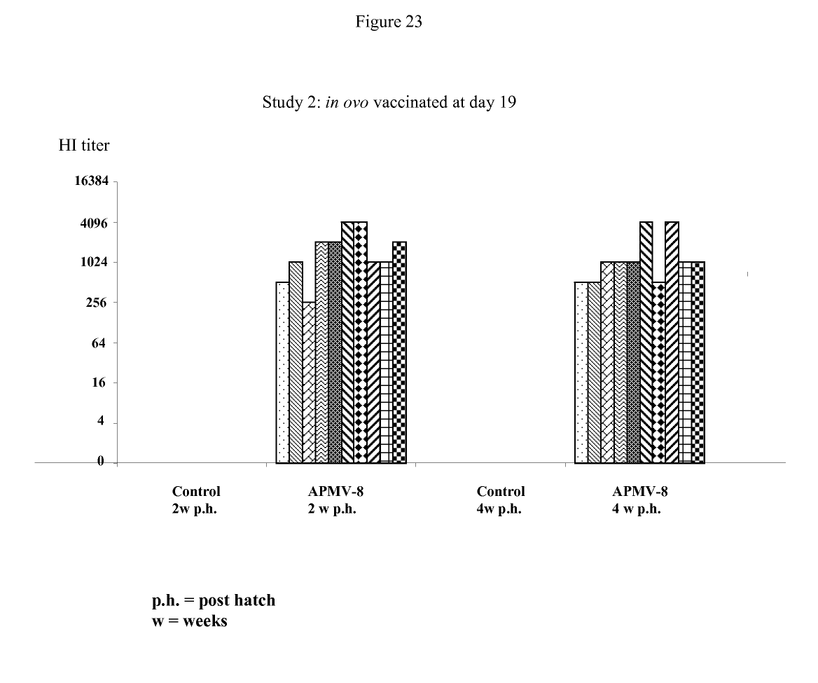

In study 2, 88 SPF (specific pathogen free) eggs were in ovo vaccinated using the INOVOJECT at day 19 of incubation. The APMV-8 virus strain was diluted in 0.9% sterile NaCl saline. The titer of the virus was 10^{5.75} EID₅₀/100 μl as observed after back titration of the diluted virus. As control, 88 SPF eggs were inoculated with 0.9% sterile NaCl saline. The volume for the inoculation was 1000 per egg. Seventy-four chickens hatched from the control group (NaCl) and seventy-six chickens hatched from the APMV-8 vaccinated group. Ten chickens from each group were transferred to one Horsefall-Bauer unit. Water and feed were provided ad libitum. Fourteen days after hatch, blood samples were taken and tested for the presence of HI antibodies directed against APMV-8 using 4 HA units and 1% of chicken red blood cells. The results (FIG. 23) showed that in ovo vaccination at day 19 of incubation resulted in an immune response with HI titers specific for APMV-8. Fourteen days after hatch, an HI titer from 256 to 4048 was observed in the APMV-8 in ovo vaccinated group. The control group did not show any HI titer specific for APMV-8. The chickens were bled again at day 28 after hatch. The HI titers for the APMV-8 vaccinated group ranged from 512 to 4096 (FIG. 23) whereas the sera of the control chickens were still APMV-8 negative.

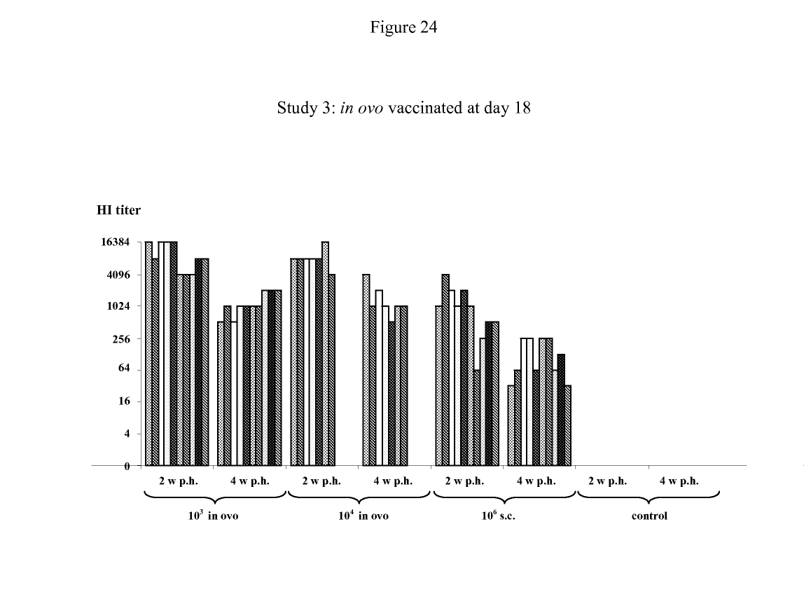

In a third study, 108 SPF eggs in group 1 and group 2 were in ovo vaccinated using the INOVOJECT at day 18 of incubation with 10^{3.5} EID₅₀ and 10^{4.5} EID₅₀, respectively. The APMV-8 virus strain was diluted in 0.9% sterile NaCl saline. In a third group 108 SPF eggs were inoculated with 0.9% sterile NaCl saline as control. Eighty-three chickens hatched from group 1, seventy-nine chickens hatched from group 2, and eighty-eight chickens hatched from group 3 (see table 8). After hatch, ten chickens from each group were transferred to one Horsefall-Bauer unit. Water and feed were provided ad libitum. After hatch, ten chickens from group 3 were vaccinated subcutaneously in the neck region with a volume of 100 μl 10⁶ EID₅₀ APMV-8 virus. Fourteen days after hatch, blood samples were taken and tested for the presence of HI antibodies directed against APMV-8 using 4 HA units and 1% of chicken red blood cells (CRBS). The results (FIG. 24) showed that in ovo vaccination at day 18 of incubation resulted in an immune response with HI titers specific for APMV-8. Fourteen days after hatch, an HI titer from 4096 to 16384 was observed in the APMV-8 in ovo vaccinated groups. The control group did not show any HI titer specific for APMV-8. The group subcutaneously vaccinated with APMV-8 showed a seroconversion with a titer ranging from 64 to 4096. The chickens were bled again at day 28 after hatch and tested for the presence of APMV-8 specific HI antibodies. The HI titer at four weeks after hatch decreased and ranged between 512 and 4096. The control group did not show any HI titer specific for APMV-8. The HI titer in the group which was subcutaneously vaccinated showed HI titers ranging between 32 and 256.

TABLE 8

| | Group 1 | Group 2 | Group 3 (control) |
|---|---|---|---|
| SPF eggs | 108 | 108 | 108 |
| Hatched chicken | 83 | 79 | 88 |
| Vaccination with | 10^{3.5} EID₅₀ | 10^{4.5} EID₅₀ | 0.9% sterile NaCl |
| APMV-8 at day 18 | 100 μl per egg | 100 μl per egg | saline |
| of incubation | | | |

### Example 6

### Development of Reverse Genetics of the APMV-8 Strain and Generation of APMV-8 Mutants Expressing Heterologous Genes

### Construction of the Expression Plasmids Containing the NP, P, and L Genes of APMV-8

For the establishment of a reverse genetics system for paramyxoviruses, the establishment of plasmids expressing the proteins involved in viral RNA replication is essential. The open reading frames (ORF) of three APMV-8 proteins (nucleoprotein NP, phosphoprotein P, RNA dependent RNA polymerase protein or protein L) were cloned into the eukaryotic expressions vector pcDNA3 (Invitrogen, California, USA). To this end, the RNA of allantoic fluid containing APMV-8 was purified using the High Pure RNA Isolation Kit (Roche, Basel, Switzerland). The purified RNA was used for reverse transcription polymerase chain reaction (RT-PCR) using the Titan One Tube RT-PCR Kit (Roche). The ORFs of the proteins were amplified using the appropriate primer pairs: [NP(NP-FP, NP-RP), P(P-FP, P-RP), L (L-FP, L-RP), see table 9]. The reactions products were separated on a 0.7% agarose gel and eluted from the gel using the QIAquick Gel Extraction Kit (Qiagen, Hilden, Germany) following the protocol provided by the manufacturer. The RT-PCR fragments were incubated with the appropriate restriction enzymes (NP and P with Eco RI/NotI; L with Kpn I/NotI), gel eluted again and ligated into the eukaryotic expression vector pcDNA3 cleaved with the appropriate restriction enzymes. The ligation reactions were transformed into Top 10F cells (Invitrogen) and plasmid DNA harvested from the Top10F cells was digested with the appropriate restriction enzymes (see above). Plasmids containing DNA fragments (pcDNA-NP, pcDNA-P, pcDNA-L) with the appropriate size were sequenced.

TABLE 9
Primer for amplification of the genes
for the proteins of the
RNP complexes

| Primer | | | | SEQ |
|---|---|---|---|---|
| name | Primer sequence^{a} | Orientation^{b} | Positions^{c} | ID NO: |
| NP-FP- | Eco RI | sense | 141-168 | 41 |
| pc3 | ccGAATTCATG | | | |
| | TCATCTGTGTTC | | | |
| | AATGAGTATCAGG | | | |
| NP-RP- | Not I | antisense | 1501-1526 | 42 |
| pc3 | ccGCGGCCGCTTA | | | |
| | CCATTCTAGCCC | | | |
| | GTTCTCGTATG | | | |
| P-FP- | Eco RI | sense | 1693-1714 | 43 |
| pc3 | ccGAATTCATGGATT | | | |
| | TCGCCAATGATGAAG | | | |
| P-RP- | Not I | antisense | 2881-2910 | 44 |
| pc3 | ccGCGGCCGCTTACG | | | |
| | CATTATATATTGCCT | | | |
| | GCTTGACTCG | | | |
| L-FP- | Kpn I | sense | 8292-8320 | 45 |
| pc3 | ccGGTACCATGGATAT | | | |
| | AAAACAAGTTGACCTG | | | |
| L-RP- | Not I | antisense | 14989- | 46 |
| pc3 | ccGCGGCCGCTTA | | 15013 | |
| | TTTCAACTTGATG | | | |
| | ATTGCACCG | | | |

^{a}The primer sequence contains the restriction enzyme cleavage sites used for cloning. The restriction sites are bold and identified. The start and stop codons are highlighted by italics. Virus specific sequences are underlined.
^{b}The orientation of the primer sequence in accordance with the viral messenger RNA.
^{c}The position are the virus specific sequences in the full length genome as shown.

### Construction of a Plasmid Containing the Full Length Genome of APMV-8

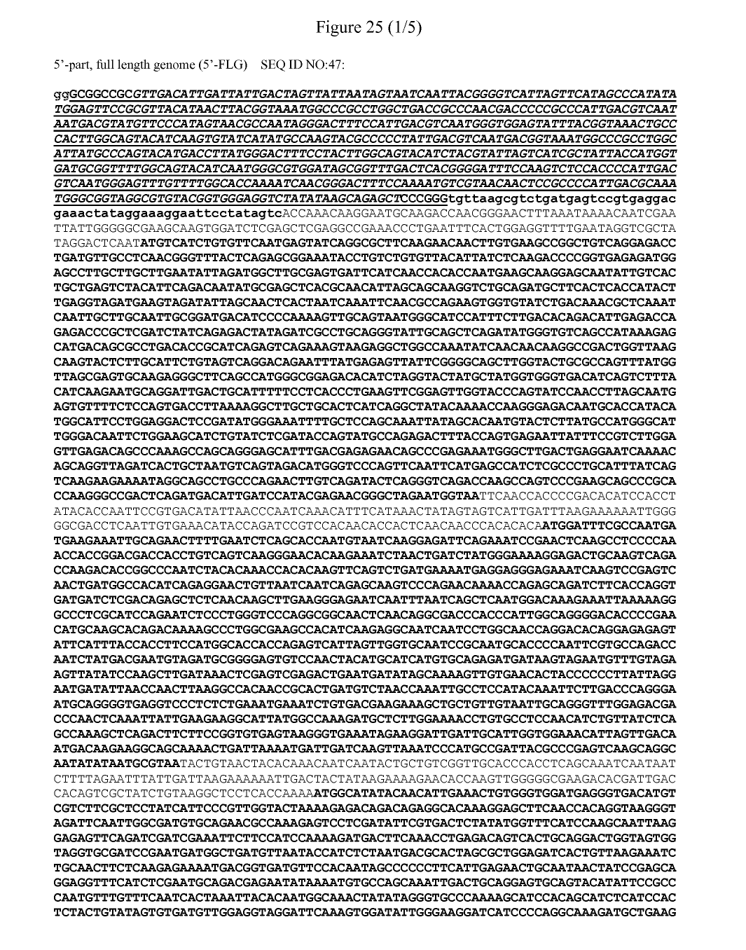

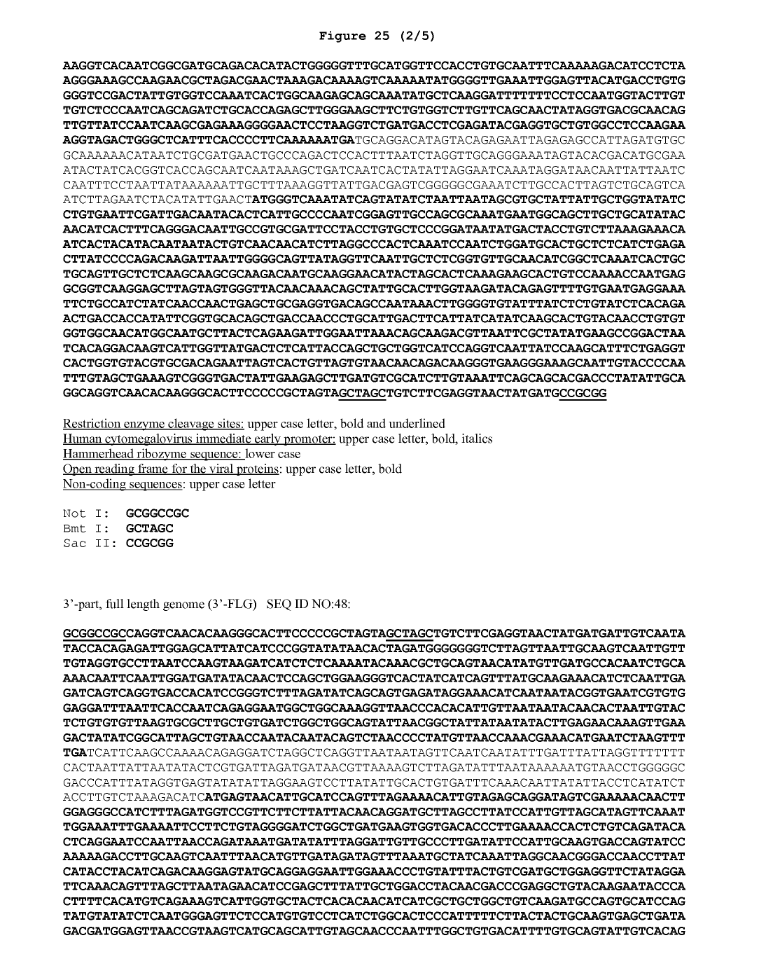

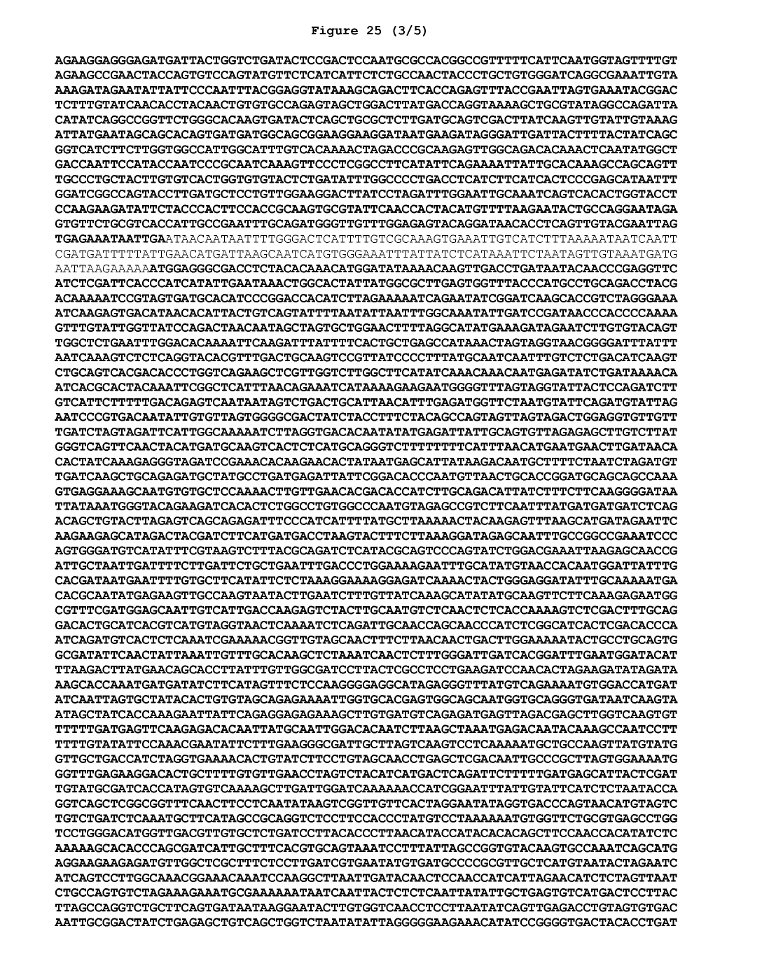

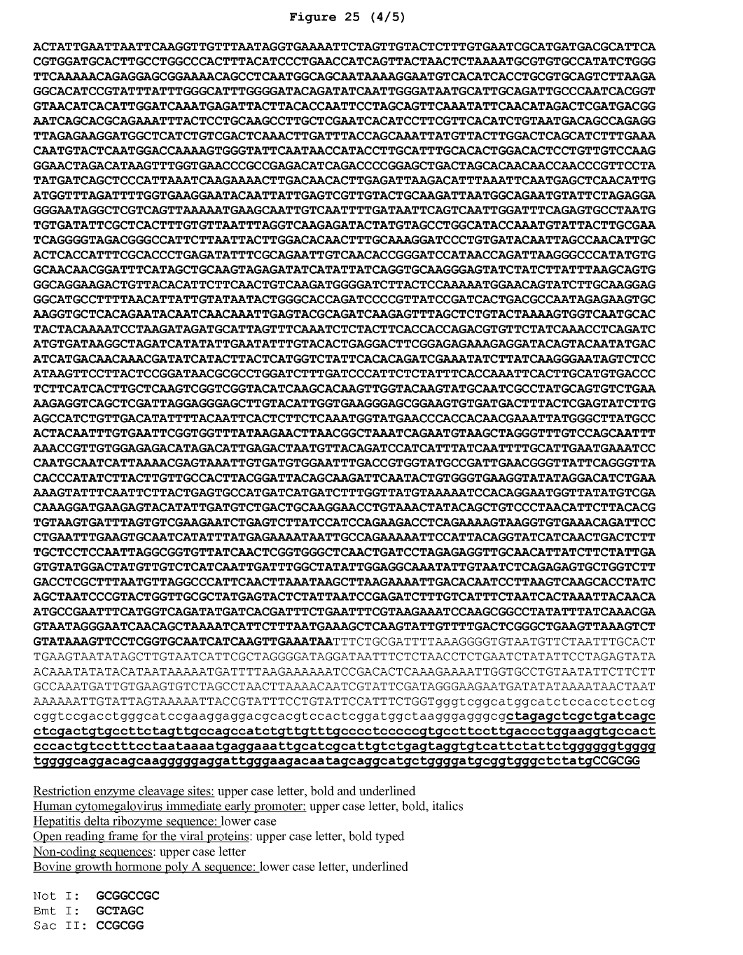

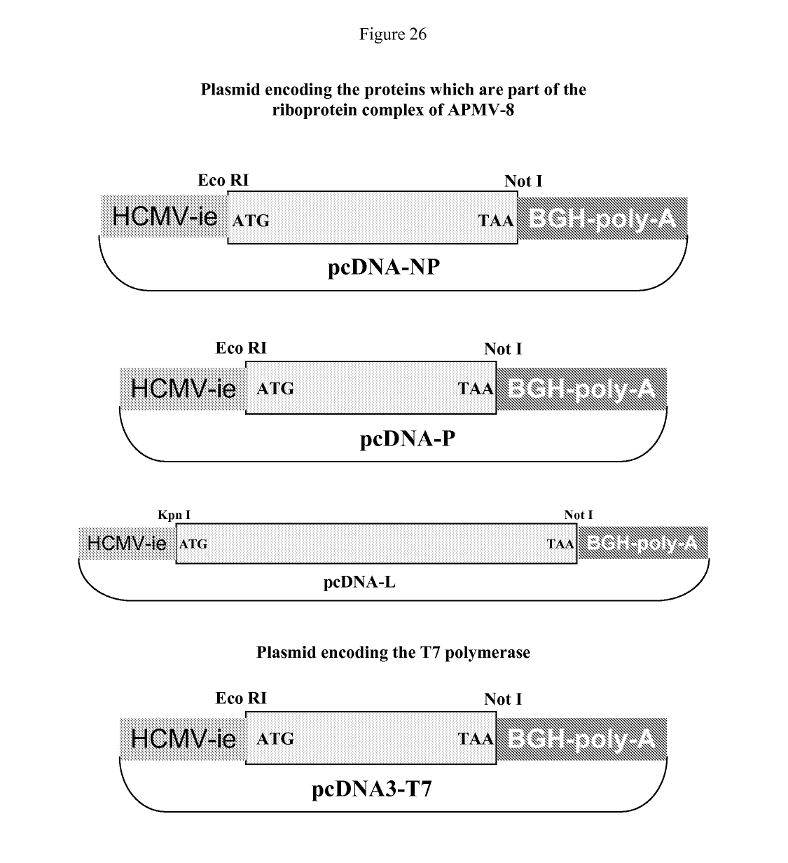

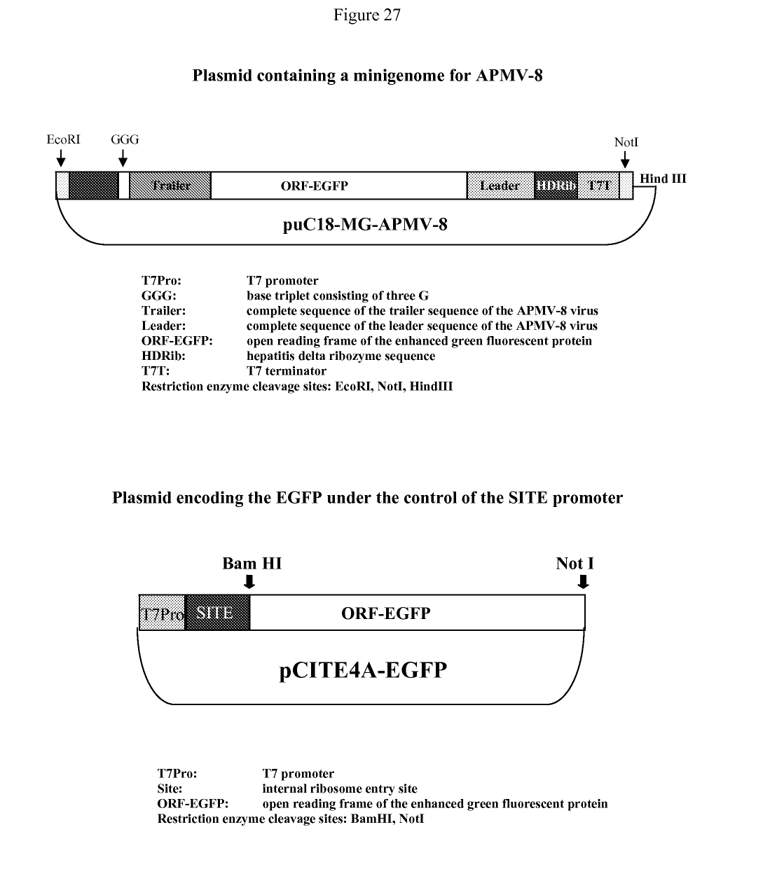

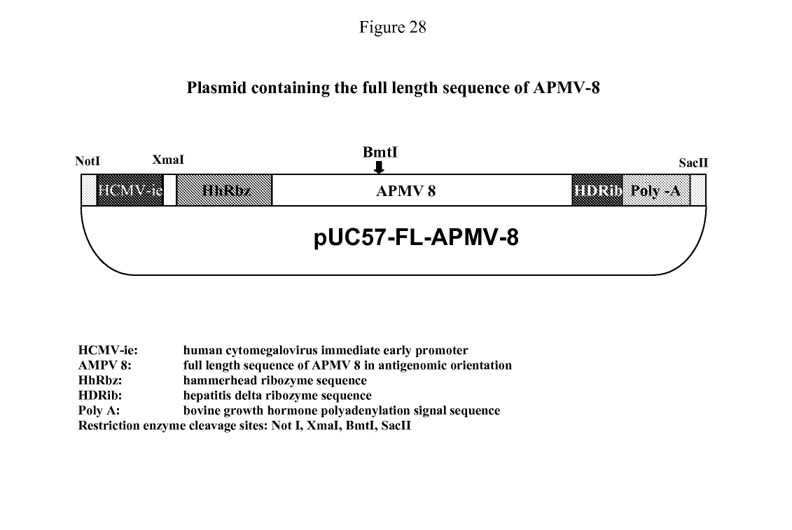

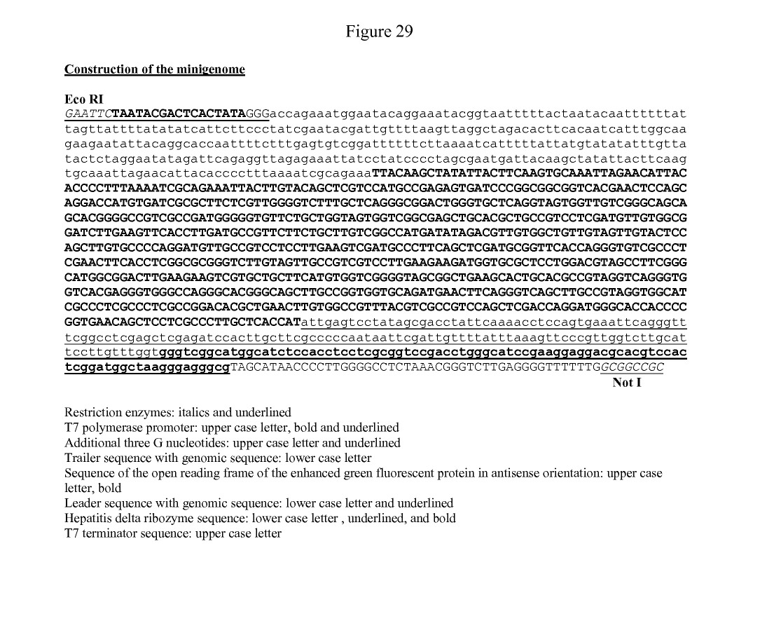

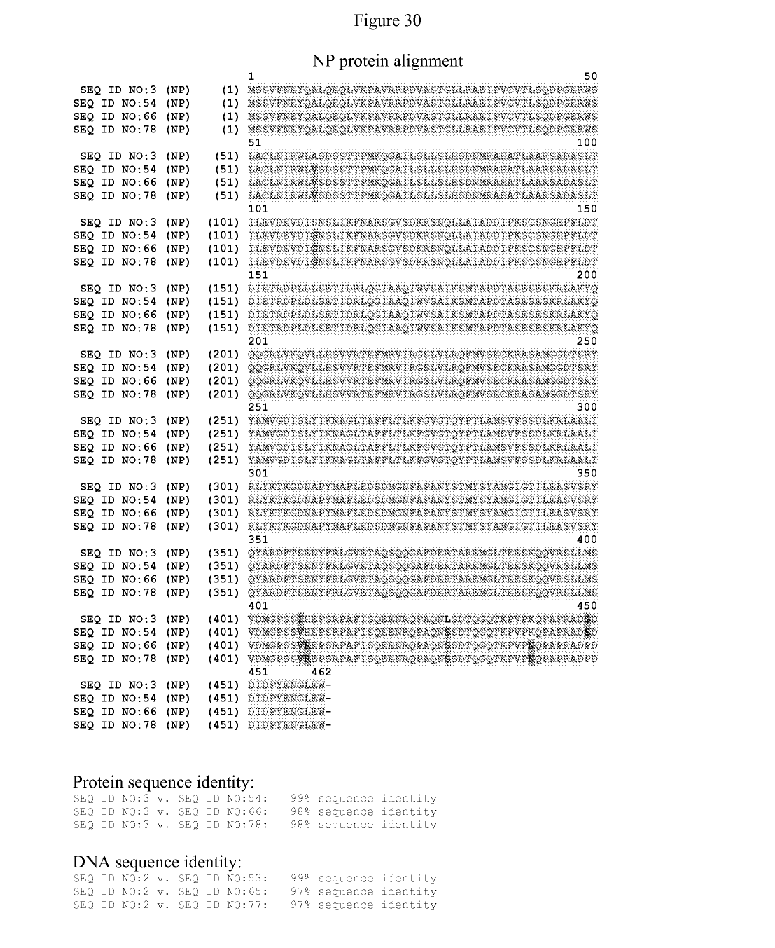

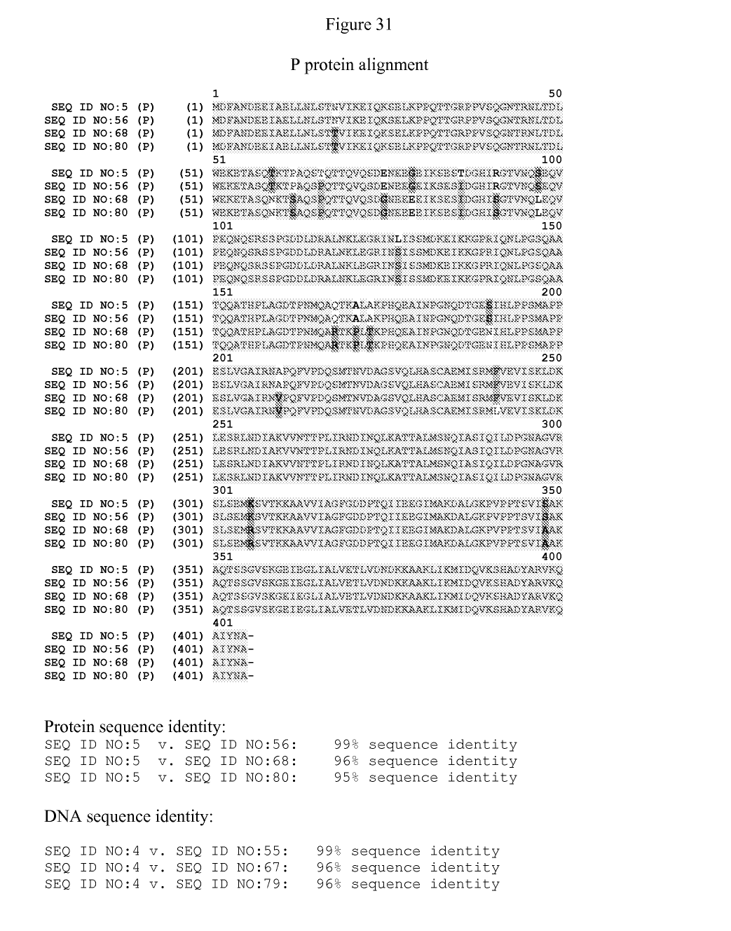

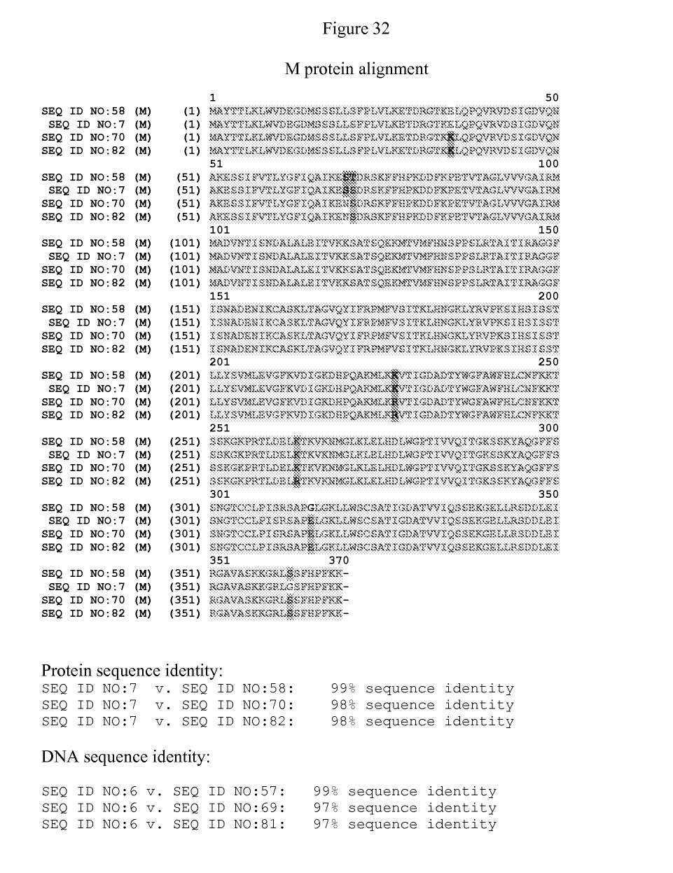

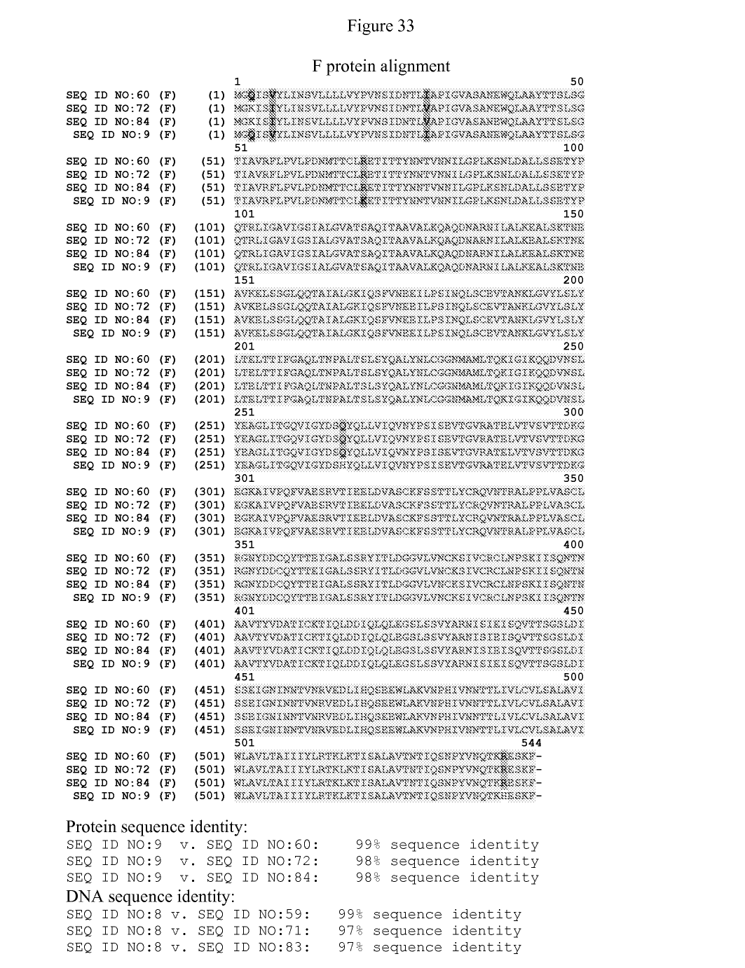

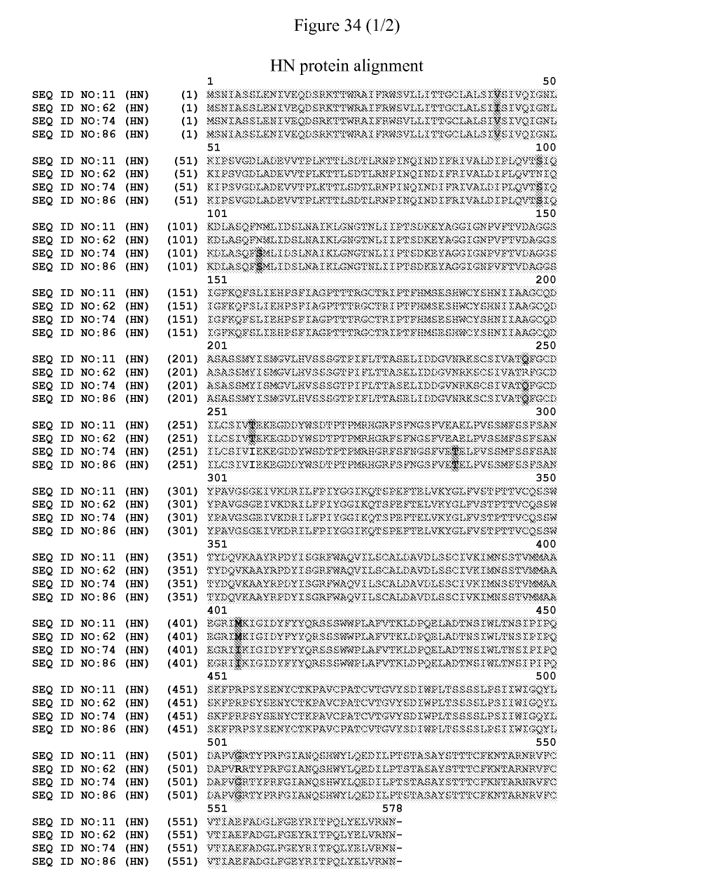

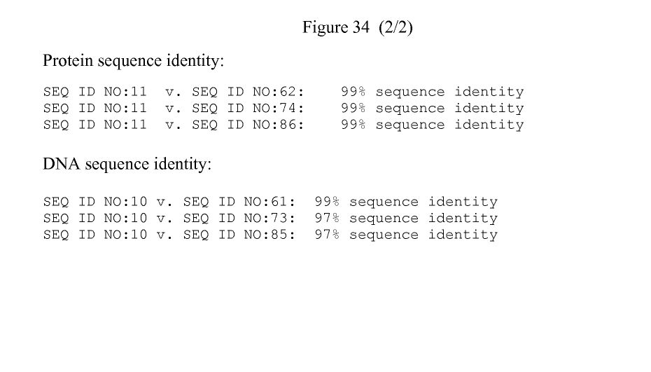

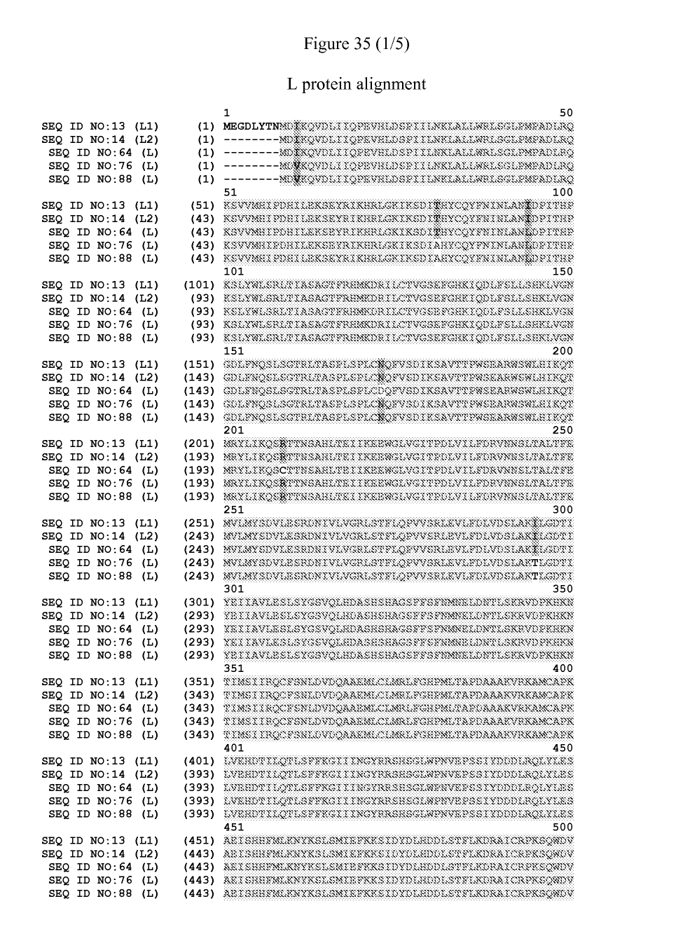

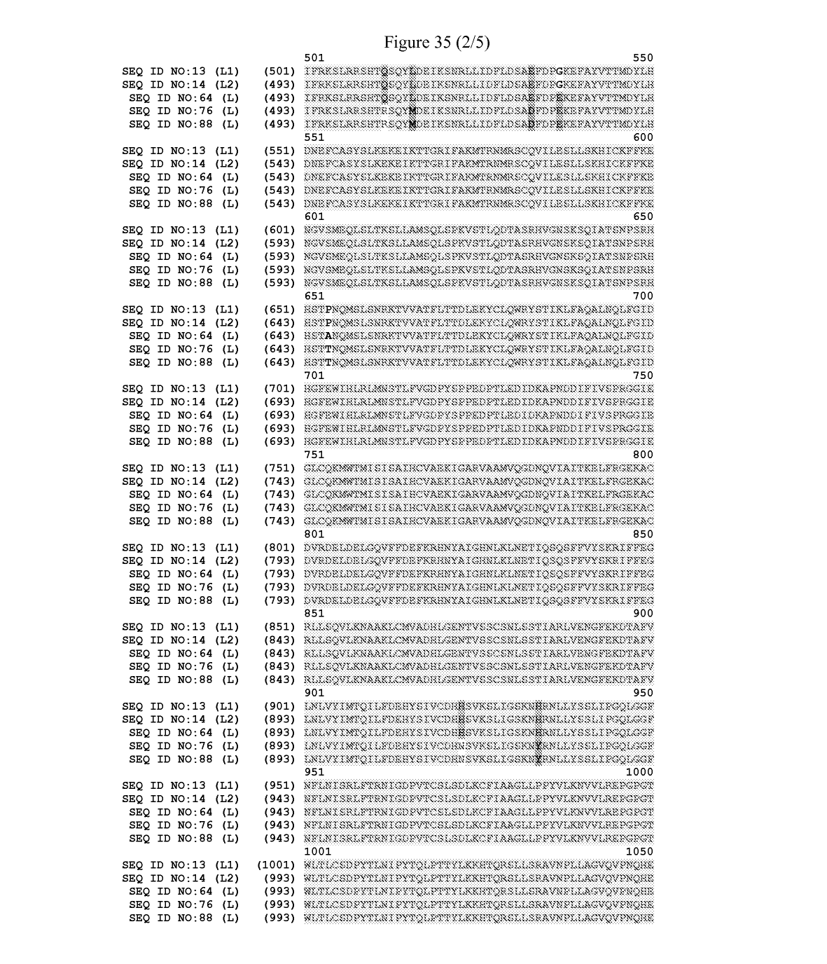

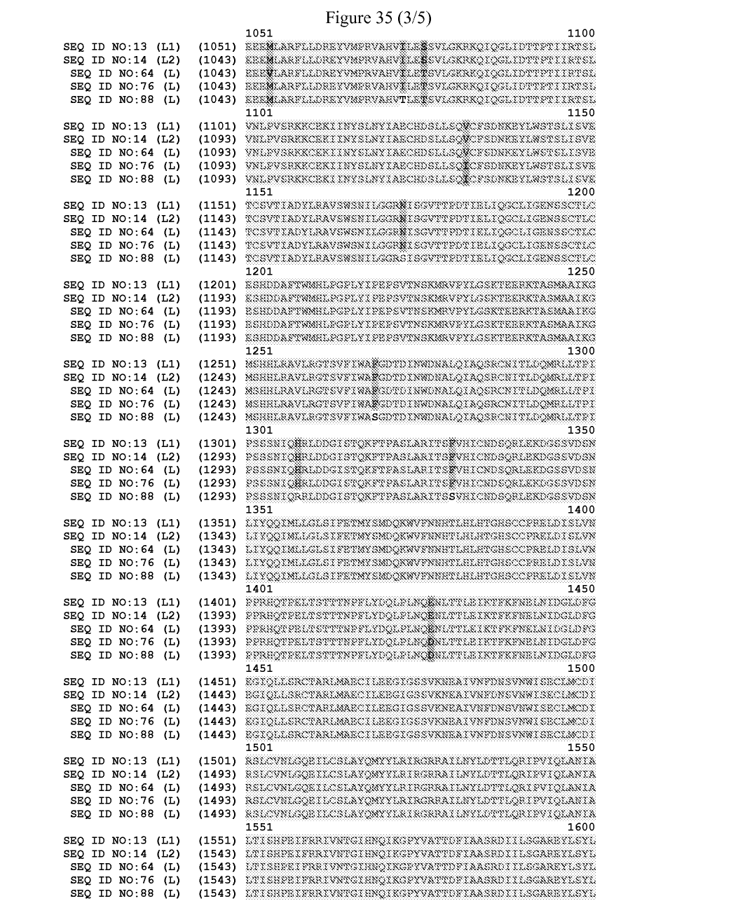

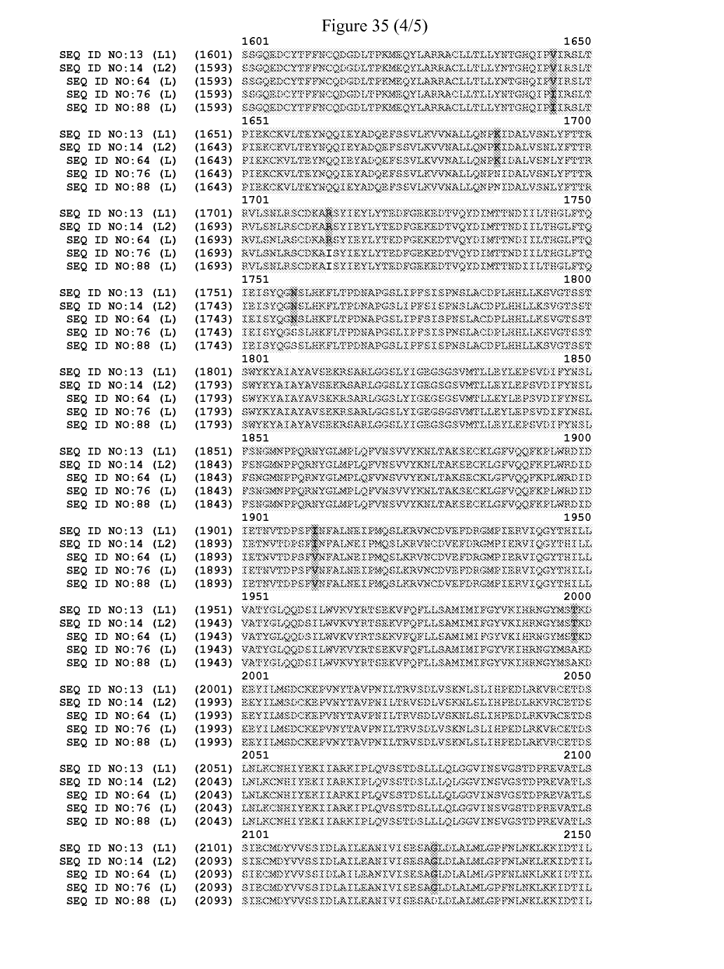

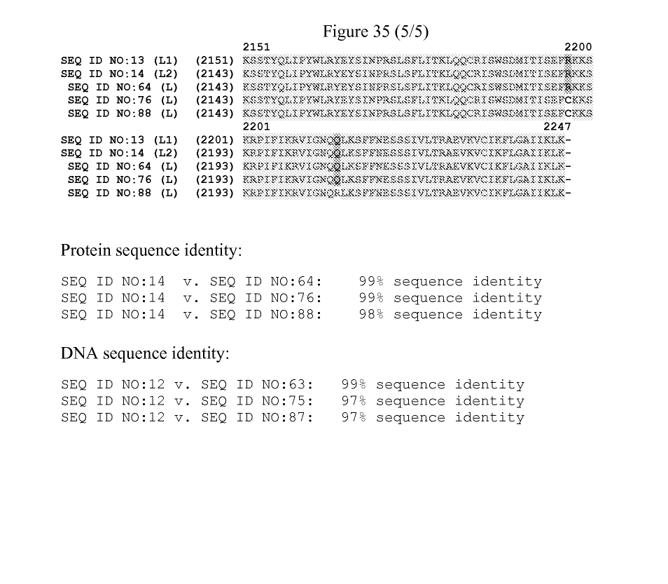

For the generation of a plasmid containing the full length APMV-8 genome, the cDNA genome of the virus was synthesized (Genscript, New York, USA) based on the generated consensus sequence in two different parts (5′-FLG, 3′-FLG, see FIG. 25). The 5′-part (5′-FLG, SEQ ID NO:47) of the viral sequence was synthesized from nucleotide 1-5564. Preceding the APMV-8 sequence is a sequence cassette consisting of the CMV-IE promoter, followed by a restriction enzyme cleavage sequence for XmaI (for possible subsequent cloning procedures) and the hammerhead ribozyme sequence. At the 5′-end and the 3′-end of the sequence, restriction enzyme cleavage sites for Not I and SacII were added, respectively. The synthesized 3′-part (3′-FLG, SEQ ID NO:48) of the sequence (nucleotide 5503-15342) is followed by the hepatitis delta ribozyme sequence and poly-A signal sequence of the bovine growth hormone. For cloning purpose, the sequence for the Not I restriction enzyme was added at the 5′-end of 3′-FLG and the sequence for the Sac II restriction enzyme was added at the 3′-end of the sequence. Within the overlapping parts of 5′-FLG and 3′-FLG (nucleotide 5503-5564), the sequence for a unique restriction enzyme (Bmt I) was located which cleaves at nucleotide 5541 of the full length sequence. Both parts of the DNA (5′-FLG, 3′-FLG) were ligated separately into the plasmid pUC57 (Genscript) resulting in the plasmids pUC57/5′-FLG and pUC57/3′-FLG. To clone both fragments together, pUC57/5′-FLG was cleaved with BmtI and Sac II and the 5′-FLG-containing plasmid was gel eluted. In parallel, pUC57/3′-FLG was cleaved with the same enzymes and the fragment 3′-FLG was eluted. The 3′-FLG was subsequently ligated into 5′-FLG-containing plasmid to obtain a plasmid which contains the full length cDNA sequence of the APMV-8 genome under the control of the CMV-IE promoter (pUC57-FL-APMV-8).

### Construction of the Plasmid Containing the Minigenome of APMV-8

The plasmid containing all functional elements of a minigenome for APMV-8 (pMG-APMV-8) was constructed using the method described by Conzelmann, et al. (J Virol. 68:713-719, 1994). Plasmid pMG-APMV-8 contains the trailer and leader region of the APMV-8 genome which is flanked by the T7 promoter and the antigenome hepatitis delta virus ribozyme sequence (Collins, et al., PNAS USA 88:9663-9667, 1991). The antigenome hepatitis delta virus ribozyme sequence is followed by a T7 transcription terminator sequence. Between the trailer and leader region the coding sequence of the enhanced green fluorescent protein in antisense orientation is located. Preceding the trailer sequence and immediate after the T7 promoter three additional G residues are located. The insert is flanked by the restriction enzyme cleavage sites Eco RI and Not I and was cloned blunt end into the plasmid pUC57. This construct was subcloned into the plasmid pUC18. To this end, plasmid pMG-APMV-8 then was cleaved with Eco RI and Hind III and the appropriate fragment was gel eluted and ligated into the appropriately cleaved plasmid pUC18 to obtained puC18-MG-APMV-8. The presence of the insert was confirmed by sequencing.

### Generation of an Expression Plasmid Allowing the Expression of the T7 Polymerase

For the generation of a plasmid encoding the T7 DNA dependent RNA polymerase (T7 polymerase) the coding sequence (GenBank accession number AY264778) was synthesized by Genscript. The T7 polymerase sequence (SEQ ID NO:49) was modified for optimization of the codon usage for expression in a eukaryotic system and to remove possible splice donor/acceptor sites in the sequence. The T7 polymerase encoding sequence was flanked by an EcoRI (5′) and NotI (3′) site. The synthesized fragment was cloned blunt end into the vector pUC57 (pCU57-T7). This plasmid was cleaved with EcoRI/NotI and the T7 polymerase-encoding fragment was gel eluted. The fragment was then cloned into the eukaryotic expressions vector pcDNA3 (Invitrogen) to obtain pcDNA3-T7. The presence of the fragment in the vector pcDNA3-T7 was verified by sequencing.

Generation of a Plasmid for the Expression of the Enhanced Green Fluorescent Protein with the Use of an Internal Ribosomal Entry Site Under the Control of a T7 Promoter.

To test the functionality of the T7 polymerase, the open reading frame of the enhanced green fluorescent protein (EGFP) was amplified by PCR using the plasmid pEGFP-N1 (Clontech, California, USA) and the primer pair:

SEQ ID NO: 50
EGFP-FP (CCGGATCCATGGTGAGCAAGGGCGAGGAGCTG)
and
SEQ ID NO: 51
EGFP-RP (CCGCGGCCGCTTACTTGTACAGCTCGTCCATGCCG)

The obtained PCR fragment was gel eluted and incubated with the restriction enzymes BamHI and NotI. The reaction product was gel eluted and ligated into the appropriately cleaved vector pCITE 4A (Novagen). The obtained plasmid (pCITE4A-EGFP) was used for subsequent experiments. Plasmids pCITE4A-EGFP and pcDNA3-T7 were transfected alone or in combination into the chicken cell line DF1 grown in 24-well-plates using Lipofectin 2000 (Invitrogen). Twenty four hours after transfection, the medium was removed and sterile phosphate buffered saline (PBS) was added. The cells were evaluated using the inverted fluorescence microscope Axiovert 40 CFL (Zeiss, Jena, Germany). Green fluorescence was only observed in wells of the tissue culture plate which was co-transfected with both plasmids. This result indicates that both plasmids, pCITE4A-EGFP and pcDNA3-T7, were functional.

### Validation of the Functionality of the Expressed Viral Proteins NP, P, and L Using the Minigenome Plasmid

DF1 cells were co-transfected with pcDNA3-T7, pUC18-MG-APMV-8, pcDNA-NP, pcDNA-P, and pcDNA-L to validate the functionality of the expressed NP, P and L proteins. Twenty four hours after transfection, the medium was removed and sterile phosphate buffered saline (PBS) was added. The cells were evaluated using the inverted fluorescence microscope Axiovert 40 CFL (Zeiss, Jena, Germany). Green fluorescence was only observed in wells of the tissue culture plate which was co-transfected with the 5 plasmids. This result indicates that the expressed viral proteins NP, P and L were functional to transcribe the APMV-8 minigenome into mRNA and express the EGFP protein encoded by pUC18-MG-APMV-8.

Rescue of AMPV8 Virus from Plasmid Containing the Full Length Sequence of APMV-8

DF1 cells were co-transfected with pUC57-FL-APMV-8, pcDNA-NP, pcDNA-P, and pcDNA-L. After 48 to 96 hours, the supernatants of the DF1 cells were inoculated in 10-day-old embryonated eggs to propagate the virus. After 3 to 5 days, the allantoic fluid was harvested and tested for hemagglutination activity (HA) using 1% chicken red blood cells. Allantoic fluid tested positive for HA activity was used for three procedures. 1) DF1 cells were infected with the allantoic fluid and tested 36 hours after infection with an APMV-8 specific antiserum for the presence of APMV-8 protein expression in an indirect immunofluorescence assay. 2) The allantoic fluid was tested for APMV-8 specificity using an APMV-8 specific chicken serum (provided by the National Central Veterinary Laboratory, Ames, Iowa, USA) by an hemagglutination inhibition assay. 3) The rescue virus was identified by RT-PCR using APMV-8 specific oligonucleotides. The absence of viral cDNA was verified by omitting the RT step during the reaction. Samples tested positive in all three assays were further propagated in embryonated SPF chicken eggs.

Propagation of APMV-8 in Cells Other than Chicken Origin

Cells from different species [hamster (Baby hamster kidney cells, BHK-21 cells), monkey (Vero cells, cell line with the origin of the kidney of an African green monkey), and canine (Madin-Darby canine kidney cells, MDCK), and quail (Quail muscle cell line QM7)] were grown in 24 well tissue culture plates and infected with a multiplicity of infection of 0.01. The cells were fixed with ice cold ethanol 24 hours after infection and analyzed for the presence of APMV-8 specific proteins by indirect immunofluorescence using an APMV-8 specific antiserum from an APMV-8 infected SPF chicken. The binding of the antibodies was visualized by using a goat anti-chicken IgY specific FITC conjugate. Non-infected cells were used as negative control. Only in the APMV-8 infected cells was green fluorescence observed. This indicated that APMV-8 was able to infect cells from species other than chicken.

The replication of APMV-8 was increased in presence of trypsin. MDCK cells were infected with APMV-8 as described above. After infection the cells were rinsed with serum-free medium and either overlaid with trypzin-containing serum-free medium in a concentration of 1 ug/ml or with serum-free medium only. Twenty four, forty eight, and ninety six hours after infection the cell supernatants were removed and the TCID50 was determined on DF1 cells using indirect immunofluorescence as described above. The obtained data indicated that in presence of trypsin APMV-8 replicated to a higher titer than in absence of this enzyme (FIG. 19B).

The results of this example showed that, like AMPV-1, APMV-8 is able to penetrate into cells of different species and to initiate its replication cycle. It is therefore a suitable vector for multiple species.

### Production of Recombinant APMV-8 Virus Expressing Foreign Genes Using the Reverse Genetics System

For the generation of a recombinant APMV-8 virus (viral vector) expressing the hemagglutinin (HA) gene of a highly pathogenic avian influenza (HPAI), the coding sequence for the HA gene of HPAI of the H5 or H7 subtype virus is inserted in the non-essential regions, for example, between the M and F genes or between the P and M genes of the APMV-8 genome in the plasmid containing the full length APMV-8 genome. To this end the coding sequence of the hemagglutinin open reading frame is flanked by all the necessary regulatory sequences of the F gene which includes the gene start sequence, the 5′ non-coding sequence, the 3′ non-coding sequence and the gene stop sequence. The construct is synthesized in a way that the restriction enzyme cleavage sites Bsu 36I and Nhe I are used for the ligation of the appropriate fragment into the existing plasmid containing full length APMV-8 genome due to their uniqueness in the plasmid construct. The resulting plasmid is designated transcription plasmid which contains the hemagglutinin gene in the nonessential region of the full length APMV-8 genome. Using this approach the coding sequences of a variety of viral and bacterial antigen can be cloned into the backbone of the APMV-8 sequence. Other possible antigens which could be inserted into the APMV-8 genome are the fusion protein of the Newcastle disease virus, the S protein of avian bronchitis virus, other hemagglutinin genes from non-H5 and non-H7 avian influenza virus, the chicken anemia virus structural protein gene VP1, glycoprotein genes from infectious laryngotracheitis virus.

### Example 7

### Vaccination of Animals

The animals are vaccinated with one, two administrations or a prime-boost regime of the composition or vaccine containing the recombinant APMV-8 virus (viral vector) as described in example 6. For chickens/avian, various administrations are performed, for example, in ovo administration at D18 or D19, subcutaneous (SC) at one-day-old, or mucosal administration (spray, drinking water, eye drop) at different ages. The dose is between 3 and 7 log 10 (preferably 4-6 log 10 EID50). For mammals, mucosal route (intra-nasal, intra-ocular, oral) or parenteral (IM, SC, needle-free, trans-dermal or intra-dermal) is used. The dose ranges from 5 to 9 log (preferably 6-8 log). Two administrations are usually performed at 3-4 weeks interval. Heterologous prime-boost (for instance, boost with proteins) would be also advantageous.

The protective efficacy induced by the composition or vaccine is evaluated against the specific pathogen challenge in the animals. The protective effect is evaluated by clinical observations and/or viral load of the specific pathogen in tissues, blood or mucosal swabs. The blood samples from the vaccinated animals are taken at various stages and tested for serology. The results show that the composition or vaccine of the present invention is immunogenic and provides protection in the vaccinated animals.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### REFERENCES

1. Alexander, 1988. Newcastle disease, p. x, 378 p. In D. J. Alexander (ed.), Developments in veterinary virology. Kluwer Academic, Boston.
2. Alexander, Characterization of viruses which represent further distinct serotypes (PMV-8 and PMV-9) of avian paramyxoviruses. Arch Virol 78:29-36.
3. Alexander, et al., 1979. Properties of a newly isolated, serologically distinct avian paramyxovirus. Arch Virol 60:105-13.
4. Alexander, 2003. Newcastle Disease, other Paramyxoviruses, and Pneumovirus Avian Paramyxoviruses 2-9, p. 63-99. In Y. M. Saif (ed.), Diseases of poultry, 11th ed. Iowa State Press, Ames, Iowa.
5. Andral, et al., 1984. Isolation of avian paramyxovirus 2 and 3 from turkeys in Brittany. Vet Rec 114:570-1.
6. Bankowski, et al., 1981, Effect of paramyxovirus yucaipa on fertility, hatchability, and poult yield of turkeys. Avian Dis 25:517-20.
7. Bankowski, et al., 1960. Isolation of an Unidentified Agent from the Respiratory Tract of Chickens. Science 132:292-293.
8. Bradshaw, et al., 1979. The Epidemiology of Yucaipa Virus in Relationship to the Acute Respiratory Disease Syndrome in Turkeys. Avian Diseases 23:539-542.
9. Capua, et al., 2004. Isolation of an avian paramyxovirus type 9 from migratory waterfowl in Italy. Vet Rec 155:156.
10. Chambers, et al., 1988. Protection of chickens from lethal influenza infection by vaccine expressed hemagglutinin. Virology 167:414-421.
11. Collins, P. L., et al., 1991. Rescue of synthetic analogs of respiratory syncytial virus genomic RNA and effect of truncations and mutations on the expression of a foreign reporter gene. Proc. Natl. Acad. Sci. USA 88:9663-9667
12. Conzelmann K K, and Schnell M., 1994. Rescue of synthetic genomic RNA analogs of rabies virus by plasmid-encoded proteins. J Virol. 68:713-719
13. Darteil, R., M. Bublot, E. Laplace, J. F. Bouquet, J. C. Audonnet and M. Riviere (1995). Herpesvirus of turkey recombinant viruses expressing infectious bursal disease virus (IBDV) VP2 immunogen induce protection against an IBDV virulent challenge in chickens. Virology 211, 481-490.
14. de Leeuw, et al., 1999. Complete nucleotide sequence of Newcastle disease virus: evidence for the existence of a new genus within the subfamily Paramyxovirinae. J Gen Virol 80:131-136.
15. Fleury, et al., 1979. Isolation of twenty-three Yucaipa-like viruses from 616 wild birds in Senegal, West Africa. Avian Dis 23:742-4.
16. Gao, et al., (2006). Protection of mice and poultry from lethal H5N1 avian influenza virus through adenovirus-based immunization. J Virol 80:1959-1964.
17. Ge, et al., (2007) Newcastle disease virus-based live attenuated vaccine completely protects chickens and mice from lethal challenge of homologous and heterologous H5N1 avian influenza viruses. Journal of Virology, 81(1), 150-158.
18. Goodman, et al., 1988. Isolation of avian paramyxovirus-2 from domestic and wild birds in Costa Rica. Avian Dis 32:713-7.
19. Gough, et al., 1984. Avian paramyxovirus type 4 isolated from a ringed teal (Calonetta leucophrys). Vet Rec 115:653.
20. Hoelscher, et al., (2008). A broadly protective vaccine against globally dispersed clade 1 and clade 2 H5N1 influenza viruses. J Infect Dis. 197:1185-1188.
21. Huang, et al., (2004) A recombinant Newcastle Disease Virus (NDV) expressing VP2 protein of Infectious Bursal Disease Virus (IBDV) protects against NDV and IBDV. Journal of Virology, 78, 10054-10063.
22. Hunt, et al., (1988). Retrovirus-expressed hemagglutinin protects against lethal influenza virus infections. J Virol 62:3014-3019.
23. Inoue K, Shoji Y, Kurane I, Iijima T, Sakai T, Morimoto K. (2003). An improved method for recovering rabies virus from cloned cDNA. J Virol Methods. 107:229-236.
24. Krishnamurthy, et al., 1998. Nucleotide sequences of the trailer, nucleocapsid protein gene and intergenic regions of Newcastle disease virus strain Beaudette C and completion of the entire genome sequence. J Gen Virol 79:2419-2424.
25. Krishnamurthy, S., Huang, Z. & Samal, S. K. (2000) Recovery of a virulent strain of Newcastle disease virus from cloned cDNA: expression of a foreign gene results in growth retardation and attenuation. Virology, 278, 168-182.
26. Lamb, et al., 2007. Paramyxoviridae: The viruses and Their Replication, p. 1449-1496. In B. N. Fields, D. M. Knipe, and P. M. Howley (ed.), Fields' virology 5th ed. Wolters Kluwer Health/Lippincott Williams & Wilkins, Philadelphia.
27. Lang, G., A. Gagnon, and J. Howell. 1975. The occurrence of Paramyxovirus yucaipa in Canadian poultry. Can Vet J 16:233-7.
28. Lipkind, et al., 1982. Isolation of yucaipa-like avian paramyxovirus from a wild mallard duck (Anas platyrhinchos) wintering in Israel. Vet Rec 110:15-6.
29. Lipkind, et al., 1979. The isolation of yucaipa-like paramyxoviruses from epizootics of a respiratory disease in turkey poultry farms in Israel. Vet Rec 105:577-8.
30. Maldonado, et al., (1995) Serological survey for avian paramyxoviruses from wildfowl in aquatic habitats in Andalusia. Journal of Wildlife Diseases, 31(1), 66-69.
31. Mayo, et al., 2002. A summary of taxonomic changes recently approved by ICTV. Arch Virol 147:1655-63.
32. Nayak B, et al., (2008). Molecular characterization and complete genome sequence of avian paramyxovirus type 4 prototype strain duck/Hong Kong/D3/75. Virol J. 20; 5:124.
33. Park, et al., (2006) Engineered viral vaccine constructs with dual specificity: Avian influenza and Newcastle disease. Proceedings of the National Academy of Sciences, 103(21), 8203-8208.
34. Peeters, et al., (1999) Rescue of Newcastle disease virus from cloned cDNA: evidence that cleavability of the fusion protein is a major determinant for virulence. Journal of Virology, 73(6), 5001-5009.
35. Redmann, et al., 1991. [Isolation of a paramyxovirus-3 from turkeys with respiratory tract disease in Germany]. Dtsch Tierarztl Wochenschr 98:138-41.
36. Reed, et al., 1938. A SIMPLE METHOD OF ESTIMATING FIFTY PER CENT ENDPOINTS. Am. J. Epidemiol. 27:493-497.
37. Römer-Oberdörfer, et al., (1999) Generation of recombinant lentogenic Newcastle disease virus from cDNA. Journal of General Virology, 80, 2987-2995.
38. Rosenberger, et al., (1974) Isolation of Newcastle disease and type-A influenza viruses from migratory waterfowl in the Atlantic flyway. Avian Diseases, 18(4), 610-613.
39. Saif, et al., 1997. Natural and Experimental Infection of Turkeys with Avian Paramyxovirus-7. Avian Diseases 41:326-329.
40. Shihmanter, et al., 1998. Isolation of avian serotype 3 paramyxoviruses from imported caged birds in Israel. Avian Dis 42:829-31.
41. Shihmanter, et al., 1998. Avian paramyxoviruses serotype 3 isolated from captive birds in Israel: clinical signs, pathology, and antigenic characterization. Avian Dis 42:418-22.
42. Shortridge, et al., 1980. Isolation and properties of viruses from poultry in Hong Kong which represent a new (sixth) distinct group of avian paramyxoviruses. J Gen Virol 49:255-262.
43. Schultz-Cherry, et al., (2000). Influenza virus (A/HK/156/97) hemagglutinin expressed by an alphavirus replicon system protects chickens against lethal infection with Hong Kong-origin H5N1 viruses. Virology 278:55-59.
44. Stallknecht, et al., (1991) Avian paramyxoviruses from migrating and resident ducks in coastal Louisiana. Journal of Wildlive Diseases. 27:123-128.
45. Stanislawek, et al., (2002) Avian paramyxoviruses and influenza viruses isolated from mallard ducks (Anas platyrhynchos) in New Zealand. Archives of Virology, V147, 1287-1302.
46. Tang M, et al., 2002. Recombinant adenovirus encoding the HA gene from swine H3N2 influenza virus partially protects mice from challenge with heterologous virus: A/HK/I/68 (H3N2). Arch Virol 147:2125-2141.
47. Taylor, et al., (1998). Protective immunity against avian influenza induced by a fowlpox virus recombinant. Vaccine 6:504-508.
48. Toro, et al., (2007). Protective avian influenza in ovo vaccination with non-replicating human adenovirus vector. Vaccine 25:2886-2891.
49. Tumova, et al., 1979. A hitherto unreported paramyxovirus of turkeys. Res Vet Sci 27:135-40.
50. Tumova, et al., 1989. Further evidence of the circulation of PMV-4 and influenza viruses with N2-1957 enzyme in the migratory waterfowls. Acta Virol 33:573-6.
51. Veits, et al., (2003). Deletion of the non-essential UL0 gene of infectious laryngotracheitis (ILT) virus leads to attenuation in chickens, and UL0 mutants expressing influenza virus haemagglutinin (H7) protect against ILT and fowl plague. Gen Virol 84:3343-3352.
52. Veits, et al., (2006) Newcastle disease virus expressing H5 hemagglutinin gene protects chickens against Newcastle disease and avian influenza. Proceedings of the National Academy of Sciences, 103(21), 8197-8202.
53. Webster, et al., 1976. Ortho- and paramyxoviruses from migrating feral ducks: characterization of a new group of influenza A viruses. J Gen Virol 32:217-25.
54. Yamane, et al., 1982. Characterization of avian paramyxoviruses isolated from feral ducks in northern Japan: the presence of three distinct viruses in nature. Microbiol Immunol 26:557-68.
55. Zhang, et al., 2007. Serological survey on prevalence of antibodies to avian paramyxovirus serotype 2 in China. Avian Dis 51:137-9.
56. Zhang, et al., 2006. Isolation, identification, and comparison of four isolates of avian paramyxovirus serotype 2 in China. Avian Dis 50:386-90.
57. Zou, et al., 2005. Complete Genome Sequence and Biological Characterizations of A Novel Goose Paramyxovirus-SF02 Isolated in China. Virus Genes 30:13-21.

1-17. (canceled)

18. A composition or vaccine comprising a recombinant APMV viral vector.

19. The composition or vaccine of claim 18, wherein the APMV is APMV-8, APMV-2, APMV-4 or APMV-6.

20. The composition or vaccine of claim 18, wherein the APMV viral vector comprises a polynucleotide having at least 90% sequence identity to a polynucleotide having the sequence as set forth in SEQ ID NO:1 or a polynucleotide complementary to a polynucleotide having at least 90% sequence identity to a polynucleotide having the sequence as set forth in SEQ ID NO:1.

21. The composition or vaccine of claim 18, wherein the APMV viral vector comprises a polynucleotide having at least 90% sequence identity to a polynucleotide encoding a polypeptide having the sequence as set forth in SEQ ID NO:3, a polynucleotide having at least 90% sequence identity to the sequence as set forth in SEQ ID NO:2, or a polynucleotide complementary to a polynucleotide having at least 90% sequence identity to the sequence as set forth in SEQ ID NO:2.

22. The composition or vaccine of claim 18, wherein the APMV viral vector comprises a polynucleotide having at least 90% sequence identity to a polynucleotide encoding a polypeptide having the sequence as set forth in SEQ ID NO:5, a polynucleotide having at least 90% sequence identity to the sequence as set forth in SEQ ID NO:4, or a polynucleotide complementary to a polynucleotide having at least 90% sequence identity to the sequence as set forth in SEQ ID NO:4.

23. The composition or vaccine of claim 18, wherein the APMV viral vector comprises a polynucleotide having at least 90% sequence identity to a polynucleotide encoding a polypeptide having the sequence as set forth in SEQ ID NO:7, a polynucleotide having at least 90% sequence identity to the sequence as set forth in SEQ ID NO:6, or a polynucleotide complementary to a polynucleotide having at least 90% sequence identity to the sequence as set forth in SEQ ID NO:6.

24. The composition or vaccine of claim 18, wherein the APMV viral vector comprises a polynucleotide having at least 90% sequence identity to a polynucleotide encoding a polypeptide having the sequence as set forth in SEQ ID NO:9, a polynucleotide having at least 90% sequence identity the sequence as set forth in SEQ ID NO:8, or a polynucleotide complementary to a polynucleotide having at least 90% sequence identity to the sequence as set forth in SEQ ID NO:8.

25. The composition or vaccine of claim 18, wherein the APMV viral vector comprises a polynucleotide having at least 90% sequence identity to a polynucleotide encoding a polypeptide having the sequence as set forth in SEQ ID NO:11, a polynucleotide having at least 90% sequence identity to the sequence as set forth in SEQ ID NO:10, or a polynucleotide complementary to a polynucleotide having at least 90% sequence identity to the sequence as set forth in SEQ ID NO:10.

26. The composition or vaccine of claim 18, wherein the APMV viral vector comprises a polynucleotide having at least 90% sequence identity to a polynucleotide encoding a polypeptide having the sequence as set forth in SEQ ID NO:13, a polynucleotide having at least 90% sequence identity to a polynucleotide encoding a polypeptide having the sequence as set forth in SEQ ID NO:14, a polynucleotide having at least 90% sequence identity to the sequence as set forth in SEQ ID NO:12, or a polynucleotide complementary to a polynucleotide having at least 90% sequence identity to the sequence as set forth in SEQ ID NO:12.

27. The composition or vaccine of claim 18, wherein the APMV viral vector further comprises an isolated polynucleotide encoding an antigen, and wherein the polynucleotide is inserted in a nonessential region of the APMV genome.

28. The composition or vaccine of claim 18, wherein the nonessential region is selected from the regions consisting of untranslated region located upstream of the NP open reading frame, intergenic regions between two open reading frames of the APMV genome, and untranslated region located downstream of the L open reading frame.

29. A method for producing recombinant APMV viral vector, wherein the method comprises the introduction into the APMV genome an isolated polynucleotide in a nonessential region of the APMV genome.

30. The method of claim 29, wherein the APMV is APMV-8, APMV-2, APMV-4 or APMV-6.

31. The method of claim 29, wherein the APMV comprises a polynucleotide having at least 90% sequence identity to a polynucleotide having the sequence as set forth in SEQ ID NO:1 or a polynucleotide complementary to a polynucleotide having at least 90% sequence identity to a polynucleotide having the sequence as set forth in SEQ ID NO:1.

32. The method of claim 29, wherein the nonessential region is selected from the regions consisting of untranslated region located upstream of the NP open reading frame, intergenic regions between two open reading frames of the APMV genome, and untranslated region located downstream of the L open reading frame.

33. The method of claim 29, wherein the method comprises the steps of:
a) preparing expression plasmids expressing NP, P and L genes;
b) preparing a transcription plasmid comprising an isolated polynucleotide in a nonessential region of the full length APMV genome;
c) transfection of the expression plasmids and transcription plasmid into a host cell;
d) rescuing/recovering the infectious APMV virus from the host cell.

34. The method of claim 29, wherein the method comprises the steps of:
a) preparing expression plasmids expressing NP, P and L genes;
b) preparing a transcription plasmid comprising an isolated polynucleotide in a nonessential region of the full length APMV genome;
c) preparing an expression plasmid expressing T7 polymerase
d) transfection of the expression plasmids and transcription plasmid into a host cell;
e) rescuing/recovering the infectious APMV virus from the host cell.

35. A method for inducing an immunological response in an animal to an antigen comprising inoculating the animal with a composition or vaccine comprising a recombinant APMV viral vector, wherein the recombinant APMV viral vector comprises and expresses the antigen of a pathogen for said animal.

36. The method of claim 35, wherein the immunological response in an animal to the antigen is induced in a prime-boost regime.

37. The method of claim 35, wherein the animal is avian, equine, canine, feline, porcine, bovine, ovine, or human.

38. An unmodified or modified APMV virus comprising a polynucleotide selected from the group consisting of:
a) a polynucleotide having at least 90% sequence identity to a polynucleotide having the sequence as set forth in SEQ ID NO:1 or a polynucleotide complementary to a polynucleotide having at least 90% sequence identity to a polynucleotide having the sequence as set forth in SEQ ID NO:1;
b) a polynucleotide having at least 90% sequence identity to a polynucleotide encoding a polypeptide having the sequence as set forth in SEQ ID NO:3, a polynucleotide having at least 90% sequence identity to a polynucleotide having the sequence as set forth in SEQ ID NO:2, or a polynucleotide complementary to a polynucleotide having at least 90% sequence identity to a polynucleotide having the sequence as set forth in SEQ ID NO:2;
c) a polynucleotide having at least 90% sequence identity to a polynucleotide encoding a polypeptide having the sequence as set forth in SEQ ID NO:5, a polynucleotide having at least 90% sequence identity to a polynucleotide having the sequence as set forth in SEQ ID NO:4, or a polynucleotide complementary to a polynucleotide having at least 90% sequence identity to a polynucleotide having the sequence as set forth in SEQ ID NO:4;
d) a polynucleotide having at least 90% sequence identity to a polynucleotide encoding a polypeptide having the sequence as set forth in SEQ ID NO:7, a polynucleotide having at least 90% sequence identity to a polynucleotide having the sequence as set forth in SEQ ID NO:6, or a polynucleotide complementary to a polynucleotide having at least 90% sequence identity to a polynucleotide having the sequence as set forth in SEQ ID NO:6;
e) a polynucleotide having at least 90% sequence identity to a polynucleotide encoding a polypeptide having the sequence as set forth in SEQ ID NO:9, a polynucleotide having at least 90% sequence identity to a polynucleotide having the sequence as set forth in SEQ ID NO:8, or a polynucleotide complementary to a polynucleotide having at least 90% sequence identity to a polynucleotide having the sequence as set forth in SEQ ID NO:8;
f) a polynucleotide having at least 90% sequence identity to a polynucleotide encoding a polypeptide having the sequence as set forth in SEQ ID NO:11, a polynucleotide having at least 90% sequence identity to a polynucleotide having the sequence as set forth in SEQ ID NO:10, or a polynucleotide complementary to a polynucleotide having at least 90% sequence identity to a polynucleotide having the sequence as set forth in SEQ ID NO:10; and
g) a polynucleotide having at least 90% sequence identity to a polynucleotide encoding a polypeptide having the sequence as set forth in SEQ ID NO:13, a polynucleotide having at least 90% sequence identity to a polynucleotide encoding a polypeptide having the sequence as set forth in SEQ ID NO:14, a polynucleotide having at least 90% sequence identity to a polynucleotide having the sequence as set forth in SEQ ID NO:12, or a polynucleotide complementary to a polynucleotide having at least 90% sequence identity to a polynucleotide having the sequence as set forth in SEQ ID NO:12.

39. The modified APMV virus of claim 38 further comprising an isolated polynucleotide inserted in a nonessential region of APMV genome.

40. The modified APMV virus of claim 38, wherein the APMV virus comprises a polynucleotide having at least 90% sequence identity to the sequence as set forth in SEQ ID NO:1 or a polynucleotide complementary to a polynucleotide having at least 90% sequence identity the sequence as set forth in SEQ ID NO:1.

41. The modified APMV virus of claim 38, wherein the APMV virus comprises a polynucleotide having at least 90% sequence identity to a polynucleotide encoding a polypeptide having the sequence as set forth in SEQ ID NO:3, a polynucleotide having at least 90% sequence identity to the sequence as set forth in SEQ ID NO:2, or a polynucleotide complementary to a polynucleotide having at least 90% sequence identity to the sequence as set forth in SEQ ID NO:2.

42. The modified APMV virus of claim 38, wherein the APMV virus comprises a polynucleotide having at least 90% sequence identity to a polynucleotide encoding a polypeptide having the sequence as set forth in SEQ ID NO:5, a polynucleotide having at least 90% sequence identity to the sequence as set forth in SEQ ID NO:4, or a polynucleotide complementary to a polynucleotide having at least 90% sequence identity to the sequence as set forth in SEQ ID NO:4.

43. The modified APMV virus of claim 38, wherein the APMV virus comprises a polynucleotide having at least 90% sequence identity to a polynucleotide encoding a polypeptide having the sequence as set forth in SEQ ID NO:7, a polynucleotide having at least 90% sequence identity to the sequence as set forth in SEQ ID NO:6, or a polynucleotide complementary to a polynucleotide having at least 90% sequence identity to the sequence as set forth in SEQ ID NO:6.

44. The modified APMV virus of claim 38, wherein the APMV virus comprises a polynucleotide having at least 90% sequence identity to a polynucleotide encoding a polypeptide having the sequence as set forth in SEQ ID NO:9, a polynucleotide having at least 90% sequence identity to the sequence as set forth in SEQ ID NO:8, or a polynucleotide complementary to a polynucleotide having at least 90% sequence identity to the sequence as set forth in SEQ ID NO:8.

45. The modified APMV virus of claim 38, wherein the APMV virus comprises a polynucleotide having at least 90% sequence identity to a polynucleotide encoding a polypeptide having the sequence as set forth in SEQ ID NO:11, a polynucleotide having at least 90% sequence identity to the sequence as set forth in SEQ ID NO:10, or a polynucleotide complementary to a polynucleotide having at least 90% sequence identity to the sequence as set forth in SEQ ID NO:10.

46. The modified APMV virus of claim 38, wherein the APMV virus comprises a polynucleotide having at least 90% sequence identity to a polynucleotide encoding a polypeptide having the sequence as set forth in SEQ ID NO:13, a polynucleotide having at least 90% sequence identity to a polynucleotide encoding a polypeptide having the sequence as set forth in SEQ ID NO:14, a polynucleotide having at least 90% sequence identity to the sequence as set forth in SEQ ID NO:12, or a polynucleotide complementary to a polynucleotide having at least 90% sequence identity to the sequence as set forth in SEQ ID NO:12.

47. An isolated polynucleotide selected from the group consisting of:
a) a polynucleotide having at least 90% sequence identity to a polynucleotide having the sequence as set forth in SEQ ID NO:1 or a polynucleotide complementary to a polynucleotide having at least 90% sequence identity to a polynucleotide having the sequence as set forth in SEQ ID NO:1;
b) a polynucleotide having at least 90% sequence identity to a polynucleotide encoding a polypeptide having the sequence as set forth in SEQ ID NO:3, a polynucleotide having at least 90% sequence identity to a polynucleotide having the sequence as set forth in SEQ ID NO:2, or a polynucleotide complementary to a polynucleotide having at least 90% sequence identity to a polynucleotide having the sequence as set forth in SEQ ID NO:2;
c) a polynucleotide having at least 90% sequence identity to a polynucleotide encoding a polypeptide having the sequence as set forth in SEQ ID NO:5, a polynucleotide having at least 90% sequence identity to a polynucleotide having the sequence as set forth in SEQ ID NO:4, or a polynucleotide complementary to a polynucleotide having at least 90% sequence identity to a polynucleotide having the sequence as set forth in SEQ ID NO:4;
d) a polynucleotide having at least 90% sequence identity to a polynucleotide encoding a polypeptide having the sequence as set forth in SEQ ID NO:7, a polynucleotide having at least 90% sequence identity to a polynucleotide having the sequence as set forth in SEQ ID NO:6, or a polynucleotide complementary to a polynucleotide having at least 90% sequence identity to a polynucleotide having the sequence as set forth in SEQ ID NO:6;
e) a polynucleotide having at least 90% sequence identity to a polynucleotide encoding a polypeptide having the sequence as set forth in SEQ ID NO:9, a polynucleotide having at least 90% sequence identity to a polynucleotide having the sequence as set forth in SEQ ID NO:8, or a polynucleotide complementary to a polynucleotide having at least 90% sequence identity to a polynucleotide having the sequence as set forth in SEQ ID NO:8;
f) a polynucleotide having at least 90% sequence identity to a polynucleotide encoding a polypeptide having the sequence as set forth in SEQ ID NO:11, a polynucleotide having at least 90% sequence identity to a polynucleotide having the sequence as set forth in SEQ ID NO:10, or a polynucleotide complementary to a polynucleotide having at least 90% sequence identity to a polynucleotide having the sequence as set forth in SEQ ID NO:10; and
g) a polynucleotide having at least 90% sequence identity to a polynucleotide encoding a polypeptide having the sequence as set forth in SEQ ID NO:13, a polynucleotide having at least 90% sequence identity to a polynucleotide encoding a polypeptide having the sequence as set forth in SEQ ID NO:14, a polynucleotide having at least 90% sequence identity to a polynucleotide having the sequence as set forth in SEQ ID NO:12, or a polynucleotide complementary to a polynucleotide having at least 90% sequence identity to a polynucleotide having the sequence as set forth in SEQ ID NO:12.

48. The composition or vaccine of claim 28, wherein the nonessential region is the intergenic region between the P gene and M gene.


SEQUENCE LISTING (88 sequences)

SEQ ID NO: 1 - DNA, 15342 bp, artificial sequence
  APMV8 genome sequence
accaaacaaggaatgcaagaccaacgggaactttaaataaaacaatcgaattattgggggcgaagcaagtggatctcgagctcgaggccgaaaccctgaatttcactggaggttttgaataggtcgctataggactcaatatgtcatctgtgttcaatgagtatcaggcgcttcaagaacaacttgtgaagccggctgtcaggagacctgatgttgcctcaacgggtttactcagagcggaaatacctgtctgtgttacattatctcaagaccccggtgagagatggagccttgcttgcttgaatattagatggcttgcgagtgattcatcaaccacaccaatgaagcaaggagcaatattgtcactgctgagtctacattcagacaatatgcgagctcacgcaacattagcagcaaggtctgcagatgcttcactcaccatacttgaggtagatgaagtagatattagcaactcactaatcaaattcaacgccagaagtggtgtatctgacaaacgctcaaatcaattgcttgcaattgcggatgacatccccaaaagttgcagtaatgggcatccatttcttgacacagacattgagaccagagacccgctcgatctatcagagactatagatcgcctgcagggtattgcagctcagatatgggtgtcagccataaagagcatgacagcgcctgacaccgcatcagagtcagaaagtaagaggctggccaaatatcaacaacaaggccgactggttaagcaagtactcttgcattctgtagtcaggacagaatttatgagagttattcggggcagcttggtactgcgccagtttatggttagcgagtgcaagagggcttcagccatgggcggagacacatctaggtactatgctatggtgggtgacatcagtctttacatcaagaatgcaggattgactgcatttttcctcaccctgaagttcggagttggtacccagtatccaaccttagcaatgagtgttttctccagtgaccttaaaaggcttgctgcactcatcaggctatacaaaaccaagggagacaatgcaccatacatggcattcctggaggactccgatatgggaaattttgctccagcaaattatagcacaatgtactcttatgccatgggcattgggacaattctggaagcatctgtatctcgataccagtatgccagagactttaccagtgagaattatttccgtcttggagttgagacagcccaaagccagcagggagcatttgacgagagaacagcccgagaaatgggcttgactgaggaatcaaaacagcaggttagatcactgctaatgtcagtagacatgggtcccagttcaattcatgagccatctcgccctgcatttatcagtcaagaagaaaataggcagcctgcccagaacttgtcagatactcagggtcagaccaagccagtcccgaagcagcccgcaccaagggccgactcagatgacattgatccatacgagaacgggctagaatggtaattcaaccaccccgacacatccacctatacaccaattccgtgacatattaacccaatcaaacatttcataaactatagtagtcattgatttaagaaaaaattgggggcgacctcaattgtgaaacataccagatccgtccacaacaccactcaacaacccacacacaatggatttcgccaatgatgaagaaattgcagaacttttgaatctcagcaccaatgtaatcaaggagattcagaaatccgaactcaagcctccccaaaccaccggacgaccacctgtcagtcaagggaacacaagaaatctaactgatctatgggaaaaggagactgcaagtcagaccaagacaccggcccaatctacacaaaccacacaagttcagtctgatgaaaatgaggagggagaaatcaagtccgagtcaactgatggccacatcagaggaactgttaatcaatcagagcaagtcccagaacaaaaccagagcagatcttcaccaggtgatgatctcgacagagctctcaacaagcttgaagggagaatcaatttaatcagctcaatggacaaagaaattaaaaagggccctcgcatccagaatctccctgggtcccaggcggcaactcaacaggcgacccacccattggcaggggacaccccgaacatgcaagcacagacaaaagccctggcgaagccacatcaagaggcaatcaatcctggcaaccaggacacaggagagagtattcatttaccaccttccatggcaccaccagagtcattagttggtgcaatccgcaatgcaccccaattcgtgccagaccaatctatgacgaatgtagatgcggggagtgtccaactacatgcatcatgtgcagagatgataagtagaatgtttgtagaagttatatccaagcttgataaactcgagtcgagactgaatgatatagcaaaagttgtgaacactaccccccttattaggaatgatattaaccaacttaaggccacaaccgcactgatgtctaaccaaattgcctccatacaaattcttgacccagggaatgcaggggtgaggtccctctctgaaatgaaatctgtgacgaagaaagctgctgttgtaattgcagggtttggagacgacccaactcaaattattgaagaaggcattatggccaaagatgctcttggaaaacctgtgcctccaacatctgttatctcagccaaagctcagacttcttccggtgtgagtaagggtgaaatagaaggattgattgcattggtggaaacattagttgacaatgacaagaaggcagcaaaactgattaaaatgattgatcaagttaaatcccatgccgattacgcccgagtcaagcaggcaatatataatgcgtaatactgtaactacacaaacaatcaatactgctgtcggttgcacccacctcagcaaatcaataatcttttagaatttattgattaagaaaaaattgactactataagaaaagaacaccaagttgggggcgaagacacgattgaccacagtcgctatctgtaaggctcctcaccaaaaatggcatatacaacattgaaactgtgggtggatgagggtgacatgtcgtcttcgctcctatcattcccgttggtactaaaagagacagacagaggcacaaaggagcttcaaccacaggtaagggtagattcaattggcgatgtgcagaacgccaaagagtcctcgatattcgtgactctatatggtttcatccaagcaattaaggagagttcagatcgatcgaaattcttccatccaaaagatgacttcaaacctgagacagtcactgcaggactggtagtggtaggtgcgatccgaatgatggctgatgttaataccatctctaatgacgcactagcgctggagatcactgttaagaaatctgcaacttctcaagagaaaatgacggtgatgttccacaatagccccccttcattgagaactgcaataactatccgagcaggaggtttcatctcgaatgcagacgagaatataaaatgtgccagcaaattgactgcaggagtgcagtacatattccgcccaatgtttgtttcaatcactaaattacacaatggcaaactatatagggtgcccaaaagcatccacagcatctcatccactctactgtatagtgtgatgttggaggtaggattcaaagtggatattgggaaggatcatccccaggcaaagatgctgaagaaggtcacaatcggcgatgcagacacatactgggggtttgcatggttccacctgtgcaatttcaaaaagacatcctctaagggaaagccaagaacgctagacgaactaaagacaaaagtcaaaaatatggggttgaaattggagttacatgacctgtggggtccgactattgtggtccaaatcactggcaagagcagcaaatatgctcaaggatttttttcctccaatggtacttgttgtctcccaatcagcagatctgcaccagagcttgggaagcttctgtggtcttgttcagcaactataggtgacgcaacagttgttatccaatcaagcgagaaaggggaactcctaaggtctgatgacctcgagatacgaggtgctgtggcctccaagaaaggtagactgggctcatttcaccccttcaaaaaatgatgcaggacatagtacagagaattagagagccattagatgtgcgcaaaaaacataatctgcgatgaactgcccagactccactttaatctaggttgcagggaaatagtacacgacatgcgaaatactatcacggtcaccagcaatcaataaagctgatcaatcactatattaggaatcaaataggataacaattattaatccaatttcctaattataaaaaattgctttaaaggttattgacgagtcgggggcgaaatcttgccacttagtctgcagtcaatcttagaatctacatattgaactatgggtcaaatatcagtatatctaattaatagcgtgctattattgctggtatatcctgtgaattcgattgacaatacactcattgccccaatcggagttgccagcgcaaatgaatggcagcttgctgcatatacaacatcactttcagggacaattgccgtgcgattcctacctgtgctcccggataatatgactacctgtcttaaagaaacaatcactacatacaataatactgtcaacaacatcttaggcccactcaaatccaatctggatgcactgctctcatctgagacttatccccagacaagattaattggggcagttataggttcaattgctctcggtgttgcaacatcggctcaaatcactgctgcagttgctctcaagcaagcgcaagacaatgcaaggaacatactagcactcaaagaagcactgtccaaaaccaatgaggcggtcaaggagcttagtagtgggttacaacaaacagctattgcacttggtaagatacagagttttgtgaatgaggaaattctgccatctatcaaccaactgagctgcgaggtgacagccaataaacttggggtgtatttatctctgtatctcacagaactgaccaccatattcggtgcacagctgaccaaccctgcattgacttcattatcatatcaagcactgtacaacctgtgtggtggcaacatggcaatgcttactcagaagattggaattaaacagcaagacgttaattcgctatatgaagccggactaatcacaggacaagtcattggttatgactctcattaccagctgctggtcatccaggtcaattatccaagcatttctgaggtcactggtgtacgtgcgacagaattagtcactgttagtgtaacaacagacaagggtgaagggaaagcaattgtaccccaatttgtagctgaaagtcgggtgactattgaagagcttgatgtcgcatcttgtaaattcagcagcacgaccctatattgcaggcaggtcaacacaagggcacttcccccgctagtagctagctgtcttcgaggtaactatgatgattgtcaatataccacagagattggagcattatcatcccggtatataacactagatgggggggtcttagttaattgcaagtcaattgtttgtaggtgccttaatccaagtaagatcatctctcaaaatacaaacgctgcagtaacatatgttgatgccacaatctgcaaaacaattcaattggatgatatacaactccagctggaagggtcactatcatcagtttatgcaagaaacatctcaattgagatcagtcaggtgaccacatccgggtctttagatatcagcagtgagataggaaacatcaataatacggtgaatcgtgtggaggatttaattcaccaatcagaggaatggctggcaaaggttaacccacacattgttaataatacaacactaattgtactctgtgtgttaagtgcgcttgctgtgatctggctggcagtattaacggctattataatatacttgagaacaaagttgaagactatatcggcattagctgtaaccaatacaatacagtctaacccctatgttaaccaaacgaaacatgaatctaagttttgatcattcaagccaaaacagaggatctaggctcaggttaataatagttcaatcaatatttgatttattaggtttttttcactaattattaatatactcgtgattagatgataacgttaaaagtcttagatatttaataaaaaatgtaacctgggggcgacccatttataggtgagtatatattaggaagtccttatattgcactgtgatttcaaacaattatattacctcatatctaccttgtctaaagacatcatgagtaacattgcatccagtttagaaaacattgtagagcaggatagtcgaaaaacaacttggagggccatctttagatggtccgttcttcttattacaacaggatgcttagccttatccattgttagcatagttcaaattggaaatttgaaaattccttctgtaggggatctggctgatgaagtggtgacacccttgaaaaccactctgtcagatacactcaggaatccaattaaccagataaatgatatatttaggattgttgcccttgatattccattgcaagtgaccagtatccaaaaagaccttgcaagtcaatttaacatgttgatagatagtttaaatgctatcaaattaggcaacgggaccaaccttatcatacctacatcagacaaggagtatgcaggaggaattggaaaccctgtatttactgtcgatgctggaggttctataggattcaaacagtttagcttaatagaacatccgagctttattgctggacctacaacgacccgaggctgtacaagaatacccacttttcacatgtcagaaagtcattggtgctactcacacaacatcatcgctgctggctgtcaagatgccagtgcatccagtatgtatatctcaatgggagttctccatgtgtcctcatctggcactcccatttttcttactactgcaagtgagctgatagacgatggagttaaccgtaagtcatgcagcattgtagcaacccaatttggctgtgacattttgtgcagtattgtcacagagaaggagggagatgattactggtctgatactccgactccaatgcgccacggccgtttttcattcaatggtagttttgtagaagccgaactaccagtgtccagtatgttctcatcattctctgccaactaccctgctgtgggatcaggcgaaattgtaaaagatagaatattattcccaatttacggaggtataaagcagacttcaccagagtttaccgaattagtgaaatacggactctttgtatcaacacctacaactgtgtgccagagtagctggacttatgaccaggtaaaagctgcgtataggccagattacatatcaggccggttctgggcacaagtgatactcagctgcgctcttgatgcagtcgacttatcaagttgtattgtaaagattatgaatagcagcacagtgatgatggcagcggaaggaaggataatgaagatagggattgattacttttactatcagcggtcatcttcttggtggccattggcatttgtcacaaaactagacccgcaagagttggcagacacaaactcaatatggctgaccaattccataccaatcccgcaatcaaagttccctcggccttcatattcagaaaattattgcacaaagccagcagtttgccctgctacttgtgtcactggtgtgtactctgatatttggcccctgacctcatcttcatcactcccgagcataatttggatcggccagtaccttgatgctcctgttggaaggacttatcctagatttggaattgcaaatcagtcacactggtacctccaagaagatattctacccacttccaccgcaagtgcgtattcaaccactacatgttttaagaatactgccaggaatagagtgttctgcgtcaccattgccgaatttgcagatgggttgtttggagagtacaggataacacctcagttgtacgaattagtgagaaataattgaataacaataattttgggactcattttgtcgcaaagtgaaattgtcatctttaaaaataatcaattcgatgatttttattgaacatgattaagcaatcatgtgggaaatttattatctcataaattctaatagttgtaaatgatgaattaagaaaaaatggagggcgacctctacacaaacatggatataaaacaagttgacctgataatacaacccgaggttcatctcgattcacccatcatattgaataaactggcactattatggcgcttgagtggtttacccatgcctgcagacctacgacaaaaatccgtagtgatgcacatcccggaccacatcttagaaaaatcagaatatcggatcaagcaccgtctagggaaaatcaagagtgacataacacattactgtcagtattttaatattaatttggcaaatattgatccgataacccaccccaaaagtttgtattggttatccagactaacaatagctagtgctggaacttttaggcatatgaaagatagaatcttgtgtacagttggctctgaatttggacacaaaattcaagatttattttcactgctgagccataaactagtaggtaacggggatttatttaatcaaagtctctcaggtacacgtttgactgcaagtccgttatcccctttatgcaatcaatttgtctctgacatcaagtctgcagtcacgacaccctggtcagaagctcgttggtcttggcttcatatcaaacaaacaatgagatatctgataaaacaatcacgcactacaaattcggctcatttaacagaaatcataaaagaagaatggggtttagtaggtattactccagatcttgtcattctttttgacagagtcaataatagtctgactgcattaacatttgagatggttctaatgtattcagatgtattagaatcccgtgacaatattgtgttagtggggcgactatctacctttctacagccagtagttagtagactggaggtgttgtttgatctagtagattcattggcaaaaatcttaggtgacacaatatatgagattattgcagtgttagagagcttgtcttatgggtcagttcaactacatgatgcaagtcactctcatgcagggtcttttttttcatttaacatgaatgaacttgataacacactatcaaagagggtagatccgaaacacaagaacactataatgagcattataagacaatgcttttctaatctagatgttgatcaagctgcagagatgctatgcctgatgagattattcggacacccaatgttaactgcaccggatgcagcagccaaagtgaggaaagcaatgtgtgctccaaaacttgttgaacacgacaccatcttgcagacattatctttcttcaaggggataattataaatgggtacagaagatcacactctggcctgtggcccaatgtagagccgtcttcaatttatgatgatgatctcagacagctgtacttagagtcagcagagatttcccatcattttatgcttaaaaactacaagagtttaagcatgatagaattcaagaagagcatagactacgatcttcatgatgacctaagtactttcttaaaggatagagcaatttgccggccgaaatcccagtgggatgtcatatttcgtaagtctttacgcagatctcatacgcagtcccagtatctggacgaaattaagagcaaccgattgctaattgattttcttgattctgctgaatttgaccctggaaaagaatttgcatatgtaaccacaatggattatttgcacgataatgaattttgtgcttcatattctctaaaggaaaaggagatcaaaactactgggaggatatttgcaaaaatgacacgcaatatgagaagttgccaagtaatacttgaatctttgttatcaaagcatatatgcaagttcttcaaagagaatggcgtttcgatggagcaattgtcattgaccaagagtctacttgcaatgtctcaactctcaccaaaagtctcgactttgcaggacactgcatcacgtcatgtaggtaactcaaaatctcagattgcaaccagcaacccatctcggcatcactcgacacccaatcagatgtcactctcaaatcgaaaaacggttgtagcaactttcttaacaactgacttggaaaaatactgcctgcagtggcgatattcaactattaaattgtttgcacaagctctaaatcaactctttgggattgatcacggatttgaatggatacatttaagacttatgaacagcaccttatttgttggcgatccttactcgcctcctgaagatccaacactagaagatatagataaagcaccaaatgatgatatcttcatagtttctccaaggggaggcatagagggtttatgtcagaaaatgtggaccatgatatcaattagtgctatacactgtgtagcagagaaaattggtgcacgagtggcagcaatggtgcagggtgataatcaagtaatagctatcaccaaagaattattcagaggagagaaagcttgtgatgtcagagatgagttagacgagcttggtcaagtgttttttgatgagttcaagagacacaattatgcaattggacacaatcttaagctaaatgagacaatacaaagccaatccttttttgtatattccaaacgaatattctttgaagggcgattgcttagtcaagtcctcaaaaatgctgccaagttatgtatggttgctgaccatctaggtgaaaacactgtatcttcctgtagcaacctgagctcgacaattgcccgcttagtggaaaatgggtttgagaaggacactgcttttgtgttgaacctagtctacatcatgactcagattctttttgatgagcattactcgattgtatgcgatcaccatagtgtcaaaagcttgattggatcaaaaaaccatcggaatttattgtattcatctctaataccaggtcagctcggcggtttcaacttcctcaatataagtcggttgttcactaggaatataggtgacccagtaacatgtagtctgtctgatctcaaatgcttcatagccgcaggtctccttccaccctatgtcctaaaaaatgtggttctgcgtgagcctggtcctgggacatggttgacgttgtgctctgatccttacacccttaacataccatacacacagcttccaaccacatatctcaaaaagcacacccagcgatcattgctttcacgtgcagtaaatcctttattagccggtgtacaagtgccaaatcagcatgaggaagaagagatgttggctcgctttctccttgatcgtgaatatgtgatgccccgcgttgctcatgtaatactagaatcatcagtccttggcaaacggaaacaaatccaaggcttaattgatacaactccaaccatcattagaacatctctagttaatctgccagtgtctagaaagaaatgcgaaaaaataatcaattactctctcaattatattgctgagtgtcatgactccttacttagccaggtctgcttcagtgataataaggaatacttgtggtcaacctccttaatatcagttgagacctgtagtgtgacaattgcggactatctgagagctgtcagctggtctaatatattagggggaagaaacatatccggggtgactacacctgatactattgaattaattcaaggttgtttaataggtgaaaattctagttgtactctttgtgaatcgcatgatgacgcattcacgtggatgcacttgcctggcccactttacatccctgaaccatcagttactaactctaaaatgcgtgtgccatatctgggttcaaaaacagaggagcggaaaacagcctcaatggcagcaataaaaggaatgtcacatcacctgcgtgcagtcttaagaggcacatccgtatttatttgggcatttggggatacagatatcaattgggataatgcattgcagattgcccaatcacggtgtaacatcacattggatcaaatgagattacttacaccaattcctagcagttcaaatattcaacatagactcgatgacggaatcagcacgcagaaatttactcctgcaagccttgctcgaatcacatccttcgttcacatctgtaatgacagccagaggttagagaaggatggctcatctgtcgactcaaacttgatttaccagcaaattatgttacttggactcagcatctttgaaacaatgtactcaatggaccaaaagtgggtattcaataaccataccttgcatttgcacactggacactcctgttgtccaagggaactagacataagtttggtgaacccgccgagacatcagaccccggagctgactagcacaacaaccaacccgttcctatatgatcagctcccattaaatcaagaaaacttgacaacacttgagattaagacatttaaattcaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagacgggccattcttaattacttggacacaactttgcaaaggatccctgtgatacaattagccaacattgcactcaccatttcgcaccctgagatatttcgcagaattgtcaacaccgggatccataaccagattaagggcccatatgtggcaacaacggatttcatagctgcaagtagagatatcatattatcaggtgcaagggagtatctatcttatttaagcagtgggcaggaagactgttacacattcttcaactgtcaagatggggatcttactccaaaaatggaacagtatcttgcaaggagggcatgccttttaacattattgtataatactgggcaccagatccccgttatccgatcactgacgccaatagagaagtgcaaggtgctcacagaatacaatcaacaaattgagtacgcagatcaagagtttagctctgtactaaaagtggtcaatgcactactacaaaatcctaagatagatgcattagtttcaaatctctacttcaccaccagacgtgttctatcaaacctcagatcatgtgataaggctagatcatatattgaatatttgtacactgaggacttcggagagaaagaggatacagtacaatatgacatcatgacaacaaacgatatcatacttactcatggtctattcacacagatcgaaatatcttatcaagggaatagtctccataagttccttactccggataacgcgcctggatctttgatcccattctctatttcaccaaattcacttgcatgtgaccctcttcatcacttgctcaagtcggtcggtacatcaagcacaagttggtacaagtatgcaatcgcctatgcagtgtctgaaaagaggtcagctcgattaggagggagcttgtacattggtgaagggagcggaagtgtgatgactttactcgagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctagggtttgtccagcaatttaaaccgttgtggagagacatagacattgagactaatgttacagatccatcatttatcaattttgcattgaatgaaatcccaatgcaatcattaaaacgagtaaattgtgatgtggaatttgaccgtggtatgccgattgaacgggttattcagggttacacccatatcttacttgttgccacttacggattacagcaagattcaatactgtgggtgaaggtatataggacatctgaaaaagtatttcaattcttactgagtgccatgatcatgatctttggttatgtaaaaatccacaggaatggttatatgtcgacaaaggatgaagagtacatattgatgtctgactgcaaggaacctgtaaactatacagctgtccctaacattcttacacgtgtaagtgatttagtgtcgaagaatctgagtcttatccatccagaagacctcagaaaagtaaggtgtgaaacagattccctgaatttgaagtgcaatcatatttatgagaaaataattgccagaaaaattccattacaggtatcatcaactgactctttgctcctccaattaggcggtgttatcaactcggtgggctcaactgatcctagagaggttgcaacattatcttctattgagtgtatggactatgttgtctcatcaattgatttggctatattggaggcaaatattgtaatctcagagagtgctggtcttgacctcgctttaatgttaggcccattcaacttaaataagcttaagaaaattgacacaatccttaagtcaagcacctatcagctaatcccgtactggttgcgctatgagtactctattaatccgagatctttgtcatttctaatcactaaattacaacaatgccgaatttcatggtcagatatgatcacgatttctgaatttcgtaagaaatccaagcggcctatatttatcaaacgagtaatagggaatcaacagctaaaatcattctttaatgaaagctcaagtattgttttgactcgggctgaagttaaagtctgtataaagttcctcggtgcaatcatcaagttgaaataatttctgcgattttaaaggggtgtaatgttctaatttgcacttgaagtaatatagcttgtaatcattcgctaggggataggataatttctctaacctctgaatctatattcctagagtataacaaatatatacataataaaaatgattttaagaaaaaatccgacactcaaagaaaattggtgcctgtaatattcttcttgccaaatgattgtgaagtgtctagcctaacttaaaacaatcgtattcgatagggaagaatgatatataaaataactaataaaaaattgtattagtaaaaattaccgtatttcctgtattccatttctggt

SEQ ID NO: 2 - DNA, 1386 bp, artificial sequence
  APMV8 NP gene
atgtcatctgtgttcaatgagtatcaggcgcttcaagaacaacttgtgaagccggctgtcaggagacctgatgttgcctcaacgggtttactcagagcggaaatacctgtctgtgttacattatctcaagaccccggtgagagatggagccttgcttgcttgaatattagatggcttgcgagtgattcatcaaccacaccaatgaagcaaggagcaatattgtcactgctgagtctacattcagacaatatgcgagctcacgcaacattagcagcaaggtctgcagatgcttcactcaccatacttgaggtagatgaagtagatattagcaactcactaatcaaattcaacgccagaagtggtgtatctgacaaacgctcaaatcaattgcttgcaattgcggatgacatccccaaaagttgcagtaatgggcatccatttcttgacacagacattgagaccagagacccgctcgatctatcagagactatagatcgcctgcagggtattgcagctcagatatgggtgtcagccataaagagcatgacagcgcctgacaccgcatcagagtcagaaagtaagaggctggccaaatatcaacaacaaggccgactggttaagcaagtactcttgcattctgtagtcaggacagaatttatgagagttattcggggcagcttggtactgcgccagtttatggttagcgagtgcaagagggcttcagccatgggcggagacacatctaggtactatgctatggtgggtgacatcagtctttacatcaagaatgcaggattgactgcatttttcctcaccctgaagttcggagttggtacccagtatccaaccttagcaatgagtgttttctccagtgaccttaaaaggcttgctgcactcatcaggctatacaaaaccaagggagacaatgcaccatacatggcattcctggaggactccgatatgggaaattttgctccagcaaattatagcacaatgtactcttatgccatgggcattgggacaattctggaagcatctgtatctcgataccagtatgccagagactttaccagtgagaattatttccgtcttggagttgagacagcccaaagccagcagggagcatttgacgagagaacagcccgagaaatgggcttgactgaggaatcaaaacagcaggttagatcactgctaatgtcagtagacatgggtcccagttcaattcatgagccatctcgccctgcatttatcagtcaagaagaaaataggcagcctgcccagaacttgtcagatactcagggtcagaccaagccagtcccgaagcagcccgcaccaagggccgactcagatgacattgatccatacgagaacgggctagaatggtaa

SEQ ID NO: 3 - protein, 461 aa, artificial sequence
  APMV8 NP protein
MSSVFNEYQALQEQLVKPAVRRPDVASTGLLRAEIPVCVTLSQDPGERWSLACLNIRWLASDSSTTPMKQGAILSLLSLHSDNMRAHATLAARSADASLTILEVDEVDISNSLIKFNARSGVSDKRSNQLLAIADDIPKSCSNGHPFLDTDIETRDPLDLSETIDRLQGIAAQIWVSAIKSMTAPDTASESESKRLAKYQQQGRLVKQVLLHSVVRTEFMRVIRGSLVLRQFMVSECKRASAMGGDTSRYYAMVGDISLYIKNAGLTAFFLTLKFGVGTQYPTLAMSVFSSDLKRLAALIRLYKTKGDNAPYMAFLEDSDMGNFAPANYSTMYSYAMGIGTILEASVSRYQYARDFTSENYFRLGVETAQSQQGAFDERTAREMGLTEESKQQVRSLLMSVDMGPSSIHEPSRPAFISQEENRQPAQNLSDTQGQTKPVPKQPAPRADSDDIDPYENGLEW

SEQ ID NO: 4 - DNA, 1218 bp, artificial sequence
  APMV8 P gene
atggatttcgccaatgatgaagaaattgcagaacttttgaatctcagcaccaatgtaatcaaggagattcagaaatccgaactcaagcctccccaaaccaccggacgaccacctgtcagtcaagggaacacaagaaatctaactgatctatgggaaaaggagactgcaagtcagaccaagacaccggcccaatctacacaaaccacacaagttcagtctgatgaaaatgaggagggagaaatcaagtccgagtcaactgatggccacatcagaggaactgttaatcaatcagagcaagtcccagaacaaaaccagagcagatcttcaccaggtgatgatctcgacagagctctcaacaagcttgaagggagaatcaatttaatcagctcaatggacaaagaaattaaaaagggccctcgcatccagaatctccctgggtcccaggcggcaactcaacaggcgacccacccattggcaggggacaccccgaacatgcaagcacagacaaaagccctggcgaagccacatcaagaggcaatcaatcctggcaaccaggacacaggagagagtattcatttaccaccttccatggcaccaccagagtcattagttggtgcaatccgcaatgcaccccaattcgtgccagaccaatctatgacgaatgtagatgcggggagtgtccaactacatgcatcatgtgcagagatgataagtagaatgtttgtagaagttatatccaagcttgataaactcgagtcgagactgaatgatatagcaaaagttgtgaacactaccccccttattaggaatgatattaaccaacttaaggccacaaccgcactgatgtctaaccaaattgcctccatacaaattcttgacccagggaatgcaggggtgaggtccctctctgaaatgaaatctgtgacgaagaaagctgctgttgtaattgcagggtttggagacgacccaactcaaattattgaagaaggcattatggccaaagatgctcttggaaaacctgtgcctccaacatctgttatctcagccaaagctcagacttcttccggtgtgagtaagggtgaaatagaaggattgattgcattggtggaaacattagttgacaatgacaagaaggcagcaaaactgattaaaatgattgatcaagttaaatcccatgccgattacgcccgagtcaagcaggcaatatataatgcgtaa

SEQ ID NO: 5 - protein, 405 aa, artificial sequence
  APMV8 P protein
MDFANDEEIAELLNLSTNVIKEIQKSELKPPQTTGRPPVSQGNTRNLTDLWEKETASQTKTPAQSTQTTQVQSDENEEGEIKSESTDGHIRGTVNQSEQVPEQNQSRSSPGDDLDRALNKLEGRINLISSMDKEIKKGPRIQNLPGSQAATQQATHPLAGDTPNMQAQTKALAKPHQEAINPGNQDTGESIHLPPSMAPPESLVGAIRNAPQFVPDQSMTNVDAGSVQLHASCAEMISRMFVEVISKLDKLESRLNDIAKVVNTTPLIRNDINQLKATTALMSNQIASIQILDPGNAGVRSLSEMKSVTKKAAVVIAGFGDDPTQIIEEGIMAKDALGKPVPPTSVISAKAQTSSGVSKGEIEGLIALVETLVDNDKKAAKLIKMIDQVKSHADYARVKQAIYNA

SEQ ID NO: 6 - DNA, 1110 bp, artificial sequence
  APMV8 M gene
atggcatatacaacattgaaactgtgggtggatgagggtgacatgtcgtcttcgctcctatcattcccgttggtactaaaagagacagacagaggcacaaaggagcttcaaccacaggtaagggtagattcaattggcgatgtgcagaacgccaaagagtcctcgatattcgtgactctatatggtttcatccaagcaattaaggagagttcagatcgatcgaaattcttccatccaaaagatgacttcaaacctgagacagtcactgcaggactggtagtggtaggtgcgatccgaatgatggctgatgttaataccatctctaatgacgcactagcgctggagatcactgttaagaaatctgcaacttctcaagagaaaatgacggtgatgttccacaatagccccccttcattgagaactgcaataactatccgagcaggaggtttcatctcgaatgcagacgagaatataaaatgtgccagcaaattgactgcaggagtgcagtacatattccgcccaatgtttgtttcaatcactaaattacacaatggcaaactatatagggtgcccaaaagcatccacagcatctcatccactctactgtatagtgtgatgttggaggtaggattcaaagtggatattgggaaggatcatccccaggcaaagatgctgaagaaggtcacaatcggcgatgcagacacatactgggggtttgcatggttccacctgtgcaatttcaaaaagacatcctctaagggaaagccaagaacgctagacgaactaaagacaaaagtcaaaaatatggggttgaaattggagttacatgacctgtggggtccgactattgtggtccaaatcactggcaagagcagcaaatatgctcaaggatttttttcctccaatggtacttgttgtctcccaatcagcagatctgcaccagagcttgggaagcttctgtggtcttgttcagcaactataggtgacgcaacagttgttatccaatcaagcgagaaaggggaactcctaaggtctgatgacctcgagatacgaggtgctgtggcctccaagaaaggtagactgggctcatttcaccccttcaaaaaatga

SEQ ID NO: 7 - protein, 369 aa, artificial sequence
  APMV8 M protein
MAYTTLKLWVDEGDMSSSLLSFPLVLKETDRGTKELQPQVRVDSIGDVQNAKESSIFVTLYGFIQAIKESSDRSKFFHPKDDFKPETVTAGLVVVGAIRMMADVNTISNDALALEITVKKSATSQEKMTVMFHNSPPSLRTAITIRAGGFISNADENIKCASKLTAGVQYIFRPMFVSITKLHNGKLYRVPKSIHSISSTLLYSVMLEVGFKVDIGKDHPQAKMLKKVTIGDADTYWGFAWFHLCNFKKTSSKGKPRTLDELKTKVKNMGLKLELHDLWGPTIVVQITGKSSKYAQGFFSSNGTCCLPISRSAPELGKLLWSCSATIGDATVVIQSSEKGELLRSDDLEIRGAVASKKGRLGSFHPFKK

SEQ ID NO: 8 - DNA, 1632 bp, artificial sequence
  APMV8 F gene
atgggtcaaatatcagtatatctaattaatagcgtgctattattgctggtatatcctgtgaattcgattgacaatacactcattgccccaatcggagttgccagcgcaaatgaatggcagcttgctgcatatacaacatcactttcagggacaattgccgtgcgattcctacctgtgctcccggataatatgactacctgtcttaaagaaacaatcactacatacaataatactgtcaacaacatcttaggcccactcaaatccaatctggatgcactgctctcatctgagacttatccccagacaagattaattggggcagttataggttcaattgctctcggtgttgcaacatcggctcaaatcactgctgcagttgctctcaagcaagcgcaagacaatgcaaggaacatactagcactcaaagaagcactgtccaaaaccaatgaggcggtcaaggagcttagtagtgggttacaacaaacagctattgcacttggtaagatacagagttttgtgaatgaggaaattctgccatctatcaaccaactgagctgcgaggtgacagccaataaacttggggtgtatttatctctgtatctcacagaactgaccaccatattcggtgcacagctgaccaaccctgcattgacttcattatcatatcaagcactgtacaacctgtgtggtggcaacatggcaatgcttactcagaagattggaattaaacagcaagacgttaattcgctatatgaagccggactaatcacaggacaagtcattggttatgactctcattaccagctgctggtcatccaggtcaattatccaagcatttctgaggtcactggtgtacgtgcgacagaattagtcactgttagtgtaacaacagacaagggtgaagggaaagcaattgtaccccaatttgtagctgaaagtcgggtgactattgaagagcttgatgtcgcatcttgtaaattcagcagcacgaccctatattgcaggcaggtcaacacaagggcacttcccccgctagtagctagctgtcttcgaggtaactatgatgattgtcaatataccacagagattggagcattatcatcccggtatataacactagatgggggggtcttagttaattgcaagtcaattgtttgtaggtgccttaatccaagtaagatcatctctcaaaatacaaacgctgcagtaacatatgttgatgccacaatctgcaaaacaattcaattggatgatatacaactccagctggaagggtcactatcatcagtttatgcaagaaacatctcaattgagatcagtcaggtgaccacatccgggtctttagatatcagcagtgagataggaaacatcaataatacggtgaatcgtgtggaggatttaattcaccaatcagaggaatggctggcaaaggttaacccacacattgttaataatacaacactaattgtactctgtgtgttaagtgcgcttgctgtgatctggctggcagtattaacggctattataatatacttgagaacaaagttgaagactatatcggcattagctgtaaccaatacaatacagtctaacccctatgttaaccaaacgaaacatgaatctaagttttga

SEQ ID NO: 9 - protein, 543 aa, artificial sequence
  APMV8 F protein
MGQISVYLINSVLLLLVYPVNSIDNTLIAPIGVASANEWQLAAYTTSLSGTIAVRFLPVLPDNMTTCLKETITTYNNTVNNILGPLKSNLDALLSSETYPQTRLIGAVIGSIALGVATSAQITAAVALKQAQDNARNILALKEALSKTNEAVKELSSGLQQTAIALGKIQSFVNEEILPSINQLSCEVTANKLGVYLSLYLTELTTIFGAQLTNPALTSLSYQALYNLCGGNMAMLTQKIGIKQQDVNSLYEAGLITGQVIGYDSHYQLLVIQVNYPSISEVTGVRATELVTVSVTTDKGEGKAIVPQFVAESRVTIEELDVASCKFSSTTLYCRQVNTRALPPLVASCLRGNYDDCQYTTEIGALSSRYITLDGGVLVNCKSIVCRCLNPSKIISQNTNAAVTYVDATICKTIQLDDIQLQLEGSLSSVYARNISIEISQVTTSGSLDISSEIGNINNTVNRVEDLIHQSEEWLAKVNPHIVNNTTLIVLCVLSALAVIWLAVLTAIIIYLRTKLKTISALAVTNTIQSNPYVNQTKHESKF

SEQ ID NO: 10 - DNA, 1734 bp, artificial sequence
  APMV8 HN gene
atgagtaacattgcatccagtttagaaaacattgtagagcaggatagtcgaaaaacaacttggagggccatctttagatggtccgttcttcttattacaacaggatgcttagccttatccattgttagcatagttcaaattggaaatttgaaaattccttctgtaggggatctggctgatgaagtggtgacacccttgaaaaccactctgtcagatacactcaggaatccaattaaccagataaatgatatatttaggattgttgcccttgatattccattgcaagtgaccagtatccaaaaagaccttgcaagtcaatttaacatgttgatagatagtttaaatgctatcaaattaggcaacgggaccaaccttatcatacctacatcagacaaggagtatgcaggaggaattggaaaccctgtatttactgtcgatgctggaggttctataggattcaaacagtttagcttaatagaacatccgagctttattgctggacctacaacgacccgaggctgtacaagaatacccacttttcacatgtcagaaagtcattggtgctactcacacaacatcatcgctgctggctgtcaagatgccagtgcatccagtatgtatatctcaatgggagttctccatgtgtcctcatctggcactcccatttttcttactactgcaagtgagctgatagacgatggagttaaccgtaagtcatgcagcattgtagcaacccaatttggctgtgacattttgtgcagtattgtcacagagaaggagggagatgattactggtctgatactccgactccaatgcgccacggccgtttttcattcaatggtagttttgtagaagccgaactaccagtgtccagtatgttctcatcattctctgccaactaccctgctgtgggatcaggcgaaattgtaaaagatagaatattattcccaatttacggaggtataaagcagacttcaccagagtttaccgaattagtgaaatacggactctttgtatcaacacctacaactgtgtgccagagtagctggacttatgaccaggtaaaagctgcgtataggccagattacatatcaggccggttctgggcacaagtgatactcagctgcgctcttgatgcagtcgacttatcaagttgtattgtaaagattatgaatagcagcacagtgatgatggcagcggaaggaaggataatgaagatagggattgattacttttactatcagcggtcatcttcttggtggccattggcatttgtcacaaaactagacccgcaagagttggcagacacaaactcaatatggctgaccaattccataccaatcccgcaatcaaagttccctcggccttcatattcagaaaattattgcacaaagccagcagtttgccctgctacttgtgtcactggtgtgtactctgatatttggcccctgacctcatcttcatcactcccgagcataatttggatcggccagtaccttgatgctcctgttggaaggacttatcctagatttggaattgcaaatcagtcacactggtacctccaagaagatattctacccacttccaccgcaagtgcgtattcaaccactacatgttttaagaatactgccaggaatagagtgttctgcgtcaccattgccgaatttgcagatgggttgtttggagagtacaggataacacctcagttgtacgaattagtgagaaataattga

SEQ ID NO: 11 - protein, 577 aa, artificial sequence
  APMV8 HN protein
MSNIASSLENIVEQDSRKTTWRAIFRWSVLLITTGCLALSIVSIVQIGNLKIPSVGDLADEVVTPLKTTLSDTLRNPINQINDIFRIVALDIPLQVTSIQKDLASQFNMLIDSLNAIKLGNGTNLIIPTSDKEYAGGIGNPVFTVDAGGSIGFKQFSLIEHPSFIAGPTTTRGCTRIPTFHMSESHWCYSHNIIAAGCQDASASSMYISMGVLHVSSSGTPIFLTTASELIDDGVNRKSCSIVATQFGCDILCSIVTEKEGDDYWSDTPTPMRHGRFSFNGSFVEAELPVSSMFSSFSANYPAVGSGEIVKDRILFPIYGGIKQTSPEFTELVKYGLFVSTPTTVCQSSWTYDQVKAAYRPDYISGRFWAQVILSCALDAVDLSSCIVKIMNSSTVMMAAEGRIMKIGIDYFYYQRSSSWWPLAFVTKLDPQELADTNSIWLTNSIPIPQSKFPRPSYSENYCTKPAVCPATCVTGVYSDIWPLTSSSSLPSIIWIGQYLDAPVGRTYPRFGIANQSHWYLQEDILPTSTASAYSTTTCFKNTARNRVFCVTIAEFADGLFGEYRITPQLYELVRNN

SEQ ID NO: 12 - DNA, 6741 bp, artificial sequence
  APMV8 L gene
atggagggcgacctctacacaaacatggatataaaacaagttgacctgataatacaacccgaggttcatctcgattcacccatcatattgaataaactggcactattatggcgcttgagtggtttacccatgcctgcagacctacgacaaaaatccgtagtgatgcacatcccggaccacatcttagaaaaatcagaatatcggatcaagcaccgtctagggaaaatcaagagtgacataacacattactgtcagtattttaatattaatttggcaaatattgatccgataacccaccccaaaagtttgtattggttatccagactaacaatagctagtgctggaacttttaggcatatgaaagatagaatcttgtgtacagttggctctgaatttggacacaaaattcaagatttattttcactgctgagccataaactagtaggtaacggggatttatttaatcaaagtctctcaggtacacgtttgactgcaagtccgttatcccctttatgcaatcaatttgtctctgacatcaagtctgcagtcacgacaccctggtcagaagctcgttggtcttggcttcatatcaaacaaacaatgagatatctgataaaacaatcacgcactacaaattcggctcatttaacagaaatcataaaagaagaatggggtttagtaggtattactccagatcttgtcattctttttgacagagtcaataatagtctgactgcattaacatttgagatggttctaatgtattcagatgtattagaatcccgtgacaatattgtgttagtggggcgactatctacctttctacagccagtagttagtagactggaggtgttgtttgatctagtagattcattggcaaaaatcttaggtgacacaatatatgagattattgcagtgttagagagcttgtcttatgggtcagttcaactacatgatgcaagtcactctcatgcagggtcttttttttcatttaacatgaatgaacttgataacacactatcaaagagggtagatccgaaacacaagaacactataatgagcattataagacaatgcttttctaatctagatgttgatcaagctgcagagatgctatgcctgatgagattattcggacacccaatgttaactgcaccggatgcagcagccaaagtgaggaaagcaatgtgtgctccaaaacttgttgaacacgacaccatcttgcagacattatctttcttcaaggggataattataaatgggtacagaagatcacactctggcctgtggcccaatgtagagccgtcttcaatttatgatgatgatctcagacagctgtacttagagtcagcagagatttcccatcattttatgcttaaaaactacaagagtttaagcatgatagaattcaagaagagcatagactacgatcttcatgatgacctaagtactttcttaaaggatagagcaatttgccggccgaaatcccagtgggatgtcatatttcgtaagtctttacgcagatctcatacgcagtcccagtatctggacgaaattaagagcaaccgattgctaattgattttcttgattctgctgaatttgaccctggaaaagaatttgcatatgtaaccacaatggattatttgcacgataatgaattttgtgcttcatattctctaaaggaaaaggagatcaaaactactgggaggatatttgcaaaaatgacacgcaatatgagaagttgccaagtaatacttgaatctttgttatcaaagcatatatgcaagttcttcaaagagaatggcgtttcgatggagcaattgtcattgaccaagagtctacttgcaatgtctcaactctcaccaaaagtctcgactttgcaggacactgcatcacgtcatgtaggtaactcaaaatctcagattgcaaccagcaacccatctcggcatcactcgacacccaatcagatgtcactctcaaatcgaaaaacggttgtagcaactttcttaacaactgacttggaaaaatactgcctgcagtggcgatattcaactattaaattgtttgcacaagctctaaatcaactctttgggattgatcacggatttgaatggatacatttaagacttatgaacagcaccttatttgttggcgatccttactcgcctcctgaagatccaacactagaagatatagataaagcaccaaatgatgatatcttcatagtttctccaaggggaggcatagagggtttatgtcagaaaatgtggaccatgatatcaattagtgctatacactgtgtagcagagaaaattggtgcacgagtggcagcaatggtgcagggtgataatcaagtaatagctatcaccaaagaattattcagaggagagaaagcttgtgatgtcagagatgagttagacgagcttggtcaagtgttttttgatgagttcaagagacacaattatgcaattggacacaatcttaagctaaatgagacaatacaaagccaatccttttttgtatattccaaacgaatattctttgaagggcgattgcttagtcaagtcctcaaaaatgctgccaagttatgtatggttgctgaccatctaggtgaaaacactgtatcttcctgtagcaacctgagctcgacaattgcccgcttagtggaaaatgggtttgagaaggacactgcttttgtgttgaacctagtctacatcatgactcagattctttttgatgagcattactcgattgtatgcgatcaccatagtgtcaaaagcttgattggatcaaaaaaccatcggaatttattgtattcatctctaataccaggtcagctcggcggtttcaacttcctcaatataagtcggttgttcactaggaatataggtgacccagtaacatgtagtctgtctgatctcaaatgcttcatagccgcaggtctccttccaccctatgtcctaaaaaatgtggttctgcgtgagcctggtcctgggacatggttgacgttgtgctctgatccttacacccttaacataccatacacacagcttccaaccacatatctcaaaaagcacacccagcgatcattgctttcacgtgcagtaaatcctttattagccggtgtacaagtgccaaatcagcatgaggaagaagagatgttggctcgctttctccttgatcgtgaatatgtgatgccccgcgttgctcatgtaatactagaatcatcagtccttggcaaacggaaacaaatccaaggcttaattgatacaactccaaccatcattagaacatctctagttaatctgccagtgtctagaaagaaatgcgaaaaaataatcaattactctctcaattatattgctgagtgtcatgactccttacttagccaggtctgcttcagtgataataaggaatacttgtggtcaacctccttaatatcagttgagacctgtagtgtgacaattgcggactatctgagagctgtcagctggtctaatatattagggggaagaaacatatccggggtgactacacctgatactattgaattaattcaaggttgtttaataggtgaaaattctagttgtactctttgtgaatcgcatgatgacgcattcacgtggatgcacttgcctggcccactttacatccctgaaccatcagttactaactctaaaatgcgtgtgccatatctgggttcaaaaacagaggagcggaaaacagcctcaatggcagcaataaaaggaatgtcacatcacctgcgtgcagtcttaagaggcacatccgtatttatttgggcatttggggatacagatatcaattgggataatgcattgcagattgcccaatcacggtgtaacatcacattggatcaaatgagattacttacaccaattcctagcagttcaaatattcaacatagactcgatgacggaatcagcacgcagaaatttactcctgcaagccttgctcgaatcacatccttcgttcacatctgtaatgacagccagaggttagagaaggatggctcatctgtcgactcaaacttgatttaccagcaaattatgttacttggactcagcatctttgaaacaatgtactcaatggaccaaaagtgggtattcaataaccataccttgcatttgcacactggacactcctgttgtccaagggaactagacataagtttggtgaacccgccgagacatcagaccccggagctgactagcacaacaaccaacccgttcctatatgatcagctcccattaaatcaagaaaacttgacaacacttgagattaagacatttaaattcaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagacgggccattcttaattacttggacacaactttgcaaaggatccctgtgatacaattagccaacattgcactcaccatttcgcaccctgagatatttcgcagaattgtcaacaccgggatccataaccagattaagggcccatatgtggcaacaacggatttcatagctgcaagtagagatatcatattatcaggtgcaagggagtatctatcttatttaagcagtgggcaggaagactgttacacattcttcaactgtcaagatggggatcttactccaaaaatggaacagtatcttgcaaggagggcatgccttttaacattattgtataatactgggcaccagatccccgttatccgatcactgacgccaatagagaagtgcaaggtgctcacagaatacaatcaacaaattgagtacgcagatcaagagtttagctctgtactaaaagtggtcaatgcactactacaaaatcctaagatagatgcattagtttcaaatctctacttcaccaccagacgtgttctatcaaacctcagatcatgtgataaggctagatcatatattgaatatttgtacactgaggacttcggagagaaagaggatacagtacaatatgacatcatgacaacaaacgatatcatacttactcatggtctattcacacagatcgaaatatcttatcaagggaatagtctccataagttccttactccggataacgcgcctggatctttgatcccattctctatttcaccaaattcacttgcatgtgaccctcttcatcacttgctcaagtcggtcggtacatcaagcacaagttggtacaagtatgcaatcgcctatgcagtgtctgaaaagaggtcagctcgattaggagggagcttgtacattggtgaagggagcggaagtgtgatgactttactcgagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctagggtttgtccagcaatttaaaccgttgtggagagacatagacattgagactaatgttacagatccatcatttatcaattttgcattgaatgaaatcccaatgcaatcattaaaacgagtaaattgtgatgtggaatttgaccgtggtatgccgattgaacgggttattcagggttacacccatatcttacttgttgccacttacggattacagcaagattcaatactgtgggtgaaggtatataggacatctgaaaaagtatttcaattcttactgagtgccatgatcatgatctttggttatgtaaaaatccacaggaatggttatatgtcgacaaaggatgaagagtacatattgatgtctgactgcaaggaacctgtaaactatacagctgtccctaacattcttacacgtgtaagtgatttagtgtcgaagaatctgagtcttatccatccagaagacctcagaaaagtaaggtgtgaaacagattccctgaatttgaagtgcaatcatatttatgagaaaataattgccagaaaaattccattacaggtatcatcaactgactctttgctcctccaattaggcggtgttatcaactcggtgggctcaactgatcctagagaggttgcaacattatcttctattgagtgtatggactatgttgtctcatcaattgatttggctatattggaggcaaatattgtaatctcagagagtgctggtcttgacctcgctttaatgttaggcccattcaacttaaataagcttaagaaaattgacacaatccttaagtcaagcacctatcagctaatcccgtactggttgcgctatgagtactctattaatccgagatctttgtcatttctaatcactaaattacaacaatgccgaatttcatggtcagatatgatcacgatttctgaatttcgtaagaaatccaagcggcctatatttatcaaacgagtaatagggaatcaacagctaaaatcattctttaatgaaagctcaagtattgttttgactcgggctgaagttaaagtctgtataaagttcctcggtgcaatcatcaagttgaaataa

SEQ ID NO: 13 - protein, 2246 aa, artificial sequence
  APMV8 L protein1
MEGDLYTNMDIKQVDLIIQPEVHLDSPIILNKLALLWRLSGLPMPADLRQKSVVMHIPDHILEKSEYRIKHRLGKIKSDITHYCQYFNINLANIDPITHPKSLYWLSRLTIASAGTFRHMKDRILCTVGSEFGHKIQDLFSLLSHKLVGNGDLFNQSLSGTRLTASPLSPLCNQFVSDIKSAVTTPWSEARWSWLHIKQTMRYLIKQSRTTNSAHLTEIIKEEWGLVGITPDLVILFDRVNNSLTALTFEMVLMYSDVLESRDNIVLVGRLSTFLQPVVSRLEVLFDLVDSLAKILGDTIYEIIAVLESLSYGSVQLHDASHSHAGSFFSFNMNELDNTLSKRVDPKHKNTIMSIIRQCFSNLDVDQAAEMLCLMRLFGHPMLTAPDAAAKVRKAMCAPKLVEHDTILQTLSFFKGIIINGYRRSHSGLWPNVEPSSIYDDDLRQLYLESAEISHHFMLKNYKSLSMIEFKKSIDYDLHDDLSTFLKDRAICRPKSQWDVIFRKSLRRSHTQSQYLDEIKSNRLLIDFLDSAEFDPGKEFAYVTTMDYLHDNEFCASYSLKEKEIKTTGRIFAKMTRNMRSCQVILESLLSKHICKFFKENGVSMEQLSLTKSLLAMSQLSPKVSTLQDTASRHVGNSKSQIATSNPSRHHSTPNQMSLSNRKTVVATFLTTDLEKYCLQWRYSTIKLFAQALNQLFGIDHGFEWIHLRLMNSTLFVGDPYSPPEDPTLEDIDKAPNDDIFIVSPRGGIEGLCQKMWTMISISAIHCVAEKIGARVAAMVQGDNQVIAITKELFRGEKACDVRDELDELGQVFFDEFKRHNYAIGHNLKLNETIQSQSFFVYSKRIFFEGRLLSQVLKNAAKLCMVADHLGENTVSSCSNLSSTIARLVENGFEKDTAFVLNLVYIMTQILFDEHYSIVCDHHSVKSLIGSKNHRNLLYSSLIPGQLGGFNFLNISRLFTRNIGDPVTCSLSDLKCFIAAGLLPPYVLKNVVLREPGPGTWLTLCSDPYTLNIPYTQLPTTYLKKHTQRSLLSRAVNPLLAGVQVPNQHEEEEMLARFLLDREYVMPRVAHVILESSVLGKRKQIQGLIDTTPTIIRTSLVNLPVSRKKCEKIINYSLNYIAECHDSLLSQVCFSDNKEYLWSTSLISVETCSVTIADYLRAVSWSNILGGRNISGVTTPDTIELIQGCLIGENSSCTLCESHDDAFTWMHLPGPLYIPEPSVTNSKMRVPYLGSKTEERKTASMAAIKGMSHHLRAVLRGTSVFIWAFGDTDINWDNALQIAQSRCNITLDQMRLLTPIPSSSNIQHRLDDGISTQKFTPASLARITSFVHICNDSQRLEKDGSSVDSNLIYQQIMLLGLSIFETMYSMDQKWVFNNHTLHLHTGHSCCPRELDISLVNPPRHQTPELTSTTTNPFLYDQLPLNQENLTTLEIKTFKFNELNIDGLDFGEGIQLLSRCTARLMAECILEEGIGSSVKNEAIVNFDNSVNWISECLMCDIRSLCVNLGQEILCSLAYQMYYLRIRGRRAILNYLDTTLQRIPVIQLANIALTISHPEIFRRIVNTGIHNQIKGPYVATTDFIAASRDIILSGAREYLSYLSSGQEDCYTFFNCQDGDLTPKMEQYLARRACLLTLLYNTGHQIPVIRSLTPIEKCKVLTEYNQQIEYADQEFSSVLKVVNALLQNPKIDALVSNLYFTTRRVLSNLRSCDKARSYIEYLYTEDFGEKEDTVQYDIMTTNDIILTHGLFTQIEISYQGNSLHKFLTPDNAPGSLIPFSISPNSLACDPLHHLLKSVGTSSTSWYKYAIAYAVSEKRSARLGGSLYIGEGSGSVMTLLEYLEPSVDIFYNSLFSNGMNPPQRNYGLMPLQFVNSVVYKNLTAKSECKLGFVQQFKPLWRDIDIETNVTDPSFINFALNEIPMQSLKRVNCDVEFDRGMPIERVIQGYTHILLVATYGLQQDSILWVKVYRTSEKVFQFLLSAMIMIFGYVKIHRNGYMSTKDEEYILMSDCKEPVNYTAVPNILTRVSDLVSKNLSLIHPEDLRKVRCETDSLNLKCNHIYEKIIARKIPLQVSSTDSLLLQLGGVINSVGSTDPREVATLSSIECMDYVVSSIDLAILEANIVISESAGLDLALMLGPFNLNKLKKIDTILKSSTYQLIPYWLRYEYSINPRSLSFLITKLQQCRISWSDMITISEFRKKSKRPIFIKRVIGNQQLKSFFNESSSIVLTRAEVKVCIKFLGAIIKLK

SEQ ID NO: 14 - protein, 2238 aa, artificial sequence
  APMV8 L protein2
MDIKQVDLIIQPEVHLDSPIILNKLALLWRLSGLPMPADLRQKSVVMHIPDHILEKSEYRIKHRLGKIKSDITHYCQYFNINLANIDPITHPKSLYWLSRLTIASAGTFRHMKDRILCTVGSEFGHKIQDLFSLLSHKLVGNGDLFNQSLSGTRLTASPLSPLCNQFVSDIKSAVTTPWSEARWSWLHIKQTMRYLIKQSRTTNSAHLTEIIKEEWGLVGITPDLVILFDRVNNSLTALTFEMVLMYSDVLESRDNIVLVGRLSTFLQPVVSRLEVLFDLVDSLAKILGDTIYEIIAVLESLSYGSVQLHDASHSHAGSFFSFNMNELDNTLSKRVDPKHKNTIMSIIRQCFSNLDVDQAAEMLCLMRLFGHPMLTAPDAAAKVRKAMCAPKLVEHDTILQTLSFFKGIIINGYRRSHSGLWPNVEPSSIYDDDLRQLYLESAEISHHFMLKNYKSLSMIEFKKSIDYDLHDDLSTFLKDRAICRPKSQWDVIFRKSLRRSHTQSQYLDEIKSNRLLIDFLDSAEFDPGKEFAYVTTMDYLHDNEFCASYSLKEKEIKTTGRIFAKMTRNMRSCQVILESLLSKHICKFFKENGVSMEQLSLTKSLLAMSQLSPKVSTLQDTASRHVGNSKSQIATSNPSRHHSTPNQMSLSNRKTVVATFLTTDLEKYCLQWRYSTIKLFAQALNQLFGIDHGFEWIHLRLMNSTLFVGDPYSPPEDPTLEDIDKAPNDDIFIVSPRGGIEGLCQKMWTMISISAIHCVAEKIGARVAAMVQGDNQVIAITKELFRGEKACDVRDELDELGQVFFDEFKRHNYAIGHNLKLNETIQSQSFFVYSKRIFFEGRLLSQVLKNAAKLCMVADHLGENTVSSCSNLSSTIARLVENGFEKDTAFVLNLVYIMTQILFDEHYSIVCDHHSVKSLIGSKNHRNLLYSSLIPGQLGGFNFLNISRLFTRNIGDPVTCSLSDLKCFIAAGLLPPYVLKNVVLREPGPGTWLTLCSDPYTLNIPYTQLPTTYLKKHTQRSLLSRAVNPLLAGVQVPNQHEEEEMLARFLLDREYVMPRVAHVILESSVLGKRKQIQGLIDTTPTIIRTSLVNLPVSRKKCEKIINYSLNYIAECHDSLLSQVCFSDNKEYLWSTSLISVETCSVTIADYLRAVSWSNILGGRNISGVTTPDTIELIQGCLIGENSSCTLCESHDDAFTWMHLPGPLYIPEPSVTNSKMRVPYLGSKTEERKTASMAAIKGMSHHLRAVLRGTSVFIWAFGDTDINWDNALQIAQSRCNITLDQMRLLTPIPSSSNIQHRLDDGISTQKFTPASLARITSFVHICNDSQRLEKDGSSVDSNLIYQQIMLLGLSIFETMYSMDQKWVFNNHTLHLHTGHSCCPRELDISLVNPPRHQTPELTSTTTNPFLYDQLPLNQENLTTLEIKTFKFNELNIDGLDFGEGIQLLSRCTARLMAECILEEGIGSSVKNEAIVNFDNSVNWISECLMCDIRSLCVNLGQEILCSLAYQMYYLRIRGRRAILNYLDTTLQRIPVIQLANIALTISHPEIFRRIVNTGIHNQIKGPYVATTDFIAASRDIILSGAREYLSYLSSGQEDCYTFFNCQDGDLTPKMEQYLARRACLLTLLYNTGHQIPVIRSLTPIEKCKVLTEYNQQIEYADQEFSSVLKVVNALLQNPKIDALVSNLYFTTRRVLSNLRSCDKARSYIEYLYTEDFGEKEDTVQYDIMTTNDIILTHGLFTQIEISYQGNSLHKFLTPDNAPGSLIPFSISPNSLACDPLHHLLKSVGTSSTSWYKYAIAYAVSEKRSARLGGSLYIGEGSGSVMTLLEYLEPSVDIFYNSLFSNGMNPPQRNYGLMPLQFVNSVVYKNLTAKSECKLGFVQQFKPLWRDIDIETNVTDPSFINFALNEIPMQSLKRVNCDVEFDRGMPIERVIQGYTHILLVATYGLQQDSILWVKVYRTSEKVFQFLLSAMIMIFGYVKIHRNGYMSTKDEEYILMSDCKEPVNYTAVPNILTRVSDLVSKNLSLIHPEDLRKVRCETDSLNLKCNHIYEKIIARKIPLQVSSTDSLLLQLGGVINSVGSTDPREVATLSSIECMDYVVSSIDLAILEANIVISESAGLDLALMLGPFNLNKLKKIDTILKSSTYQLIPYWLRYEYSINPRSLSFLITKLQQCRISWSDMITISEFRKKSKRPIFIKRVIGNQQLKSFFNESSSIVLTRAEVKVCIKFLGAIIKLK

SEQ ID NO: 15 - DNA, 15186 bp, artificial sequence
  APMV1 genome sequence
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccattgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcagaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaatgaagggcctggcagctgctgcccaacgggtctccgacgataccagcagcatatacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagttcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatggtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaaggcacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctttgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtccccaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcattaatctcttgattaagaaaaaatgtaagtggcaatgagatacaaggcaaaacagctcatggtaaataatacgggtaggacatggcgagctccggtcctgaaagggcagagcatcagattatcctaccagagccacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaacaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaagacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacaggtgtcaccttacattcacatatccaatgattctcaaaggctgttcactgaagaaggagtcaaagaggggaatgtggtttaccaacagagtcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgctcttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagattatcgctagtcacattgacacagttatccggtctgtgatatatatggaagctgagggtgatctcgctgacacagtatttctatttaccccttacaatctctctactgacgggaaaaagaggacatcacttatacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtattgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 16 - DNA, 14904 bp, artificial sequence
  APMV8 genome sequence
accaaacaaggaataggtaagcaacgtaaatcttagataaaaccatagaatccgtgggggcgacatcgcctgaagccgatctcgagatcgataactccggttaattggtctcagcgtgaggagcttatctgtctgtggcaatgtcttctgtgttttcagaataccaggctcttcaggaccaactggtcaagcctgccactcgaagggctgatgtggcatcgactggattgttgagagcggagataccagtttgtgtaaccttgtctcaggacccaactgatagatggaacctcgcatgtctcaatctgcgatggctgataagtgagtcctctactactcccatgagacaaggggcgatcctgtcactgctgagcttgcactctgacaacatgcgagctcacgcaacccttgcagcgagatccgctgatgctgccatcactgtgcttgaggttgacgccatagacatggcggatggcacaatcacttttaatgccagaagtggagtatccgagaggcgcagcacacagctcatggcaatcgcaaaagatctgccccgctcttgttccaatgactcaccattcaaagatgacactatcgaggatcgcgacccccttgacctgtccgagactatcgatagactgcaggggattgctgcccaaatctggatagcggccatcaagagcatgactgccccggatactgctgcggagtcagaaggcaagaggcttgcaaagtaccaacaacaaggccgcttggtgcgacaggtgttagtgcatgatgcggtgcgtgcggaattcctacgtgtcatcagaggcagcctggtcttacggcaattcatggtatcagaatgtaagagggcagcatccatgggtagcgagacatctaggtactatgccatggtgggtgacatcagcctctacatcaagaatgcaggacttaccgccttcttcttgacactcagatttggtattgggacacactaccccactcttgccatgagtgtgttctctggagaactgaagaagatgtcgtccttgatcaggctgtataagtcaaaaggggaaaatgctgcatacatggcattcctggaggatgcggacatgggaaactttgcgcctgctaactttagtactctctactcctatgcaatgggggtaggtacagtgctggaagcatcagttgcgaaataccagttcgctcgagagttcaccagtgagacatacttcaggcttggggttgagaccgcacagaaccaacagtgcgctctagatgaaaagaccgccaaggagatggggcttactgatgaagccagaaagcaggtgcaagcattggctagcaacatcgagcaggggcaacattcaatgcccatgcaacaacagcccacattcatgagtcagccctaccaggatgacgatcgtgaccagccaagcaccagcagaccagagccaagaccatcgcaattgacaagccaatcagcagcacaggacaatgatgcggcctcattagattggtgaccgcaatcagctcagccaagccattgttggacgcaggacattcaaatcatacattgccctaagagtattaaagtgatttaagaaaaaaggaccctgggggcgaagttgtcccaatccaggcaggcgctgaaaccgaatccctccaacctccgagccccaggcgaccatggagttcaccgatgatgccgaaattgctgagctgttggacctcgggacctcagtgatccaagagctgcagcgagccgaagtcaagggcccgcaaacaaccggaaagcccaaagttcccccggggaacactaagagcctggctactctctgggagcatgagactagcacccaagggagtgcattgggcacacccgagaacaacacccaggcacccgatgacaacaacgcaggtgcagatacgccagcgactaccgacgtccatcgcactctggataccatagacaccgacacaccaccggaagggagcaagcccagctccactaactcccaacccggtgatgaccttgacaaggctctttcgaagctagaggcgcgcgccaagctcggaccagatagggccagacaggttaaaaaggggaaggagatcgggtcgagcacagggacgagggaggcagccagtcaccacatggaagggagccgacagtcggagccaggagcgggcagccgagcacagccacaaggccatggcgaccgggacacaggagggagtactcattcatctctcgagatgggagactggaagtcacaagctggtgcaacccagtctgctctcccattagaagcgagcccaggagagaaaagtgcacatgtggaacttgcccagaatcctgcattttatgcaggcaacccaactgatgcaattatggggttgacaaagaaagtcaatgatctagagacaaaattggctgaggtattgcgtctgttaggaatactccccggaataaagaatgagattagtcagctgaaagcaaccgtggctctgatgtcaaatcagattgcctccattcagattcttgatcctgggaatgccggagtcaaatcccttaatgagatgaaagccctgtcaaaagcagccagcatagttgtggcaggtccaggagtccttcctcctgaggtcacagaaggaggactgatcgcgaaagatgagctagcaaggcccatccccatccaaccgcaacgagactccaaacccaaagacgacccgcacacatcaccaaatgatgtccttgctgtacgcgctatgatcgacacccttgtggatgatgagaagaagagaaagagattaaaccaggcccttgacaaggcaaagaccaaggatgacgtcttaagggtcaagcggcagatatacaatgcctaggagtccatttgtctaaagaacctccaatcatatcaccagtttcgtgccacatgcttccctgccgagaatctagccgacacaaaaactaaatcatagtttaacaaaaaagaagtttgggggcgaagtctcacatcatagagcacccttgcattctaaaatggctcaaacaaccgtcaggctgtatatcgatgaagctagtcccgacattgaactgttgtcttacccactgataatgaaagacacaggacatgggaccaaagagttgcagcagcaaatcagagttgcagagatcggtgcattgcagggagggaagaatgaatcagttttcatcaatgcatatggctttgttcagcaatgcaaagttaaaccgggggcaacccaattcttccaggtagatgcagctacaaagccagaagtggtcactgcagggatgattataatcggtgcagtcaagggggtggcaggcatcactaagctggcagaagaggtgttcgagctggacatctccatcaagaagtccgcatcattccatgagaaggttgcggtgtcctttaatactgtgccactatcactcatgaattcgaccgcatgcagaaatctgggttatgtcacaaacgctgaggaggcgatcaaatgcccgagcaaaatacaagcgggtgtgacgtacaaatttaagataatgtttgtctccttgacacgactgcataacgggaaattgtaccgtgtccccaaggcagtgtatgctgtagaggcatcagctctatataaagtgcaactggaagtcgggttcaagcttgacgtggccaaggatcacccacacgttaagatgttgaagaaagtggaacggaatggtgagactctgtatcttggttatgcatggttccacctgtgcaacttcaagaagacaaatgccaagggtgagtcccggacaatctccaacctagaagggaaagtcagagctatggggatcaaggtttccttgtacgacttatgggggcctactttggtggtgcaaatcacaggtaagaccagcaagtatgcacaaggtttcttttcaaccacaggtacctgctgcctcccagtgtcgaaggctgcccctgagctggccaaacttatgtggtcctgcaatgcaacaatcgttgaagctgcagtgattatccaagggagtgataggagggcagtcgtgacctcagaggacttggaagtatacggggcagttgcaaaagagaagcaggctgcaaaaggatttcacccgttccgcaagtgacacgtggggccgcacacctcattaccccagaagcccgggcaactgcaaattcacgcttatataatccaattaccatgatctagaactgcaatcgatactaatcgctcattgatcgtattaagaaaaaacttaactacataacttcaacattgggggcgacagctccagactaagtgggtggctaagctctgactgataaggaatcatgaatcaagcactcgtgattttgttggtatctttccagctcggcgttgccttagataactcagtgttggctccaataggagtagctagcgcacaggagtggcaactggcggcatatacaacgaccctcacagggaccatcgcagtgagatttatcccggtcctgcctgggaacctatcaacatgtgcacaggagacgctgcaggaatataatagaactgtgactaatatcttaggcccgttgagagagaacttggatgctctcctatctgacttcgataaacctgcatcgaggttcgtgggcgccatcattgggtcggtggccttgggggtagcaacagctgcacaaatcacagccgccgtggctctcaatcaagcacaagagaatgcccggaatatatggcgtctcaaggaatcgataaagaaaaccaatgcggctgtgttggaattgaaggatggacttgcaacgactgctatagctttggacaaagtgcaaaagtttatcaatgatgatattataccacagattaaggacattgactgccaggtagttgcaaataaattaggcgtctacctctccttatacttaacagagcttacaactgtatttggttctcagatcactaatcctgcattatcaacgctctcttaccaggcgctgtacagcttatgtggaggggatatgggaaagctaactgagctgatcggtgtcaatgcaaaggatgtgggatccctctacgaggctaacctcataaccggccaaatcgttggatatgaccctgaactacagataatcctcatacaagtatcttacccaagtgtgtctgaagtgacaggagtccgggctactgagttagtcactgtcagtgtcactacaccaaaaggagaagggcaggcaattgttccgagatatgtggcacagagtagagtgctgacagaggagttggatgtctcgacttgtaggtttagcaaaacaactctttattgtaggtcgattctcacacggcccctaccaactttgatcgccagctgcctgtcagggaagtacgacgattgtcagtacacaacagagataggagcgctatcttcgagattcatcacagtcaatggtggagtccttgcaaactgcagagcaattgtgtgtaagtgtgtctcacccccgcatataataccacaaaacgacattggctccgtaacagttattgactcaagtatatgcaaggaagttgtcttagagagtgtgcagcttaggttagaaggaaagctgtcatcccaatacttctccaacgtgacaattgacctttcccaaatcacaacgtcagggtcgctggatataagcagtgaaattggtagcattaacaacacagttaatcgggtcgacgagttaatcaaggaatccaacgagtggctgaacgctgtgaacccccgccttgtgaacaatacgagcatcatagtcctctgtgtccttgccgccctgattattgtctggctaatagcgctgacagtatgcttctgttactccgcaagatactcagctaagtcaaaacagatgaggggcgctatgacagggatcgataatccatatgtaatacagagtgcaactaagatgtagagaggttgaataagcctaaacatgatatgatttaagaaaaaattggaaggtgggggcgacagcccattcaatgaagggtgtacactccaacttgatcttgtgacttgatcatcatactcgaggcaccatggatttcccatctagggagaacctggcagcaggtgacatatcggggcggaagacttggagattactgttccggatcctcacattgagcataggtgtggtctgtcttgccatcaatattgccacaattgcaaaattggatcacctggataacatggcttcgaacacatggacaacaactgaggctgaccgtgtgatatctagcatcacgactccgctcaaagtccctgtcaaccagattaatgacatgtttcggattgtagcgcttgacctacctctgcagatgacatcattacagaaagaaataacatcccaagtcgggttcttggctgaaagtatcaacaatgttttatccaagaatggatctgcaggcctggttcttgttaatgaccctgaatatgcaggggggatcgctgtcagcttgtaccaaggagatgcatctgcaggcctaaatttccagcccatttctttaatagaacatccaagttttgtccctggtcctactactgctaagggctgtataaggatcccgaccttccatatgggcccttcacattggtgttactcacataacatcattgcatcaggttgccaggatgcgagccactccagtatgtatatctctctgggggtgctgaaagcatcgcagaccgggtcgcctatcttcttgacaacggccagccatctcgtggatgacaacatcaaccggaagtcatgcagcatcgtagcctcaaaatacggttgtgatatcctatgcagtattgtgattgaaacagagaatgaggattataggtctgatccggctactagcatgattataggtaggctgttcttcaacgggtcatacacagagagcaagattaacacagggtccatcttcagtctattctctgctaactaccctgcggtggggtcgggtattgtagtcggggatgaagccgcattcccaatatatggtggggtcaagcagaacacatggttgttcaaccagctcaaggattttggttacttcacccataatgatgtgtacaagtgcaatcggactgatatacagcaaactatcctggatgcatacaggccacctaaaatctcaggaaggttatgggtacaaggcatcctattgtgcccagtttcactgagacctgatcctggctgtcgcttaaaggtgttcaataccagcaatgtgatgatgggggcagaagcgaggttgatccaagtaggctcaaccgtgtatctataccaacgctcatcctcatggtgggtggtaggactgacttacaaattagatgtgtcagaaataacttcacagacaggtaacacactcaaccatgtagaccccattgcccatacaaagttcccaagaccatctttcaggcgagatgcgtgtgcgaggccaaacatatgccctgctgtctgtgtctccggagtttatcaggacatttggccgatcagtacagccaccaataacagcaacattgtgtgggttggacagtacttagaagcattctattccaggaaagacccaagaatagggatagcaacccagtatgagtggaaagtcaccaaccagctgttcaattcgaatactgagggagggtactcaaccacaacatgcttccggaacaccaaacgggacaaggcatattgtgtagtgatatcagagtacgctgatggggtgttcggatcatacaggatcgttcctcagcttatagagattagaacaaccaccggtaaatctgagtgatgcatcaatcctaaattggaatgaccaatcaaaagctacgtagtgtctaacagcattgcgaagcctggtttaagaaaaaacttgggggcgaatgcccatcaaccatggatcaaactcaagctgacactataatacaacctgaagtccatctgaattcaccacttgttcgcgcaaaattggttcttctatggaaattgactgggttacctttgccgtctgatttgagatcatttgtactaactacacatgcagctgatgaccaaatcgcaaaaaatgagactaggatcaaggccaaaattaattccctaatcgataacttaatcaaacactgcaaggcaaggcaagtggcactttcagggttgacacctgtcgtacatccaacaactctacagtggttgctatccatcacatgtgaacgagcagaccaccttgcaaaagtacgcgagaaatcagttaagcaagcaatgtcagagaagcaacacgggtttagacatctcttttcggcagtaagtcatcagttagttggaaacgccacactgttctgtgcacaagactctagcaccgtgaatgtcgactctccttgctcatcaggttgtgagaggctgataatagactctattggagccttacaaacacgatggacaagatgtaggtgggcttggcttcacattaaacaggtaatgagataccaggtgcttcagagtcgcctacacgctcatgccaattctgttagcacatggtctgaggcgtgggggttcattgggatcacaccagatatagtccttattgtagactataagagcaaaatgtttactatcctgaccttcgaaatgatgctgatgtattcagatgtcatagagggtcgtgataatgtggtagctgtaggaagtatgtcaccaaacctacagcctgtggtggagaggattgaggtgctgtttgatgtagtggacaccttggcgaggaggattcatgatcctatttatgatctggttgctgccttagaaagcatggcatacgctgccgtccaattgcacgatgctagtgagacacacgcaggggaattcttttcgttcaatttgacagaaatagagtccactcttgcccccttgctggatcctggccaagtcctatcggtgatgaggactatcagttattgttacagtgggctatcgcctgaccaagctgcagagttgctctgtgtgatgcgcttatttggacaccctctgctctccgcacaacaagcagccaaaaaagtccgggagtctatgtgtgcccctaaactgttagagcatgatgcaatactgcaaactctatctttcttcaagggaatcataatcaatggctacaggaaaagtcattctggagtatggcctgcaattgacccagattctatagtggacgatgaccttagacagctgtattacgagtcggcagaaatttcacatgctttcatgcttaagaaatatcggtaccttagtatgattgagttccgcaagagcatagagtttgacttaaatgatgacctgagcacattccttaaagacaaagcaatctgcaggccaaaagatcaatgggcacgcatcttccggaaatcattgttcccttgcaaaacgaaccttggcactagtatagatgttaaaagtaatcgactgttgatagattttttggagtcacatgacttcaatcctgaggaagaaatgaagtatgtgactacgctagcatacctggcagataatcaattctcagcatcatattcactgaaggagaaagagatcaagactactggccggatcttcgccaaaatgaccaggaaaatgaggagctgtcaagtaatattggaatcactattgtccagtcacgtctgcaaattctttaaggagaacggtgtgtcaatggaacaactgtctttgacaaagagcttgcttgcaatgtcacagttagcacccaggatatcttcagttcgccaggcgacagcacgtagacaggacccaggactcagccactctaatggttgtaatcacattgtaggagacttaggcccacaccagcaggacagaccggcccggaagagtgtagtcgcaaccttccttacaacagatcttcaaaaatattgcttgaattggcgatatgggagtatcaagcttttcgcccaagccttaaaccagctattcggaatcgagcatgggtttgaatggatacacctgagactgatgaatagcaccctgtttgtcggggacccattctcgcctcctgaaagcaaagtgctgagtgatcttgatgatgcgcccaattcagacatatttatcgtgtccgccagaggggggattgaagggttatgccagaagctgtggaccatgatttcaataagcataatccattgcgtggctgagaagataggagcaagggttgcggcgatggttcagggagataatcaggtaattgcaatcacgagagagctgtataagggagagacttacacgcagattcagccggagttagatcgattaggcaatgcattttttgctgaattcaaaagacacaactatgcaatgggacataatctgaagcccaaagagacaatccaaagtcaatcattctttgtgtattcgaaacggattttctgggaagggagaattcttagtcaagcactgaagaatgctaccaaactatgcttcattgcagatcacctcggggataatactgtctcatcatgcagcaatctagcctctacgataacccgcttggttgagaatgggtatgaaaaggacacagcattcattctgaatatcatctcagcaatgactcagttgctgattgatgagcaatattccctacaaggagactactcagctgtgagaaaactgattgggtcatcaaattaccgtaatctcttagtggcgtcgctcatgcctggtcaggttggcggctataatttcttgaatatcagtcgcctattcacacgcaatattggtgatccagtaacatgcgccatagcagatctgaagtggttcattaggagcgggttaatcccagagttcatcctgaagaatatattactacgagatcccggagacgatatgtggagtactctatgtgctgacccttacgcattaaatatcccctacactcagctacccacaacatacctgaagaagcatactcagagggcattactatccgattctaataatccgcttcttgcaggggtgcaattggacaatcaatacattgaagaggaggagtttgcacgattccttttggatcgggaatccgtgatgcctcgagtggcacacacaatcatggagtcaagtatactagggaagagaaagaacatccagggtttaatcgacactacccctacaatcattaagactgcactcatgaggcagcccatatctcgtagaaagtgtgataaaatagttaattactcgattaactacctgactgagtgccacgattcattattgtcctgtaggacattcgagccaaggaaggaaataatatgggagtcagctatgatctcagtagaaacttgcagtgtcacaattgcggagttcctgcgcgccaccagctggtccaacatcctgaacggtaggactatttcgggtgtaacatctccagacactatagagctgctcaaggggtcattaattggagagaatgcccattgtattctttgtgagcagggagacgagacattcacgtggatgcacttagccgggcccatctatataccagacccgggggtgaccgcatccaagatgagagtgccgtatcttgggtcaaagacagaggaaaggcgtacggcatccatggccaccattaagggcatgtctcaccacctaaaggccgctttgcgaggagcctctgtgatggtgtgggcctttggtgatactgaagaaagttgggaacatgcctgccttgtggccaatacaaggtgcaagattaatcttccgcagctacgcctgctgaccccgacaccaagcagctctaacatccaacatcgactaaatgatggtatcagcgtgcaaaaatttacacctgctagcttatcccgagtggcgtcatttgttcacatttgcaacgatttccaaaagctagagagagatggatcttccgtagactctaacttgatatatcagcaaatcatgctgactggtctaagtattatggagacacttcatcctatgcacgtctcatgggtatacaacaatcagacaattcacttacataccggaacatcgtgttgtcctagggaaatagagacaagcattgttaatcccgctaggggagaattcccaacaataactctcacaactaacaatcagtttctgtttgattgtaatcccatacatgatgaggcacttacaaaactgtcagtaagtgagttcaagttccaggagcttaatatagactcaatgcagggttacagtgctgtgaacctgctgagcagatgtgtggctaagctgataggggaatgcattctggaagacggtatcggatcgtcaatcaagaatgaagcaatgatatcatttgataactctatcaactggatttctgaagcactcaatagtgacctgcgtttggtattcctccagctggggcaagaactactttgtgacctggcgtaccaaatgtactatctgagggtcatcggctatcattccatcgtggcatatctgcagaatactctagaaagaattcctgttatccaactcgcaaacatggcactcaccatatcccacccagaagtatggaggagagtgacagtgagcggattcaaccaaggttaccggagtccctatctggccactgtcgactttatcgccgcatgtcgtgatatcattgtgcaaggtgcccagcattatatggctgatttgttgtcaggagtagagtgccaatatacattctttaatgttcaagacggcgatctgacaccgaagatggaacaatttttagcccggcgcatgtgcttgtttgtattgttaactgggacgatccgaccactcccaatcatacgatcccttaatgcgattgagaaatgtgcaattctcactcagttcttgtattacctaccgtcagtcgacatggcagtagcagacaaggctcgtgtgttatatcaactgtcaataaatccgaaaatagatgctttagtctccaacctttatttcaccacaaggaggttgctttcaaatatcaggggagattcttcttcacgagcgcaaattgcattcctctacgaggaggaagtaatcgttgatgtgcctgcatctaatcaatttgatcagtaccatcgtgaccccatcctaagaggaggtctatttttctctctctccttaaaaatggaaaggatgtctctgaaccgatttgcagtacagaccctgccaacccaggggtctaactcgcagggttcacgacagaccttgtggcgtgcctcaccgttagcacactgccttaaatcagtagggcaggtaagtaccagctggtacaagtatgctgtagtgggggcgtctgtagagaaagtccaaccaacaagatcaacaagcctctacatcggggagggcagtgggagtgtcatgacattattagagtatctggaccctgctacaattatcttctacaactcgctattcagcaatagcatgaaccctccacaaaggaatttcggactgatgcccacacagtttcaggactcagtcgtgtataaaaacatatcagcaggagttgactgcaagtacgggtttaagcaagtctttcaaccattatggcgtgatgtagatcaagaaacaaatgtggtagagacggcgttcctaaactatgtgatggaagtagtgccagtccactcttcgaagcgtgtcgtatgtgaagttgagtttgacagggggatgcctgacgagatagtaataacagggtacatacacgtgctgatggtgaccgcatacagtctgcatcgaggagggcgtctaataatcaaggtctatcgtcactccgaggctgtattccaattcgtactctctgcgatagtcatgatgtttggggggcttgatatacaccggaactcgtacatgtcaactaacaaagaggagtacatcatcatagctgcggcgccggaggcattaaactattcctctgtaccagcaatattgcagagggtgaagtctgttattgaccagcagcttacattaatctctcctatagatctagaaagattgcgccatgagactgagtctctccgtgagaaggagaataatctagtaatatctctgacgagagggaagtatcaactccggccgacacagactgatatgcttctatcatacctaggtgggagattcatcaccctattcggacagtctgctagggatttgatggccactgatgttgctgaccttgatgctaggaagattgcattagttgatctactgatggtggaatccaacattattttaagtgagagcacagacttggaccttgcactgttgctgagcccgtttaacttagacaaagggcggaagatagttaccctagcaaaggctactacccgccaattgctgcccgtgtatatcgcatcagagataatgtgcaatcggcaggcattcacacacctgacatcaattatacagcgtggtgtcataagaatagaaaacatgcttgctacaacggaatttgtccgacagtcagttcgcccccagttcataaaggaggtgataactatagcccaagtcaaccaccttttttcagatctatccaaactcgtgctttctcgatctgaagtcaagcaagcacttaaatttgtcggttgctgtatgaagttcagaaatgcaagcaattaaacaggattgttattgtcaaatcaccggttactatagtcaaattaatatgtaaagttccctctttcaagagtgattaagaaaaaacgcgtcaaaggtggcggtttcactgatttgctcttggaagttgggcatcctccagccaatatatcggtgccgaaatcgaaagtctgacagctgatttggaatataagcactgcataatcactgagttacgttgctttgctattccatgtctggt

SEQ ID NO: 17 - DNA, 16272 bp, artificial sequence
  APMV3 genome sequence
actaaacagaaaattaatacttgttagtagtcgccgattaatccgctaatatattaggagcggaagtcctacactccgcctccgaccacgaattgaaatcatcatggctggaatttttaatacttatgagctctttgttaaggatcagacatgtatgcacaagagggctgcaagcctaatatcgggaggtcagcttaaaagcaatataccagtcttcatcaccacaaaagatgatccagctgtccgatgggaccttgtatgttttaatctccgacttgttgttagtgaatcatctacctcggtgatccggcagggggctcttatatcattgctttccattactgctagcaatatgagggctcttgcagcaattgctgggcagactgatgaatcgatgattaatattatagaagttgtggactttgatggacttgagccccagtgtgacactcgcagtggcttagacgctcagaagcaggagatgtttaaggatattgcccgtgatatgccgagggcactatccagcgggaccccgttccagcacggaaatgcggaagcgaatgggcctgaagacacacacatgttcctccgatccgcaattagcgttctgacacagatctggatcctggttgcaaaagccatgactaacattgatggcagcaacgaggcaagcgatcgccgccttgccaagtatattcaacagaatagaattgacaggcgattcatgcttgctcaagctaccaggactgtatgtcagcaggtcataaaggattctttgaccttgagaaggttccttgtaactgagcttcgaaagtcgcggggggcattgcatagcgggtcatcttactatgctatgattggagacatgcaggcatacatatttaatgcaggccttacaccattcctcactaccctcaggtacgggatcgggactaaataccatgcccttgcaattagctcacttgcaggtgacctgaataagattaaggggttactgactctatacaaagagaaagggagtgatgcgggctatatggcacttctagaggatgctgactgcatgcaatttgcgccaggtaattactcactattatattcatatgcgatgggagtagcaagtgtacatgatgaaggcatgaggaattaccaatacgcccggcgcttcctccataaggggatgtaccaatttggacgtgatatcgcaacacaacagcaacatgcacttgatgaatccctcgcccaggagatgagaatcacagaggctgacagggcaaatctcaaggtaatgatggcaaacattggggaagcatcaagcttcagtgatatgcctaatccaggagctagcggcattcctgccttcaatgaccgccctgatgagatcttctctgagccaggctatcgcaagccccctcaagatcagccgctgataaagctcccagatctcgaggaagaagagcaggacgaattcgacatgtagagagccgcccaccacccagagccagcgacaaacccaggcaaaacaacccaccatctccgacccggaacacaacgcaacatcgaggaccccgggcagccgacaccgcagccgcaagacctccgcatcatgaacgaccagcatccaaacatctgtcctccctattctatcagtttaataaaaaaggctttcgtgcacagacagagatcctgacataattcagccggggggattcaacttaggagcggaagtatccctcctaacctgcacctgcatttcaatatggatcttgagtttagtagtgaggaagcagtcgcagccctcttggatgtgagttcctcaaccataaccgaggttctgagcaagcaaagtattcctgaccctagctttctcacgtccacggaggcatccggtgagcaagttccagattcgaaggtcgccaatcacaaagccagtgtcgataagaccccagatcaaggccaaccatcggcaacgccttcggctcctcctgagactgccgaaaacatcaatacaccatcttgcgaggacggtttaccccctaacttcttcatcccaagggtagagagttaccacactaacctttttaaagggggcccccaattggactcaacggagcggccgcctggtcaccaaatagtttgcggagatcaagacgcggacctctcccgagccgggatagcgagaaagaagaagaagcagaagcattgcaaagcgctcaacttttcggaatccccactcacagacaatcaggccatagaagggagtattcaatctcatggagccctggatcaggaaccttccagacagagacctggtgcaacccagcctgctctccagtcaccgccttcccaaaacaatacaagtgtgcatgcagacagtgcccaagattctgcgatctctgtttcaatcccgctgactatggtggaatcattgatctctcaagtgtcaaagttgtcggaccaagtctcgcagatacagaagttggtgagtacccttcctcaaattaagactgatattgcttcgattcggaacatgcaagcagctttggaaggacagctcagtatgattcgtatattggatccaggaaactgttcggaatcatcactcaatgcgctgagagggttcagtggtaaggcacctgttattgtgagtggtccaggaaatcccaatcaatgcatcagccaaggctcatccaccacaatttgcctggatgagttagcccggcctgtgccaaatcctatacaggtgaaacttcaagatgatcagaaggacctatctgcacagcgacatgctgtcattgcattactcgaaaccagaatccccccgggaccaaaacgtgacaaattactgtcattggttgcaattgcaaagactgcaagtgatttaatcaagattaagagaatggcagtcctcggccagtgatatcaccctatggcaaccaggctacaaatgtaagcagtttccgatatgtaccgaatcaataacacgaccccttccgcaaaagcaagcgcgagcatgacacactcagcttcagccaattctagcttcactaagcaggtgatctgaccatctggctttacttccgctttataaaaaactcctagatctaacatggaggcagaccgagctaatgcggagggagtacacaaggggaatcaaaggagcggaaggtactaagtcactccaccgcctgccctcgcccatccgcagcctcccccaacccgcggcccaggctccattcaaaaaccatggccgctgctctcgggaacatgcacccgtcatcgtcgattactcttatgcatgatgacccgtccatccagacgcagttgctagccttccctctgataagtgagaagacagagacagggacgacaaagctgcagcctcaagtgagaatgcaatctttcctatcaaccgatagccagaagtatcatctggtctttatcaacacatatggtttcattgcggaggattttaattgtagtccagcaaacggatttgtgcctgcactatttcaacccaagtctaaagtcttatcgtcagcgatggttacacttggggctgtccctgctgacactgttctccaggacctacaacgggacttaatcgcaatgagatttaaagtccgaaagagtgcgtcacgaatggagctaatattattctccacagacaatgtccctgcaacattaacagggtcatcagtctggaagaataaaggagtaatagctgatacagcaactgcagttaaagcgcccggtcgcatctcatgcgatgcggtatgcagttattgcattacattcatctctttttgcttcttccactcatctgctctttttaaggtgcccaagcctctgctgaatttcgagacggccatagcatattcccttgtactccaggtcgagcttgagttcccaaatatcaaagataccttacacgagaagtatctcaaagttaaagactcgaagtggtattgtacaattgacatccatattgggaatcttttaaagagaaccgccaagcaacgaaggaggacaccttcagagataatgcagaaagtccgtaagatgggtttcaggattgggttatacgatttgtggggtccaacaattgtggttgaattaactgggtcctcaagtaagtccctacacggattcttctcgagtgagcggctggcctgccacccggtatcacagtataatcctcatgttggacagcttatctgggcacacgatgtatccatcacggggtgccacatgattatatctgaacttgagaaaaagaaggcacttgcaatggctgacctgaccgtgagtgatgcagtagctgtcaacacgagcatcaagagcttatcgcctttcagactgttcaaaaaatataagggcaagacaactccaagggtgtgtgcttataatttgcatattctagattataatcattaaccgcgtccaaatgatcagaccgcataattaacagctcagccaggactcgcaaatcctgcataggctcaaataacaagacgacatatatactcacacaacaaggtgtaatatctagaaggaatcagaattgagacaatatctaggttatcagcgctactgccctgcaactcactatgtatgcttggcattaataaaaaacattgtgagaatgcaatttaaggacgaaagtaggagcggaacacgctcaactgaacccaagcgcacaccagccccccggcaccgcaccggaaggacgcacaacagccgaccgcccccaccagggcagaggccatgcaaccaggctcggcactccacctcccgcacctgtacataataattgccttagtcagcgatggcactttaggccaaacagctaaaattgataggctgatacaggcagggattgtcttgggtagtggaaaggagctccatatctcacaagattcagggacactggatttattcgtgaggttgttaccagtactcccttcgaatttgtcccattgccagcttgaagcaataacacagtataataagacggtgactaggttattggccccaatagggaagaacctagaacaggtactacaagcgagaccgcgtgggaggctgttcggtccaataatagggtccatcgcgcttggtgttgccacatcagcacagattaccgctgctattgcactcgtccgtgcacagcaaaatgctaatgatatattagcacttaaaaacgctctccaatccagcaatgaggcaatccgtcaactcacgtatggccaggacaaacagttacttgcaatttctaaaatacaaaaagctgtgaatgagcagatcctccctgcactcgatcagctggactgtgctgtccttggaaccaaactagctgtgcagcttaatctgtatctcattgagatgactaccattttcggcgagcaaatcaataatccagtcttggcgaccatcccattaagttacattttacgcctgaccggtgcggagttgaacaacgtcctcatgaagcaagcccgatcgtccttaagccttgtacagttagtttcaaaaggcttacttagtggacaagtaattggctatgacccatctgttcaaggtctgataataagggtgaatctgatgcgaacgcagaagatcgatagagcactagtttatcagccgtatgtattaccaatcactcttaactctaacatagtaacaccaatagccccagaatgcgtaattcagaaagggacgatcatagagggtatgtcacggaaggattgtacagagttggagcaagacataatttgtaggacagttacgacatacacgcttgccagagacaccagattgtgtttacaaggcaatatctcttcttgtaggtatcaacagtcaggcacccaactacacaccccatttattacgtacaacggggcagttatcgcaaactgtgatttagtctcttgtcggtgtctccgtcctcctatgattatcacccaagtaaaagggtaccctcttaccataatcacaaggtccgtatgtcaagaattatccgttgacaacttagtcctgaatattgagacacaccataatttctcccttaatccaacaattatcgaccctctaacaagagtaatagccactacaccgttagagatagattccttaatccaagaggcacaagatcatgcgaatgcagcgctagctaaagtggaagagagtgataaatatctcagggcagttactggcggtaattattcgaattggtatatcgttcttgtcatcgtcttactatttgggaatttgggctggtcgctgttgttaacagtacttctatgtaggtcacggaaacaacagcgacgttatcaacaagatgattccgtcgggtcagaaagaggtgttggggttgggaccatccagtatatgtcataagtggtgtcagacatcacaatccaagtcactcagttatctactccgctggctcggagccatcgacaacgggggggagccctcatccggggacaatccacaacctgatactaccagacagtggctccgcacgatggcagcagcccacaacgtcctgcatgtaaatatcattgcgttactttaattaaaaaacctcaaggtgacccatcctggcccatccgattggaggagcggaaggttttaaggtaatcaggcaaattgtttccaacatggaatcccctccttctggcaaggatgcgccagccttccgcgagcctaagcgaacttgcagactctgttacagggcgacgactctctcccttaatctcaccatcgttgtattatcaattataagcatttatgtatcaactcaaactggggcaaacaactcttgtgtcaatcccacaatcgtaactcctgattatttaactggcagcacgacaggctcagttgaagatcttgctgacttagagagtcaactccgggagatacgtagagacacagggattaatctcccagttcagatcgataatacagaaaatctaatactgacaactctggcgagtatcaactcgaatttacgctttctgcaaaatgcaacaactgagagccagacctgcctatctcctgtcaatgaccctcgctttgtagctggcataaataggatcccagcagggtcgatggcatataatgacttcagcaacctcattgagcacgtaaatttcataccgtcgccgacaacgttgtcaggctgcaccaggataccatcattttcactctctaagacgcactggtgttatacccacaatgttatatcaaacgggtgccttgaccatgctgcaagttcacaatacatctcaatagggatcgttgataccggcctaaataatgagccctattttagaacaatgtcttcaaagtcactgaatgacggattaaatagaaagagttgctctgtgaccgcagccgctaatgcatgttggttactctgtagcgtagtaacagaatacgaagctgcggactatcgatcacgaactcctactgctatggttctggggcgattcgattttaatggtgaatacacagaaatagctgtcccctcctcactattcgatggccggtttgcatctaactatccaggtgtgggatcgggaactcaagtcaatggaacattatatttccctttatatggaggggtccttaatgggtcagatatagaaacggcaaacaaggggaaatccttcaggcctcagaatcctaagaataggtgtccagactcggaggcgatccaaagctttagagcgcaagatagctattacccgacgagatttgggaaggtgctgatccaacaggcaatcattgcatgtaggatcagtaacaaaagttgtactgatttctatctcctgtacttcgacaacaacagagtgatgatgggtgcagaggcacggctatattatcttaataaccaattatatctatatcaacgctcgtcgagctggtggccgcatcctttgttctatagcatctcattaccgtcatgtcaagcccttgctgtctgtcaaattacagaggcccatctgacactgacttacgccacgtcgaggccaggaatgtctatctgtacaggagcatcaaggtgtccaaataattgcgtagacggtgtatatactgatgtatggccattgacaaagaacgatgctcaggaccccaatctcttctacactgtatacttaaataattcaactcggcggattagcccaaccataagtctatacacttatgaccgcaggatcaaatctaagctggcagtgggtagtgatataggagcagcttataccacgagtacctgtttcggccgatcagatacaggggcggtttattgcttaactataatggagaccgttaacacaatatttggtcagtaccggatcgtgccaatattacttagagtgacaagccggtaatggattgttcctagtgaagtggggcctatggttactccgtaacggagatgacctgatttatgctatgtaactaattttgctcaatcgggagggtgatcggccgggtatgtccacaatgacaatgactcctattcccttgctagagctagcttcctctttaatccaatacaatccctacctgtagtttaataaaaaaatcctctatataagatccagacgaaggctatataagctaatagcttaggatggaaacgacaggagcggaacatatccctcctaccatgtcttctcataatattatcctccctgaccaccacttaaattcccctattgtactaaataaattgatgtactattgtaaactccttaatatattgcctgaccctaagtcgccatggtacgagaagattaagtcttggactaattgctgcatccgtgtgtcagacagcaatcgcatgactttgtcacgcgcctccaagctacgagaactattggcaacgtacggcgtatattcgaagaaccaccagcaatgttataccaccataatccatccccaatccctatcacccatcatgcagactgtctctcaactgggacgctcaatcccgacctgggccaggatccgaaaggagatcacacacagtatcatagcccaaacgaataaatttgaatccttattccacaacatctcaaaggacctgacagggaaaaccaacttgtttggcggattctgtgatttacatgggagcataagcactgctgcaaagcgcttaatgaatcaaccgggtctctacttggattcacctgatgcccatgcatgtcaatttttgttccagcttaaaacctgtcaacgggaattgattctacttatgaggcagaatgccacaacagagctgattagggtttttgactatcctgagctacgggttcttgtgacacctgaatattcggtctgggtttttacacggaccaaacaagttacattactaacattcgattgcttgttaatgtactgcgatctttcagacggccggcacaacatcctattcacttgtaagcttttgtctcacttaaaccacctcgggtgtcggataagggacctgctggtgctaattgattcacttgccgagaaacaccccttaatagtttatgatgtagttgcaagtctggagtcgttatcatatggtgctatacagctacatgacaaggtagctggttatgctggaacatttttctcatttatccttgcagaaatacaggattccttagaaacagtgttggatcaaggtaatagtgaatctatcatatcccaaataaggaacatatactcaggtctcacagtaaacgaggctgcagaattgctatgtgttatgaggctttggggccaccctgccctaaacagtgtagacgctgcaagcaaggtgagacagagtatgtgcgctggcaagctgcttaaatttgatacgatccagttagtccttgcattcttcaatacactaataattaatggatataggagaaagcaccatgggcgatggccgaatgtttgtagtgactctataattgggaatgaactcaagcggatgtattttgatcaatgtgagatcccccacgacttcacattaaagaactaccgggaattaagtcttcttgaattcgagtgcacatttcccatagaattatcagataaactgaacatatttttaaaagataaggcaatagcattcccgaagtccaggtggacatctcctttcaaagcagatatcacaccgcgccatttgctgcaagcgcctgagtttaaaacccgtgccaacagactactgctatcattcttacaactcgaggaattctccatcgaatcagaactagaatatgtgactaccctggcttacctggatgatgatgagttcaacgtatcttactccctcaaagagaaggaggtaaagacagatggtcgaatcttcgctaagctcacaaggaaaatgcggagctgtcaggtgatattcgaagaattgctagcagagcatgtctctccattatttaaagacaacggcgttactatggccgaattgtcacttactaagagcctactcgctattagcaacctaagctccaccttatttgagacgcaaacaaggcaaggcgatcggaatgcaagattcacgcatgcgcacttcataacaactgacctgcaaaagtactgtttaaactggaggtaccagagtgtaaaactgttcgcacgtcaattgaaccggttatttggtctgcagcatgggtttgaatggatccattgcatactaatgaagtccactatgtatgtcgcagacccctttaaccctccacatagcaatgcacgtgaggcactcgatgacaacttaaatagcgacatattcattgtatcacctaggggggccatcgagggactatgccaaaaaatgtggacaatcatctcaatctcggcgatccatgcatccgctgctgtggctgggctacgggtggcatctatggtccaaggtgataaccaagttattggggtaacacgcgagttcctagctgggcatgatcaaacttttgtagacgatcaattgacggtttcacttgagaacttcactcagatattcaaggaagtcaactacgggctaggtcataatctcaagctgagggaaacaatcaagtcgagccacatgttcatctactcaaagaggatcttctacgacggccgagtgcttccacaacttctaaagaacattcgtaagttaaccctatcagctacaactacaggggagaactgtttaacgagttgcggggacctatcatcatgcatcacacgatgtatagagaatgggttccctaaagatgcagcattcgttttgaatcaattagtgattaggatccagatacttgccgatcacttttattccatccttggaggatgtttctctgggttgaaccaggcggatatccgtctccttctatatgagggtgcaattttaccggcacaattaggagggttcaacaacctcaacatgtcacggctattctgtcggaatattggtgacccattagtcgcgtccatcgcagacaccaagagatttgtgaagtgccaactgctcgttcctcacattttagactctgtcgttgctattaccgataggaaggggtcttttacgacactgatgatggatccatattcaataaacttggattatatccagcaaccagagacacgattgaagcgacatgtgcaaaaggttttactacaagagtcagtgaatccgctactccaaggggtcttcctagagtctcaacaagaggaagaggaacaacttgcagcgttcttgctggaccgagaagtcgtaatgccaagggttgcgcacgctatatttgagtgtaccagcttaggtaggcgccgacatatccagggcctcatcgacacgacaaagaccattattgcacttgcgttggatacacagcagttaagttatactaaacgtgaacagattgtgacgtacaatgcaacttatatgaggtccttagcaagcatgctcagttcaagccataatcagacacgtcggtctgtgattggtcacgcatccttcaatatcaccgactgctcggtcatcctggctcaacaagtgagacgtgcaagctgggcgcccttgctgaattggcggtcattagaaggcttagaagtacccgaccctattgagtcagtagccggctacctgggtttggattctaacaactgtttcttatgctgccacgagcagaacagctattcctggtttttccttccccgaatgtgccattttgatgattcgagacaatctaactccatacaaagggtaccttacattggttctaagactgacgagcgccagatgtctacgataaatctacttgagaaaactacctgtcatgctagagcagctacgaggcttgcatcattatacatttgggcctttggggactcagacgatagttgggatgcagtcgagactctatctaacagtcgatgccagatcacccgagagcacttgcaagctctgtgccctatgccctcttcagtgaatctccaccatcgattgaacgacggaataacacaagtgaagtttatgccatcgaccaatagcagggtctctcgatttgttcatatttccaatgaccggcagaattacgtattggatgactccgttactgacagtaatctgatttaccaacaagtcatgttgctaggacttagtatacttgaaacatatttcagagaacctacagcgacgaacttatcaagcttggtgctgcaccttcacactgatgtatcttgttgcttacgagaatgcccgatgacacaatatgcgccccccctgcgagacatcccagagttaactataactgcgtcaaatcctttcttatttgatcaagaacctattagcgaagcagacctgtgtagactctcaaagatagcattccgtagagcaggagacaactacgatgcatacgaccagtttcaactaagagcaacactggccgcaactacaggtaaggatgtggcagcaacaatctttggtcctctagctgcagtatcagcaaaaaatgatgcaatagtcacaaatgactatagcgggaattggatttccgagtttaggtatagtgatctctatcttttgagtgttagcctgggctacgagatccttttgattttcgcgtatcaactttactacctacggatacggtaccggcaaaatattgtatgctatatggaatctgtattccgacgttgtcattctttatgcttgggcgacttaatccagactatttcgcactctgaaatactggttgggttaaatgcagcgggctttaatcttaccttggatcgaagtgatttgaaggagaatcaactctcgcggcttgctgtgaaatacctcacattgtgtgtccagactgcaataagtaatttggaggtaggatcagaacccctttgtattattggaggtcaattagatgatgacatatcattccaagttgcccacttcctctgtcgacgactgtgcatcatcagcttggtccactcaaatgtacagaacttacccccgattagggataatgaagttgatgtaaaatccaagttgatctacgaccatcttaagctagttgctaccacactcaataatagggaccagtcgtatctgctaacactactgaataagcctaacatcgagcttcatactccacaggtttacttcattatgaggaagtgtctgggattattaaaagtctatggacctgtacctcaaaagcaaccacttccaacctccccagtagtatcactaccaaataagtgcaaatcgaaatggaagcttgaggaggtaattgacagcattgagtcacctaagtcttttaactgggttcctgatacgacgattccattggatgctgagcaaaatcccccaaatcctaacagggtcatcgataagataaacattctaagatctctgagcccgcgtcactcagtctggtaccgtaaccggcaataccggtacgtgttgagccagttagggcatgaccagctaggtggtgcaaccctatatcttggtgaagggagcggctccacaatcttgaacatagagccaaaagtcaggagtgataagatatattatcatacctatttctctgcagacaaaagccctgcacagcgcaatttcatcccccagccaactacttttctgcgatctaacttttatcactacgaattagagccgtctggatgtgagttcatcaattgttggtccgaggatgtaaatgcaacgaatcttacggaactgcgttgcataaatcacatattaactgtgattccagtcggatctctaacacgtattatttgtgacgtggaattggcaaatgacacgtcaatacaatcagtggccaccgcatacatgaacttaagcattcttgcacatgctctcctcaaccagggaggcgtytgcatctgtaggtgccacctgttagacacgtcccatcttgcgattgtttcgtttgtacttaaaacattatcaagccagctggcgatttcgttttcaggatattgtggaaccaatgacccatcttgtgtcataggagtcacgaaagagtgctccattagcctggatgtcttaagttcgattgcagctgcatttataaatgaattgccctcaaatgagctgccggtccctcaatcattattaacgctactagggtgttatcaagaccagttagagagccttagtgcactcattgaaaaaacatggatccaggagatccgggaggcctcatttaatgcatgtgagatggagtggataggacttttgggaacagatgcgctgggcgatgtcgacacatttatcagttatcgcaatgattcgtgtaattccatccctgatctactgacgccagctgtgagtgccctcttatttgaattaatcagcctgacacctgagcttcctggtacggaggaccagccgagtaggcgtgtaatcagcattggccaagcttttaacctgactatttcgggaaaagttaatacgatgatcagatcatgctgtgagcagtgcattaagttattagctgccaatgtctccatccttagtgatgtagatttgtcgtattttgtgagggggattagagatggttcttttgctttggggttgttaatcacccagcgtcaaatactcaaggcatcgagagcaccaaagtacctcaaaacgcatcaggtccagaactggattgcgttgttactggaggtcaaactagaggaaatcttctctcgccactaccggaaagtgctattgagaactctacggctgttatccctctacgggctcctgcagcagaaggaaagttgatcatgatagatgtctctctgatttcgcctcagccctatatacagatacctgctactgctgttgtaactatgcaaatataggctaacctcggtccggagtattgaggtaatggggagactcgtaattggagaaagaagaagaacggggcagataccagaccccgggatacactactataagctacttgggtgactcaggacgtcactaataagagtacactggttttaacaataacactgatagtgtaacactgaatattgtaaacagtctcagggattatttaattaaaaagacattgcggatcatcatgtcaggcctgaaccaatcagaaacccatcctacaggcatgagtcgattaatccagtttaatttagtccaagatgattagtactcttagatgttactaagtatcaggatttagcgtactagattaaacgtctttgaaagtcattttaccctgcaaggtgcataagcatatgcaggggaatacccaccactggttcgttagtctaatttattcaaagatgccaccaactgagtgcactgggagtctgattgagctgaatcaacctaaagctcaagctttcctgtgatgtagcatgcagtgtgctactgctgcatagagccaatcaggggcgtcttgaaggcctccaccttcctcaggcaacgataaagcagctcactgctacacccaccatgcaaatacaaggatatgtaggtccatcaccctgcttgctaatgtggtcggctccaggaccatggccatagcgctatccgtctaattcaatccgcctcatcatatgcgtatacaatcctccactgagaaatgcacctgctcaatccaatgacaaactacaacaacacgagatcattatggggtcgattcccttgaatcagaaaggtaaaaacggcagcggagatggagaataccagacctaatcggcaaagaacaaaaggttgaagactgatttaaaaatcattataactttttgtttagt

SEQ ID NO: 18 - DNA, 15054 bp, artificial sequence
  APMV4 genome sequence
acgaaaaagaagaataaaaggcagaagccttttaaaaggaaccctgggctgtcgtaggtgtgggaaggttgtattccgagtgcgcctccgaggcatctactctacacctatcacaatggctggtgtcttctcccagtatgagaggtttgtggacaatcaatcccaagtgtcaaggaaggatcatcggtccttagcaggaggatgccttaaagttaacatccctatgcttgtcactgcatctgaagaccccaccactcgttggcaactagcatgcttatctctaaggctcctgatctccaactcatcaaccagtgctatccgtcagggggcaatactgactctcatgtcattaccatcacaaaacatgagagcaacagcagctattgctggttccacaaatgcagctgttatcaacaccatggaagtcttaagtgtcaacgactggaccccatccttcgaccctaggagcggtctttctgaggaagatgctcaagttttcagagacatggcaagagatctgccccctcagttcacctctggatcacccttcacatcagcattggcggaggggttcactcctgaagatactcatgacctgatggaggccttgaccagtgtgctgatacagatctggatcctggtggctaaggccatgaccaacattgacggctctggggaggccaatgaaagacgtcttgcaaagtacatccaaaaaggacagcttaatcgtcagtttgcaattggtaatcctgcccgtctgataatccaacagacaatcaaaagctccttaactgtccgtaggttcttggtctctgagcttcgtgcgtcacgaggtgcagtaaaagaaggatccccttactatgcagctgttggggatatccacgcttacatctttaatgcgggattgacaccattcttgaccaccttaagatacgggataggcaccaagtacgccgctgttgcactcagtgtgttcgctgcagatattgcaaagttgaagagcctacttaccctgtaccaggacaagggtgtagaagctggatacatggcactccttgaggatccagactccatgcactttgcacctggaaacttcccacacatgtactcctatgcaatgggggtagcttcttaccatgatcctagcatgcgccaataccaatacgccaggaggttcctcagccgtcctttctacttactaggaagggacatggccgccaagaacacaggcacgctggatgagcaactggcgaaggaactgcaagtatcagagagagatcgcgccgcattatccgctgcgattcaatcagcgatggaggggggagagtccgacgacttcccactgtcgggatccatgccggctctctctgagaatgcgcaaccagttacccccagacctcaacagtcccagctctctcccccccaatcatcaaacatgccccaatcagcacccaggaccccagactatcaacccgactttgaactgtaggcttcatcaccgcaccaacaacagcccaagaagaccacccctccccccacacatctcacccagccacccataaagactcagtcccacgccccagcatctccttcatttaattaaaaaccgaccaacagggtggggaaggagagtcattggctactgccaattgtgtgcagcaatggattttactgacattgatgctgtcaactcattgatcgaatcatcatcggcaatcatagactccatacagcatggagggctgcaaccagcgggcaccgtcggcctatcgcagatcccaaaagggataaccagcgcattaaccaaggcctgggaggctgaggcggcaactgccggtaatggggacacccaacacaaatctgacagtccggaggatcatcaggccaacgacacagattcccctgaagacacaggtactgaccagaccacccaggaggccaacatcgttgagacaccccaccccgaggtgctgtcagcagccaaagccagactcaagaggcccaaagcagggagggacacccgcgacaactcccctgcgcaacccgatcatcttttaaaggggggcctcctgagcccacaaccagcagcatcatgggtgcaaaatccacccagtcatggaggtcccggcaccgccgatccccgcccatcacaaactcaggatcattcccccaccggagagaaatggcgattgtcaccgacaaagcaaccggagacattgaactggtggagtggtgcaacccggggtgcacagcagtccgaattgaacccaccagactcgactgtgtatgcggacactgccccaccatctgtagcctctgcatgtatgacgactgatcaggtacaactactaatgaaggaggttgctgacataaaatcactccttcaggcgttagtgaggaacctcgctgtcttgccccaattgaggaatgaggttgcagcaatcagaacatcacaggccatgatagaggggacactcaattcgatcaagattcttgaccctgggaattatcaggaatcatcactaaacagttggttcaaacctcgccaagatcacactgttgttgtgtctggaccagggaatccattggccatgccaaccccagtccaagacaacaccatattcctggacgagctagccagacctcatcctagtgtggtcaatccttccccacccatcaccaacaccaatgttgaccttggcccacagaagcaggctgcaatagcctatatctccgctaaatgcaaggatccggggaaacgagatcagctatcaaggctcattgagcgagcaaccaccccaagtgagatcaacaaagttaaaagacaagcccttgggctctagatcactcgatcacccctcatggtgatcacaacaataatcagaacccttccgaaccacatgaccaacccagcccaccgcccacaccgtccatcgacatcccttgccaaacatcctgccgtagctgatttattcaaaagagctcatttgatatgacctggtaatcataaaatagggtggggaaggtgctttgcctgtaagggggctccctcatcttcagacacgtgcccgccatctcaccaacagtgcaatggcagacatggacacggtgtatatcaatctgatggcagatgacccaacccaccaaaaagaactgctgtcctttcctctcatccctgtgaccggtcctgacgggaagaaggaactccaacaccagatccggacccaatccttgctcgcctcagacaaacaaactgaacggttcatcttcctcaacacttacggattcatctatgacaccacaccggacaagacaactttttccaccccagagcatattaatcagcctaagaggacgacggtgagtgccgcgatgatgaccattggcctggttcccgccaatatacccctgaacgaactaacggctactgtgttcagccttaaagtaagagtgaggaaaagtgcgaggtatcgggaagtggtctggtatcaatgcaatccagtaccggccctgcttgcagccaccaggtttggtcgccaaggaggtctcgagtcgagcactggagtcagtgtaaaggctcccgagaagatagattgtgagaaggattatacctactacccttatttcttatctgtgtgctacatcgccacctccaacctgttcaaggtaccgaggatggttgctaatgcaaccaacagtcaattataccaccttaccatgcaggtcacatttgcctttccaaaaaacatccctccagccaaccagaaactcctgacacaggtggatgagggattcgagggcactgtggattgccattttgggaacatgctgaaaaaggatcggaaagggaacatgaggacactgtcccaggcggcagataaggtcagacgaatgaatattcttgttggtatctttgacttgcatgggccaacgctcttcctggagtataccgggaaactgacaaaggctctgctagggttcatgtccaccagccgaacagcaatcatccccatatctcagctcaatcccatgctgagtcaactcatgtggagcagtgatgcccagatagtaaagttaagggttgtcataactacatccaaacgcggcccgtgcgggggtgagcaggagtatgtgctggatcccaaattcacagttaagaaagaaaaggctcgactcaaccctttcgagaaggcagcctaatgatttaatccgcaagatcccagaaatcagaccactctatactatccactgatcactggaaatgtaattgtacagttgatgaatctgtgaagaatcaattaaaaaaccggatccttattagggtggggaagtagttgattgggtgtctaaacaaaagcatttcttcacacctccccgccacgaaacaaccacaatgaggctatcaaacacaatcttgaccttgattctcatcatacttaccggctatttgataggtgtccactccaccgatgtgaatgagaaaccaaagtccgaagggattaggggtgatcttacaccaggtgcgggtattttcgtaactcaagtccgacagctccagatctaccaacagtctgggtaccatgatcttgtcatcagattgttacctcttctaccaacagagcttaatgattgtcaaagggaagttgtcacagagtacaataacactgtatcacagctgttgcagcctatcaaaaccaacctggatactttgttggcagatggtagcacaagggatgttgatatacagccgcgattcattggggcaataatagccacaggtgccctggctgtagcaacggtagctgaggtaactgcagctcaagcactatctcagtcaaaaacgaatgctcaaaatattctcaagttgagagatagtattcaggccaccaaccaagcagtttttgaaatttcacagggactcgaagcaactgcaaccgtgctatcaaaactgcaaactgagctcaatgagaatatcatcccaagtctgaacaacttgtcctgtgctgccatggggaatcgccttggtgtatcactctcactctatttgaccttaatgaccactctatttggggaccagatcacaaacccagtgctgacgccaatctcttacagcaccctatcggcaatggcgggtggtcacattggtccagtgatgagtaagatattagccggatctgtcacaagtcagttgggggcagaacaactgattgctagtggcttaatacagtcacaggtagtaggttatgattcccagtatcagctgttggttatcagggtcaaccttgtacggattcaggaagtccagaatactagggttgtatcactaagaacactagcagtcaatagggatggtggactttacagagcccaggtgccacccgaggtagttgagcgatctggcattgcagagcggttttatgcagatgattgtgttctaactacaactgattacatctgctcatcgatccgatcttctcggcttaatccagagttagtcaagtgtctcagtggggcacttgattcatgcacatttgagagggaaagtgcattactgtcaactcccttctttgtatacaacaaggcagtcgtcgcaaattgtaaagcagcgacatgtagatgtaataaaccgccatctatcattgcccaatactctgcatcagctctagtaaccatcaccaccgacacttgtgctgaccttgaaattgagggttatcgtttcaacatacagactgaatccaactcatgggttgcaccaaacttcacggtctcaacctcacaaatagtatcggttgatccaatagacatatcctctgacattgccaaaattaacaattctatcgaggctgcgcgagagcagctggaactgagcaaccagatcctttcccgaatcaacccacggattgtgaacgacgaatcactaatagctattatcgtgacaattgttgtgcttagtctccttgtaattggtcttattattgttctcggtgtgatgtacaagaatcttaagaaagtccaacgagctcaagctgctatgatgatgcagcaaatgagctcatcacagcctgtgaccaccaaattggggacacccttctaggtgaataatcatatcaatccattcaataatgagcgggacataccaatcaccaacgactgtgtcacaaggccggttaggaatgcaccggatctctctccttcctttttaattaaaaacggttgaactgagggtgagggggggggtgtgcatggtagggtggggaaggtagccaattcctgcccattgggccgaccgtaccaagagaagtcaacagaagtatagatgcagggcgacatggagggtagccgtgataacctcacagtagatgatgaattaaagacaacatggaggttagcttatagagttgtatccctcctattgatggtgagtgccttgataatctctatagtaatcctgacgagagataacagccaaagcataatcacggcgatcaaccagtcgtatgacgcagactcaaagtggcaaacagggatagaagggaaaatcacctcaatcatgactgatacgctcgataccaggaatgcagctcttctccacattccactccagctcaatacacttgaggcaaacctgttgtccgccctcggaggttacacgggaattggccccggagatctagagcactgtcgttatccggttcatgactccgcttacctgcatggagtcaatcgattactcatcaatcaaacagctgactacacagcagaaggccccctggatcatgtgaacttcattccggcaccagttacgactactggatgcacaaggatcccatccttttctgtatcatcatccatttggtgctatacacacaatgtgattgaaacaggttgcaatgaccactcaggtagtaatcaatatatcagtatgggggtgattaagagggctggcaacggcttaccttacttctcaacagtcgtgagtaagtatctgaccgatgggttgaatagaaaaagctgttccgtagctgcgggatccgggcattgttacctcctttgtagcctagtgtcagagcccgaacctgatgactatgtgtcaccagatcccacaccgatgaggttaggggtgctaacaagggatgggtcttacactgaacaggtggtacccgaaagaatatttaagaacatatggagcgcaaactaccctggggtagggtcaggtgctatagcaggaaataaggtgttattcccattttacggcggagtgaagaatggatcaacccctgaggtgatgaataggggaagatattactacatccaggatccaaatgactattgccctgacccgctgcaagatcagatcttaagggcagaacaatcgtattatcctactcgatttggtaggaggatggtaatgcagggagtcctaacatgtccagtatccaacaattcaacaatagccagccaatgccaatcttactatttcaacaactcattaggattcatcggggcggaatctaggatctattacctcaatggtaacatttacctttatcaaagaagctcgagctggtggcctcacccccaaatttacctacttgattccaggattgcaagtccgggtacgcagaacattgactcaggcgttaacctcaagatgttaaatgttactgtcattacacgaccatcatctggcttttgtaatagtcagtcaagatgccctaatgactgcttattcggggtttattcagatgtctggcctcttagccttacctcagacagcatatttgcatttacaatgtacttacaagggaagacgacacgtattgacccagcttgggcgctattctccaatcatgtaattgggcatgaggctcgtttgttcaacaaggaggttagtgctgcttattctaccaccacttgtttttcggacaccatccaaaaccaggtgtattgtctgagtatacttgaagtcagaagtgagctcttgggggcattcaagatagtgccattcctctatcgtgtcttataggcacctgcttggtcaagaaccctgagcagccataaaattaacacttgatcttccttaaaaacacctatctaaattactgtctgagatccctgattagttaccctttcaatcaatcaattaatttttaattaaaaacggaaaaatgggcctagttccaaggaaaggatgggacccattagggtggggaaggattactttgttccttgactcgcacccacgtacacccaatcccattcctgtcaagaaggaacccttcccaaactcaccttgcaatgtccaatcaggcagctgagattatactacccaccttccatcttttatcacccttgatcgagaataagtgcttctactacatgcaattacttggtctcgtgttaccacatgatcactggagatggagggcattcgtcaattttacagtggatcaagcacaccttaaaaatcgtaatccccgcttaatggcccacatcgatcacactaaggatagactaagggctcatggtgtcttgggtttccaccagactcagacaagtgagagccgtttccgtgtcttgctccatcctgaaactttaccttggctatcagcaatgggaggatgcatcaaccaggttcccaaggcatggcggaacactctgaaatctatcgagcacagtgtgaagcaggaggcgactcaactgaagttactcatggaaaaaacctcactaaagctaacaggagtatcttacttattctccaattgcaatcccgggaaaactgcagcgggaactatgcccgtactaagtgagatggcatcagaactcttgtcaaatcccatctcccaattccaatcaacatgggggtgtgctgcttcagggtggcaccatgtagtcagcatcatgaggctccaacagtatcaaagaaggacaggtaaggaagagaaagcaatcactgaagttcagtatggctcggacacctgtctcattaatgcagactacaccgtcgttttttccgcacaggaccgtgtcatagcagtcttgcctttcgatgttgtcctcatgatgcaagacctgcttgaatcccgacggaatgtcttgttctgtgcccgctttatgtatcccagaagccaactacatgagaggataagtacaatactggcccttggagaccaactcgggagaaaagcaccccaagtcctgtatgatttcgtagctaccctcgaatcatttgcatacgctgctgtccaacttcatgacaacaaccctatctacggtggggctttctttgagttcaatatccaagaactggaagctattttgtcccctgcacttaataaggatcaagtcaacttctacataagtcaagttgtctcagcatacagtaaccttcccccatctgaatcagcagaattgctatgcttactacgcctgtggggtcatcccttgctaaacagtcttgatgcagcaaagaaagtcagagaatctatgtgtgctgggaaggttcttgattataatgctattcgactagttttgtctttttatcatacgttattaatcaatgggtatcggaagaaacataagggtcgctggccaaatgtgaatcaacattcactactcaacccgatagtgaagcagctttactttgatcaggaggagatcccacactctgttgcccttgagcactatttagatatctcgatgatagaatttgagaagacttttgaagtggaactatctgatagtctaagcatctttctgaaggataagtcgatagctttggataaacaagaatggcacagtggttttgtctcagaagtgactccaaagcacctacgaatgtctcgtcatgatcgcaagtctaccaataggctattgttagcctttattaactcccctgaattcgatgttaaggaagagcttaaatatttgactacaggtgagtatgccactgacccaaatttcaatgtctcttactcactgaaagagaaggaagttaagaaagaagggcgcattttcgcaaagatgtcacagaaaatgagagcatgccaggttatttgtgaagagttactagcacatcatgtggctcctttgtttaaagagaatggtgttacacaatcggagctatccctgacaaagaatttgttggctattagccaactgagttacaactcgatggccgctaaggtgcgattgctgaggccaggggacaagttcaccgctgcacactatatgaccacagacctaaaaaagtactgccttaactggcggcaccagtcagtcaaattgttcgccagaagcctggatcgactatttgggttagaccatgctttttcttggatacacgtccgtctcaccaatagcactatgtacgttgctgacccattcaatccaccagactcagatgcatgcacaaatttagacgacaataagaacactgggatttttattataagtgctcgaggtggtatagaaggccttcaacagaaactatggactggcatatcaattgcaatcgcccaggcggcagcagccctcgagggcttacgaattgctgccactttgcagggggataaccaggttttagcgattacgaaagaattcatgaccccagtctcggaggatgtaatccacgagcagctatctgaagcgatgtcgcgatacaagaggactttcacataccttaattatttaatggggcaccaattgaaggataaagaaaccatccaatccagtgacttcttcgtttactccaaaaggatcttcttcaatgggtcaatcctaagtcaatgcctcaagaacttcagtaaactcactaccaatgccactacccttgctgagaacactgtagccggctgcagtgacatctcctcatgcatagcccgttgtgtggaaaacgggttgcctaaggatgctgcatatgttcagaatataatcatgactcggcttcaactgttgctagatcactactattctatgcatggtggcataaactcagagttagagcagccaactctaagtatccctgtccgaaacgcaacctatttaccatctcaattaggcggttacaatcatttgaatatgacccgactattctgtcgcaatatcggtgacccgcttactagttcttgggcagagtcaaaaagactaatggatgttggccttctcagtcgtaagttcttagaggggatattatggagacccccgggaagtgggacattttcaacactcatgcttgatccgttcgcacttaacattgattacttaaggccaccagagacaataatccgaaaacacacccaaaaagtcttgttgcaggattgtcctaatcctctattagcaggtgtagttgacccgaactacaaccaggaattagaattattagctcagttcctgcttgatcgggaaaccgttattcccagggctgcccatgccatctttgaactgtctgtcttgggaaggaaaaaacatatacaaggattggttgatactacaaaaacaattattcagtgctcattagaaagacagccactgtcctggaggaaagttgagaacattgtaacctacaatgcgcagtatttcctcggggccacccagcaggttgacaccaatatctcagaaaggcagtgggtgatgccaggtaatttcaagaagcttgtatctcttgacgattgctcagtcacgttgtccactgtgtcacggcgcatttcttgggccaatctacttaactggagggctatagatggtttggaaactccagatgtgatagagagtattgatggccgccttgtgcaatcatccaatcaatgcggcctatgtaatcaaggattgggctcctactcctggttcttcttgccctccgggtgtgtgttcgaccgtccacaagattctcgagtggttccaaagatgccatacgtgggatccaaaacggatgagagacagactgcgtcagtgcaggctatacagggatccacatgtcaccttagagcagcattgagacttgtatcactctacctttgggcctatggagattctgacatatcatggctagaagccgcgacattggctcaaacacggtgcaatatttctcttgatgacctgcggatcctgagccctcttccttcctcggcaaatttacaccacagattgaatgacggggtaacacaagtgaaattcatgcccgccacatcgagccgggtgtcaaagttcgtccaaatttgcaatgacaaccagaatcttatccgtgatgatgggagtgttgattccaatatgatttatcagcaggttatgatattagggcttggagagattgaatgtttgttagctgacccaatcgatacaaacccagaacaactgattcttcacctacactctgataattcttgctgtctccgggagatgccaacgaccggttttgtacctgctttaggattgaccccatgcttaactgtcccaaagcacaatccgtatatttatgatgatagcccaatacccggtgatttggatcagaggctcattcaaaccaaattctttatgggttctgacaatctagataatcttgatatctaccagcagcgagctttactgagtcggtgtgtggcttatgacattatccaatcagtattcgcttgcgatgcaccagtatctcagaagaatgatgcaatccttcacactgactaccatgaaaattggatctcagagttccgatggggtgaccctcgcataatccaagtaacagcaggttacgagttaattctgttccttgcataccagctttattatctcagagtgaggggtgaccgtgcaatcctgtgttatattgataggatactcaacaggatggtatcttccaatctaggcagtctcatccagacgctctctcatccggagattaggaggagattttcattgagtgatcaagggttccttgtcgaaagggagctagagccaggtaagccactggtaaaacaagcggttatgttcctaagggactcagtccgctgcgctttagcaactatcaaggcaggaattgagcctgagatctcccgaggtggctgtacccaggatgagctgagctttacccttaagcacttactatgtcggcgtctctgtataattgctctcatgcattcggaagcaaagaacttggtcaaagttagaaaccttccagtagaggaaaaaaccgccttactataccagatgttgatcactgaggccaatgccaggagatcagggtctgctagtatcatcataagcttagtttcagcaccccagtgggacattcatacaccagcgttgtattttgtatcaaagaaaatgctggggatgctcaaaaggtcaaccacacccttggatataagtgacctttctgagagccagaacctcacaccaacagaattgaatgatgttcctggtcacatggcagaggaatttccctgtttgtttagcagttataacgctacatatgaagacacaattacttacaatccaatgactgaaaaactcgcagtgcacttggacaatggttccaccccttccagagcgcttggtcgtcactacatcctgcgaccccttgggctttactcgtctgcatggtaccggtctgcagcactattagcgtcaggggccctcagtgggttgcctgaggggtcaagcctgtacttgggagaggggtatgggaccaccatgactctacttgagcccgttgtcaagtcctcaactgtttactaccatacattgtttgacccaacccggaatccttcacagcggaactacaaaccagaaccgcgggtattcactgattccatttggtacaaggatgatttcacacgaccacctggtggcattgtaaatctatggggtgaagacgtacgtcagagtgatattacacagaaagacacggttaatttcatattatctcgggtcccgccaaaatcactcaaattgatacacgttgatattgagttctccccagactctgatgtacggacgctactatctggctattcccattgtgcactattggcctactggctactgcaacctggagggcgatttgcggttagagttttcttaagtgaccatatcatagtcaacttggtcactgccattctgtccgcttttgactctaatctggtgtgcattgcgtcaggattgacacacaaggatgatggggcaggttatatttgtgcaaagaagcttgcaaatgttgaggcttcaagaattgagtattacttgaggatggtccacggctgtgttgactcattaaaaattcctcatcaattaggaatcattaaatgggctgagggtgaagtgtcccgacttaccaaaaaggcggatgatgaaataaactggcggttaggtgatccagttaccagatcatttgatccggtttctgagctaataattgcgcgaacagggggatcagtattaatggaatacgggacttttactaacctcaggtgtgcgaacttggcagatacatataaacttttggcttcaattgtagagaccaccttaatggaaataagggttgagcaagatcagttggaagatgattcgaggagacaaatccaggtagtccctgcttttaatacaagatccgggggaaggatccgtacattgattgagtgtgctcagctgcaggtcatagatgttatctgtgtgaacatagatcacctctttcccaaacaccgacatgctcttgtcacacaacttacttaccagtcagtgtgccttggggacttgattgaaggcccccaaattaagacatatctaagggccaggaagtggatccaacgtaggggactcaatgagacaattaaccatatcatcactggacaagtgtcgcggaataaggcaagggattttttcaagaggcgcctgaagttggttggcttttcgctctgtggcggttggggctacctctcactttagctgcttagattgttgattattatgaataatcggagtcgaaatcgtaaatagaaagacataaaattgcaaataagcaatgatcgtattaatatttaataaaaaatatgtcttttatttcgt

SEQ ID NO: 19 - DNA, 16236 bp, artificial sequence
  APMV6 genome sequence
accaaacaaggaaaccatatgcttggggaatttacgagaacgcttataaaatcgtgagggggaagctggtggactccgggtccggagtcggtggacctgagtctagtagcttccctgctgtgccaagatgtcgtcggtgttcactgactacgctaagctgcaagatgcccttgtggccccttcaaaaaggaaggtggatagtgcaccaagcggattgttaagggttgggatccctgtgtgtgtcctactctccgaggatcctgaagaacgatggagctttgtttgcttttgcatgaggtgggtggtgagcgattcagccacagaggcaatgcgtgttggtgcaatgctatctatcctcagcgcacacgccagcaacatgcggagccatgtcgcacttgcagcaagatgtggcgacgccgacatcaacatacttgaggttgaggcaattgaccaccaaaaccagaccattcgtttcactgggcgcagcaatgtgactgacgggagagcacgccagatgtatgcgattgcccaagatctgcctccctcctataacaatggtagcccttttgtaaatagagacattgaggacaattatccgaccgacatgtctgagctgctcaacatggtttacagtgttgcaactcaaatctgggtggcagctatgaagagcatgactgctccagacacatcctcagagtctgaagggaggcggttggccaaatacatccagcaaaacagagtgatccggagtacgattttagctcctgcaactcgcggtgaatgcacccggataatacggagctccctagtcatccgccactttctaataactgagatcaagcgtgccacatcaatgggttccaacacgacgagatattatgccacagttggggatgctgcagcttacttcaagaacgcgggtatggctgcattcttcttaactctgcggtttggaattgggaccaagtactccacacttgcagtttcagcgctgtccgccgacatgaagaaactccagagcttgatccgagtataccagagcaaaggtgaggatggaccctatatggcatttctggaagactccgaccttatgagcttcgcccctggaaactatccactcatgtattcatatgcaatgggagtagggtccattcttgaggcaagtattgctcgatatcagtttgctcgatcattcatgaatgacacgttctatcgattgggtgttgaaactgcacaacgaaaccaaggttcacttgatgagaatctagcgaaggagctgcaactatctggggccgaacggagggcagtgcaggaacttgtgaccagcctggacctagctggagaggccccagtgccccagcgtcaaccaacattcctcaatgaccaggagtatgaggatgatccccccgccaggagacagagaattgaggatactccagacgatgacggagccagtcaagctccacccacaccaggagcaggtctcaccccatactctgataatgccagtggcctggacatctaaacgaccagtacttagtataacaattgatcaaggttaatccaaagtatgcaaatccaatactccaatcgataacaagatcacatgtagactttaagaaaaaacaagggtgagggggaagttcctggtacgcgggctgggcccatagtgactcagctagcaccatggacttctccaatgaccaggagattgcagaattactcgagctgagttcggatgtgataaagagcatccaacatgccgagacccagccagcacacactgtcggcaaatctgccattcggaaaggaaatacatccgagctgcgagcagcctgggaagccgagacacaaccaacccaaacagaaaacaagtctgaggaacacccagagcaaactgcccgggatcccgacagcaagggcaacacaggaaattcgcaaccacgatccaacgcagaggagacaccccaatcagaaagtcacgacaggcaagtcgctgctccacccccagacaccacaataggggttaacgggaccaatgggcttgaagctgctctaaaaaagctagaaaaacaagggaaaggtcccgggaaaggccaagtggatcgcaacacccctcagagagatccaaccactgcctcgggttcaaaaaaggggaaagggggcgagccaaggagcaatgccctccatcagggttacccacaggggaccaacttgatcctgcccactcagaggccctctcatgccagactggcgcagcaagcatcacaggagataactcgccatgcgctgcaaccccaggactccggcggcatagaagggaattctccatttcttggagacacggccagtgcatcctggccgagtggtgcaacccagtctgtgcacctgtcacacctgaacccagagcattcaaatgcatttgcgggagatgccctcgagtatgcatcaactgtcgcaacgatagtggagactctgaaatttgtagttagcaggttagaagcacttgaaaatagggtggcggagctcaccaaatttgtctctcccattcagcaaatcaaagcagacatgcagattgtaaagacatcctgcgctgtcattgagggccaacttgccacagtgcaaatattggagccgggccactcatcgattcgctcacttgaggagatgaagcaatacaccaagccaggaattgtcgtccaaacagggatgactcaagacataagcgccgtcatgagggacggcacgatcgtaaaagatgctcttgctcgcccagttaatccggacaagtggtcagcaacaatcaatgctcaatcaacaacaatgaaggtaacccaagaagatataaagacggtgtatacactattggacaattttggcatcactggcccaaaaagagcgaaaattgaggcggaattggctaatgtcagtgaccgggacgcactagtaaggattaagaaacgtgttatgaatgcataagcagccaaaaaatcacaacaattagtacagatggcctcccaatgtcacatcatgattctattgtcaaatcacagcattctttcttcctgatcacacccaacaatttgctttggacacccttgacactgattaataaaaaagtgagggggaactggtggtgtccggactgggcaacccagggtcacccggtccgaaccaaacacccgccagttcctccaccggcacagcgcaccactgactgcaccgaccccaaccatggccacgtcagaactcaacctctatatcgacaaggactcaccccaggtgagattgctagcattccccatcatcatgaaacccaaagaaaatggggttagagagctgcaaccgcaactgaggactcagtacctcggtgacatcaccggaggaaagaaaagcgcgatatttgtgaattgctatgggttcgttgaagatcacggggggcgagacagcggattctcacccatcagtgaagaatccaaaggatcgacagtcactgcagcctgcatcactcttggcagcatcgagtatgatagtgacatcaaggaagtggcaaaggcctgctataatcttcaggtgtcagttaggatgtccgctgattcaactcagaaggttgtttacacaatcaatgctaaacctgcactgttgttctcctcccgtgttgtcagagctgggggttgtgtggttgcagcagagggtgcaatcaagtgccccgagaagatgacatccgatcgcctttacaagttccgcgtaatgtttgtttcattgaccttcttacatcgcagcagccttttcaaagttagccgtacggtgctgtcaatgaggaattctgctctaatagcagtacaggccgaagtgaagctgggattcgatctgccactagaccatccaatggcaaaatatctgagcaaagaggatggacagctctttgcgactgtgtgggtacatttgtgcaacttcaagcgcacagacagacggggagtagaccgatcggtggagaacatcagaaacaaagtacgggccatggggctgaagctcaccttgtgtgatctgtggggtcccacacttgtttgtgaagctacagggaagatgagcaagtacgcgctaggtttcttctcggagaccaaggttggctgtcatccaatctggaaatgcaactcgactgtcgcgaagatcatgtggtcatgcacaacctggatcgcatcagcaaaggccatcatacaggcctcctctgctcgtgccctgttgacatcagaggacatagaagccaagggagccatctccactgacaaaaagaaaacagatgggtttaatcccttcatcaagacagcaaagtagtcatctgaatctcatcagtgaacccactggcatatgttcagctgtgccttccttgataatcactaaatcaatacgcagagtgctctttgattaagatctcgattgttccaataagtggatcatttatactttgaagattgaacttcctaactattccttccttagaagtccagtcatattaatcaaaaaaatcagtttgctggtaaagtcgtatactgcaggatccaatacctctcaccaatgagtagctgagggggaaggcatgggagcccgactggggcccttaacaatggcacccggccggtatgtgatcattttcaatctcatccttctccatagggccgtctcactagacaattcaaggttactgcagcaggggatcatgagtgcgaccgagagagaaattaaagtgtacacgaactccataaccggaagtattgctgtgagattaattcccaacttacctcaagaagtgcttaagtgctccgctgggcagatcaaatcgtacaatgacacccttaaccgaatttttacacctattaaggcgaaccttgagaggttactggccacaccaagtatgcttgaagacaaccagaaccctgctccagaacctcgtctgattggagcaattataggcacagcagcactggggttggcaacagcagctcaggttacagctgcccttgcccttaaccaggcccaggataatgccaaggccatcttgaacctcaaagagtccataacaaagacaaacgaggctgtgcttgagcttaaagatgcaacagggcaaattgcaatagcgctagataagactcaaagattcataaatgacaatatcttaccggcgattaataatctgacatgtgaagtagcaggtgctaaagtaggtgtggaactgtcattatacttgaccgagttaagcactgtgtttggatcgcagataaccaatccagcactctcaactttatccattcaagccctcatgtcactctgtggtaatgattttaattacctcctgaatctaatgggggccaaacactctgatctgggtgcactttatgaggcaaacttaatcaatggcagaatcattcagtatgaccaagcaagccaaatcatggttattcaggtctccgtgcccagcatatcatcaatttcggggttgcgactgacagagttgttcactctgagcattgaaacaccggtcggcgagggcaaggcagtagtaccccagtttgttgtggaatctggccagcttcttgaagagattgacacccaggcatgcacactcactgacaccactgcttattgtactatagttagaacaaaaccattaccggaactagtcgcacaatgcctccgaggggatgagtctagatgccaatatacgactggaataggtatgcttgaatctcgatttggggtgtttgatggacttgttattgctaattgtaaggccaccatctgccgatgtctagcccctgagatgataataactcaaaacaagggactccctctcacagtcatatcacaagagacttgcaagagaatcctgatagatggagttactctacagatcgaagctcaagttagtgggtcgtattccaggaatataacagtcgggaacagccaaattgccccatccggaccccttgacatctcaagcgaactcgggaaggtcaaccaaagtctatctaatgtcgaagatctcattgaccagagcaatcagctcttaaatagggtgaatccaaacatagtaaacaacaccgcaatcatagtcacaatagtactgctagtcctcttggtattatggtgtttggccctaacgattagtatcctgtatgtatcaaaacacgctgtgcggatgataaagacagttccgaatccgtatgtaatgcaagcaaagtcgccgggaagtgccacacagttctaacaatatagctggtcctgatgattaaaccatatacctgattacatgataaaaatatgtcgagggatgacattgatgagaccccttattctctctcaaactaagacagtaatccatctagaatgcaatgatcctacttcctttactttaatcaaaaaatgcagaataatctaacagcccaaccaaactacccagaaagaaacgcctgaggggggaaggaggttgactgcaacctcaattgatcagaggttgtagtattaattctccataaccctcaagatgagaccacaagtggcggtttgggctttgcgcttattggccaccggcctagctatgatctccttagtgttctgcctgaaccaggtaattatgcaggtgctaattagggacattggggacttgttgacatcctcagaaataaagactacacgtgagacactgcgtgagcatctctcatctattactcttttcatgtcgtttgcgctgacttgctcaataagtgggtgcgttcttagcctggttgcattatatccaagcaagaacattaatggcactaacactcagccacaagtagaggaggccagatcggagaacctgtctcactcttccatgcacacaatcaataggccagcgacccctcccccaccgtattatgtagcaatacaactcagcgctgagatgcaacctgggtaccatccaggtgattgatcctcctagcatgttggcagagtctaccccaccaagatctgttcttgtaccacttttttgatttaagaaaaaattgtgatttatataaaaacataatggctgagggggaagcctggtgtcaccgctggtgaccatttcccagccggtggcaatggcttcctcaagcgatatgaggcagagtcaggcaactttatatgagggtgaccctaatagcaaaaggacatggaggaccgtgtaccgggttatcaccatattgttggatataaccgtcctttgtgtcggcatagtggcagtagtcaggatgtcaaccattacgacaaaggatattgataacagtatttcatcgtccattacttccctgagtgccgattaccagccaatatggtcagatacccatcagaaagtcaacagtattttcaaggaagtcggaatcactatccccgtcacactcgacaagatgcaagtggaaatgggaacagcagttaacataatcactgatgctgtaaggcaactacaaggagtcaatgggtcagcaggatttagcattaccaattcaccagaatatagtggagggatagacacactgatataccctcttaactcacttaatgggaaggctttagccgtatcagacctactggaacatccgagcttcataccagcgcctaccacctctcatggttgcacccgcattcctacattccacctaggataccgtcattggtgttatagtcacaacacaatagaatctggttgtcacgatgcagaagaaagcattatgtacgtatctatgggtgcggtaggggtcggccatcgtgggagacccgtgtttacgacaagtgcagcaacaatcctagatgatggaaggaacaggaaaagttgtagcatcatagcaaaccctaatgggtgtgatgtcttgtgcagcttggttaagcaaacagagaacgaggactacgctgaccctacaccgaccccaatgatccacggtaggctccacttcaatggcacgtacaccgagtctgaactcaaccctggcctatttaataatcattgggtcgctcaatatccagcagttggcagcggtgtcgtcagccacgggaaactattctttccgctttacggagggatatcaccgaagtcaaaactgttcaacgagcttaagtcatttgcttacttcacccataatgccgaattgaaatgtgagaacctgacagagagacaaaaagaagatctatataacgcatataggcctgggaaaatagcaggatctctctgggctcaaggggttgtaacatgtaatctgaccaatctagctgattgtaaagttgcaattgcgaacacgagcaccatgatgatggctgccgaggggaggttacaacttgtgcaagataggattgtcttctaccaaagatcctcatcatggtggccagtcctaatatattatgatatccctattagtgaccttatcagtgccgatcatttagggatagtaaactggactccatatccacaatctaaatttccgaggcccacctggacaaagggcgtatgcgagaaaccggcaatatgtcccgctgtgtgtgtaacgggtgtttaccaggatatttgggtagtcagtatagggtcccagagcaatgagactgttgtggttggcggatacttagatgctgcagcagcccgtcaggatccatggattgcagcagccaaccagtataactggctggttaggcgtcgcctttttacatctcagactgaagcagcatactcatcaaccacttgcttcagaaacacgaagcaggatagagtgttctgcctgactataatggaagttacagacaacctactcggagactggaggatcgccccgctgttatatgaggttactgtggctgataagcaacagggtaatcgcaattacgcgccaatgagaaggatggggacggataagttccaatattatacaccaggtgataaatatactcctcagcattgatgactcactgcagcttgtacataacaattctcttgcttcctctatttgcagagtaaatcagaagaatgacggtcggtgattgaccgaacttaattagatggtgacatacagcccgcattcatcttgatttaataaaaaactggggagctgttataacatggcagactgacggggcaagacccgcggagacaaagaatgcagtgagggggaaggcaggctgggatcacgtcccagctgtagccttccccgattcaatctacttagtatcgacaagtcaattctgctcacagaggtcatctgaaagggttgttgtgatggatccacaagtccaaatacaccacatcatcaagccagagtgccatctcaactcacctgtggtggagaagaaactgacgctattatggaagctcacaggtttaccgttgccacctgaccttaatagttgcgttacacacaaggatgtgacgtgggatgaagtgctccggttggaggctaatttgacaaaggaattacggcaattggtgcgaggcctgaccaatagaatgcatgaaagaggggagtttattgactcatataaacctttatgccatccacgtacgttaagttggttgactaatatcagcttgattaagagtgacaacattctagcaggccataagaaaatgctagtccggattggcggtatgttgcatgaacaaacagaccaattgtttgtcactcttggcaggaaattagcaggtgacccttgcttgttccatcaactaggccacctagctggatgtccacccaattccagatttgaggaacaggtaggaggctgcagtttgtggtcacccataagcgatccagccctggtcacaggtggtgaatatgccaactgtgtgtatgcgtggtacttaatacgtcaaaccatgcggtacatggccctccagagaaagcaaacaagagtgcaatcacagcagaatgtcctaattggatcagatacaatcgtgggaattcatccagaactagtgataattactggaattagagacaggacattcacctgtttgacttttgatatggtgctaatgtacgcagatgtggtggaaggccgtgccatgacaaagttggtcgcactcaccgagccgacaatggtagaagtcattcagagagtcgaaaaattgtggttcctagttgacagcatctttgaggaaatcggtggtgcaggttacaatattgttgcatctctggagagcttggcatatggtgctgttcaactgtgggataaatcactggaacatgctggtgagtttttctcattcaatcttactgagataaggagtgagctagagaaccatttagatcctggtatggcatttagagtagttgagcaggtgcggttgctatatactggactgagtgtgaaccaagcaggtgaaatgttatgcattttacgtcactgggggcatcccttgctgtgcgctgtgaaggcggcaaagaaagtcagagaatcgatgtgtgcaccgaaattaacctctctagataccacactcaaagtgttagcattctttattgcagatattatcaatggacatagacggtcacattcagggttatggccaagtgtcagacaagagtcacttgtgtccccattgctccagaacctctatagagaatccgccgagcttcagtacgcgattgtgcttaagcactatagagaagtatcccttattgaattccaaaaaagtattgattttgacttagttgaagatctgagcgtgttccttaaggataaagccatctgtcgaccaaagagtaactggttagctgtattcaggaagtccctactccccggacatttgaaagataagctgcaatctgagggcccttctaaccggcttctgcttgattttttgcaatcaagcgaatttgacccggctaaggagttcgagtacgtgacatcactggagtatcttcaggacccagagttctgcgcatcttattccttaaaggaaagggaagtcaagactgatgggcgcatatttgcaaaaatgactagaaaaatgaggaactgtcaagtcttgctagagagtttgctcgcatgccacatatgcgattatttcaaagagaacggagtagtacaagagcaaatcagcttaacgaaatcactgcttgcgatgtcgcaacttgctcctcgtgtgtctgagtatcaagggagagttctccgttcaactgataggtgcagtagagctacagctacacctgggcaggacacaagcccaggcgagggggtcaggcgacggaaaacgattatagcatcattcttgactactgacctacagaaatattgtctcaattggaggtacactgtagtaaaaccttttgcacagaggcttaaccagttatttgggataccccacggctttgagtggattcacctccgcttgatgaatacgaccatgtttgtaggagacccacataatgtccctcagttttcatcaacacatgacttagaatcccaagaaaatgatggaatatttattgtatcacctcggggtggtatagaagggctatgccaaaaaatgtggaccatgatctccattgcggcaattcatctagcagccacagaatcaggttgtcgggttgcatctatggtccaaggggacaatcaagcaattgcaatcaccacagagatcgaagagggtgaggacgcatccgtagcatcaataaggttaaaagaaatatctgagaggttttttagagtgttcagagagataaacagaggtatagggcacaacttaaaagtccaagaaacaattcatagtgagtcattcttcgtgtactcgaaacggatcttctttgaggggaagatcctcagtcaactactaaaaaatgcaagtaggttagtgttggtatccgagaccgtgggtgagaattgtgtcggcaattgctcaaatatcagttccacagttgctaggctcattgaaaatggattggataagagagtcgcatgggggctcaatatcatgatgatcgtaaaacaaattctttttgacattgatttctccttggagcctgaaccatctcaaggcttgattcatgctattcgccaagatccaaacaacataaaaaacatctctatcactcctgctcaattgggtggattaaattttctagccctatctcggctatttacaaggaacataggagaccccgtctcgtcagccatggcagatatgaagttctacatacaggtcggattattatcccctcatctgctaaggaatgcaatttttagggaacccggagatggaacgtggacaacactgtgcgccgacccgtactcattaaaccaaccatatgtgcaattgccaacgtcgtacttaaaaaagcacacacaacgcatgctgctcactgcctcaacaaatcctttattgcaaggcacccgagtagagaatcagtacattgaggaagaaagactagcaaagttccttctggatcgagaactggttatgccacgtgtggcgcatacagtctttgagaccactgttgccgggagacgaaagcatctgcaaggattgattgacaccacaccgaccattattaaatatgcccttcatcaccaccctatttctttcaagaaaagcatgctgatatcatcttactcagctgactacattatgtcgttcatcgatactatcgcaacagtggaatacccaaagcgtgacaccatgaagctctggaacagaggactaataggtgtcgacacctgcgcggtcacacttgcggattacgcaagaacatattcgtggtgggagatcctaaagggaaggtcgataaagggagttaccacacctgatacattagaactttgctctgggagcttaatagagcaaggccatccgtgtgctcagtgcacaatgggtgatgaatccttttcatggttcttcctcccaggaaatattgatattgaaaggccagacttttctagggtggcccagagaatcgcttatgtcggctcaaagacggaagaaaggcgggcagcttcactgacaacaatcaaagggatgtcaactcaccttagggcggcattaagaggagcgagtgtttacatctgggcgtatggagacagtgacaagaattgggacgatgctaccaagctcgctaacacaagatgtgtaatatctgaagaccatctacgtgccctttgtccaatcccaagttcagcaaacatacagcatagactgatggatggaataagtgtaacgaagttcactcccgcgtctctagcaagggtgtcatcatatattcatatttcaaatgaccggcatcagagtagaattgacggtcaagtgatcgaatcaaatgtgattttccaacaggttatgcttctcggtctcggcatttttgagacatttcaccctttgtctcacaggtttgtgactaaccccatgacactccacttacacacagggtactcatgttgcataagagaagctgataatggtgatttcttagaatccccggctagtgtaccagacatgactatcacgactggcaataagtttctttttgaccccatgcccatccaggatgacgatgctgcaaaactacaggtatcttcattcaagtactgtgagatgggccttgaagtgcttgacccaccaggacttgtaaccttactatccctagtgactgcacgcatctctattgatacatccataggggagagcgcgtacaactcgatacacaatgatgctattgtatcattcgacaattccatcaactggatatctgagtacacgtactgcgatcttagactactggcagtagcaatggctcgggagttttgcgacaatctctcttatcagctttactatctgagagttaaagggcgacgggcaatccgggattatatccgtcaagccctctcgaggataccagggttacagctcgctaatatagccttgactatatctcatccgggaatttgggcaagactgaggctaatcggggcagtaagtgctgggaatagccccattagtgcaaccgtaaattatcctgctgctgtgtgtgagctcatactatggggttatgaccaatatactgcacaactactagatggttatgaattagaaatcatagtcccaaattataaggatgatgacttgaacaggaaagttgaacatatattagcaagacgggcttgtctgctgagtctactgtgtgagtatccaggaaaatacccgaacatcaaagaccttgaacctattgagaaatgcactgccctgtctgacctgaataaattgtggatggcgacagatcacagaactcgggaatgtttttcagggatatctcagatatttgattcacccaagttaaatccattcatcactaatctttacttcttgagtaggaagctgctcaatgcaattagaagcagcacggactgtagggcctacgttgaaaacctttatgaagatattgacattgaactaacatctctcactgaggttgtacccttaggagaggatgatcaaatgatcactgggcctctgcgctttgaccttgaactaaaagaactcaccccagattttactatcacttggtgttgctttgactccacggcagcactgatgtcacggtgcattaatcatgccacagaaggtgcagagcgctacatccgaagaacggtcgggacagcttcaacatcttggtacaaagcagcaggaatattaactacacctggcttcctcaacctccctaaaggcaatggattgtatctcgctgagtcatcaggggccattatgactgtgatggagcatcttgtctgctctaataaaatctggtataacaccttgtttagcaatgagctcaacccacctcagaggaatttcggtcctaacccaattcagtttgaagaaagtattgtggggaaacacattgcagctgggattccttgcaaggcaggacacgtgcaagagttcgaggtactctggagagaggtagatgaagagacagatctgacatccatgagatgtgtgaattttatcatgtcgaaagttgaacagcactcgtgtcatattgtatgctgtgatttagaattggctatgggaactcccctggaagtggcccaatctgcatatacacacatcataaccctcgccttacattgcctaatgattagcggaaaattagtactgaagttgtatttctcacaaaatgccttattacaccatgttctctccttgttacttgtattaccatttcatgtaacaatccacactaatgggtattgctctcaccgaggctctgaagggtatatcatcgccacgaggacaggggttgctctgggttcaaatgtgtcccaggtactaggcagtgtgactgagatggtacggaaaggtcagacccttgtccccgtaaaggtacttacagcgatctccaatggatttaaaactgtatcaagctctttaggcaggcttaggggtgagctctattcgccatcgtgtagcattccgcagtcggctacagacatgttcctcattcaacttggagggaaggtgcagtcagattggaatacaaactctcgaggctatagagtgggtgataccgaactcgtattacaggacattatatcaatattgagcacactactaaaagaaataatacatgtaagggaatccagggagtcagtggacagggtgctgttgcttggggcatacaacctgcaagtatccggaaaagtaagaacaatggctgcggctgcaacaagaaacatattgcatctccatatagttagacttattggggactcaatgtccaatataaggagactagtacctctgctagataagggctttatagtaatatcagacatgtatagtgtgaaagatttcttgagaaaaactgagtcccctaagtacttcttgaacaagcttggcaagagcgagattgcacagatatttgaggtagagtccaagattattctgagcagggcagagatcaagaatattttgaaaatgatagggattgtagcaaaacagcactcagagtaatctcttcaactttgcgccgcttgaatcctgaactgtggacgcgcacgcttaagcgcaccagcctgacgtgacgattgatataatccttggtatgaatcaccaatcatctggaactcacttacttcccgaaatcacccatagaccggtatcggtatcggagattattatttaataaaaaacctggaaagtcaacaaggatcatagtcaaaaagcttatgatttccttgtttggt

SEQ ID NO: 20 - DNA, 15480 bp, artificial sequence
  APMV7 genome sequence
accaaacaaggactgcataagcagtgtaaaacttttaataaaaaataactttcgtgagggtgaatcgatcatcgctcgaagccgatatcgactcacccaaattagctgcttgtataaggatccgaatatcaattggaatcatgtcatcgatttttactgattataccaatttgcaagagcaattagtcagaccggtaggccggaaggttgataatgcttcaagtggcttgttgaaagttgagataccagtctgcgtcctgaattcacaggacccagttgagagacaccagttcgcagtattatgtacaaggtggatctcaagttcaattgccacaactcctgtcaagcaaggtgccctgctttctcttctcagtttgcacacagaaaacatgcgagcgcatgttctattagcagcccggtcaggagatgctaatataacaattctagaagttgatcatgtagatgttgaaaagggagaattacaatttaatgcaaggagtggtgtctcatctgataaagctgatcggctgctggctgtcgcaatgaatcttattgcaggttgtcagaataactcaccatttgtcgacccatcgattgagggtgatgaaccaactgatatgactgaatttttagagctggcttatgggttagcggttcaagcatgggtagctgcaataaagagtatgacggcaccagatactgctgcggagagtgaggggcggcgattagcaaaataccagcagcaaggtcgtttaacacgacgtgctgctcttcaagcaaccgtgaggggggagttgcagcggataatcaggggttctctggtagttcgacacttccttataggagaaatcagaagagcaggaagtatgggagaacagacaacagcctattatgccatggtgggagatgtcagccaatacataaagaattcaggaatgactgcattcttcctgacattacgatttggggtgggtaccaagtatcctccccttgcaatggctgcattttcaggagatctcactaaactccagagcctgatcagactatatcgaaataaaggtgacatagggccttatatggccctactcgaagatcctgacatgggcaactttgctcctgcaaattacaccttgctctattcatatgcaatgggcattggttctgtattggaggctagtatcggtagataccagtatgcgagaacattcctgaatgaatcattctttaggttgggggcctcaactgctcaacagcaacaaggagcactggatgagaaattggctaacgagatggggctatcagaccaggcaagggcagcagtttccagattagttaatgagatggatatggatcagcaagtagcccccacaccagttaatccagtctttgcaggagatcaagcagccccacaggcaaatcctccagcccaaccaagacagaatgacacaccacagcagcctgctcctcttcagcagccaattcgaattgccatgcctcaaaattatgatgatatgccagacttagagatgtagacagaaccccaatcaagcaacaattggcattaagatctaagctgaatgtatgagcacacgagtacccaagtatatttgttagcagttgcatgaaatcattatccatattattgatttgcaatatagaaaattactgataaacaattaagaatcatttaataaaaaaattccacaaaaattaaaaaaattgtgagggggaacacctttcagtcggtcaactgctgctaataacctgcaattatcacgtggattgaatatggaattcagtaatgatgccgaggttgccgcgctcctggatcttggagatagcatcattcagggcattcagcatgcaacaatggctgatccgggaacactagggaagtcagctattcctgcaggtaataccaaacgcttagagaaattatgggagaaagaatctgttcctaatcatgataatatgattcactcttccatgagtgcagaacctataagcggggaactacctgaggaaaacgctaaaactgaaccaacagggactcaagaaatgccagaacaaattcaaaagaatgacaatctccaacctgcatccatcgataacatattgagcagcattaatgcattagagtcaaaacaggttaaaaaagggttagtgctatcgccccaatcactgaaaggtgtgtcccccttaatcaagaaccaggatctgaagaacaccatgcaggacctggaaaccaaacccaaggctgtaacgactgtaaatccattagcaaaccgacaagtgtcacctggaagcctggtcatagacgagagtattcctttgcttggagtgcaggaacaaacaaatttattgtctcctcgtggtgtaacccaacttgcgccccaatcagaccctatcctacagtcgaacgatgcaggtgcgggaattgcccaaaattctgccctggatgtcaatcagctctgggatgtaatcaatcagcaacacaagatgctgataaacctacaaaatcaagtaacaaagatcactgagctggttgctttaattccaattcttcgaagtgatattcaggctgtaaagggaagttgcgcattattagaagcacagctagcatctataagaatactagatcctgggaacatcggggtatcttcattagatgatcttaaaacagcagggaaacaaagtgtagttattaatcaagggagctatactgatgcaaaggatctgatggttgggggaggattgattcttgatgaacttgctagacctactaaattagtcaatccaaagccacaacaatcttccaaaatattggatcaggcagaaattgaaagtgtcaaggccctaatccatacctacactcacgatgataagaagcggaacaaattcttaactgcacttgacaaggtgacaacccaggatcagctaactcgcatcaagcagcaagtattaaatcaatagatagacaattagcattcattcaagctatactcatttaagtgctttgattgtgttgcggaaactatattgagataatttagtcttacatgcaaaataacattaaaaattaattatgagcaatcttgatttttctaactcataatcaacctccttctctataaaggcatacttagtattgcaaaaagagaaaattaagaaaaaaagaaaaagaaaattgagggagaccgcttgatagatctgtgatcggtctcataacctcaaattaaaatggaatctatatctctggggttatatgttgatgaaagtgatccagcatgctcattacttgcattccccataatcatgcagactacaagtgaaggaaagaaggtcttacaaccgcaagtcagaataaaccgtctagggagtatatcgatagaaggagttcgggcaatgttcataaatacatatggcttcattgaggagaggcctacggaaaggacaggtttctttcagccaggcgaaaaaaatcagcagcaagttgtgacagctggtatgctgacattgggccaaataaggaccaatatagacccggacgaaattggagaggcatgcttgagactcaaagtgaatgctaaaaaatcagcagcaagtgaggagaagatagtatttagcattcttgaaaagcctcccgccctgatgactgcacctgtagtacaagatgggggcttaattgctaaagcagaaggatcaatcaaatgcccaggtaagatgatgagtgaaattcactactcatttagagtaatgtttgtgagtatcacaatgctggataatcagagcctatacagagtaccaacagccatcagctcgttcaaaaataaagctctatattctattcagttagaggtattgctggaagttgatgtgaagcctgagagcccccagtgtaaatttctagcagaccagaaagggaagaaagttgcttctgtatggttccatctctgcaattctaaaaagacgaatgccagcgggaaaccgagatcattagaggatatgagaaagaaggtccgagatatgggaatcaaagtgtctctggccgacctttggggccctacgatcatcgtcagggccacagggaagatgagtaaatatatgctaggatttttctctacctcagggacttcatgtcatccagtaacaaagagttcaccagatttggcaaaaatattatggtcatgctcaagcacaatcatcaaagcaaatgccattgttcaagggtcagtcaaagtcgatgtcctgaccctcgaagatatccaagtttccagtgctgcaaaaatcaacaaatcaggaatagggaagtttaatccatttaagaaataaagtcatatgcagattaaaatttgatcaagattggtcttagcaaattaactgaatgtaattataaaatacctcagtaaaatgctaatgaatcagtggatgatattgaattagcagattgaaaattaaagaaaaccttatgagggcgaatgagcttagatgatttaataaaggagactaatccaacatttccctcaaattaacaaaatcagaaagtaaaaagaaagggagcaatgagagtacgacctttaataataatcctggtgcttttagtgttgctgtggttaaatattctacccgtaattggcttagacaattcaaagattgcacaagcaggtattatcagtgcacaagaatatgcagttaatgtgtattcacagagtaatgaggcttacattgcactgcgcactgtgccatatatacctccacacaatctctcttgtttccaggatttaatcaacacatacaatacaacgattcaaaacatattctcaccaattcaggatcaaatcacatctataacatcggcgtcaacgctcccctcatcaagatttgcaggattagtagtcggtgcaatcgctctcggagtagcgacatctgcacaaataactgcagccgtggcactcacaaaggcacagcagaacgctcaagaaataatacgattacgtgattctatccaaaatactatcaatgctgtgaatgacataacagtagggttaagttcaataggagtagcactaagcaaggtccaaaactacttgaatgatgtgataaaccctgctctgcagaacctgagctgccaggtttctgcattaaacttagggatccaattaaatctttatttaaccgaaattacaactatctttggaccgcaaattacaaatccatcattgaccccattgtcaattcaggcattatacaccctagcaggagataacctgatgcaatttcttaccaggtatggctatggagagacaagtgttagcagtattctcgagtcaggactaatatcagcacaaattgtatcttttgataaacagacaggcattgcaatattgtatgtcacattaccatcaattgcgactctttccggttctagagttaccaaattgatgtcagttagtgtccaaactggagttggagagggttctgctattgtaccatcatacgttattcagcagggaacagtaatagaagaatttattcctgacagttgcatcttcacaagatcagatgtttattgtactcaattgtacagtaaattattgcctgatagcatattgcaatgcctccagggatcaatggcagattgccaatttactcgctcattgggttcatttgcaaacagattcatgaccgttgcaggtggggtgatagcaaattgtcagacagtcctgtgccgatgctataatccagttatgattattccccagaacaatggaattgctgtcactctgatagatggtagtttatgtaaagaacttgaattggaggggataagactaacaatggcagacccagtatttgcttcatactctcgtgatctgattataaatgggaatcaatttgctccgtctgatgctttagacattagtagcgaattaggtcaactgaataactcaattagctcagcaactgataatttacagaaggcacaggaatcattgaataagagtatcattccagctgcgacttccagctggttaattatattactatttgtattagtatcaatctcattagtgataggatgtatctccatttattttatatataaacattcaaccacaaatagatcacgaaatctctcaagtgacatcatcagtaatccttatatacagaaagctaattgatgaattaatttctaaaaaataatttgatgttctaataggagaatgcaatatcaatatgtccattataatatacttgattgattgaaagatctgataataatagtttataagacactaagtaagagttaaatgctaaagcaagttgattcctaaatttctgcacaataggaccatactatatcatattagataattaataaaaaacgccctatcctgagggcgaaaggccgatcattagtgactttaaccgttgctctcccaatttaaaatatatttcacatggagtcaatcgggaaaggaacctggagaactgtgtatagagtccttacgattctattagatgtagtgatcattattctctctgtgattgctctgatttcattgggtctgaagccaggtgagaggatcatcaatgaagtcaatggatctatccataatcaacttgttcccttatcggggattacttccgatattcaggcaaaagtcagcagcatatatcggagcaacttgctaagtatcccactacaacttgatcaaatcaaccaggcaatatcatcatctgctaggcaaattgctgatacaatcaactcgtttctcgctctgaatggcagtggaacttttatttatacaaattcacctgagtttgcaaatggtttcaatagagcaatgttcccaaccctaaatcaaagcttaaatatgctaacacctggtaatctaattgaatttactaattttattccaactccaacaacaaaatcaggatgtatcagaataccatcattttcaatgtcatcaagtcactggtgttatacccataatatcattgctagtggatgtcaggatcattcaaccagtagtgaatacatatcgatgggggttgttgaagtgactgatcaggcttacccgaactttcggacaactctttctattacattagctgataatctaaacagaaagtcatgtagcattgcagcaactgggttcgggtgtgatatattatgtagtgttgtcactgagacagaaaatgatgattatcaatcaccagaaccgactcagatgatctatggaagattattttttaatggcacatattcagagatgtcattgaatgtgaaccaaatgttcgcagattgggttgcaaattatccagcagttggatcaggagtagagttagcagattttgtcattttcccactctatggaggtgttaaaatcacttcaaccctaggagcatctttaagccagtattactatattcccaaggtgcccacagtcaattgctctgagacagatgcacaacaaatagagaaggcaaaagcatcctattcaccacctaaagtggctccaaatatctgggctcaggcagtcgttaggtgcaataaatctgttaatcttgcaaattcatgtgaaattctgacatttaacactagcactatgatgatgggtgctgagggaagactcttgatgataggaaagaatgtatacttttatcaacgatctagttcgtattggccagtgggaattatatataaattagatctacaagaattgacaacattttcatcaaatcaattgctgtcaacaataccaattccatttgagaaattccctagacctgcatctactgctggtgtatgttcaaaaccaaatgtgtgtcctgcagtatgccagactggtgtttatcaagatctctgggtactatatgatcttggcaaattagaaaataccacagcagtaggattgtatctaaactcagcagtaggccgaatgaacccttttattgggattgcaaatacgctatcttggtataatacaactagattattcgcacagggtactccagcatcatattcaacaacgacctgcttcaaaaatactaagattgacacggcatactgcttatcaatattagaattaagtgattctttgttaggatcatggagaattacaccattattgtacaatatcactttaagtattatgagctagatcctgttttaacattgaatcgtatgaacttataagactgaaggatgtctgttggtattaagcatcataaaacacggttgtttttgatttgacacctaatcgtactcaatactctccatagatttaatctaacagatttagatactattgatcatataggcatagatggtatatgggcaattagattgaactgagttaaatccgattgatacttatcaaattaagatctagattatttaataaaaaatctaagttagaaaatgagggggacctcattatggagttcagacaatctgatcaaataatacatcctgaagtgcatctagattcacctattattgggaataaaatactctatttatggcgaattacaggcttacctactccgcctgttcttgagcttaactctactatatcgcctgaagtctggacaaacttgaaagccaatgatcctagagtagcctttaaatgggacaaactaagaccacggttgctaacatgggcagcacatcaagggatatcactatcggatctgatccctattacacatcctgagtcattgcagtggttaacaacaatatcctgtcctaaaattgatgaaaattttgcgttaattaagaagtgccttcttagaacaagggactatacagcatcaggatttaagaatttattccaaatgatctcacagaaattgacgtcgacgaatattctattttgcgcagaaaatccgacaactccccccatctccgacgaagcatcctgggcattaaagaatcctgagcactggtttaatacaccttggtcatcttgttgtatgttttggttacatgtgaaacagactatgaggaacttaattagaatacaacgatctcaaccagaatcacaaagcatatacagtatcacggttgataacttgtttgttggattgactcctgacttgtgtgtcatagctgattctcaaagacaatcaattacagtactgtcatttgagtgtgtattgatgtattgtgacttaattgaaggtcgtaacaatgtttatgacctctgtcaattgtctcctgtgctaagtcctcttcaagatagaattttacttttactgagattaattgattctttagcatatgacatcggagcgccaatttttgatgtaattgcttctcttgaatctttagcatatggagctattcagctatatgattacgacacagaggcagccggtgattttttctcatttaatttaagagaaatttcccaggtcatagaagagagcaaatgtaggaatcaaacccatactataatcagtgcaattagtaagatttacacagggatcaatcctgatcaagcagctgaaatgctgtgtatcatgagactgtggggtcacccattgctttatgcatccaaggctgcatctaaggttcgcgagtcaatgtgtgcacctaaagttatccaatttgatgcaatgctgcttgtattagcattctttaagagaagcatcataaatggatatagacgaaagcatggtgggctatggccgaacatcatagttgagtcacttctttctgcagaacttgtcgcggcacatcatgatgcagttgaattgacagacacttttgttattaaacactatagagaagtagccatgattgacttcaaaaaatcattcgactacgatataggggatgacttaagtttatacctcaaggataaagcaatttgtcgacagaaatcagagtggcttaatatcttcaagggtcaattgcttgagcccgctgtacgatcgaagcgaattcgtggaataggtgaaaaccgattactgttacatttcttgaattcagtcgattttgatcctgaacaagaattcaaatacgtcactgatatggagtacctctacgatgaaacattctgtgcatcctattcactgaaggaaaaagaagtgaaaagagatggaagaatattcgcaaaaatgacaccaaaaatgagaagctgtcaagttttattagaggcattgttagcaaaacatgtaagcgaacttttcaaggagaatggagtctcaatggagcagatatccctcacaaagtcattggtagccatgtcacaattagctccccgagtgaatatgagaggtgggagagcagctagatcaacagacgttaaaatcaatcaacgaagggtcaagtcaatcaaagagcatgttaaatcgagaaatgattcgaatcaagagaaaattgtaattgcaggttatctgactactgatttacaaaaatactgcctcaattggagatatgaatcaataaaattatttgcaagagcacttaaccaattatttggaataccccatggatttgaatggatacacttaaggctcataagaagtacaatgtttgttggggatccttacaatcctcctgcatcaatccaatctttggatctcgatgaacagcctaatgatgatatttttattgtctcgccacgtggtgggattgaaggattatgtcagaagatgtggacactcatctcaattgcattaattcaagctgcagctgcaaaaataggatgtcgggttacaagtatggtacagggagataatcaggttattgctatcaccagagaagtgcgagtgggggaacctgtgagggaggcgtcacgagaactcagattattgtgtgatgagttcttcactgaattcaaacaattaaactacggaatagggcacaatcttaaagcaaaagaaactatcaagagtcaatcgttttttgtatatagcaagagagttttctttgagggaagagtgttaagtcagatattgaagaatgcctcaaaattgaatctaatttctgactgtctggctgaaaatacagttgcttcatgtagcaatatttcttctactgtagcaaggctaatagagaatggccttgggaaagacgtagccttcattttaaactttcagactattataaggcaactgatttttgatgaagtatatacgatttcattgaactatagtacagcaagacggcaggtgggaagcgagaatcctcacgcattggctatagccgctttgattcctggtcaacttgggggattcaatttcctaaacgttgctaggttatttacacggaatatcggggatccaatcacttgctcattgagtgatatcaaatggtttgcaaaagttggattgatgcctgagtacatccttaaaaacattgttttgagggcaccaggttcaggaacatggacaactttagtcgctgatccctactccttaaacattacgtacacaaaattgcctacgtcgtacctaaagaaacatacacagaggacattagttgctgattcccctaatccgttgcttcagggggtgtttctattaaatcagcagcaggaggatgaagcattatgtaaatttcttcttgaccgagaacaagtgatgccacgagctgcccatgtaatctatgatcagtcagttctcggccggaggaaatatttacaagggcttgttgatactacacagacaatcataaggtatgcactccaaaaaatgccggtatcatacaaaaagagtgaaaaaatccaaaattacaatctcctctacatacaatcactttttgatgaggtcttgacacagaatgtcattcatagtggattggatactatatggaaaagagatctaattagcattgagacctgttctgtcacacttgccaattttacgaggacttgctcgtggtctaatattctacagggcaggcaaattgttggagttacaactccagacacgatagaattgtgtaccggttctttgatttcttgcaacagtgcatgtgagttttgtagaattggagataaaagctactcttggtttcatacaccagggggtatctcatttgatacaatgagccctggcaatctgattcaaagagtgccgtacctaggatcaaagactgatgaacagcgagctgcctctctaacaaccatcaaggggatggattaccatctgagacaagctcttcgaggagcatcattgtatgtgtgggcatatggagagactgatcagaattggttagatgcgctgaagttagcaaacacccggtgcaatgtaacattacaagctttgactgcactctgcccaataccgagtaccgcaaatctacaacaccggcttgcggatggaataagtacagttaaattcacacctgcaagtttgtcacgaatagcagcttatattcacatttgtaatgaccaacaaaagcatgataacctagggaatagttttgaatcaaatctgatttaccagcaaataatgcttcttggaacaggaatatttgaaacaattttcccactatcagttcaatatatccacgaggaacaaacacttcacttgcacactggattttcctgttgtgtcagggaagctgacacaatgattatagatgagagcagaactggattcccaggattgacagtgactaagagtaataagtttttattcaaccctgaccctattcctgcagtgtgggcagataaaatattcacgactgaatttagattcttcgagtacaatatagagaatcaaggaacttatgaactaataaaatttctttcttcttgctgcgcgaaagttgttacagaatcgctagttcaggatactttccatagttctgtcaaaaatgatgcaataattgcgtatgacaattcaattaattacatcagtgagctacaacaatgtgacattgttctgtttagcagtgaacttggaaaggaattacttctagatttagcttaccagctgtactaccttcgaattagatcgaaacgaggtataattagttacttgaaggtactgctgactcggcttccaattattcagtttgcaccgcttgcgttgacaatatcacatcctgtaatctacgagcgattacgccaacggaggttggttatggaaccgttgcaaccttatttggcttcgatagattatgtcaaagccgcaagagagcttgttttgattggtgcttcttcttacctctcaatgcttgagacaggtttagataccacttacaacatatacagtcatttagacggggattcagagggcaagattgatcaggcgatggcaaggagactgtgcctaatcacattattagtgaatcctggatatgcattacctgtgatcaaaggactaactgcaattgagaaatgtagactattaacagattttttacaatcagatatcatttctgtttctttatctgagcagattgcaacacttattctaacaccaaagattgaagtgcacccgacaaatttatactatatgatgcggaagaccttgaatctaatccggtcacgagatgatacagttgtgatcatggcagaattgtataatatagatcaagagtctgcgataatgagggttgaatcagaagaggacggccctgtagacaaaatgaatcttgcacccatactaaggcttgtgccaatcacattcaaatcaatggacttgcatgccttaactgggctaggtagaaaagaggtggaactgatgggtagcccagtttgcaaaatcactcagagattagataagtacatctatcgcacaattggcaccatatctactgcatggtataaagcaagtagtttaatcgccagtgacatacttaaggggggcccattgggggacagcttatatttatgtgagggaagtggtagtagtatgacatgtttggaatattgtttcccttcgaaaacaatctggtataattcattcttctcaaatgagctaaatccacctcaacggaacatcggcccattaccaacacaattttgttcaagcattgtctatcacaatttgaatgctgaagtcccgtgctctgcagggtttatccaagatttcaaagtactctgggccgacaaatcagtggagactgatatttctacaactgaatgtgtgaatttcatcctaagcaaagttgaacttgaaacatgcaaattgatacatgcagaccttgatctacctattgagaccccaagatctgtctggatggcttgtgtcacaaatacattcattttgggaaatgccttattgaagtcaggagggaaattggtcatgaaattatatgcagtagatgagctcctcttttcatcttgcttaggattcgcatggtgccttatggacgatataaatatcctccgaaatggctacttcaatgacaaatcaaaggaatgctacctcattgggacaaaaaaggtgacaatcccgcaccagaaaatccaggatatccagcagcaaataaataagattgctagtcaagggttaagtgtcatacctgaagctgtaattcatgacatttacaaccagcttgaggacagtattagatgtgagaaaaaattcaaaaatgataatgcaccgacttggtccaatgggatcctcaattcgacagatctattactaataagacttggagggaaaccaattggggaatcactattagagttaacatccatacaaggcatggattatgatgatttaacaggggatataattcaagtaatagacacagcgctaaatgagattattcacctcaagtctgatacttcgagcttagatcttgtactgctaatgtctccttacaatctggcacttggagggaaaataagcacaattctgaaatctgttgttcaccagactctaatactcaggattatccaatctaggcagaataaggatataccattaaaaggatggttgtctctgttgaatcaaggagtcatctcactatcttcattgatcccgttgcatgattatctgaggaagagtaagttgagaaaatttatagttcaaaaattaggccaacaggaattacaagcattttggcagagcaggtctcaacaaatgctgagtagaagtgagaccaagttgctaataaaagtgctgagtgctgcttggaagggattgttgtaaaattgtaaatatacactgcatgtatataaattggttgctacccttatcagctaaccacaggtgtaaattttcatatggaatgcatatcaataaagataggcatttaaattatacaatgataacatattttaggttgacaacaatcattgatataatcaccaatagtagctctattacttatttgttaataataaatggtacactttgaatttaagaaaaaattagaattgctatattttatcgctatagtgggcctgtcggctgcgttagcggtaagacaaagaggacttgtcttttaaaaatttattaaaaaatcattaattgatcatattgctttccttgtttggt

SEQ ID NO: 21 - DNA, 31 bp, artificial sequence
  APMV-polyT oligo
ttttttttttttttttttaccaaacarrgaa

SEQ ID NO: 22 - DNA, 24 bp, artificial sequence
  8NPf1 oligo
caggagacctgatgttgcctcaac

SEQ ID NO: 23 - DNA, 25 bp, artificial sequence
  8NPr oligo
gcaggcgatctatagtctctgatag

SEQ ID NO: 24 - DNA, 15 bp, artificial sequence
  NP gene start sequence
cccccgcuucuguca

SEQ ID NO: 25 - RNA, 15 nt, artificial sequence
  P gene start sequence
cccccgcuggaguua

SEQ ID NO: 26 - DNA, 15 bp, artificial sequence
  M gene start sequence
cccccgcuucugugc

SEQ ID NO: 27 - DNA, 15 bp, artificial sequence
  F gene start sequence
cccccgcuuuagaac

SEQ ID NO: 28 - DNA, 15 bp, artificial sequence
  HN gene start sequence
cccccgcuggguaaa

SEQ ID NO: 29 - DNA, 15 bp, artificial sequence
  L gene start sequence
cucccgcuggagaug

SEQ ID NO: 30 - DNA, 16 bp, artificial sequence
  NP gene end sequence
aacuaaauucuuuuuu

SEQ ID NO: 31 - DNA, 16 bp, artificial sequence
  P gene end sequence
uaacuaauucuuuuuu

SEQ ID NO: 32 - DNA, 16 bp, artificial sequence
  M gene end sequence
aggauuaauauuuuuu

SEQ ID NO: 33 - DNA, 16 bp, artificial sequence
  F gene end sequence
cuauaaauuauuuuuu

SEQ ID NO: 34 - DNA, 16 bp, artificial sequence
  HN gene end sequence
uacuuaauucuuuuuu

SEQ ID NO: 35 - DNA, 16 bp, artificial sequence
  L gene (1) end sequence
acuaaaauucuuuuuu

SEQ ID NO: 36 - DNA, 16 bp, artificial sequence
  L gene(2) end sequence
uuauugauuuuuuuuu

SEQ ID NO: 37 - DNA, 15342 bp, artificial sequence
  AMPV8 genome FJ215863
accaaacaaggaatgcaagaccaacgggaactttaaataaaacaatcgaattattgggggcgaagcaagtggatctcgagctcgaggccgaaaccctgaatttcactggaggttttgaataggtcgctataggactcaatatgtcatctgtattcaatgagtatcaggcgcttcaagaacaacttgtaaagccggctgtcaggagacctgatgttgcctcaacgggtttactcagagcggaaatacctgtctgtgtcacattatctcaagaccccggtgagagatggagccttgcttgcttgaatatcagatggcttgtgagtgattcatcaaccacaccaatgaagcaaggagcaatattgtcactgctgagtctacattcagacaatatgcgagctcacgcaacattagcagcaaggtctgcagatgcttcactcaccatacttgaggtagatgaagtagatattggcaactcactaatcaaattcaacgccagaagtggtgtatctgataaacgctcaaatcaattgcttgcaattgcggatgacatccccaaaagttgcagtaatgggcatccatttcttgacacagacattgagaccagagacccgctcgatctatcagagactatagaccgcctgcagggtattgcagctcagatatgggtgtcagccataaagagcatgacagcgcctgacaccgcatcagagtcagaaagtaagaggctggccaaatatcaacaacaaggccgactggttaagcaagtactcttgcattctgtagtcaggacagaatttatgagagttattcggggcagcttggtactgcgccagtttatggttagcgagtgcaagagggcttcagccatgggcggagacacatctaggtactatgctatggtgggcgacatcagtctgtacatcaagaatgcaggattgactgcatttttcctcaccctgaagttcggagttggtacccagtatccaaccttagcaatgagtgttttctccagtgaccttaaaaggcttgctgcactaatcaggctatacaaaaccaagggagacaatgcaccatacatggcattcctggaggactccgatatgggaaattttgctccagcaaattatagcacaatgtactcttatgccatgggcattgggacaattctggaagcatctgtatctcgataccagtatgccagagactttaccagtgagaattatttccgtcttggagttgagacagcccaaagccagcagggagcatttgacgagagaacagcccgagaaatgggcttgactgaggaatcaaaacagcaggttagatcactgctaatgtcagtagacatgggtcccagttcagttcatgagccatctcgccctgcatttatcagtcaagaagaaaataggcagcctgcccagaactcgtcagatactcagggtcagaccaagccagtcccgaagcagcccgcaccaagggccgactcagatgacattgatccatacgagaacgggctagaatggtaattcaaccaccccgacacatccacctatacaccaattctgtgacatattaacccaatcaaacatttcataaactatagtagtcattgatttaagaaaaaattgggggcgacctcaattgtgaaacataccagatccgtccacaacaccactcaacaacccacacacaatggatttcgccaatgatgaagaaattgcagaacttttgaatctcagcaccaatgtaatcaaggagattcagaaatccgaactcaagcctccccaaaccaccggacgaccacctgtcagtcaagggaacacaagaaatctaactgatctatgggaaaaagagactgcaagtcagaccaagacaccggcccaatctccacaaaccacacaagttcagtctgatgaaaatgaggagggagaaatcaagtccgagtcaattgatggccacatcagaggaactgttaatcaatcagagcaagtcccagaacaaaaccagagcagatcttcaccaggtgatgatctcgacagagctctcaacaagcttgaagggagaatcaattcaatcagctcaatggacaaagaaattaaaaagggccctcgcatccagaatctccctgggtcccaggcggcaactcaacaggcgacccacccattggcaggggacaccccgaacatgcaagcacagacaaaagccctggcgaagccacatcaagaggcaatcaatcctggcaaccaggacacaggagagagtattcatttaccaccttccatggcaccaccagagtcattagttggtgcaatccgcaatgcaccccaattcgtgccagaccaatctatgacgaatgtagatgcggggagtgtccaactacatgcatcatgtgcagagatgataagtagaatgtttgtagaagttatatccaagcttgataaactcgagtcgagactgaatgatatagcaaaagttgtgaacaccaccccccttattaggaatgatattaaccaacttaaggccacaaccgcactgatgtccaaccaaattgcttccatacaaattcttgacccagggaatgcaggggtgaggtccctctctgaaatgaaatctgtgacgaagaaagctgctgttgtaattgcagggtttggagacgacccaactcaaattattgaagaaggcattatggccaaagatgctcttggaaaacctgtgcctccaacatctgttatctcagctaaagctcagacttcttccggtgtgagtaagggtgaaatagaaggattgattgcattggtggaaacattagttgacaatgacaagaaggcagcaaaactgattaaaatgattgatcaagttaaatcccacgccgattacgcccgagtcaagcaggcaatatataatgcgtaatactgtaactatacaaacaatcaatactgctgtcggttgcacccacctcagcaaatcaataatcttttagaatttattgattaagaaaaaattgactactataaggaaagaacaccaagttgggggcgaagacacgattgaccacagtcgctatctgtaaggctcctcaccaaaaatggcatatacaacattgaaactgtgggtggatgagggtgacatgtcgtcttcgctcctatcattcccgttggtactaaaagagacagacagaggcacaaaggagcttcaaccacaggtaagggtagattcaattggcgatgtgcagaacgccaaagagtcctcgatattcgtgactctatatggtttcatccaagcaattaaggagagtacagatcgatcgaaattcttccatccaaaagatgacttcaaacctgagacagtcactgcaggactggtagtggtaggtgcgatccgaatgatggctgatgttaataccatctctaatgacgcactagcgctggagatcactgttaagaaatctgcaacttctcaagagaaaatgacggtgatgttccacaatagccccccttcattgagaactgcaataactatccgagcaggaggtttcatctcgaatgcagacgagaatataaaatgtgccagcaagttgactgcaggagtgcagtacatattccgcccaatgtttgtttcaatcactaaattacacaatggcaaactatatagggtgcccaaaagcatccacagcatctcgtccactctactgtatagtgtgatgttggaggtaggattcaaagtggatattgggaaggatcatccccaggcaaagatgctgaagaaggtcacaatcggcgatgcagacacatactgggggtttgcatggttccacctgtgcaatttcaaaaagacatcctctaagggaaagccaagaacgctagacgaactaaagacaaaagtcaaaaacatggggttgaaattggagttacatgacttgtggggtccgactattgtggtccaaatcactggcaagagcagcaaatatgctcaaggatttttttcctccaatggtacttgttgcctcccaatcagcagatctgcaccagggcttgggaagcttctgtggtcctgttcagcaactatcggtgacgcaacagttgttatccagtcaagcgagaaaggggaactcctaaggtctgatgacctcgagatacgaggtgctgtggcctccaagaaaggtagactgagctcatttcaccccttcaagaaatgatgcaggacatagtacagagaattagagagccattagatgtgcgcaaaaaacataacctgcgatgaactgcccagactccactttaacctaggttgcagggaaatagtacacgacatgcacaatactatcacggtcaccagcaatcaataaagttgatcaatcactatattaggaatcaaataggataacaattattaatccaatttcctaattataaaaaattgctttaaaggttattgacgagtcgggggcgaaatcttgccacttagtctgcagtcaatcttagaatctacatattgaactatgggtcaaatatcagtatatctaattaatagcgtgctattattgctggtatatcctgtgaattcgattgacaatacactcattgccccaatcggagttgccagcgcaaatgaatggcagcttgctgcatatacaacatcactttcagggacaattgccgtgcgattcctacctgtgctcccggataatatgactacctgtcttagagaaacaatcactacatataataatactgtcaacaacatcttaggcccactcaaatccaatctggatgcactgctctcatctgagacttatccccagacaagattaattggggcagttataggttcaattgctctcggtgttgcaacatcggctcaaatcactgctgcagttgctctcaagcaagcgcaagacaatgcaaggaacatactagcactcaaagaagcactgtccaaaaccaatgaggcggtcaaggagcttagtagtgggttacaacaaacagctattgcacttggtaagatacagagttttgtgaatgaggaaattctgccatctatcaaccaactgagctgcgaggtgacagccaataaacttggggtgtatttatctctgtatctcacagaactgaccaccatattcggtgcacagctgactaaccctgcattgacttcattatcatatcaagcgctgtacaacctgtgtggtggcaacatggcaatgcttactcagaagattggaattaaacaacaagacgttaattcgctatatgaagccggactaatcacaggacaagtcattggttatgactctcagtaccagctgctggtcatccaggtcaattatccaagcatttctgaggtcactggtgtacgtgcaacagaattagtcactgttagtgtaacaacagacaagggtgaagggaaagcaattgtaccccaatttgtagctgaaagtcgggtgactattgaagagcttgatgtagcatcttgtaaattcagcagcacgaccctatattgcaggcaggtcaacacaagggcacttcccccgctagtagctagctgtcttcgaggtaactatgatgattgtcaatataccacagagattggagcattatcatcccggtatataacactagatgggggggtcttagttaattgtaagtcaattgtttgtaggtgccttaatccaagtaagatcatctctcaaaatacaaacgctgcagtaacatatgttgatgccacaatctgcaaaacaattcaattggatgatatacaactccagctggaagggtcactatcatcagtttatgcaagaaacatctcaattgagatcagtcaggtgaccacctccgggtctttagatatcagcagtgagataggaaacatcaataatacggtgaatcgtgtggaggatttaattcaccaatcagaggaatggctggcaaaggttaacccacacattgttaataatacaacactaatcgtactctgtgtgttaagtgcgcttgctgtgatctggctggcagtattaacggctattataatatacttgagaacaaagttgaagactatatcggcattagctgtaaccaatacaatacagtctaacccctatgttaaccaaacgaaacgtgaatctaagttttgatcattcaagccaaaacagaggatctaggctcgggttaataatagttcaatcaatgtttgatttattaggtttttcccactaattattaatatattcgtgattagatgataacgttaaaagtcttaaatatttaataaaaaatgtaacctgggggcgacccatttataggttagtatatattaggaagtccttatattgcactgtaatttcaaacaattatattacctcatatctaccttgtctaaagacatcatgagtaacattgcatccagtttagaaaacattgtagagcaggatagtcgaaaaacaacttggagagccatctttagatggtccgttcttcttatcacaacaggatgcttagccttatccattattagcatagttcaaattggaaatttgaaaattccttctgtaggggatctggctgatgaagtggtgacacccttgaaaaccactctgtctgatacactcaggaatccaattaaccagataaatgacatatttaggattgttgcccttgatattccattgcaagtgaccaatatccaaaaagaccttgcaagtcaatttaacatgttgatagatagtttaaatgctatcaaattaggcaacgggaccaaccttatcatacctacatcagacaaggagtatgcaggaggaattggaaaccctgtatttactgtcgatgctggaggttctataggattcaaacagtttagcttaatagaacatccgagctttattgctggacctacaacgacccgaggctgtacaagaatacccacttttcacatgtcagaaagtcattggtgctactcacacaacatcatcgctgctggctgtcaagatgccagtgcatccagtatgtatatctcaatgggagttctccatgtgtcctcatctggcactcccatttttcttactactgcaagtgagctgatagacgatggagttaaccgtaagtcatgcagcattgtagcaacccgatttggctgtgacattttgtgcagtattgtcacagagaaggagggagatgattactggtctgatactccgactccaatgcgccacggccgtttttcattcaatggtagttttgtagaagccgaactaccagtgtccagtatgttctcgtcattctctgccaactaccctgctgtgggatcaggcgaaattgtaaaagatagaatattattcccaatttacggaggtataaagcagacttcaccagagtttaccgaattagtgaaatacggactctttgtatcaacacctacaactgtgtgccagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcggaaggaaggataatgaagatagggattgattacttttactatcagcggtcatcttcttggtggccattggcatttgttacaaaactagacccgcaagaattggcagacacaaactcaatatggctgaccaattccataccaatcccgcaatcaaagttccctcggccttcatattcagaaaattattgcacaaagccagcagtttgccctgctacttgtgtcactggtgtgtactctgatatttggcccctgacctcatcttcatcactcccgagcataatttggatcggccagtaccttgatgctcctgttagaaggacttatcccagatttggaattgcaaatcagtcacactggtacctccaagaagatattctacccacttccaccgcaagtgcgtattcaaccactacatgttttaagaatactgccaggaatagagtgttctgcgtcaccattgccgaatttgcagatgggttgtttggagagtacaggataacacctcagttgtacgaattagtgagaaataattgaataacgataattttgagactcattttgtcgcaaagtgaaattgtcatctttaaaaataatcaattctatgatttttattgaacatgattaagcaatcatgtgggaaatttattatctcataaattctaatagttgtaaatgacgaattaagaaaaaatggagggcgacctctacacaaacatggatataaaacaagttgacctgataatacaacccgaggttcatctcgattcacccatcatattgaataaactggcactattatggcgcttgagtggtttacccatgcctgcagacctacgacaaaaatccgtagtgatgcacatcccggaccacatcttagaaaaatcagaatatcggatcaagcaccgtctagggaaaatcaagagtgacataacacattactgccagtattttaatattaatttggcaaatcttgatccgataacccaccccaaaagtttgtattggttatccagactaacaatagctagtgctggaacctttagacatatgaaagatagaatcttgtgtacagttggctctgaattcggacacaaaattcaagatttattttcactgttgagccataaactagtaggtaacggtgatttatttaatcaaagtctctcaggtacacgtttgactgcgagtccgttatcccctttatgcgatcaatttgtctctgacatcaagtctgcagtcacgacaccctggtcagaagctcgttggtcttggcttcatatcaaacaaacaatgagatatctaataaaacaatcatgcactacaaattcggctcatttaacagaaatcataaaagaggaatggggtttagtaggtattactccagatcttgtcattctttttgacagagtcaataatagtctgactgcattaacatttgagatggttctaatgtattcagatgtattagaatcccgtgacaatattgtgttagtggggcgactatctacctttctacagccagtagttagtagactggaggtgttgtttgatctagtagattcattggcaaaaatcttaggtgacacaatatatgagattattgcagtgttagagagcttgtcttatgggtcagttcaactacatgatgcaagtcactctcatgcagggtcttttttttcatttaacatgaatgaacttgataacacactatcaaagagggtagatccgaaacacaagaacaccataatgagcattataagacaatgcttttctaatctagatgttgatcaagctgcagagatgctatgcctgatgagattattcggacacccaatgttaactgcaccggatgcagcagccaaagtgaggaaagcaatgtgtgctccaaaacttgttgaacacgacaccatcttgcagacattatctttcttcaaggggataattataaatgggtacagaagatcacactctggcctgtggcccaatgtagagccgtcttcaatttatgatgatgatctcagacagctgtacttagagtcagcagagatttcccatcatttcatgcttaaaaactacaagagtttgagcatgatagaattcaagaagagcatagactacgatcttcatgatgacttaagtactttcttaaaggatagagcaatttgccggccgaaatcccagtgggatgtcatatttcgtaaatctttacgcagatctcatacgcagtcccagtatctagacgaaattaagagcaaccggttgctaattgattttcttgattctgctgaatttgaccctgaaaaagaatttgcatatgtaaccacaatggattatttgcacgataatgaattttgtgcttcatattctctaaaggaaaaggagatcaaaactactgggaggatatttgcaaaaatgacacgcaatatgagaagttgccaagtaatacttgaatctttgttatcaaagcatatatgcaagttcttcaaagagaatggcgtttcgatggagcaattgtcattgaccaagagtctacttgcaatgtctcaactctcaccaaaagtctcgactttgcaggacactgcatcacgtcatgtaggcaactcaaaatctcagattgcaaccagcaacccatctcggcatcactcgacagccaatcagatgtcactctcaaatcgaaaaacggttgtagcaactttcttaacaactgacttggaaaaatactgcctgcagtggcgatactcaactattaaattgtttgcacaagctctaaatcaactctttgggattgatcacggatttgaatggatacatttaagacttatgaacagcaccttatttgttggcgatccttactcgcctcctgaagatccaacactagaagatatagataaagcaccaaatgatgatatcttcatagtttctccaaggggaggcatagagggtttatgtcagaaaatgtggaccatgatatcaattagtgctatacactgtgtagcagagaaaattggtgcacgagtggcagcaatggtgcagggtgataatcaagtaatagctatcaccaaagaactattcagaggagaaaaagcttgtgatgtcagagatgagttagacgagcttggtcaagtgttttttgatgagttcaagagacacaattatgcaattggacacaatcttaagctaaatgagacaatacaaagccaatccttttttgtatattccaaacgaatattctttgaagggcgattgcttagtcaagtcctcaaaaatgctgccaagttatgtatggttgctgaccatctaggtgaaaacactgtatcttcctgtagcaacctgagctcgacaattgcccgcttggtggaaaatgggtttgagaaggacactgcttttgtgttgaacctagtctacatcatgactcagattctttttgatgagcattactcgattgtatgcgatcaccatagtgtcaaaagtttgattggatcaaaaaaccatcggaatttattgtactcatctctaataccaggtcagctcggcggtttcaacttcctcaatataagtcggttgttcactaggaatataggtgacccagtaacatgtagtctgtctgatctcaaatgcttcatagccgcaggtctccttccaccctatgtcctaaaaaatgtggttctgcgtgagcctggtcctgggacatggttgacgttgtgctctgatccttacacccttaacataccatacacacagcttccaaccacatatctcaaaaagcacacccagcgatcattgctttcacgtgcagtaaatcctttattagccggtgtacaagtgccaaatcagcatgaggaagaagaggtgttggctcgctttctccttgatcgtgaatatgtgatgccccgcgttgctcatgtaatactagaaacatcggtccttggcaaacggaaacaaatccaaggcttaattgatacaactccaaccatcattagaacatctctagttaatctgccagtgtctagaaagaaatgcgaaaaaataatcaattactctctcaattatattgctgagtgtcatgactccttacttagccaggtctgcttcagtgataataaggaatacttgtggtcaacctccttaatatcagttgagacatgtagtgtgacaatcgcggactatctgagagctgtcagctggtctaatatattagggggaagaaacatatccggggtgactacacctgatactattgaattaattcaaggttgtttaataggtgaaaattctagttgtactctttgtgaatcgcatgatgacgcattcacgtggatgcacttgcctggcccactttacatccctgaaccatcagttactaactctaaaatgcgtgtgccatatctgggttcgaaaacagaggagcgtaaaacagcctcaatggcagcaataaaaggaatgtcacatcacctgcgtgcagtcttaagaggcacatccgtatttatttgggcatttggggatacagatattaattgggataatgcattgcagattgcccaatcacggtgtaacatcacattggatcaaatgagattacttacaccaattcctagcagttcaaatattcaacatagactcgatgacggaatcagcacgcagaaatttactcctgcaagccttgctcgaatcacatccttcgttcacatctgtaatgacagccagaggttagagaaggatggctcatctgttgactcaaacttgatttaccagcaaattatgttacttggactcagcatctttgaaacaatgtactcaatggaccaaaagtgggtattcaataaccataccttgcatttgcacactggacactcctgttgtccaagggaactagacataagtttggtgaacccgccgagacatcagaccccggagctgactagcacaacaaccaacccgttcctatatgatcagctcccattaaatcaagaaaacttgacaacacttgagattaagacatttaaattcaatgagctcaacattgatggtttagattttggtgaaggaatacaattattgagtcgttgtactgcaagattgatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagacgggccattcttaattacttggacacaactttgcaaaggatccctgtgatacaattagccaacattgcactcaccatttcgcaccctgagatatttcgcagaattgtcaacaccgggatccataaccagattaagggcccatatgtcgcaacaacggatttcatagctgcaagtagagatatcatattatcaggtgcaagggagtatctatcttatttaagcagtgggcaggaagactgttacacattcttcaactgtcaagatggggatcttactccaaaaatggaacagtatcttgcaaggagggcatgccttttaacattattgtataatactgggcaccagatccccgttatccgatcactgacaccaatagagaagtgcaaggtgctcacagaatacaatcaacaaattgagtatgcagatcaagagtttagctctgtattaaaagtggtcaatgcactactacaaaatcctaagatagatgcattagtttcaaatctctacttcaccaccagacgtgttctatcaaacctcagatcatgtgataaggctagatcatatattgaatatttgtacactgaggacttcggagagaaagaggatacagtacaatatgacatcatgacaacaaacgatatcatacttactcatggtctattcacacagatcgaaatatcttatcaagggaatagtctccataagttccttactccggataacgcgcctggatctttgatcccattctctatttcaccaaattcacttgcatgtgatcctcttcatcacttgctcaagtcggtcggtacatcaagcacaagttggtacaagtatgcaatcgcctatgcagtgtctgaaaagaggtcagctcgattaggagggagcttgtacattggtgaagggagcggaagtgtgatgactttactcgagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctagggtttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttatacccatatcttacttgttgctacttacggattacagcaagattcaatactgtgggtgaaggtatataggacatctgaaaaagtatttcaattcttactgagtgccatgatcatgatctttggttatgtcaaaatccacaggaatggttatatgtcgacaaaggatgaagagtacatattgatgtctgactgcaaggaacctgtaaactatacagctgtccctaacattcttacacgtgtaagtgatttagtgtcgaagaatctgagtcttatccatccagaagacctcagaaaagtaaggtgtgaaacagattccctgaatttgaagtgcaatcatatttatgagaaaataattgccagaaaaattccattacaggtatcatcaactgattctttgctcctccaattaggcggtgttatcaactcggtgggctcaactgatcctagagaggttgcaacattatcttctattgagtgtatggactatgttgtctcatcaattgatttggctatattggaggcaaatattgtaatctcagagagtgctggtcttgacctcgctttaatgttaggcccattcaacttgaataagcttaagaaaattgatacaatccttaagtcaagcacctatcagctaatcccgtactggttgcgctatgagtactctattaatccgagatctttgtcatttctaatcactaaattacaacaatgccgaatttcatggtcagatatgatcacgatttctgaatttcgtaagaaatccaagcggcctatatttatcaaacgagtaatagggaatcaacagctaaaatcattctttaatgaaagctcaagtattgttttgactcgggctgaagttaaagtctgtataaagttcctcggtgcaatcatcaagttgaaataatttctgcgattttaaaggggtgtaatgttctaatttgcacttggagtaatatagcttgtaatcattcgataggggataggataatttctctaacctctgaatctatatccctagagtataacaaatatatacataataaaaatgattttaagaaaaaatccgacactcaaagaaaattggtgcctgtaatattcttcttgccagatgattgtggagtgtctagcctaacttaaaacaatcgtattcgatagggaagaatggtatataaaataactaataaaaaaatgtattagtaaaaattaccgtatttcctgtattccatttctggt

SEQ ID NO: 38 - DNA, 15342 bp, artificial sequence
  APMV8 genome FJ215864
accaaacaaggaatgcaagaccaacgggaactttaaataaaacaatcgaatcattgggggcgaagcaagtggatctcgggctcgaggccgaaacactggatttcgctggaggttttgaataggtcgctataagactcaatatgtcatctgtattcaatgaatatcaggcacttcaagaacaacttgtaaagccggctgtcaggagacctgatgttgcctcaacaggtttactcagggcggaaatacctgtctgtgttacattgtctcaagaccccggtgagagatggagccttgcttgccttaatatccgatggcttgtgagtgattcatcaaccacaccaatgaagcagggagcaatattgtcactgctgagtctacattcagacaatatgcgagctcacgcaacattagcagcaaggtctgcagatgcttcactcaccatacttgaggtagatgaagtagatattggcaactccctaatcaaattcaacgctagaagtggtgtatctgataaacgatcaaatcaattgcttgcaattgcggatgacatccccaaaagttgcagtaatgggcatccatttcttgacacagacattgagaccagagacccgctcgatctatcagagaccatagaccgcctgcagggtattgcagctcagatatgggtgtcagccataaagagcatgacagcgcctgacaccgcatcagagtcagaaagtaagaggctggccaaataccaacaacaaggccgactggttaagcaagtacttttgcattctgtagtcaggacagaatttatgagagttattcggggcagcttggtactgcgccagtttatggttagcgagtgcaagagggcttcagccatgggcggagacacatctaggtactatgctatggtgggtgacatcagtctgtacatcaagaatgcaggattgactgcatttttcctcaccctgaagttcggggttggtacccagtatccaaccttagcaatgagtgttttctccagtgaccttaaaagacttgctgcactcatcaggctgtacaaaaccaagggagacaatgcaccatacatggcattcctggaggactccgatatgggaaattttgctccagcaaattatagcacaatgtactcttatgccatgggcattgggacgattctggaagcatctgtatctcgataccagtatgctagagactttaccagtgagaattatttccgtcttggagttgagacagcccaaagccagcagggagcgtttgacgagagaacagcccgagagatgggcttgactgaggaatccaaacagcaggttagatcactgctaatgtcagtagacatgggtcccagttcagttcgcgagccatcccgccctgcattcatcagtcaagaagaaaataggcagcctgcccagaattcttcagatactcagggtcagaccaagccagtcccgaatcaacccgcaccaagggccgacccagatgacattgatccatacgagaacgggctagaatggtaattcaatcacctcgacacatccacctatacaccaattctgtgacatattaacctaatcaaacatttcataaactatagtagtcattgatttaagaaaaaattgggggcgacctcaactgtgaaacacgccagatctgtccacaacaccactcaacaacccacacaagatggacttcgccaatgatgaagaaattgcagaacttctgaacctcagcaccactgtaatcaaggagattcagaaatctgaactcaagcctccccaaaccactgggcgaccacctgtcagtcaagggaacacaagaaatctaactgatctatgggaaaaggagactgcaagtcagaacaagacatcggctcaatctccacaaaccacacaagttcagtctgatggaaatgaggaggaagaaatcaaatcagagtcaattgatggccacatcagtggaactgttaatcaattagagcaagtcccagaacaaaaccagagcagatcttcaccaggtgatgatctcgacagagctctcaacaagcttgaagggagaatcaactcaatcagctcaatggataaagaaattaaaaagggccctcgcatccagaatctccctgggtcccaagcagcaactcaacaggcgacccacccattggcaggggacaccccgaacatgcaagcacggacaaaacccctgaccaagccacatcaagaggcaatcaatcctggcaaccaggacacaggagagaatattcatttaccaccttccatggcaccaccagagtcattagttggtgcaatccgcaatgtaccccaattcgtgccagaccaatctatgacgaatgtagatgcggggagtgtccaactacatgcatcatgtgcagagatgataagtagaatgtttgtagaagttatatctaagcttgataaactcgagtcgagactgaatgatatagcaaaagttgtaaacaccaccccccttatcaggaatgatattaaccaacttaaggccacaactgcactgatgtccaaccaaattgcttccatacaaattcttgacccagggaatgcaggggtgaggtccctctctgaaatgagatctgtgacgaagaaagctgctgttgtaattgcaggatttggagacgacccaactcaaattattgaagaaggtatcatggccaaagatgctcttggaaaacctgtgcctccaacatctgttatcgcagccaaagctcagacttcttccggtgtgagtaagggtgaaatagaaggattgattgcattggtggaaacattagttgacaatgacaagaaggcagcgaaactgattaaaatgattgatcaagttaaatcccacgccgattacgcccgagtcaagcaggcaatatataatgcataatattgtaattatacaaacaatcaatactgctgtcggttgcacccaccttagcaaatcaataatcttttaaaattgattgattaagaaaaaattgactacaataaggaaagaacaccaagttgggggcgaagtcacgattgaccacagtcgctatctgtaaggctcctcaccaaaaatggcatatacaacactaaaactgtgggtggatgagggtgacatgtcgtcttcgcttctatcattcccgttggtactaaaagagacagacagaggcacaaagaagcttcaaccacaggtaagggtagattcaattggcgatgtgcagaatgccaaagagtcctcgatattcgtgactctatatggtttcatccaagcaattaaggagaattcagatcgatcgaaattcttccatccaaaagatgacttcaaacctgagacagtcactgcaggactggtagtagtgggtgcaatccgaatgatggctgatgtcaataccatctctaatgatgcactagcgctggagatcactgttaagaaatctgcaacttctcaagagaaaatgacggtgatgttccacaatagccccccttcattgagaactgcaataactatccgagcaggaggtttcatctcgaatgcagacgaaaatataaaatgtgccagcaagttgactgcaggagtgcagtacatattccgtccaatgtttgtttcaatcactaaattacacaatggcaaactatatagggtgcccaaaagtatccacagcatctcgtctaccctactgtatagtgtgatgttggaggtaggattcaaagtggacatcgggaaggatcatccccaggcaaaaatgctgaagagggtcacaattggcgatgcagacacatactggggatttgcatggttccacctgtgcaatttcaaaaagacatcctctaagggaaagccgagaacgctagacgaactgaagacaaaagtcaaaaatatggggttgaaattggagttacatgacctatggggtccgactattgtggtccaaatcactggcaagagcagcaaatatgctcaaggatttttttcttccaatggtacttgttgcctcccaatcagcagatctgcaccagagcttgggaagcttctgtggtcctgctcagcaactattggtgacgcaacagttgttatccaatcaagcgagaagggggaactcctaaggtctgatgatctcgagatacgaggtgctgtggcctccaagaaaggtagactgagctcatttcaccccttcaaaaaatgatgcaggacatagtacagagaatgaaagggccatcagatgtgcgaaaaaaactaaatctgaaaaaaactgcccagactccacattaatctaggttgcagggaaataatacccgacatgcacaatactatcacggtcaccagcaatcagcaaagttgatcaatcactatataaggaatcaagtgggataacaattattaatccaatttcataattataaaaaattgctttaaaggttactgacgagtcgggggcgaaaccttgccacttaagctgcagtcaattttagaatctacatattgaattatgggtaaaatatcaatatatctaattaatagcgtgctattattgctggtatatcctgtgaattcgattgacaatacactcgttgccccaatcggagtcgccagcgcaaatgaatggcagcttgctgcatatacaacatcactttcagggacaattgccgtgcgattcctacctgtgctcccggataatatgactacctgtcttagagaaacaataactacatataataatactgtcaacaacatcttaggcccactcaaatccaatctggatgcactgctctcatctgagacttatccccagacaagattaattggggcagttataggttcaattgctcttggtgttgcaacatcggctcaaatcactgctgcagtcgctctcaagcaagcacaagataatgcaagaaacatactggcactcaaagaggcactgtccaaaactaatgaggcggtcaaggagcttagcagtggattgcaacaaacagctattgcacttggtaagatacagagctttgtgaatgaggaaattctgccatctatcaaccaactgagctgcgaggtgacagccaataaacttggggtgtatttatctctgtatctcacagaactgaccactatattcggtgcacagttgactaaccctgcattgacttcattatcatatcaagcgctgtacaacctgtgtggtggcaacatggcaatgcttactcagaagattggaattaaacagcaagacgttaattcgctatatgaagccggactaatcacaggacaagtcattggttatgactctcagtaccagctgctggtcatccaggtcaattatccaagcatttctgaggtaactggtgtgcgtgcgacagaattagtcactgttagtgtaacaacagacaagggtgaagggaaagcaattgtaccccaatttgtagctgaaagtcgggtgactattgaggagcttgatgtagcatcttgtaaattcagcagcacaaccctatactgcaggcaggtcaacacaagggcacttcccccgctagtggctagctgtctccgaggtaactatgatgattgtcaatataccacagagattggagcattatcatcccggtatataacactagatggaggggtcttagtcaattgtaagtcaattgtttgtaggtgccttaatccaagtaagatcatctctcaaaatacaaatgctgcagtaacatatgttgatgctacaatatgcaaaacaattcaattggatgacatacaactccagttggaagggtcactatcatcagtttatgcaaggaacatctcaattgagatcagtcaggtgactacctccggttctttggatatcagcagtgagatagggaacatcaataatacggtgaatcgtgtggaggatttaatccaccaatcggaggaatggctggcaaaagttaacccacacattgttaataatactacactaattgtactctgtgtgttaagtgcgcttgctgtgatctggctggcagtattaacggctattataatatacttgagaacaaagttgaagactatatcggcattggctgtaaccaatacaatacagtctaatccctatgttaaccaaacgaaacgtgaatctaagttttgatcattcaggccaaaacagagggtctaggctcgggttaataaaagttcaatcaatgtttgatttattaggctttccctactaattattaatgtatttgtgattatatgataacgttaaaagtcttaaatatttaataaaaaatgtaacctgggggcgacctatttacaggctagtatatattaggaagtcctcatattgcactataatctcaaacaattatattacctcgtatccaccttgtctaaagacatcatgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtatttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattgaatcacgataattttgggactcatttaattgcagagtgaaattgtcatcttaggaaataatcaattccatgatttttattgaacatgatcaagcaatcatgtgggaaatttattatcacataacttctaatagttttaaatgacgaattaagaaaaaatggagggcgacctctacacaaacatggatgtaaaacaagttgacctaataatacaacccgaggttcatctcgattcacccatcatattgaataaactggcactattatggcgcttgagtggtttacccatgcctgcagacttacgacaaaaatccgtagtgatgcacatcccagaccacatcttagaaaaatcagaatatcggatcaagcaccgtctagggaaaatcaagagtgacatagcacattactgtcagtattttaatattaatttggcaaatcttgatccgataacccaccccaaaagtttgtattggttatccagactaacaatagctagtgctggaacctttagacatatgaaagatagaatcttatgtacagttggctccgaattcggacacaaaattcaagatttattttcactgctgagccataaattagtaggtaacggtgatttatttaatcaaagtctctcaggtacacgtttgactgcgagtccgttatcccctttatgcaatcaatttgtctctgacatcaagtctgcagtcacgacaccctggtcagaagctcgttggtcttggcttcatatcaaacaaacaatgagatacctgataaaacaatcacgcactacaaattcagctcatttaacagaaattataaaagaggaatggggtttagtaggtattactccagatcttgtcattctttttgacagagtcaataatagtctaactgcattaacatttgagatggttctaatgtattcagatgtattagaatcccgtgacaatattgtgctagtggggcgattatctacttttctgcagccagtagttagtagactggaggtgttgtttgatctagtagattcattggcaaaaaccttaggtgacacaatatacgaaattattgcggtgttagagagcttgtcttatgggtccgttcaactacatgatgcaagtcactctcatgcagggtctttcttttcatttaacatgaatgaacttgataacacactatcaaagagggtggatccgaaacacaagaacaccataatgagcattataagacaatgcttttctaatctagatgttgatcaagctgcagagatgctatgcctgatgagattatttggacacccaatgttaactgcaccggatgcagcagccaaagtaaggaaagcaatgtgtgctccaaaacttgttgaacatgacaccatcttgcagacattatccttcttcaagggaataattataaatgggtacagaagatcacactctggcctgtggcccaatgtagagccgtcttcaatctatgatgatgatctcagacagctgtacttagagtcagcagagatttcccatcatttcatgcttaaaaactacaagagtttgagcatgatagaattcaagaagagcatagactacgatcttcacgacgacttaagtactttcttaaaggatagagcaatttgccggccaaaatcccagtgggatgttatattccgtaagtctttacgcagatcccacacgcggtcccagtatatggacgaaattaagagcaaccgattgctaattgattttcttgattctgctgattttgaccctgaaaaggaatttgcatatgtaaccacaatggattatttgcacgataatgaattttgtgcttcatattctctaaaggaaaaggagatcaaaactaccgggaggatatttgcaaaaatgacacgcaatatgagaagttgccaagtgatacttgaatctctgttatcaaaacatatatgcaagttcttcaaagagaacggcgtttcgatggagcaattgtcattgaccaagagtctacttgcaatgtctcaactctcaccaaaagtctcgactctgcaggacactgcatcacgtcatgtaggcaactcaaaatctcagatcgcaaccagcaacccatctcggcatcactcaacaaccaatcagatgtcactctcaaatcggaaaacggttgtagcaactttcttaacaactgatttggaaaaatactgcctgcagtggcgatactcgactattaagttgtttgcacaagctctaaatcaactctttgggattgatcacggatttgaatggatacatttaagactcatgaacagcaccttatttgtcggtgatccttactcgcctcctgaagatccaacactagaggatatagataaagcaccaaatgacgatatcttcatagtttctccaaggggaggcatagagggtttatgtcagaagatgtggaccatgatatcaattagtgcgatacactgtgtagcagagaaaattggtgcacgagtggcagcaatggtgcagggtgataatcaagtaatagctatcaccaaagaactattcagaggagagaaagcctgtgatgtcagagatgagttagacgagctcggtcaggtgttttttgatgagttcaagaggcacaattatgcaattggacacaaccttaagctaaatgagacaatacaaagccaatccttttttgtatattccaaacgaatattctttgaagggcgattgcttagtcaagtcctcaaaaatgctgccaagttatgtatggttgctgaccatctaggtgaaaacacagtatcttcctgtagcaacctgagctctacaattgcccggttggtggaaaatgggtttgagaaggacactgcttttgtgttgaacctagtctacatcatgactcaaattctttttgatgagcattactcgattgtatgcgatcacaatagtgtcaaaagcttgatcggatcaaaaaactatcggaatctattgtactcatctctaataccaggtcagctcggtggtttcaacttcctcaatataagtcggttgttcactaggaatataggtgacccagtaacatgtagtctgtctgatctcaaatgcttcatagccgcaggtctccttccaccctatgtacttaaaaatgtggttctgcgtgagcctggtcctgggacatggttgacgttgtgctctgatccttacacccttaacataccatacacacagctaccaaccacatatctcaaaaagcacacccagcgatcgttgctttcacgtgcagtaaatcctttattagcaggtgtacaagtgccaaatcagcatgaggaagaagagatgttggctcgctttctccttgatcgtgaatatgtgatgccccgcgttgctcatgtaatactagaaacatcggtccttggcaaacggaaacaaatccaaggcttaattgatacaactccaactatcattagaacatctctagttaatctaccagtgtctaggaagaaatgcgaaaaaataatcaattattctctcaattatattgctgagtgtcatgactccttacttagtcagatctgcttcagtgataataaggaatacttgtggtccacctccttaatatcagttgagacctgtagtgtgacaattgcggactatttgagagctgtcagctggtctaatatattagggggaagaaacatatccggggtgactacacctgatactattgaattaattcaaggttgtttaataggtgaaaattccagttgtactctttgtgaatcgcatgacgacgcattcacatggatgcacttgcctggcccactttacatccctgaaccatcagttactaactctaaaatgcgtgtgccatatctgggttcaaaaacagaggagcgtaaaacagcttcaatggcagcaataaaaggaatgtcacatcacctgcgtgcagtcttaagaggtacatccgtatttatttgggcatttggggacacagatattaattgggataatgcattgcagattgcccaatcacggtgtaacatcacattggatcaaatgagattacttacaccaattcctagcagttcaaatatccaacatagactcgatgacggaatcagcacgcagaaatttactcctgcaagccttgctcgaatcacatcctttgttcacatctgtaatgacagccaaaggttagagaaggatggctcctctgtcgactcaaacttgatttaccagcaaattatgttacttggactcagcatctttgaaacaatgtactcaatggaccaaaagtgggtattcaataaccataccttacatttgcacactggacactcctgttgtccaagggaactagacataagtttagtgaacccgccaagacatcagaccccggagctgactagcacaacaaccaacccgttcctatatgatcagctcccactaaatcaggataatctgacaacacttgagattaagacattcaaatttaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagaagggccattcttaattacttggacacaactttgcaaaggatccctgtgatacaattagccaacattgcactcaccatttcacaccctgagatatttcgcagaattgtcaacaccgggatccataaccagattaagggcccatatgtggcaacaacagatttcatagctgcaagtagagatatcatattatcaggtgcaagggagtatctatcttatctaagcagtggacaggaagactgttacacattcttcaactgtcaagatggggatcttactccaaaaatggaacagtatcttgcaaggagggcatgccttttaacattactgtataatactgggcaccagatccccattatccgatcactgacaccaatagagaagtgcaaggtgctcacagaatacaatcaacaaattgagtatgcagatcaagagtttagctctgtattgaaagtggtcaatgcactactacaaaatcctaatatagatgcattggtttcaaatctctacttcaccaccagacgtgttttatcaaacctcagatcatgtgataaggctatatcatatattgaatatttgtacactgaggacttcggagaaaaagaagatacagtacaatatgacatcatgacaacaaacgatatcatacttactcatggtctattcacacagatcgaaatatcttaccaagggagtagtctccataaattcctaactccggataacgcgcctggatcattgatcccattctctatttcaccaaattcgcttgcatgtgatcctcttcaccacttactcaagtcggtcggtacatcaagcacaagctggtacaagtatgcaatcgcctatgcagtgtctgaaaagaggtcggctcgattaggagggagcttgtacattggtgaagggagcggaagtgtgatgactttgctagagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctaggatttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttacactcatatcttacttgttgctacttacggattgcagcaagattcaatactgtgggtgaaagtatataggacatctgaaaaagtatttcagttcttactgagtgccatgatcatgatctttggttatgtcaaaatccacaggaatggttatatgtcggcaaaggatgaggagtacatattgatgtctgactgcaaggaacctgtaaactatacagctgtccctaacattcttacacgtgtaagtgatttagtgtcgaagaatctgagtcttatccatccagaagacctcagaaaggtaaggtgtgaaacagattccctgaatttgaagtgcaatcatatttatgagaaaataattgctagaaaaattccattacaggtatcatcaactgattctttgctcctccagttaggcggtgtcatcaactcggtgggctcaactgatcctagagaggttgcaacattatcttccattgagtgtatggactatgttgtctcatcaattgatttggctatattagaggcaaatattgtgatctcagagagtgctggtcttgacctcgctttaatgttaggcccattcaacttgaataagcttaagaaaattgacacaatccttaagtcaagcacctatcagctaatcccgtattggttgcgctatgagtactctattaatccgagatctttgtcatttctaatcactaaattacaacaatgccgaatttcatggtcagatatgataacaatctctgaattttgcaagaaatccaagcggcctatatttattaaacgagtaatagggaatcaacagctgaaatcattctttaatgaaagctcaagtattgttttgacccgggctgaagtcaaagtctgtataaagttcctcggtgcaatcatcaagttgaaataatttctgtgttttttaaggggtataatattctaagttgcacttgaagtaatatagcttgtaatcattcgctaggggatagaataattcctataatctctgaatatatatctctaggttataacaaatatatacataataaaaatgattttaagaaaaaatccgactctcaaagaagattggtgcctgtaatattcttcttgccagatgattatggagtgtctagcctaacttaaaacaatcgtattcgatagggaagaatgacatataaagtaactaataaaaaattgtattagtgaaaattaccgtatttcctgtattccatttctggt

SEQ ID NO: 39 - DNA, 15342 bp, artificial sequence
  APMV8 genome FJ619036
accaaacaaggaatgcaagaccaacgggaactttaaataaaacaatcgaatcattgggggcgaagcaagtggatctcgggctcgaggccgaaacactggatttcgctggaggttttgaataggtcgctataagactcaatatgtcatctgtattcaatgaatatcaggcacttcaagaacaacttgtaaagccggctgtcaggagacctgatgttgcctcaacaggtttactcagggcggaaatacctgtctgtgttacattgtctcaagaccccggtgagagatggagccttgcttgccttaatatccgatggcttgtgagtgattcatcaaccacaccaatgaagcagggagcaatattgtcactgctgagtctacattcagacaatatgcgagctcacgcaacattagcagcaaggtctgcagatgcttcactcaccatacttgaggtagatgaagtagatattggcaactccctaatcaaattcaacgctagaagtggtgtatctgataaacgatcaaatcaattgcttgcaattgcggatgacatccccaaaagttgcagtaatgggcatccatttcttgacacagacattgagaccagagacccgctcgatctatcagagaccatagaccgcctgcagggtattgcagctcagatatgggtgtcagccataaagagcatgacagcgcctgacaccgcatcagagtcagaaagtaagaggctggccaaataccaacaacaaggccgactggttaagcaagtacttttgcattctgtagtcaggacagaatttatgagagttattcggggcagcttggtactgcgccagtttatggttagcgagtgcaagagggcttcagccatgggcggagacacatctaggtactatgctatggtgggtgacatcagtctgtacatcaagaatgcaggattgactgcatttttcctcaccctgaagttcggggttggtacccagtatccaaccttagcaatgagtgttttctccagtgaccttaaaagacttgctgcactcatcaggctgtacaaaaccaagggagacaatgcaccatacatggcattcctggaggactccgatatgggaaattttgctccagcaaattatagcacaatgtactcttatgccatgggcattgggacgattctggaagcatctgtatctcgataccagtatgctagagactttaccagtgagaattatttccgtcttggagttgagacagcccaaagccagcagggagcgtttgacgagagaacagcccgagagatgggcttgactgaggaatccaaacagcaggttagatcactgctaatgtcagtagacatgggtcccagttcagttcgcgagccatcccgccctgcattcatcagtcaagaagaaaataggcagcctgcccagaattcttcagatactcagggtcagaccaagccagtcccgaatcaacccgcaccaagggccgacccagatgacattgatccatacgagaacgggctagaatggtaattcaatcacctcgacacatccacctatacaccaattctgtgacatattaacctaatcaaacatttcataaactatagtagtcattgatttaagaaaaaattgggggcgacctcaactgtgaaacacgccagatctgtccacaacaccactcaacaacccacacaagatggacttcgccaatgatgaagaaattgcagaacttctgaacctcagcaccactgtaatcaaggagattcagaaatctgaactcaagcctccccaaaccactgggcgaccacctgtcagtcaagggaacacaagaaatctaactgatctatgggaaaaggagactgcaagtcagaacaagacatcggctcaatctccacaaaccacacaagttcagtctgatggaaatgaggaggaagaaatcaaatcagagtcaattgatggccacatcagtggaactgttaatcaattagagcaagtcccagaacaaaaccagagcagatcttcaccaggtgatgatctcgacagagctctcaacaagcttgaagggagaatcaactcaatcagctcaatggataaagaaattaaaaagggccctcgcatccagaatctccctgggtcccaagcagcaactcaacaggcgacccacccattggcaggggacaccccgaacatgcaggcacggacaaaacccctgaccaagccacatcaagaggcaatcaatcctggcaaccaggacacaggagagaatattcatttaccaccttccatggcaccaccagagtcattagttggtgcaatccgcaatgtaccccaattcgtgccagaccaatctatgacgaatgtagatgcggggagtgtccaactacatgcatcatgtgcagagatgataagtagaatgcttgtagaagttatatctaagcttgataaactcgagtcgagactgaatgatatagcaaaagttgtaaacaccaccccccttatcaggaatgatattaaccaacttaaggccacaactgcactgatgtccaaccaaattgcttccatacaaattcttgacccagggaatgcaggggtgaggtccctctctgaaatgagatctgtgacgaagaaagctgctgttgtaattgcaggatttggagacgacccaactcaaattattgaagaaggtatcatggccaaagatgctcttggaaaacctgtgcctccaacatctgttatcgcagccaaagctcagacttcttccggtgtgagtaagggtgaaatagaaggattgattgcattggtggaaacattagttgacaatgacaagaaggcagcgaaactgattaaaatgattgatcaagttaaatcccacgccgattacgcccgagtcaagcaggcaatatataatgcataatattgtaattatacaaacaatcaatactgctgtcggttgcacccaccttagcaaatcaataatcttttaaaattgattgattaagaaaaaattgactacaataaggaaagaacaccaagttgggggcgaagtcacgattgaccacagtcgctatctgtaaggctcctcaccaaaaatggcatatacaacactaaaactgtgggtggatgagggtgacatgtcgtcttcgcttctatcattcccgttggtactaaaagagacagacagaggcacaaagaagcttcaaccacaggtaagggtagattcaattggcgatgtgcagaatgccaaagagtcctcgatattcgtgactctatatggtttcatccaagcaattaaggagaattcagatcgatcgaaattcttccatccaaaagatgacttcaaacctgagacagtcactgcaggactggtagtagtgggtgcaatccgaatgatggctgatgtcaataccatctctaatgatgcactagcgctggagatcactgttaagaaatctgcaacttctcaagagaaaatgacggtgatgttccacaatagccccccttcattgagaactgcaataactatccgagcaggaggtttcatctcgaatgcagacgaaaatataaaatgtgccagcaagttgactgcaggagtgcagtacatattccgtccaatgtttgtttcaatcactaaattacacaatggcaaactatatagggtgcccaaaagtatccacagcatctcgtctaccctactgtatagtgtgatgttggaggtaggattcaaagtggacatcgggaaggatcatccccaggcaaaaatgctgaagagggtcacaattggcgatgcagacacatactggggatttgcatggttccacctgtgcaatttcaaaaagacatcctctaagggaaagccgagaacgctagacgaactgaggacaaaagtcaaaaatatggggttgaaattggagttacatgacctatggggtccgactattgtggtccaaatcactggcaagagcagcaaatatgctcaaggatttttttcttccaatggtacttgttgcctcccaatcagcagatctgcaccagagcttgggaagcttctgtggtcctgctcagcaactattggtgacgcaacagttgttatccaatcaagcgagaagggggaactcctaaggtctgatgatctcgagatacgaggtgctgtggcctccaagaaaggtagactgagctcatttcaccccttcaaaaaatgatgcaggacatagtacagagaatgaaagggccatcagacgtgcgaaaaaaactaaatctgaaaaaaactgcccagactccacattaatctaggttgcagggaaataatacccgacatgcacaatactatcacggtcaccagcaatcagcaaagttgatcaatcactatataaggaatcaagtgggataacaattattaatccaatttcataattataaaaaattgctttaaaggttactgacgagtcgggggcgaaaccttgccacttaagctgcagtcaattttagaatctacatattgaattatgggtaaaatatcaatatatctaattaatagcgtgctattattgctggtatatcctgtgaattcgattgacaatacactcgttgccccaatcggagtcgccagcgcaaatgaatggcagcttgctgcatatacaacatcactttcagggacaattgccgtgcgattcctacctgtgctcccggataatatgactacctgtcttagagaaacaataactacatataataatactgtcaacaacatcttaggcccactcaaatccaatctggatgcactgctctcatctgagacttatccccagacaagattaattggggcagttataggttcaattgctcttggtgttgcaacatcggctcaaatcactgctgcagtcgctctcaagcaagcacaagataatgcaagaaacatactggcactcaaagaggcactgtccaaaactaatgaggcggtcaaggagcttagcagtggattgcaacaaacagctattgcacttggtaagatacagagctttgtgaatgaggaaattctgccatctatcaaccaactgagctgcgaggtgacagccaataaacttggggtgtatttatctctgtatctcacagaactgaccactatattcggtgcacagttgactaaccctgcattgacttcattatcatatcaagcgctgtacaacctgtgtggtggcaacatggcaatgcttactcagaagattggaattaaacagcaagacgttaattcgctatatgaagccggactaatcacaggacaagtcattggttatgactctcagtaccagctgctggtcatccaggtcaattatccaagcatttctgaggtaactggtgtgcgtgcgacagaattagtcactgttagtgtaacaacagacaagggtgaagggaaagcaattgtaccccaatttgtagctgaaagtcgggtgactattgaggagcttgatgtagcatcttgtaaattcagcagcacaaccctatactgcaggcaggtcaacacaagggcacttcccccgctagtggctagctgtctccgaggtaactatgatgattgtcaatataccacagagattggagcattatcatcccggtatataacactagatggaggggtcttagtcaattgtaagtcaattgtttgtaggtgccttaatccaagtaagatcatctctcaaaatacaaatgctgcagtaacatatgttgatgctacaatatgcaaaacaattcaattggatgacatacaactccagttggaagggtcactatcatcagtttatgcaaggaacatctcaattgagatcagtcaggtgactacctccggttctttggatatcagcagtgagatagggaacatcaataatacggtgaatcgtgtggaggatttaatccaccaatcggaggaatggctggcaaaagttaacccacacattgttaataatactacactaattgtactctgtgtgttaagtgcgcttgctgtgatctggctggcagtattaacggctattataatatacttgagaacaaagttgaagactatatcggcattggctgtaaccaatacaatacagtctaatccctatgttaaccaaacgaaacgtgaatctaagttttgatcattcaggccaaaacagagggtctaggctcgggttaataaaagttcaatcaatgtttgatttattaggctttccctactaattattaatgtatttgtgattatatgataacgttaaaagtcttaaatatttaataaaaaatgtaacctgggggcgacctatttacaggctagtatatattaggaagtcctcatattgcactataatctcaaacaattatattacctcgtatccaccttgtctaaagacatcatgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtctttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattgaatcacgataattttgggactcatttaattgcagagtgaaattgtcatcttaggaaataatcaattccatgatttttattgaacatgatcaagcaatcatgtgggaaatttattatcacataacttctaatagttttaaatgacgaattaagaaaaaatggagggcgacctctacacaaacatggatgtaaaacaagttgacctaataatacaacccgaggttcatctcgattcacccatcatattgaataaactggcactattatggcgcttgagtggtttacccatgcctgcagacttacgacaaaaatccgtagtgatgcacatcccagaccacatcttagaaaaatcagaatatcggatcaagcaccgtctagggaaaatcaagagtgacatagcacattactgtcagtattttaatattaatttggcaaatcttgatccgataacccaccccaaaagtttgtattggttatccagactaacaatagctagtgctggaacctttagacatatgaaagatagaatcttatgtacagttggctccgaattcggacacaaaattcaagatttattttcactgctgagccataaattagtaggtaacggtgatttatttaatcaaagtctctcaggtacacgtttgactgcgagtccgttatcccctttatgcaatcaatttgtctctgacatcaagtctgcagtcacgacaccctggtcagaagctcgttggtcttggcttcatatcaaacaaacaatgagatacctgataaaacaatcacgcactacaaattcagctcatttaacagaaattataaaagaggaatggggtttagtaggtattactccagatcttgtcattctttttgacagagtcaataatagtctaactgcattaacatttgagatggttctaatgtattcagatgtattagaatcccgtgacaatattgtgctagtggggcgattatctacttttctgcagccagtagttagtagactggaggtgttgtttgatctagtagattcattggcaaaaaccttaggtgacacaatatacgaaattattgcggtgttagagagcttgtcttatgggtccgttcaactacatgatgcaagtcactctcatgcagggtctttcttttcatttaacatgaatgaacttgataacacactatcaaagagggtggatccgaaacacaagaacaccataatgagcattataagacaatgcttttctaatctagatgttgatcaagctgcagagatgctatgcctgatgagattatttggacacccaatgttaactgcaccggatgcagcagccaaagtaaggaaagcaatgtgtgctccaaaacttgttgaacatgacaccatcttgcagacattatccttcttcaagggaataattataaatgggtacagaagatcacactctggcctgtggcccaatgtagagccgtcttcaatctatgatgatgatctcagacagctgtacttagagtcagcagagatttcccatcatttcatgcttaaaaactacaagagtttgagcatgatagaattcaagaagagcatagactacgatcttcacgacgacttaagtactttcttaaaggatagagcaatttgccggccaaaatcccagtgggatgttatattccgtaagtctttacgcagatcccacacgcggtcccagtatatggacgaaattaagagcaaccgattgctaattgattttcttgattctgctgattttgaccctgaaaaggaatttgcatatgtaaccacaatggattatttgcacgataatgaattttgtgcttcatattctctaaaggaaaaggagatcaaaactaccgggaggatatttgcaaaaatgacacgcaatatgagaagttgccaagtgatacttgaatctctgttatcaaaacatatatgcaagttcttcaaagagaacggcgtttcgatggagcaattgtcattgaccaagagtctacttgcaatgtctcaactctcaccaaaagtctcgactctgcaggacactgcatcacgtcatgtaggcaactcaaaatctcagatcgcaaccagcaacccatctcggcatcactcaacaaccaatcagatgtcactctcaaatcggaaaacggttgtagcaactttcttaacaactgatttggaaaaatactgcctgcagtggcgatactcgactattaagttgtttgcacaagctctaaatcaactctttgggattgatcacggatttgaatggatacatttaagactcatgaacagcaccttatttgtcggtgatccttactcgcctcctgaagatccaacactagaggatatagataaagcaccaaatgacgatatcttcatagtttctccaaggggaggcatagagggtttatgtcagaagatgtggaccatgatatcaattagtgcgatacactgtgtagcagagaaaattggtgcacgagtggcagcaatggtgcagggtgataatcaagtaatagctatcaccaaagaactattcagaggagagaaagcctgtgatgtcagagatgagttagacgagctcggtcaggtgttttttgatgagttcaagaggcacaattatgcaattggacacaaccttaagctaaatgagacaatacaaagccaatccttttttgtatattccaaacgaatattctttgaagggcgattgcttagtcaagtcctcaaaaatgctgccaagttatgtatggttgctgaccatctaggtgaaaacacagtatcttcctgtagcaacctgagctctacaattgcccggttggtggaaaatgggtttgagaaggacactgcttttgtgttgaacctagtctacatcatgactcaaattctttttgatgagcattactcgattgtatgcgatcacaatagtgtcaaaagcttgatcggatcaaaaaactatcggaatctattgtactcatctctaataccaggtcagctcggtggtttcaacttcctcaatataagtcggttgttcactaggaatataggtgacccagtaacatgtagtctgtctgatctcaaatgcttcatagccgcaggtctccttccaccctatgtacttaaaaatgtggttctgcgtgagcctggtcctgggacatggttgacgttgtgctctgatccttacacccttaacataccatacacacagctaccaaccacatatctcaaaaagcacacccagcgatcgttgctttcacgtgcagtaaatcctttattagcaggtgtacaagtgccaaatcagcatgaggaagaagagatgttggctcgctttctccttgatcgtgaatatgtgatgccccgcgttgctcatgtaacactagaaacatcggtccttggcaaacggaaacaaatccaaggcttaattgatacaactccaactatcattagaacatctctagtcaatctaccagtgtctaggaagaaatgcgaaaaaataatcaattattctctcaattatattgctgagtgtcatgactccttacttagtcagatctgcttcagtgataataaggaatacttgtggtccacctccttaatatcagttgagacctgtagtgtgacaattgcggactatttgagagctgtcagctggtctaatatattagggggaagaagcatatccggggtgactacacctgatactattgaattaattcaaggttgtttaataggtgaaaattccagttgtactctttgtgaatcgcatgacgacgcattcacatggatgcacttgcctggcccactttacatccctgaaccatcagttactaactctaaaatgcgtgtgccatatctgggttcaaaaacagaggagcgtaaaacagcttcaatggcagcaataaaaggaatgtcacatcacctgcgtgcagtcttaagaggtacatccgtatttatttgggcatctggggacacagatattaattgggataatgcattgcagattgcccaatcacggtgtaacatcacattggatcaaatgagattacttacaccaattcctagcagttcaaatatccaacgtagactcgatgacggaatcagcacgcagaaatttactcctgcaagccttgctcgaatcacatcctctgttcacatctgtaatgacagccaaaggttagagaaggatggctcctctgtcgactcaaacttgatttaccagcaaattatgttacttggactcagcatctttgaaacaatgtactcaatggaccaaaagtgggtattcaataaccataccttacatttgcacactggacactcctgttgtccaagggaactagacataagtttagtgaacccgccaagacatcagaccccggagctgactagcacaacaaccaacccgttcctatatgatcagctcccactaaatcaggataatctgacaacacttgagattaagacattcaaatttaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagaagggccattcttaattacttggacacaactttgcaaaggatccctgtgatacagttagccaacattgcactcaccatttcacaccctgagatatttcgcagaattgtcaacaccgggatccataaccagattaagggcccatatgtggcaacaacagatttcatagctgcaagtagagatatcatattatcaggtgcaagggagtatctatcttatctaagcagtggacaggaagactgttacacattcttcaactgtcaagatggggatcttactccaaaaatggaacagtatcttgcaaggagggcatgccttttaacattactgtataatactgggcaccagatccccattatccgatcactgacaccaatagagaagtgcaaggtgctcacagaatacaatcaacaaattgagtatgcagatcaagagtttagctctgtattgaaagtggtcaatgcactactacaaaatcctaatatagatgcattggtttcaaatctctacttcaccaccagacgtgttttatcaaacctcagatcatgtgataaggctatatcatatattgaatatttgtacactgaggacttcggagaaaaagaagatacagtacaatatgacatcatgacaacaaacgatatcatacttactcatggtctattcacacagatcgaaatatcttaccaagggagtagtctccataaattcctaactccggataacgcgcctggatcattgatcccattctctatttcaccaaattcgcttgcatgtgatcctcttcaccacttactcaagtcggtcggtacatcaagcacaagctggtacaagtatgcaatcgcctatgcagtgtctgaaaagaggtcggctcgattaggagggagcttgtacattggtgaagggagcggaagtgtgatgactttgctagagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctaggatttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttacactcatatcttacttgttgctacttacggattgcagcaagattcaatactgtgggtgaaagtatataggacatctgaaaaagtatttcagttcttactgagtgccatgatcatgatctttggttatgtcaaaatccacaggaatggttatatgtcggcaaaggatgaggagtacatattgatgtctgactgcaaggaacctgtaaactatacagctgtccctaacattcttacacgtgtaagtgatttagtgtcgaagaatctgagtcttatccatccagaagacctcagaaaggtaaggtgtgaaacagattccctgaatttgaagtgcaatcatatttatgagaaaataattgctagaaaaattccattacaggtgtcatcaactgattctttgctcctccagttaggcggtgtcatcaactcggtgggctcaactgatcctagagaggttgcaacgttatcttccattgagtgtatggactatgttgtctcatcaattgatttggctatattagaggcaaatattgtgatctcagagagtgctgatcttgacctcgctttaatgttaggcccattcaacttgaataagcttaagaaaattgacacaatccttaagtcaagcacctatcagctaatcccgtattggttgcgctatgagtactctattaatccgagatctttgtcatttctaatcactaaattacaacaatgccgaatttcatggtcagatatgataacaatctctgaattttgcaagaaatccaagcggcctatatttattaaacgagtaatagggaatcaacggctgaaatcattctttaatgaaagctcaagtattgttttgacccgggctgaagtcaaagtctgtataaagttcctcggtgcgatcatcaagttgaaataatttctgtgttttttaaggggtatagtattctaagttgcacttgaagtaatatagcttgtaatcattcgctaggggatagaataattcctataatctctgaatatatatctctaggttataacaaatatatacataataaaattgattttaagaaaaaatccgactttcaaagaagattggtgcctgtaatattcttcttgccagatgattatggagggtctagcctaacttaaaacaatcgtattcgatagggaagaatgacatataaagtaactaataaaaaattgtattagtgaaaattaccgtatttcctgtattccatttctggt

SEQ ID NO: 40 - DNA, 15438 bp, artificial sequence
  APMV9 genome EU910942
accaaacaaagaaattgtaagatacgttaaagaccgaagtagcaactgacttcgtacgggtagaaggattgaatctcgagtgcgaacacgacgctgtgattcgaaggtccgtactaccatcatgtcctctatattcaatgagtatgagagtctgcttgaaagtcaactcaaaccgacgggctcgaacgtcttaggagagaaaggtgacactccaaaagtcgagatccctgtatttgtgctcaacagtgacaaccctgaagatcgctggaactttactaccttctgtctcagagtcgctgtgagcgaggatgctaataggcctttgcgtcagggggcactcatctctctactttgcgctcattctcaggtgatgaagaatcatgtggccatagcaggaaagcaggatgaggctctgattgtagttctagagattgatactattaatgatggtgttccagccttcaacaataggagcggtgtcacagaggaacgagctcagcgtttcgctatgatagctcaagcattaccccgtgcttgtgcaaatgggacaccgttcaccgtccaagatgcagaagatgatccagtcgaagacataacagacgcccttgatcgcatattgtcaatccaggcgcaagtatgggtgaccgtcgcaaaatccatgacagcgtacgagactgcagatgaatcagaacagaagcgattgaccaagtatgttcagcaaggtcgagtgcagaagaaatgcatgatctaccctgtatgtcggagcatgctgcagcagatcataaggcaatctttagcagtccgacggttcattgtcagtgagctgaaacgagctcggaatacagcaggaggaacatccacgtattataacttcgttgctgatgtagattcctacattaggaatgctgggttaactgcattcttcttgacccttaagtatggtgtgaatacaaagacttctgtccttgcccttagcagcttggcaggcgatcttcaaactgtcaaacagttgatgcggctgtataaagccaaaggagatgatgcaccatacatgactatactgggagacggagaccagatgagatttgcacctgctgaatacgcacagctatactcatacgctatgggaatggcatcagtcatagacaaagggacctcaaggtatcagtacgctcgtgacttcctaaaccccagcttctggaggctgggagtggagtatgcccagactcaaggaagcaacatcaacgaagagatggcatcagaactgaaactcagcccaatagctagaaggatgctgaccactgccgtcacaaaagtagcaaccggagcgtctgattattcggtacctcagcatacagcaggagtcctaactggcttgaattcaacagacggcaaccttgggtctcagaagctgcccacctcaattcagcaggatcagaatgatgatactgccatgttgaacttcatgagggccgtagcacaaggaatgaaggagacaccaattcaggctcctcccacccctggattcggatctcaacaggccgcagacgacgatgactcgcgggatcaagcagactcctgggggctctaatgaaatacggaggttgactccagcccaaacgaacctctagcaactcctaatccctcatccacctacaaactccacatctacatgaccaatccgctcacacaacacggcggaagacaccatccatccccaactgtcccaacccgaagaacatcctcaacttagcccgctaatttcacgaaccattacaaaaaacttatcaacagaaaaaactacgggtagaactgtctgccactgcgagaaagcaaacgcatcaacgcagtcagcactcatcgcagctctccatcacaccaattctagctcaggcacacgcctccagagagaaccatggcatccttcacagacgacgagatatcagatctgatggaacaaagtggtcttgtaatagatgagatcatgacatcccaagggatgcctaaagagaccctagggcgaagtgcaatcccaccagggaaaactcaggccctaactgatgcctgggagaaacacaacaagtcacagagatccaatgcggatcacagcaccggatcaaataacaaaactgatgtcaacacaccccacaatgctgagccgccacaatccaccggcgatccctccgcatctccagaaatggacggcgacacaaccccactcccaaagcaggaaaccgccgaaaagcacccctgcaaagaaggggccactggagggctgctggatatgcttgaccggattgctgccaagcaggatagagctaaaaaagggctcaatccgagatcacaagacacgggcaccctgcactcaggccaattccctacgcagacgcaagacccgacatcccgccgatcaaccaactcatcgggacacagcatggagtccagaacgcccgcccagctgccaatcccgaggagagacgacagcccgcatcaggtaagaagagaggaggagggcatcgcagagaacacagcatggtctggaatgcaaacgggattgtcaccatcagctggtgcaacccagtttgctctccagtcacctacgaaccaagagaattcacatgttcatgcgggagctgccctacagaatgccgactttgtgcaggctctcatagggatattagaaagcattcagcagagagtgagtaaaatggaatatcagatggatttagtcctgcgtcacctgtctagtatgccagccattcgaaatgacattcaacaagttaagaccgctatggcagtgcttgaggccaacattgggatgatgaaaatccttgaccctggatcagcacatatttcttcgctcaatgatcttcgagcagttgcaaggtatcatccagtccttgtagcaggccccggtgaccccaataaaacaattgctgatgataaaaccatcactgtcaatcggctctcccagccggtaactgatcagcgcagcttggtaagagaactcacacccccttccggtgatttcgaggcagaaaaatgcgcaatcaaggcgttattagctgcgagaccactacatccatcggctgcaaaacgaatgtctgataggttagatgcagccaagacatgtgaagaattgaggaaggtgaagagacagattctgaataactgacccaaatagtgtggtttccgccaatgatcaagcgtgatccgccttggacaacttttttgccgatcttaaggagagacaaatcaatttacaccgatctaaaatatcatcagacaccctcaaatcaagaaaacatagatgacagtctgcttgactcatctcttgcatctgatgctatcaattgccctaaaataccacctgacataaataccagattatctctagacctccttggttgttaagaaaaaaaagtaagtacgggtagaaacaggactcaaccgacctaccaccatggatgcttctaggatgatcagtctatatgtagaccccactagcagttctagttcaatactcgcattcccaatagtcatggaagccacaggagacggacgaaagcaaatttcaccccaatatcgcattcagagattagatcactggtcagacagcagtcgagatgcagtattcatcaccacatatgggtttatatttggataccctaaatcacgtgctgatcgaggccagcttaatgaagaaattaggcctgtgctgctctctgctgcaacgctatgtctgggcagtgtggcgaatactggagatcaggttgcaattgctcgggcatgcttgtcactacaaatatcttgcaaaaagagtgctactagtgaggagaaaatgatatttgcaatcacccaagctccgcagattttacaatcatgtcgtgctgtttcgcaaaaattcgtctccgttggatcaaataaatgtgtgaaagcacctgaaagaatcgagggaggccagcagtatgactataaggtcaacttcgtgtctctcactatagtaccaaaagatgacgtatatagggtcccaaaacctgtcctatcagtcagcagtcccactctattccgccttgccctgagtgttaacatcgcaatcgacatcaatgccgacaatcctttgtctaagacgcttattaagaccgaaagcggctttgaagcaaatttgttcctgcatgtgggtattctctcaaacattgacaagcggggaaagaaggtgacgttcgagaagttagagaagaaaatccggcggatggaactgactgcaggattaagtgatatgtttggtccgtccatcatcctgaaggccaaagggccgaggacaaagttgatgtcagcattcttttctaatacgggaacagcgtgttatccgatcgcacaagcatctcctccagtatcgaagatcttgtggagccaaagcggacacctccaggaggttaagatacttgtacaatcgggaacctcgaaaatgattgcattaacagccgatcaagaaatcacaacaacaaagctcgatcagcacgccaagattcaatcatttaacccattcaaaaagtaagttgcatggctcacgaatagctcaggtcttcttgccttaaaatcagccaatgaatatgtgataggatattcagtgtctcgaatcattaccgatcaaaaaaccccattaaatcatacacctgatcattagacaagaggtaatccaaatagcattaaaaaaaatccccaaaagaattaaaactaaaacacagcacgggtagaaagtgagctgtatatcactcaatccacaatctaccatagtgacacaatggggtacttccacctattacttatactaacagcgattgccatatctgcgcacctctgctataccacgacattggatggtagaaaactgcttggtgcaggcatagtgataacagaagagaagcaagttagggtgtacacagctgcgcaatcaggaacaattgtcttaaggtctttccgtgtggtctccttagacagatactcgtgcatggaatccactattgagtcatataacaagactgtatataacatacttgcacctctgggcgatgcaatccgccgaatacaggcaagtggtgtatcggttgagcgtatccgagagggccgcatatttggtgccatccttgggggagttgccttaggtgtagccaccgcagcacagataacagctgcaattgctttgattcaggctaacgagaacgcaaaaaacatcctgcgtattaaagacagtataactaagaccaacgaggcagtgagagatgtaactaatggcgtgtcgcagttaactatcgctgtaggtaaattacaggacttcgtcaataaggaattcaataagacaactgaggccattaattgtgtacaggcagctcaacaattaggtgtggagctaagcctctatctgaccgagatcactacagtcttcggacctcagataacctctcctgctttaagcaaattgactatccaagcgctgtataatttggcgggcgtaagcttggatgtactactgggaaggctcggagcagacaattcacagttatcatctttggttagtagtggtcttattaccggacagcccattctctacgactcggaatctcaaatattggcactgcaagtgtcactaccctccattagtgacttaaggggagtgagagcgacatacttagacacgttggctgtcaacactgcagcaggacttgcatctgctatgattccaaaggtagtaatccaatctaataatatagttgaagaattagatactacagcatgtatagcagcagaagctgacttatactgtacgaggattactacattccccattgcgtcggctgtatcagcctgcattcttggggatgtatcgcaatgcctttattcaaagactaatggcgtcttaaccactccatatgtagcagtaaaggggaaaattgtagccaattgtaagcatgtcacatgtaggtgtgtagatcctacatccatcatatctcaaaattacggtgaagcagcgactcttatcgatgatcagctatgcaaggtaatcaacttagatggtgtgtccatacagctgagcggcacatttgaatcgacttatgtgcgcaacgtctcgataagtgcaaacaaggtcattgtctcaagcagtatagatatatctaatgagctggagaatgttaacagctctttaagttcggctctggaaaaactggatgaaagtgacgctgcgctaagcaaagtaaatgttcacttaactagcacctcagctatggccacatacattgttctaactgtaattgctcttatcttggggtttgtcggcctaggattgggttgctttgctatgataaaagtaaagtctcaagcaaagacactactatggcttggtgcacatgctgaccgatcatatatactccagagtaagccggctcaatcgtccacataatacaacaacaatcaatcctgactatcatataatacatgaatcatttcttcttccgattataaaaaaataagaaacctaattaggccaatacgggtagaacaggcttccaccccgtatttcttcggctgtgatcctgtacctgagttcttcccaccaacaccaggacctctcctaaattgcatcaccatggaatcaggaatcagccaggcatctcttgtcaatgacaacatagaattaaggaatacgtggcgcacggccttccgtgtggtctccttattactcggcttcaccagcttggtgctcactgcttgcgctttacacttcgctttgaatgccgctacccctgcggatctctctagtatcccagtcgctgttgaccaaagtcatcatgaaattctacaaaccttgagtctgatgagcgacattggcaataagatttacaagcaggtagcactagatagtccagtggcgctgctcaacactgaatcaaccttaatgagcgcaattacatcactatcttatcagattaacaatgcagcgaataactcaggttgtggcgcccctgtgcatgataaggattttatcaatggagtggcaaaggaattatttgtagggtctcaatacaatgcctcgaactatcgaccctccaggttccttgagcatctaaatttcatccccgcccctactacgggaaaaggttgcaccagaattccgtcctttgatctagctgcaacacattggtgttatactcacaatgtgattcttaatggttgtaatgatcatgctcaatcttatcaatacatatccctcgggatactcaaggtgtcagccacgggaaacgtgttcttatctactctcagatctatcaacctggatgatgatgaaaaccggaaatcatgtagcatatcagcaacgccactagggtgtgacttactttgtgctaaagtcactgagagagaagaggcagattacaattcagatgcagcgacgagattagttcatggcaggttaggttttgatggggtataccatgagcaggccctgcctgtagaatcattgttcagtgactgggttgcaaactatccgtcagtcggcggaggcagttactttgataatagggtatggtttggcgtgtatggggggatcagacctggctctcagactgatctgctccagtctgagaagtacgcgatatatcgtaggtacaataatacctgccctgataataatcccacccagattgagcgggccaaatcatcttatcgtccgcagcggtttggccagcggcttgtacaacaagcaattctatcaattagagtggagccatctttgggtaatgatcctaaactatctgtgttagataatacagtcgtgttgatgggggcggaagcaaggataatgacatttggccacgtggcattaatgtatcaaagagggtcatcatattttccttctgcactattataccctctcagtttaacaaatggtagtgcagcagcatccaagcctttcatattcgagcaatatacaaggccaggtagcccaccttgtcaggccactgcaagatgtccaaattcatgtgttactggtgtctacacagacgcatacccgttattttggtctgaagatcataaagtgaatggtgtatatggtatgatgttagatgacatcacatcacggttaaacccggtagcagctatatttgataggtatggtaggagtagagtgactagggttagcagtagcagcacgaaggcagcttacactacaaatacatgctttaaggttgtcaaaacaaagagagtatactgcttgagcattgccgagatagagaatacactgtttggagaattcagaataacccctttactctccgagataatatttgacccaaaccttgaaccctcagacacgagccgtaactgaggaaaatccgttctggcagacagtggttggatagaccttgcgtcgatagccctcactgttggcactgcgtcgtccctatattcaaacaccacattagcggagtatacagatagtcggccatgatgaatcaaatgtcatgcgatttgagcataaccgaagcagaatcaggatatacccggctctaccatatcagggagaacagctggtaagctgtaatcctcaataatcctaaaaactgcaggtaatacaaaaggatcagcctatagggagcttcaacaatcgttagaaaaaaacgggtagaacatggataatccaggacaatctcgccctgatcatcaagtgattctacccgaagcgcatctttcctcaccgatcgtaaggcataagttatattatttctggagactaacaggagtaccactaccccactcagcagaatttgatacgctagtcctatccagaccatggaacaaaatattgcagagcaactcgccagaagtactgaggatgaagcggctaggtgcgaacgtccacgcgactctagatcactctcgaccaataaaggctttgatccacccggagactttagcatggctaactgatctgtctataggggtatctatctctagatttagaggaatagaaaagaaagtatctcgcctgctccatgacaatagagagaaattttgtacacttgtttctcagattcatgaaggattgttcggtggtgtaggaggggttcggaataatctgtcaccagagtttgaaagtttgctcaatggaactaacttctggtttggcgggaaatattcaaacacaaaattcacttggcttcacattaaacaattgcagagacatcttatactcacagcgcgtatgagatctgggcagcaactttacatccaattaaagcatacaaggggttatgtccatataactccagagttaactatgattacatgcaacggaaaaaaccttgttacagcacttacacctgagatggtcttaatgtatagtgacatgctagaaggaagagatatggtcataagtgttgcacagcttgtgaatggcctgaatgtcctagcagataggattgagtgtcttcttgacttgattgaccaattggcgtgcttgataaaggatgctatatatgaaataattgggattttggagggtttagcttatgcagcagtccagctgctggagccgtccggaaaattcgcaggggatttctttgaattcaatctcagagagatagctgccatattgcgagaacacatagaccctgtgttagctaacagggtacttgagtctattacctggatttacagtggtctgacagacaaccaagcagcagagatgctctgtatcctccgcttgtggggccaccctacattagagtccagaacagctgcagctgcagtgcgaaagcaaatgtgcgcgccaaaactcattgacttcgacatgatccaacaagtattggctttctttaaagggacaatcatcaatggatatagaagacaaaactcaggagtctggccaagagttaaaaaggatactatctatggatcaacactccaacagttgcatgctgactatgcagagatatcacacgaattaatgctgaaagaatacaagcgtctagcaatgcttgagtttgagaagtgtattgacatagacccagtatccaatttaagcatgttcttgaaggacaaggctatagcacacacgcgaccaaattggctggcatcttttaaaagaactttgttatccgatagacagcagctcttagcaaaggatgcaacttcgaccaatcgtctgctgatagaattcctagaatctagcaactttgacccatatcaggagatgacctatttgacaagtcttgaatttcttagagataatgacgtggcagtatcatattcgttaaaggagaaagaagttaagcccaatggtagaatcttcgcaaagcttaccaaacgactcagaaattgtcaggtgatggcagagaatatcctagcagacgaaattgcaccttttttccaagggaatggagtcattcaaagcagcatctctctgacgaaaagtatgttagcaatgagtcaactgtcatttaattgcaacagattctcgatcggaaaccgcagagaagggatcaaagagaataggacacgacaccgtgaacgaaagcgaagaaggcgagtagctacatatatcacaactgacctgcagaagtactgtctcaattggaggtatcagaccatcaagccttttgcccatgcgattaatcagctgacagggcttgatttgttttttgagtggatccaccttcgtctaatggataccactatgttcgttggagatccatacaacccaccctctgatccaacaattgaaaacctggatgatgcacccaatgatgatatctttattgtaagcggaagaggagggatcgagggattatgtcaaaagctttggactaccatatcaatatccgcaatacaattagcagccacccggtcaaagtgtagggtagcctgtatggtgcaaggtgacaatcaggtgatcgcagtgacccgagaagtaaatccagatgactcagaagatgcggtcttagatgaattacataaggccagcgacagattctttgaggaactcactcacgtgaatcatctgatcggacataacctgaaagatagagagaccatacgctcagatacttgttttatctatagcaagcgagtattcaaggatggtaagatactttctcaggccctcaagaatgctgcaaagctcgtcttaatatctggggagattggggagaacactcctatgtcatgcgggaatattgcttctacagtgtctcgtctgtgtgaaaatgggctgcccaaagatgcctgctatatgatcaattatatattaacctgtatacaatttttctttgacaatgagttttccattgtccccgcttctcagcgtggatccacagttgaatgggtggataacctttcatttgtacacgcgtatgcactgtggccaggccaatttggaggattgaacaacttacaatattctagattgtttactcgcaatatcggggacccatgcactactgcacttgcagagattaagagattagagagagctcaactaataccagggaagctaatcaagaacttgcttgctaggaagccaagcaatggaacatgggcgtctctttgtaatgatccttattcactcaatattgaaacagcaccaagcccaaatctcatcctcaagaaacatactcagagagtactatttgaatcctgcaccaatcccctattacaaggggtttatagtgaagaaaatgatacggaagaagcagaattagcagaattcttgctcaatcaagaagctatacatccgcgcgtggcacacgttataatggaggccagcgcagtcggtagaaagaagcaaattcagggactaatcgatacaactaacaccatcataaagattgcacttgggcggcgtcctcttggtgcaaggaggttaaggaagataaacagttattcttctatgcacatgttgatcttcctggatgatatattcctacctaaccatcctccatctcccttcgtctcctcagtgatgtgttctgttgccctagcggattacctacgtcagattacctggttgcctctgacaaatggtaggaagatattaggtgtaaataatccagatacccttgagttagtatcaggatcgatgctgaatctaaacggatattgtgacttatgtaatagtggagataaccaatttacgtggttccatctcccagcagatatagagctagcggacagttcatcatccaaccctccaatgcgtataccttatgtgggatccaagacccaggaaaggagaaatgcatcaatggccaagattagcaacatgtcccctcatatgaaggcagcattgagattggcgtctgtgaaggtaagggcttacggtgataatgagcataattggcaagttgcatggcagctagcaaatactcgatgtgcgatatcccttgaacatctaaaacttctagcccctctaccaactgcagggaaccttcagcatcgattggatgatagcataacccagatgacctttactcccgcttctctctatcgggtggcaccttatatccacatctccaatgactcacaaagaatgttttctgatgagggggttaaggagagcaacatcatctatcagcagataatgttattgggtctatcagctatcgaatcattgttccccttgaccactaatcatgtatatgaagaagtgacactacaccttcatactcaattcagctgctgcctgagagaggcggcccttgcggtcccatttgagctccagggcaaagtacctaggattcgtgctgctgaggggaaccaattcgtgtatgactcatccccacttttggaacctgaggctcttcaactcgatgtggctactttcaagaactatgagttggacttagaccattattcaacgatagacttgatgcatgtacttgaggttacgtgtggaaagctaataggtcagtcggtgatttcatacaatgaggacacttctataaagaatgatgcaattattgtatacgataatacccggaattggatcagtgaggcccaaaattgtgacctggtgaagttatttgagtatgctgcactagaaatcttgctggactgcgcattccaaatgtattatctaagggttcgcggatacaagaacatcctaatatacatggcagacctaattcgtaatatgcccggtatattgctctctaatattgctgccacaatctcccatcccattatccatactagactatacaatgcagggttgctggatcatgggagtgcgcaccaacttgcaagcattgattttattgaattatcagctaatttattggtaacatgtatagctcgtgtatgtactacacttctatccggtgaaaccctgatgcttgcatttccatccgttctagacgagaatttgacggagaaaatgtttcttctaatcgctcgatactgctctttgttagcgttgttgtactcatctaaggttcctataccaaatattaggggcctgactgccgaagataagtgccggatgctcacaaatcatctcatgaaccttccatctgaatttcggctgaccgaaaatcaggtacgaaatgtactgcaaccagcactgacaactttcccagcaaacctctattatatgtcaagaaagagtcttaatatcatcagagagagggagataaagatgctattattcaaatgttgttccctgccggggatgaagctacaagcacggtggcagttaatttgggatacgaaagtaaatgaccccattgttaagtggcgacgcattgaattcttatgcgagctcgatctctctggtcaggcaaggtttggagtcatactggatgaatgcatctctgatgttgataaaaacggacagggcatcctcgactttgtcccaatgactcgatacctattcaggggtgtaggccaggcatcctcatcatggtataaagctgccaatttattgtcacttcctgaagtgcgccaggcacgtttcggtaactcattgtacttagcagaaggtagcggtgcaataatgagtctgttagagctccacgtaccacatgagaagatttactacaatactctcttttataacgagatgaaccccccgcaaagacatttcggcccaacgccaactcaattccttgcatcggtcgtttacaagaaccttcaggcaggtatagtctgcaaagatgggtatgttcaggagttctgccctttatggagagacgttgccgatgaaagtgatcttgcttcagataggtgtgtctcattcattacatcagaggtgcctggaggcactgtatctctactccattgtgacatagaaacaaccctggaaccaagctgggcttacttggagcaattagccactaatatctctctaatcgggatgcacgtcctgcgagagaatggagtgttcatcatcaaagtactatacacccagagtttcttttttcatctattgctggcaatcttagctccttgtagtaaaaggatacggatcatatccaatggatactcagtacggggagattttgagtgctacctagtcgcgacaatcagttatacaggggggcatgtcttcatgcaagaggtgatccgctctgccaaggcgttagttagagggggcggtagtatcatgacaaaacaagatgaacaacaattgaatcttgctttccagaggcagctcaacaggattcgtgggatactgggacagaggatatcgataatgatacgctacttgcagcatactattgatatggcattgattgaagcgggaggccaacctgtaagaccgagcaatgttggaatcaacaaggcactcgacttaggagatgagacatatgaggaaatcatgatacagcatattgacacaacacttaagacagcaatcttcctagaacaagaagaagaactggcagacacagtctttgtgttaacaccttataacctaacggcaagaggaaaatgtaatacagtacttattgcatgcactaaacatctatttgaaacaactatattacagactacacgagacgacatggataagatagagaaattgttgtcccttatcttacaaggtcatatctcgcttcaggatctcctgccactcaagtcatatcttaaacgtagcaattgtcccaagtacctcctcgattcactaggacgtatcaggctaaaagaggtatttgaacactcatcccgcatggtactaaccagaccgatgcaaaagatgtatctcaaatgtctcggaaatgctattaagggataccttgcagtggatgcatctcattgcaattgaatcatgacgcaatctcttttatacatcatactcgtaatcaatcatagttaccatcatttttaagaaaaacagtaacgatttatggtgtcacgtatgttgccaaatctttgtttggt

SEQ ID NO: 41 - DNA, 36 bp, artificial sequence
  NP-FP-pc3 primer
ccgaattcatgtcatctgtgttcaatgagtatcagg

SEQ ID NO: 42 - DNA, 36 bp, artificial sequence
  NP-RP-pc3 primer
ccgcggccgcttaccattctagcccgttctcgtatg

SEQ ID NO: 43 - DNA, 30 bp, artificial sequence
  P-FP-pc3 primer
ccgaattcatggatttcgccaatgatgaag

SEQ ID NO: 44 - DNA, 40 bp, artificial sequence
  P-RP-pc3 primer
ccgcggccgcttacgcattatatattgcctgcttgactcg

SEQ ID NO: 45 - DNA, 40 bp, artificial sequence; identical to SEQ ID NO: 44
  L-FP-pc3 primer

SEQ ID NO: 46 - DNA, 32 bp, artificial sequence
  L-RP-pc3 primer
ccggtaccatggatataaaacaagttgacctg

SEQ ID NO: 47 - DNA, 6231 bp, artificial sequence
  5′-FLG APMV8 genome
gggcggccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcccgggtgttaagcgtctgatgagtccgtgaggacgaaactataggaaaggaattcctatagtcaccaaacaaggaatgcaagaccaacgggaactttaaataaaacaatcgaattattgggggcgaagcaagtggatctcgagctcgaggccgaaaccctgaatttcactggaggttttgaataggtcgctataggactcaatatgtcatctgtgttcaatgagtatcaggcgcttcaagaacaacttgtgaagccggctgtcaggagacctgatgttgcctcaacgggtttactcagagcggaaatacctgtctgtgttacattatctcaagaccccggtgagagatggagccttgcttgcttgaatattagatggcttgcgagtgattcatcaaccacaccaatgaagcaaggagcaatattgtcactgctgagtctacattcagacaatatgcgagctcacgcaacattagcagcaaggtctgcagatgcttcactcaccatacttgaggtagatgaagtagatattagcaactcactaatcaaattcaacgccagaagtggtgtatctgacaaacgctcaaatcaattgcttgcaattgcggatgacatccccaaaagttgcagtaatgggcatccatttcttgacacagacattgagaccagagacccgctcgatctatcagagactatagatcgcctgcagggtattgcagctcagatatgggtgtcagccataaagagcatgacagcgcctgacaccgcatcagagtcagaaagtaagaggctggccaaatatcaacaacaaggccgactggttaagcaagtactcttgcattctgtagtcaggacagaatttatgagagttattcggggcagcttggtactgcgccagtttatggttagcgagtgcaagagggcttcagccatgggcggagacacatctaggtactatgctatggtgggtgacatcagtctttacatcaagaatgcaggattgactgcatttttcctcaccctgaagttcggagttggtacccagtatccaaccttagcaatgagtgttttctccagtgaccttaaaaggcttgctgcactcatcaggctatacaaaaccaagggagacaatgcaccatacatggcattcctggaggactccgatatgggaaattttgctccagcaaattatagcacaatgtactcttatgccatgggcattgggacaattctggaagcatctgtatctcgataccagtatgccagagactttaccagtgagaattatttccgtcttggagttgagacagcccaaagccagcagggagcatttgacgagagaacagcccgagaaatgggcttgactgaggaatcaaaacagcaggttagatcactgctaatgtcagtagacatgggtcccagttcaattcatgagccatctcgccctgcatttatcagtcaagaagaaaataggcagcctgcccagaacttgtcagatactcagggtcagaccaagccagtcccgaagcagcccgcaccaagggccgactcagatgacattgatccatacgagaacgggctagaatggtaattcaaccaccccgacacatccacctatacaccaattccgtgacatattaacccaatcaaacatttcataaactatagtagtcattgatttaagaaaaaattgggggcgacctcaattgtgaaacataccagatccgtccacaacaccactcaacaacccacacacaatggatttcgccaatgatgaagaaattgcagaacttttgaatctcagcaccaatgtaatcaaggagattcagaaatccgaactcaagcctccccaaaccaccggacgaccacctgtcagtcaagggaacacaagaaatctaactgatctatgggaaaaggagactgcaagtcagaccaagacaccggcccaatctacacaaaccacacaagttcagtctgatgaaaatgaggagggagaaatcaagtccgagtcaactgatggccacatcagaggaactgttaatcaatcagagcaagtcccagaacaaaaccagagcagatcttcaccaggtgatgatctcgacagagctctcaacaagcttgaagggagaatcaatttaatcagctcaatggacaaagaaattaaaaagggccctcgcatccagaatctccctgggtcccaggcggcaactcaacaggcgacccacccattggcaggggacaccccgaacatgcaagcacagacaaaagccctggcgaagccacatcaagaggcaatcaatcctggcaaccaggacacaggagagagtattcatttaccaccttccatggcaccaccagagtcattagttggtgcaatccgcaatgcaccccaattcgtgccagaccaatctatgacgaatgtagatgcggggagtgtccaactacatgcatcatgtgcagagatgataagtagaatgtttgtagaagttatatccaagcttgataaactcgagtcgagactgaatgatatagcaaaagttgtgaacactaccccccttattaggaatgatattaaccaacttaaggccacaaccgcactgatgtctaaccaaattgcctccatacaaattcttgacccagggaatgcaggggtgaggtccctctctgaaatgaaatctgtgacgaagaaagctgctgttgtaattgcagggtttggagacgacccaactcaaattattgaagaaggcattatggccaaagatgctcttggaaaacctgtgcctccaacatctgttatctcagccaaagctcagacttcttccggtgtgagtaagggtgaaatagaaggattgattgcattggtggaaacattagttgacaatgacaagaaggcagcaaaactgattaaaatgattgatcaagttaaatcccatgccgattacgcccgagtcaagcaggcaatatataatgcgtaatactgtaactacacaaacaatcaatactgctgtcggttgcacccacctcagcaaatcaataatcttttagaatttattgattaagaaaaaattgactactataagaaaagaacaccaagttgggggcgaagacacgattgaccacagtcgctatctgtaaggctcctcaccaaaaatggcatatacaacattgaaactgtgggtggatgagggtgacatgtcgtcttcgctcctatcattcccgttggtactaaaagagacagacagaggcacaaaggagcttcaaccacaggtaagggtagattcaattggcgatgtgcagaacgccaaagagtcctcgatattcgtgactctatatggtttcatccaagcaattaaggagagttcagatcgatcgaaattcttccatccaaaagatgacttcaaacctgagacagtcactgcaggactggtagtggtaggtgcgatccgaatgatggctgatgttaataccatctctaatgacgcactagcgctggagatcactgttaagaaatctgcaacttctcaagagaaaatgacggtgatgttccacaatagccccccttcattgagaactgcaataactatccgagcaggaggtttcatctcgaatgcagacgagaatataaaatgtgccagcaaattgactgcaggagtgcagtacatattccgcccaatgtttgtttcaatcactaaattacacaatggcaaactatatagggtgcccaaaagcatccacagcatctcatccactctactgtatagtgtgatgttggaggtaggattcaaagtggatattgggaaggatcatccccaggcaaagatgctgaagaaggtcacaatcggcgatgcagacacatactgggggtttgcatggttccacctgtgcaatttcaaaaagacatcctctaagggaaagccaagaacgctagacgaactaaagacaaaagtcaaaaatatggggttgaaattggagttacatgacctgtggggtccgactattgtggtccaaatcactggcaagagcagcaaatatgctcaaggatttttttcctccaatggtacttgttgtctcccaatcagcagatctgcaccagagcttgggaagcttctgtggtcttgttcagcaactataggtgacgcaacagttgttatccaatcaagcgagaaaggggaactcctaaggtctgatgacctcgagatacgaggtgctgtggcctccaagaaaggtagactgggctcatttcaccccttcaaaaaatgatgcaggacatagtacagagaattagagagccattagatgtgcgcaaaaaacataatctgcgatgaactgcccagactccactttaatctaggttgcagggaaatagtacacgacatgcgaaatactatcacggtcaccagcaatcaataaagctgatcaatcactatattaggaatcaaataggataacaattattaatccaatttcctaattataaaaaattgctttaaaggttattgacgagtcgggggcgaaatcttgccacttagtctgcagtcaatcttagaatctacatattgaactatgggtcaaatatcagtatatctaattaatagcgtgctattattgctggtatatcctgtgaattcgattgacaatacactcattgccccaatcggagttgccagcgcaaatgaatggcagcttgctgcatatacaacatcactttcagggacaattgccgtgcgattcctacctgtgctcccggataatatgactacctgtcttaaagaaacaatcactacatacaataatactgtcaacaacatcttaggcccactcaaatccaatctggatgcactgctctcatctgagacttatccccagacaagattaattggggcagttataggttcaattgctctcggtgttgcaacatcggctcaaatcactgctgcagttgctctcaagcaagcgcaagacaatgcaaggaacatactagcactcaaagaagcactgtccaaaaccaatgaggcggtcaaggagcttagtagtgggttacaacaaacagctattgcacttggtaagatacagagttttgtgaatgaggaaattctgccatctatcaaccaactgagctgcgaggtgacagccaataaacttggggtgtatttatctctgtatctcacagaactgaccaccatattcggtgcacagctgaccaaccctgcattgacttcattatcatatcaagcactgtacaacctgtgtggtggcaacatggcaatgcttactcagaagattggaattaaacagcaagacgttaattcgctatatgaagccggactaatcacaggacaagtcattggttatgactctcattaccagctgctggtcatccaggtcaattatccaagcatttctgaggtcactggtgtacgtgcgacagaattagtcactgttagtgtaacaacagacaagggtgaagggaaagcaattgtaccccaatttgtagctgaaagtcgggtgactattgaagagcttgatgtcgcatcttgtaaattcagcagcacgaccctatattgcaggcaggtcaacacaagggcacttcccccgctagtagctagctgtcttcgaggtaactatgatgccgcgg

SEQ ID NO: 48 - DNA, 10189 bp, artificial sequence
  3′-FLG APMV8 genome
gcggccgccaggtcaacacaagggcacttcccccgctagtagctagctgtcttcgaggtaactatgatgattgtcaatataccacagagattggagcattatcatcccggtatataacactagatgggggggtcttagttaattgcaagtcaattgtttgtaggtgccttaatccaagtaagatcatctctcaaaatacaaacgctgcagtaacatatgttgatgccacaatctgcaaaacaattcaattggatgatatacaactccagctggaagggtcactatcatcagtttatgcaagaaacatctcaattgagatcagtcaggtgaccacatccgggtctttagatatcagcagtgagataggaaacatcaataatacggtgaatcgtgtggaggatttaattcaccaatcagaggaatggctggcaaaggttaacccacacattgttaataatacaacactaattgtactctgtgtgttaagtgcgcttgctgtgatctggctggcagtattaacggctattataatatacttgagaacaaagttgaagactatatcggcattagctgtaaccaatacaatacagtctaacccctatgttaaccaaacgaaacatgaatctaagttttgatcattcaagccaaaacagaggatctaggctcaggttaataatagttcaatcaatatttgatttattaggtttttttcactaattattaatatactcgtgattagatgataacgttaaaagtcttagatatttaataaaaaatgtaacctgggggcgacccatttataggtgagtatatattaggaagtccttatattgcactgtgatttcaaacaattatattacctcatatctaccttgtctaaagacatcatgagtaacattgcatccagtttagaaaacattgtagagcaggatagtcgaaaaacaacttggagggccatctttagatggtccgttcttcttattacaacaggatgcttagccttatccattgttagcatagttcaaattggaaatttgaaaattccttctgtaggggatctggctgatgaagtggtgacacccttgaaaaccactctgtcagatacactcaggaatccaattaaccagataaatgatatatttaggattgttgcccttgatattccattgcaagtgaccagtatccaaaaagaccttgcaagtcaatttaacatgttgatagatagtttaaatgctatcaaattaggcaacgggaccaaccttatcatacctacatcagacaaggagtatgcaggaggaattggaaaccctgtatttactgtcgatgctggaggttctataggattcaaacagtttagcttaatagaacatccgagctttattgctggacctacaacgacccgaggctgtacaagaatacccacttttcacatgtcagaaagtcattggtgctactcacacaacatcatcgctgctggctgtcaagatgccagtgcatccagtatgtatatctcaatgggagttctccatgtgtcctcatctggcactcccatttttcttactactgcaagtgagctgatagacgatggagttaaccgtaagtcatgcagcattgtagcaacccaatttggctgtgacattttgtgcagtattgtcacagagaaggagggagatgattactggtctgatactccgactccaatgcgccacggccgtttttcattcaatggtagttttgtagaagccgaactaccagtgtccagtatgttctcatcattctctgccaactaccctgctgtgggatcaggcgaaattgtaaaagatagaatattattcccaatttacggaggtataaagcagacttcaccagagtttaccgaattagtgaaatacggactctttgtatcaacacctacaactgtgtgccagagtagctggacttatgaccaggtaaaagctgcgtataggccagattacatatcaggccggttctgggcacaagtgatactcagctgcgctcttgatgcagtcgacttatcaagttgtattgtaaagattatgaatagcagcacagtgatgatggcagcggaaggaaggataatgaagatagggattgattacttttactatcagcggtcatcttcttggtggccattggcatttgtcacaaaactagacccgcaagagttggcagacacaaactcaatatggctgaccaattccataccaatcccgcaatcaaagttccctcggccttcatattcagaaaattattgcacaaagccagcagtttgccctgctacttgtgtcactggtgtgtactctgatatttggcccctgacctcatcttcatcactcccgagcataatttggatcggccagtaccttgatgctcctgttggaaggacttatcctagatttggaattgcaaatcagtcacactggtacctccaagaagatattctacccacttccaccgcaagtgcgtattcaaccactacatgttttaagaatactgccaggaatagagtgttctgcgtcaccattgccgaatttgcagatgggttgtttggagagtacaggataacacctcagttgtacgaattagtgagaaataattgaataacaataattttgggactcattttgtcgcaaagtgaaattgtcatctttaaaaataatcaattcgatgatttttattgaacatgattaagcaatcatgtgggaaatttattatctcataaattctaatagttgtaaatgatgaattaagaaaaaatggagggcgacctctacacaaacatggatataaaacaagttgacctgataatacaacccgaggttcatctcgattcacccatcatattgaataaactggcactattatggcgcttgagtggtttacccatgcctgcagacctacgacaaaaatccgtagtgatgcacatcccggaccacatcttagaaaaatcagaatatcggatcaagcaccgtctagggaaaatcaagagtgacataacacattactgtcagtattttaatattaatttggcaaatattgatccgataacccaccccaaaagtttgtattggttatccagactaacaatagctagtgctggaacttttaggcatatgaaagatagaatcttgtgtacagttggctctgaatttggacacaaaattcaagatttattttcactgctgagccataaactagtaggtaacggggatttatttaatcaaagtctctcaggtacacgtttgactgcaagtccgttatcccctttatgcaatcaatttgtctctgacatcaagtctgcagtcacgacaccctggtcagaagctcgttggtcttggcttcatatcaaacaaacaatgagatatctgataaaacaatcacgcactacaaattcggctcatttaacagaaatcataaaagaagaatggggtttagtaggtattactccagatcttgtcattctttttgacagagtcaataatagtctgactgcattaacatttgagatggttctaatgtattcagatgtattagaatcccgtgacaatattgtgttagtggggcgactatctacctttctacagccagtagttagtagactggaggtgttgtttgatctagtagattcattggcaaaaatcttaggtgacacaatatatgagattattgcagtgttagagagcttgtcttatgggtcagttcaactacatgatgcaagtcactctcatgcagggtcttttttttcatttaacatgaatgaacttgataacacactatcaaagagggtagatccgaaacacaagaacactataatgagcattataagacaatgcttttctaatctagatgttgatcaagctgcagagatgctatgcctgatgagattattcggacacccaatgttaactgcaccggatgcagcagccaaagtgaggaaagcaatgtgtgctccaaaacttgttgaacacgacaccatcttgcagacattatctttcttcaaggggataattataaatgggtacagaagatcacactctggcctgtggcccaatgtagagccgtcttcaatttatgatgatgatctcagacagctgtacttagagtcagcagagatttcccatcattttatgcttaaaaactacaagagtttaagcatgatagaattcaagaagagcatagactacgatcttcatgatgacctaagtactttcttaaaggatagagcaatttgccggccgaaatcccagtgggatgtcatatttcgtaagtctttacgcagatctcatacgcagtcccagtatctggacgaaattaagagcaaccgattgctaattgattttcttgattctgctgaatttgaccctggaaaagaatttgcatatgtaaccacaatggattatttgcacgataatgaattttgtgcttcatattctctaaaggaaaaggagatcaaaactactgggaggatatttgcaaaaatgacacgcaatatgagaagttgccaagtaatacttgaatctttgttatcaaagcatatatgcaagttcttcaaagagaatggcgtttcgatggagcaattgtcattgaccaagagtctacttgcaatgtctcaactctcaccaaaagtctcgactttgcaggacactgcatcacgtcatgtaggtaactcaaaatctcagattgcaaccagcaacccatctcggcatcactcgacacccaatcagatgtcactctcaaatcgaaaaacggttgtagcaactttcttaacaactgacttggaaaaatactgcctgcagtggcgatattcaactattaaattgtttgcacaagctctaaatcaactctttgggattgatcacggatttgaatggatacatttaagacttatgaacagcaccttatttgttggcgatccttactcgcctcctgaagatccaacactagaagatatagataaagcaccaaatgatgatatcttcatagtttctccaaggggaggcatagagggtttatgtcagaaaatgtggaccatgatatcaattagtgctatacactgtgtagcagagaaaattggtgcacgagtggcagcaatggtgcagggtgataatcaagtaatagctatcaccaaagaattattcagaggagagaaagcttgtgatgtcagagatgagttagacgagcttggtcaagtgttttttgatgagttcaagagacacaattatgcaattggacacaatcttaagctaaatgagacaatacaaagccaatccttttttgtatattccaaacgaatattctttgaagggcgattgcttagtcaagtcctcaaaaatgctgccaagttatgtatggttgctgaccatctaggtgaaaacactgtatcttcctgtagcaacctgagctcgacaattgcccgcttagtggaaaatgggtttgagaaggacactgcttttgtgttgaacctagtctacatcatgactcagattctttttgatgagcattactcgattgtatgcgatcaccatagtgtcaaaagcttgattggatcaaaaaaccatcggaatttattgtattcatctctaataccaggtcagctcggcggtttcaacttcctcaatataagtcggttgttcactaggaatataggtgacccagtaacatgtagtctgtctgatctcaaatgcttcatagccgcaggtctccttccaccctatgtcctaaaaaatgtggttctgcgtgagcctggtcctgggacatggttgacgttgtgctctgatccttacacccttaacataccatacacacagcttccaaccacatatctcaaaaagcacacccagcgatcattgctttcacgtgcagtaaatcctttattagccggtgtacaagtgccaaatcagcatgaggaagaagagatgttggctcgctttctccttgatcgtgaatatgtgatgccccgcgttgctcatgtaatactagaatcatcagtccttggcaaacggaaacaaatccaaggcttaattgatacaactccaaccatcattagaacatctctagttaatctgccagtgtctagaaagaaatgcgaaaaaataatcaattactctctcaattatattgctgagtgtcatgactccttacttagccaggtctgcttcagtgataataaggaatacttgtggtcaacctccttaatatcagttgagacctgtagtgtgacaattgcggactatctgagagctgtcagctggtctaatatattagggggaagaaacatatccggggtgactacacctgatactattgaattaattcaaggttgtttaataggtgaaaattctagttgtactctttgtgaatcgcatgatgacgcattcacgtggatgcacttgcctggcccactttacatccctgaaccatcagttactaactctaaaatgcgtgtgccatatctgggttcaaaaacagaggagcggaaaacagcctcaatggcagcaataaaaggaatgtcacatcacctgcgtgcagtcttaagaggcacatccgtatttatttgggcatttggggatacagatatcaattgggataatgcattgcagattgcccaatcacggtgtaacatcacattggatcaaatgagattacttacaccaattcctagcagttcaaatattcaacatagactcgatgacggaatcagcacgcagaaatttactcctgcaagccttgctcgaatcacatccttcgttcacatctgtaatgacagccagaggttagagaaggatggctcatctgtcgactcaaacttgatttaccagcaaattatgttacttggactcagcatctttgaaacaatgtactcaatggaccaaaagtgggtattcaataaccataccttgcatttgcacactggacactcctgttgtccaagggaactagacataagtttggtgaacccgccgagacatcagaccccggagctgactagcacaacaaccaacccgttcctatatgatcagctcccattaaatcaagaaaacttgacaacacttgagattaagacatttaaattcaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagacgggccattcttaattacttggacacaactttgcaaaggatccctgtgatacaattagccaacattgcactcaccatttcgcaccctgagatatttcgcagaattgtcaacaccgggatccataaccagattaagggcccatatgtggcaacaacggatttcatagctgcaagtagagatatcatattatcaggtgcaagggagtatctatcttatttaagcagtgggcaggaagactgttacacattcttcaactgtcaagatggggatcttactccaaaaatggaacagtatcttgcaaggagggcatgccttttaacattattgtataatactgggcaccagatccccgttatccgatcactgacgccaatagagaagtgcaaggtgctcacagaatacaatcaacaaattgagtacgcagatcaagagtttagctctgtactaaaagtggtcaatgcactactacaaaatcctaagatagatgcattagtttcaaatctctacttcaccaccagacgtgttctatcaaacctcagatcatgtgataaggctagatcatatattgaatatttgtacactgaggacttcggagagaaagaggatacagtacaatatgacatcatgacaacaaacgatatcatacttactcatggtctattcacacagatcgaaatatcttatcaagggaatagtctccataagttccttactccggataacgcgcctggatctttgatcccattctctatttcaccaaattcacttgcatgtgaccctcttcatcacttgctcaagtcggtcggtacatcaagcacaagttggtacaagtatgcaatcgcctatgcagtgtctgaaaagaggtcagctcgattaggagggagcttgtacattggtgaagggagcggaagtgtgatgactttactcgagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctagggtttgtccagcaatttaaaccgttgtggagagacatagacattgagactaatgttacagatccatcatttatcaattttgcattgaatgaaatcccaatgcaatcattaaaacgagtaaattgtgatgtggaatttgaccgtggtatgccgattgaacgggttattcagggttacacccatatcttacttgttgccacttacggattacagcaagattcaatactgtgggtgaaggtatataggacatctgaaaaagtatttcaattcttactgagtgccatgatcatgatctttggttatgtaaaaatccacaggaatggttatatgtcgacaaaggatgaagagtacatattgatgtctgactgcaaggaacctgtaaactatacagctgtccctaacattcttacacgtgtaagtgatttagtgtcgaagaatctgagtcttatccatccagaagacctcagaaaagtaaggtgtgaaacagattccctgaatttgaagtgcaatcatatttatgagaaaataattgccagaaaaattccattacaggtatcatcaactgactctttgctcctccaattaggcggtgttatcaactcggtgggctcaactgatcctagagaggttgcaacattatcttctattgagtgtatggactatgttgtctcatcaattgatttggctatattggaggcaaatattgtaatctcagagagtgctggtcttgacctcgctttaatgttaggcccattcaacttaaataagcttaagaaaattgacacaatccttaagtcaagcacctatcagctaatcccgtactggttgcgctatgagtactctattaatccgagatctttgtcatttctaatcactaaattacaacaatgccgaatttcatggtcagatatgatcacgatttctgaatttcgtaagaaatccaagcggcctatatttatcaaacgagtaatagggaatcaacagctaaaatcattctttaatgaaagctcaagtattgttttgactcgggctgaagttaaagtctgtataaagttcctcggtgcaatcatcaagttgaaataatttctgcgattttaaaggggtgtaatgttctaatttgcacttgaagtaatatagcttgtaatcattcgctaggggataggataatttctctaacctctgaatctatattcctagagtataacaaatatatacataataaaaatgattttaagaaaaaatccgacactcaaagaaaattggtgcctgtaatattcttcttgccaaatgattgtgaagtgtctagcctaacttaaaacaatcgtattcgatagggaagaatgatatataaaataactaataaaaaattgtattagtaaaaattaccgtatttcctgtattccatttctggtgggtcggcatggcatctccacctcctcgcggtccgacctgggcatccgaaggaggacgcacgtccactcggatggctaagggagggcgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgccgcgg

SEQ ID NO: 49 - DNA, 2652 bp, artificial sequence
  T7 polymerase condon-optimized
atgaatacaattaacatcgccaaaaacgacttcagcgatatcgaactggccgccatcccattcaatacactggccgatcactatggcgagcgcctggccagagagcagctggccctggagcacgagtcctacgagatgggcgaggcccgcttccggaagatgttcgagcggcagctgaaggccggcgaagtggccgacaacgccgccgccaagcccctgatcaccaccctgctgcccaaaatgatcgcccgcattaatgactggttcgaagaggtgaaagccaaaagggggaaacgccccaccgccttccagttcctgcaggagatcaaacccgaggccgtggcctatatcaccattaagacaacactggcctgtctgacaagcgccgataacacaacagtgcaggccgccgcctctgccatcggccgggccatcgaggacgaggccagattcggccgcattcgggacctggaggccaaacacttcaaaaagaacgtggaagagcagctgaacaagcgggtgggacacgtgtacaagaaagccttcatgcaggtggtggaggccgatatgctgtctaaagggctgctgggaggcgaggcctggagctcctggcacaaggaagatagcatccacgtgggagtgagatgcatcgagatgctgatcgagtctaccgggatggtgtccctgcacagacagaacgccggggtggtgggccaggatagcgagacaattgagctggcccccgagtatgccgaagccattgccacacgggccggcgccctggccggaatctccccaatgttccagccatgtgtggtgctgcctaagccctggacaggaatcacaggcgggggatactgggccaatggcaggaggccactggccctggtgaggacacactccaagaaggccctgatgagatatgaagacgtgtatatgcctgaggtgtataaggccatcaatatcgcccagaacaccgcctggaaaattaataagaaggtgctggccgtggccaatgtgatcacaaagtggaagcactgcccagtggagaatatcccagccattgagcgcgaagagctgcccatgaagccagagaacattgatatgaacccagaggccctgatcgcctggaagagagccgccgccgccgtgtatcgcaaggataaagccaggaagtcccgccggatcagcctggagttcatgctggagcaggccaataagtttgccaaccacaaggccatttggtttccttataatatggactggcgcgggagagtgtacgccgtgtctatgttcaatccacagggaaacgatatgaccaagggactgctgaccctggccaagggcaagcctattgggaaagagggctactattggctgaagatccacggggccaactgcgccggagtggataaggtgccttttcccgagcgcatcaagttcatcgaggaaaaccacgaaaacatcattgcctgcgccaagagccccctggaaaacacatggtgggccgagcaggacagccccttttgctttctggccttttgctttgagtacgccggcgtgcagcaccacggcctgtcctacaactgctccctgcccctggccttcgatggatcttgttctggcatccagcacttttccgccatgctgcgcgacgaggtggggggcagggccgtgaatctgctgcctagcgagaccgtgcaggatatctacggcatcgtggccaagaaggtgaacgagattctgcaggccgatgccattaacggaacagacaacgaggtggtgaccgtgaccgatgaaaatacaggcgagatttccgagaaagtgaagctgggcaccaaggccctggccggacagtggctggcctatagcgtgacccggtccgtgaccaagcgctctgtgatgacactggcctacgggtctaaggaatttggatttcggcagcaggtgctggaagacaccattcagcccgccatcgactccggaaagggcctgatgtttacccagccaaatcaggccgccgggtacatggccaagctgatctgggaaagcgtgtccgtgacagtggtggccgccgtggaagccatgaactggctgaagtctgccgccaagctgctggccgccgaagtgaaggacaagaaaaccggggaaattctgcggaaaagatgtgccgtgcactgggtgaccccagacggattcccagtgtggcaggagtacaagaagccaattcagacaaggctgaacctgatgttcctggggcagtttcgcctgcagccaacaatcaacacaaataaggatagcgagatcgacgcccacaagcaggaatccggcattgcccctaatttcgtgcacagccaggacggctctcacctgcgcaagacagtggtgtgggcccacgagaagtacggcattgaatcttttgccctgatccacgactctttcggcaccatcccagccgatgccgccaacctgttcaaggccgtgagagagaccatggtggacacctacgagagctgcgatgtgctggccgacttttacgaccagttcgccgatcagctgcacgaaagccagctggataagatgcctgccctgcccgccaaggggaacctgaacctgagagacatcctggagtccgatttcgccttcgcctga

SEQ ID NO: 50 - DNA, 32 bp, artificial sequence
  EGFP-FP primer
ccggatccatggtgagcaagggcgaggagctg

SEQ ID NO: 51 - DNA, 35 bp, artificial sequence
  EGFP-RP primer
ccgcggccgcttacttgtacagctcgtccatgccg

SEQ ID NO: 52 - DNA, 1419 bp, artificial sequence
  APMV8 minigenome
gaattctaatacgactcactatagggaccagaaatggaatacaggaaatacggtaatttttactaatacaattttttattagttattttatatatcattcttccctatcgaatacgattgttttaagttaggctagacacttcacaatcatttggcaagaagaatattacaggcaccaattttctttgagtgtcggattttttcttaaaatcatttttattatgtatatatttgttatactctaggaatatagattcagaggttagagaaattatcctatcccctagcgaatgattacaagctatattacttcaagtgcaaattagaacattacacccctttaaaatcgcagaaattacaagctatattacttcaagtgcaaattagaacattacacccctttaaaatcgcagaaattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatattgagtcctatagcgacctattcaaaacctccagtgaaattcagggtttcggcctcgagctcgagatccacttgcttcgcccccaataattcgattgttttatttaaagttcccgttggtcttgcattccttgtttggtgggtcggcatggcatctccacctcctcgcggtccgacctgggcatccgaaggaggacgcacgtccactcggatggctaagggagggcgtagcataaccccttggggcctctaaacgggtcttgaggggttttttggcggccgc

SEQ ID NO: 53 - DNA, 1386 bp, artificial sequence
  NP gene of FJ215863
atgtcatctgtattcaatgagtatcaggcgcttcaagaacaacttgtaaagccggctgtcaggagacctgatgttgcctcaacgggtttactcagagcggaaatacctgtctgtgtcacattatctcaagaccccggtgagagatggagccttgcttgcttgaatatcagatggcttgtgagtgattcatcaaccacaccaatgaagcaaggagcaatattgtcactgctgagtctacattcagacaatatgcgagctcacgcaacattagcagcaaggtctgcagatgcttcactcaccatacttgaggtagatgaagtagatattggcaactcactaatcaaattcaacgccagaagtggtgtatctgataaacgctcaaatcaattgcttgcaattgcggatgacatccccaaaagttgcagtaatgggcatccatttcttgacacagacattgagaccagagacccgctcgatctatcagagactatagaccgcctgcagggtattgcagctcagatatgggtgtcagccataaagagcatgacagcgcctgacaccgcatcagagtcagaaagtaagaggctggccaaatatcaacaacaaggccgactggttaagcaagtactcttgcattctgtagtcaggacagaatttatgagagttattcggggcagcttggtactgcgccagtttatggttagcgagtgcaagagggcttcagccatgggcggagacacatctaggtactatgctatggtgggcgacatcagtctgtacatcaagaatgcaggattgactgcatttttcctcaccctgaagttcggagttggtacccagtatccaaccttagcaatgagtgttttctccagtgaccttaaaaggcttgctgcactaatcaggctatacaaaaccaagggagacaatgcaccatacatggcattcctggaggactccgatatgggaaattttgctccagcaaattatagcacaatgtactcttatgccatgggcattgggacaattctggaagcatctgtatctcgataccagtatgccagagactttaccagtgagaattatttccgtcttggagttgagacagcccaaagccagcagggagcatttgacgagagaacagcccgagaaatgggcttgactgaggaatcaaaacagcaggttagatcactgctaatgtcagtagacatgggtcccagttcagttcatgagccatctcgccctgcatttatcagtcaagaagaaaataggcagcctgcccagaactcgtcagatactcagggtcagaccaagccagtcccgaagcagcccgcaccaagggccgactcagatgacattgatccatacgagaacgggctagaatggtaa

SEQ ID NO: 54 - protein, 461 aa, artificial sequence
  NP protein ACO48297
MSSVFNEYQALQEQLVKPAVRRPDVASTGLLRAEIPVCVTLSQDPGERWSLACLNIRWLVSDSSTTPMKQGAILSLLSLHSDNMRAHATLAARSADASLTILEVDEVDIGNSLIKFNARSGVSDKRSNQLLAIADDIPKSCSNGHPFLDTDIETRDPLDLSETIDRLQGIAAQIWVSAIKSMTAPDTASESESKRLAKYQQQGRLVKQVLLHSVVRTEFMRVIRGSLVLRQFMVSECKRASAMGGDTSRYYAMVGDISLYIKNAGLTAFFLTLKFGVGTQYPTLAMSVFSSDLKRLAALIRLYKTKGDNAPYMAFLEDSDMGNFAPANYSTMYSYAMGIGTILEASVSRYQYARDFTSENYFRLGVETAQSQQGAFDERTAREMGLTEESKQQVRSLLMSVDMGPSSVHEPSRPAFISQEENRQPAQNSSDTQGQTKPVPKQPAPRADSDDIDPYENGLEW

SEQ ID NO: 55 - DNA, 1218 bp, artificial sequence
  P gene of FJ215863
atggatttcgccaatgatgaagaaattgcagaacttttgaatctcagcaccaatgtaatcaaggagattcagaaatccgaactcaagcctccccaaaccaccggacgaccacctgtcagtcaagggaacacaagaaatctaactgatctatgggaaaaagagactgcaagtcagaccaagacaccggcccaatctccacaaaccacacaagttcagtctgatgaaaatgaggagggagaaatcaagtccgagtcaattgatggccacatcagaggaactgttaatcaatcagagcaagtcccagaacaaaaccagagcagatcttcaccaggtgatgatctcgacagagctctcaacaagcttgaagggagaatcaattcaatcagctcaatggacaaagaaattaaaaagggccctcgcatccagaatctccctgggtcccaggcggcaactcaacaggcgacccacccattggcaggggacaccccgaacatgcaagcacagacaaaagccctggcgaagccacatcaagaggcaatcaatcctggcaaccaggacacaggagagagtattcatttaccaccttccatggcaccaccagagtcattagttggtgcaatccgcaatgcaccccaattcgtgccagaccaatctatgacgaatgtagatgcggggagtgtccaactacatgcatcatgtgcagagatgataagtagaatgtttgtagaagttatatccaagcttgataaactcgagtcgagactgaatgatatagcaaaagttgtgaacaccaccccccttattaggaatgatattaaccaacttaaggccacaaccgcactgatgtccaaccaaattgcttccatacaaattcttgacccagggaatgcaggggtgaggtccctctctgaaatgaaatctgtgacgaagaaagctgctgttgtaattgcagggtttggagacgacccaactcaaattattgaagaaggcattatggccaaagatgctcttggaaaacctgtgcctccaacatctgttatctcagctaaagctcagacttcttccggtgtgagtaagggtgaaatagaaggattgattgcattggtggaaacattagttgacaatgacaagaaggcagcaaaactgattaaaatgattgatcaagttaaatcccacgccgattacgcccgagtcaagcaggcaatatataatgcgtaa

SEQ ID NO: 56 - protein, 405 aa, artificial sequence
  P protein ACO48298
MDFANDEEIAELLNLSTNVIKEIQKSELKPPQTTGRPPVSQGNTRNLTDLWEKETASQTKTPAQSPQTTQVQSDENEEGEIKSESIDGHIRGTVNQSEQVPEQNQSRSSPGDDLDRALNKLEGRINSISSMDKEIKKGPRIQNLPGSQAATQQATHPLAGDTPNMQAQTKALAKPHQEAINPGNQDTGESIHLPPSMAPPESLVGAIRNAPQFVPDQSMTNVDAGSVQLHASCAEMISRMFVEVISKLDKLESRLNDIAKVVNTTPLIRNDINQLKATTALMSNQIASIQILDPGNAGVRSLSEMKSVTKKAAVVIAGFGDDPTQIIEEGIMAKDALGKPVPPTSVISAKAQTSSGVSKGEIEGLIALVETLVDNDKKAAKLIKMIDQVKSHADYARVKQAIYNA

SEQ ID NO: 57 - DNA, 1110 bp, artificial sequence
  M gene of FJ215863
atggcatatacaacattgaaactgtgggtggatgagggtgacatgtcgtcttcgctcctatcattcccgttggtactaaaagagacagacagaggcacaaaggagcttcaaccacaggtaagggtagattcaattggcgatgtgcagaacgccaaagagtcctcgatattcgtgactctatatggtttcatccaagcaattaaggagagtacagatcgatcgaaattcttccatccaaaagatgacttcaaacctgagacagtcactgcaggactggtagtggtaggtgcgatccgaatgatggctgatgttaataccatctctaatgacgcactagcgctggagatcactgttaagaaatctgcaacttctcaagagaaaatgacggtgatgttccacaatagccccccttcattgagaactgcaataactatccgagcaggaggtttcatctcgaatgcagacgagaatataaaatgtgccagcaagttgactgcaggagtgcagtacatattccgcccaatgtttgtttcaatcactaaattacacaatggcaaactatatagggtgcccaaaagcatccacagcatctcgtccactctactgtatagtgtgatgttggaggtaggattcaaagtggatattgggaaggatcatccccaggcaaagatgctgaagaaggtcacaatcggcgatgcagacacatactgggggtttgcatggttccacctgtgcaatttcaaaaagacatcctctaagggaaagccaagaacgctagacgaactaaagacaaaagtcaaaaacatggggttgaaattggagttacatgacttgtggggtccgactattgtggtccaaatcactggcaagagcagcaaatatgctcaaggatttttttcctccaatggtacttgttgcctcccaatcagcagatctgcaccagggcttgggaagcttctgtggtcctgttcagcaactatcggtgacgcaacagttgttatccagtcaagcgagaaaggggaactcctaaggtctgatgacctcgagatacgaggtgctgtggcctccaagaaaggtagactgagctcatttcaccccttcaagaaatga

SEQ ID NO: 58 - protein, 369 aa, artificial sequence
  M protein of ACO48299
MAYTTLKLWVDEGDMSSSLLSFPLVLKETDRGTKELQPQVRVDSIGDVQNAKESSIFVTLYGFIQAIKESTDRSKFFHPKDDFKPETVTAGLVVVGAIRMMADVNTISNDALALEITVKKSATSQEKMTVMFHNSPPSLRTAITIRAGGFISNADENIKCASKLTAGVQYIFRPMFVSITKLHNGKLYRVPKSIHSISSTLLYSVMLEVGFKVDIGKDHPQAKMLKKVTIGDADTYWGFAWFHLCNFKKTSSKGKPRTLDELKTKVKNMGLKLELHDLWGPTIVVQITGKSSKYAQGFFSSNGTCCLPISRSAPGLGKLLWSCSATIGDATVVIQSSEKGELLRSDDLEIRGAVASKKGRLSSFHPFKK

SEQ ID NO: 59 - DNA, 1632 bp, artificial sequence
  F gene of FJ215863
atgggtcaaatatcagtatatctaattaatagcgtgctattattgctggtatatcctgtgaattcgattgacaatacactcattgccccaatcggagttgccagcgcaaatgaatggcagcttgctgcatatacaacatcactttcagggacaattgccgtgcgattcctacctgtgctcccggataatatgactacctgtcttagagaaacaatcactacatataataatactgtcaacaacatcttaggcccactcaaatccaatctggatgcactgctctcatctgagacttatccccagacaagattaattggggcagttataggttcaattgctctcggtgttgcaacatcggctcaaatcactgctgcagttgctctcaagcaagcgcaagacaatgcaaggaacatactagcactcaaagaagcactgtccaaaaccaatgaggcggtcaaggagcttagtagtgggttacaacaaacagctattgcacttggtaagatacagagttttgtgaatgaggaaattctgccatctatcaaccaactgagctgcgaggtgacagccaataaacttggggtgtatttatctctgtatctcacagaactgaccaccatattcggtgcacagctgactaaccctgcattgacttcattatcatatcaagcgctgtacaacctgtgtggtggcaacatggcaatgcttactcagaagattggaattaaacaacaagacgttaattcgctatatgaagccggactaatcacaggacaagtcattggttatgactctcagtaccagctgctggtcatccaggtcaattatccaagcatttctgaggtcactggtgtacgtgcaacagaattagtcactgttagtgtaacaacagacaagggtgaagggaaagcaattgtaccccaatttgtagctgaaagtcgggtgactattgaagagcttgatgtagcatcttgtaaattcagcagcacgaccctatattgcaggcaggtcaacacaagggcacttcccccgctagtagctagctgtcttcgaggtaactatgatgattgtcaatataccacagagattggagcattatcatcccggtatataacactagatgggggggtcttagttaattgtaagtcaattgtttgtaggtgccttaatccaagtaagatcatctctcaaaatacaaacgctgcagtaacatatgttgatgccacaatctgcaaaacaattcaattggatgatatacaactccagctggaagggtcactatcatcagtttatgcaagaaacatctcaattgagatcagtcaggtgaccacctccgggtctttagatatcagcagtgagataggaaacatcaataatacggtgaatcgtgtggaggatttaattcaccaatcagaggaatggctggcaaaggttaacccacacattgttaataatacaacactaatcgtactctgtgtgttaagtgcgcttgctgtgatctggctggcagtattaacggctattataatatacttgagaacaaagttgaagactatatcggcattagctgtaaccaatacaatacagtctaacccctatgttaaccaaacgaaacgtgaatctaagttttga

SEQ ID NO: 60 - protein, 543 aa, artificial sequence
  F protein of ACO48300
MGQISVYLINSVLLLLVYPVNSIDNTLIAPIGVASANEWQLAAYTTSLSGTIAVRFLPVLPDNMTTCLRETITTYNNTVNNILGPLKSNLDALLSSETYPQTRLIGAVIGSIALGVATSAQITAAVALKQAQDNARNILALKEALSKTNEAVKELSSGLQQTAIALGKIQSFVNEEILPSINQLSCEVTANKLGVYLSLYLTELTTIFGAQLTNPALTSLSYQALYNLCGGNMAMLTQKIGIKQQDVNSLYEAGLITGQVIGYDSQYQLLVIQVNYPSISEVTGVRATELVTVSVTTDKGEGKAIVPQFVAESRVTIEELDVASCKFSSTTLYCRQVNTRALPPLVASCLRGNYDDCQYTTEIGALSSRYITLDGGVLVNCKSIVCRCLNPSKIISQNTNAAVTYVDATICKTIQLDDIQLQLEGSLSSVYARNISIEISQVTTSGSLDISSEIGNINNTVNRVEDLIHQSEEWLAKVNPHIVNNTTLIVLCVLSALAVIWLAVLTAIIIYLRTKLKTISALAVTNTIQSNPYVNQTKRESKF

SEQ ID NO: 61 - DNA, 1734 bp, artificial sequence
  HN gene of FJ215863
atgagtaacattgcatccagtttagaaaacattgtagagcaggatagtcgaaaaacaacttggagagccatctttagatggtccgttcttcttatcacaacaggatgcttagccttatccattattagcatagttcaaattggaaatttgaaaattccttctgtaggggatctggctgatgaagtggtgacacccttgaaaaccactctgtctgatacactcaggaatccaattaaccagataaatgacatatttaggattgttgcccttgatattccattgcaagtgaccaatatccaaaaagaccttgcaagtcaatttaacatgttgatagatagtttaaatgctatcaaattaggcaacgggaccaaccttatcatacctacatcagacaaggagtatgcaggaggaattggaaaccctgtatttactgtcgatgctggaggttctataggattcaaacagtttagcttaatagaacatccgagctttattgctggacctacaacgacccgaggctgtacaagaatacccacttttcacatgtcagaaagtcattggtgctactcacacaacatcatcgctgctggctgtcaagatgccagtgcatccagtatgtatatctcaatgggagttctccatgtgtcctcatctggcactcccatttttcttactactgcaagtgagctgatagacgatggagttaaccgtaagtcatgcagcattgtagcaacccgatttggctgtgacattttgtgcagtattgtcacagagaaggagggagatgattactggtctgatactccgactccaatgcgccacggccgtttttcattcaatggtagttttgtagaagccgaactaccagtgtccagtatgttctcgtcattctctgccaactaccctgctgtgggatcaggcgaaattgtaaaagatagaatattattcccaatttacggaggtataaagcagacttcaccagagtttaccgaattagtgaaatacggactctttgtatcaacacctacaactgtgtgccagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcggaaggaaggataatgaagatagggattgattacttttactatcagcggtcatcttcttggtggccattggcatttgttacaaaactagacccgcaagaattggcagacacaaactcaatatggctgaccaattccataccaatcccgcaatcaaagttccctcggccttcatattcagaaaattattgcacaaagccagcagtttgccctgctacttgtgtcactggtgtgtactctgatatttggcccctgacctcatcttcatcactcccgagcataatttggatcggccagtaccttgatgctcctgttagaaggacttatcccagatttggaattgcaaatcagtcacactggtacctccaagaagatattctacccacttccaccgcaagtgcgtattcaaccactacatgttttaagaatactgccaggaatagagtgttctgcgtcaccattgccgaatttgcagatgggttgtttggagagtacaggataacacctcagttgtacgaattagtgagaaataattga

SEQ ID NO: 62 - protein, 577 aa, artificial sequence
  HN protein of ACO48301
MSNIASSLENIVEQDSRKTTWRAIFRWSVLLITTGCLALSIISIVQIGNLKIPSVGDLADEVVTPLKTTLSDTLRNPINQINDIFRIVALDIPLQVTNIQKDLASQFNMLIDSLNAIKLGNGTNLIIPTSDKEYAGGIGNPVFTVDAGGSIGFKQFSLIEHPSFIAGPTTTRGCTRIPTFHMSESHWCYSHNIIAAGCQDASASSMYISMGVLHVSSSGTPIFLTTASELIDDGVNRKSCSIVATRFGCDILCSIVTEKEGDDYWSDTPTPMRHGRFSFNGSFVEAELPVSSMFSSFSANYPAVGSGEIVKDRILFPIYGGIKQTSPEFTELVKYGLFVSTPTTVCQSSWTYDQVKAAYRPDYISGRFWAQVILSCALDAVDLSSCIVKIMNSSTVMMAAEGRIMKIGIDYFYYQRSSSWWPLAFVTKLDPQELADTNSIWLTNSIPIPQSKFPRPSYSENYCTKPAVCPATCVTGVYSDIWPLTSSSSLPSIIWIGQYLDAPVRRTYPRFGIANQSHWYLQEDILPTSTASAYSTTTCFKNTARNRVFCVTIAEFADGLFGEYRITPQLYELVRNN

SEQ ID NO: 63 - DNA, 6717 bp, artificial sequence
  L gene of FJ215863
atggatataaaacaagttgacctgataatacaacccgaggttcatctcgattcacccatcatattgaataaactggcactattatggcgcttgagtggtttacccatgcctgcagacctacgacaaaaatccgtagtgatgcacatcccggaccacatcttagaaaaatcagaatatcggatcaagcaccgtctagggaaaatcaagagtgacataacacattactgccagtattttaatattaatttggcaaatcttgatccgataacccaccccaaaagtttgtattggttatccagactaacaatagctagtgctggaacctttagacatatgaaagatagaatcttgtgtacagttggctctgaattcggacacaaaattcaagatttattttcactgttgagccataaactagtaggtaacggtgatttatttaatcaaagtctctcaggtacacgtttgactgcgagtccgttatcccctttatgcgatcaatttgtctctgacatcaagtctgcagtcacgacaccctggtcagaagctcgttggtcttggcttcatatcaaacaaacaatgagatatctaataaaacaatcatgcactacaaattcggctcatttaacagaaatcataaaagaggaatggggtttagtaggtattactccagatcttgtcattctttttgacagagtcaataatagtctgactgcattaacatttgagatggttctaatgtattcagatgtattagaatcccgtgacaatattgtgttagtggggcgactatctacctttctacagccagtagttagtagactggaggtgttgtttgatctagtagattcattggcaaaaatcttaggtgacacaatatatgagattattgcagtgttagagagcttgtcttatgggtcagttcaactacatgatgcaagtcactctcatgcagggtcttttttttcatttaacatgaatgaacttgataacacactatcaaagagggtagatccgaaacacaagaacaccataatgagcattataagacaatgcttttctaatctagatgttgatcaagctgcagagatgctatgcctgatgagattattcggacacccaatgttaactgcaccggatgcagcagccaaagtgaggaaagcaatgtgtgctccaaaacttgttgaacacgacaccatcttgcagacattatctttcttcaaggggataattataaatgggtacagaagatcacactctggcctgtggcccaatgtagagccgtcttcaatttatgatgatgatctcagacagctgtacttagagtcagcagagatttcccatcatttcatgcttaaaaactacaagagtttgagcatgatagaattcaagaagagcatagactacgatcttcatgatgacttaagtactttcttaaaggatagagcaatttgccggccgaaatcccagtgggatgtcatatttcgtaaatctttacgcagatctcatacgcagtcccagtatctagacgaaattaagagcaaccggttgctaattgattttcttgattctgctgaatttgaccctgaaaaagaatttgcatatgtaaccacaatggattatttgcacgataatgaattttgtgcttcatattctctaaaggaaaaggagatcaaaactactgggaggatatttgcaaaaatgacacgcaatatgagaagttgccaagtaatacttgaatctttgttatcaaagcatatatgcaagttcttcaaagagaatggcgtttcgatggagcaattgtcattgaccaagagtctacttgcaatgtctcaactctcaccaaaagtctcgactttgcaggacactgcatcacgtcatgtaggcaactcaaaatctcagattgcaaccagcaacccatctcggcatcactcgacagccaatcagatgtcactctcaaatcgaaaaacggttgtagcaactttcttaacaactgacttggaaaaatactgcctgcagtggcgatactcaactattaaattgtttgcacaagctctaaatcaactctttgggattgatcacggatttgaatggatacatttaagacttatgaacagcaccttatttgttggcgatccttactcgcctcctgaagatccaacactagaagatatagataaagcaccaaatgatgatatcttcatagtttctccaaggggaggcatagagggtttatgtcagaaaatgtggaccatgatatcaattagtgctatacactgtgtagcagagaaaattggtgcacgagtggcagcaatggtgcagggtgataatcaagtaatagctatcaccaaagaactattcagaggagaaaaagcttgtgatgtcagagatgagttagacgagcttggtcaagtgttttttgatgagttcaagagacacaattatgcaattggacacaatcttaagctaaatgagacaatacaaagccaatccttttttgtatattccaaacgaatattctttgaagggcgattgcttagtcaagtcctcaaaaatgctgccaagttatgtatggttgctgaccatctaggtgaaaacactgtatcttcctgtagcaacctgagctcgacaattgcccgcttggtggaaaatgggtttgagaaggacactgcttttgtgttgaacctagtctacatcatgactcagattctttttgatgagcattactcgattgtatgcgatcaccatagtgtcaaaagtttgattggatcaaaaaaccatcggaatttattgtactcatctctaataccaggtcagctcggcggtttcaacttcctcaatataagtcggttgttcactaggaatataggtgacccagtaacatgtagtctgtctgatctcaaatgcttcatagccgcaggtctccttccaccctatgtcctaaaaaatgtggttctgcgtgagcctggtcctgggacatggttgacgttgtgctctgatccttacacccttaacataccatacacacagcttccaaccacatatctcaaaaagcacacccagcgatcattgctttcacgtgcagtaaatcctttattagccggtgtacaagtgccaaatcagcatgaggaagaagaggtgttggctcgctttctccttgatcgtgaatatgtgatgccccgcgttgctcatgtaatactagaaacatcggtccttggcaaacggaaacaaatccaaggcttaattgatacaactccaaccatcattagaacatctctagttaatctgccagtgtctagaaagaaatgcgaaaaaataatcaattactctctcaattatattgctgagtgtcatgactccttacttagccaggtctgcttcagtgataataaggaatacttgtggtcaacctccttaatatcagttgagacatgtagtgtgacaatcgcggactatctgagagctgtcagctggtctaatatattagggggaagaaacatatccggggtgactacacctgatactattgaattaattcaaggttgtttaataggtgaaaattctagttgtactctttgtgaatcgcatgatgacgcattcacgtggatgcacttgcctggcccactttacatccctgaaccatcagttactaactctaaaatgcgtgtgccatatctgggttcgaaaacagaggagcgtaaaacagcctcaatggcagcaataaaaggaatgtcacatcacctgcgtgcagtcttaagaggcacatccgtatttatttgggcatttggggatacagatattaattgggataatgcattgcagattgcccaatcacggtgtaacatcacattggatcaaatgagattacttacaccaattcctagcagttcaaatattcaacatagactcgatgacggaatcagcacgcagaaatttactcctgcaagccttgctcgaatcacatccttcgttcacatctgtaatgacagccagaggttagagaaggatggctcatctgttgactcaaacttgatttaccagcaaattatgttacttggactcagcatctttgaaacaatgtactcaatggaccaaaagtgggtattcaataaccataccttgcatttgcacactggacactcctgttgtccaagggaactagacataagtttggtgaacccgccgagacatcagaccccggagctgactagcacaacaaccaacccgttcctatatgatcagctcccattaaatcaagaaaacttgacaacacttgagattaagacatttaaattcaatgagctcaacattgatggtttagattttggtgaaggaatacaattattgagtcgttgtactgcaagattgatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagacgggccattcttaattacttggacacaactttgcaaaggatccctgtgatacaattagccaacattgcactcaccatttcgcaccctgagatatttcgcagaattgtcaacaccgggatccataaccagattaagggcccatatgtcgcaacaacggatttcatagctgcaagtagagatatcatattatcaggtgcaagggagtatctatcttatttaagcagtgggcaggaagactgttacacattcttcaactgtcaagatggggatcttactccaaaaatggaacagtatcttgcaaggagggcatgccttttaacattattgtataatactgggcaccagatccccgttatccgatcactgacaccaatagagaagtgcaaggtgctcacagaatacaatcaacaaattgagtatgcagatcaagagtttagctctgtattaaaagtggtcaatgcactactacaaaatcctaagatagatgcattagtttcaaatctctacttcaccaccagacgtgttctatcaaacctcagatcatgtgataaggctagatcatatattgaatatttgtacactgaggacttcggagagaaagaggatacagtacaatatgacatcatgacaacaaacgatatcatacttactcatggtctattcacacagatcgaaatatcttatcaagggaatagtctccataagttccttactccggataacgcgcctggatctttgatcccattctctatttcaccaaattcacttgcatgtgatcctcttcatcacttgctcaagtcggtcggtacatcaagcacaagttggtacaagtatgcaatcgcctatgcagtgtctgaaaagaggtcagctcgattaggagggagcttgtacattggtgaagggagcggaagtgtgatgactttactcgagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctagggtttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttatacccatatcttacttgttgctacttacggattacagcaagattcaatactgtgggtgaaggtatataggacatctgaaaaagtatttcaattcttactgagtgccatgatcatgatctttggttatgtcaaaatccacaggaatggttatatgtcgacaaaggatgaagagtacatattgatgtctgactgcaaggaacctgtaaactatacagctgtccctaacattcttacacgtgtaagtgatttagtgtcgaagaatctgagtcttatccatccagaagacctcagaaaagtaaggtgtgaaacagattccctgaatttgaagtgcaatcatatttatgagaaaataattgccagaaaaattccattacaggtatcatcaactgattctttgctcctccaattaggcggtgttatcaactcggtgggctcaactgatcctagagaggttgcaacattatcttctattgagtgtatggactatgttgtctcatcaattgatttggctatattggaggcaaatattgtaatctcagagagtgctggtcttgacctcgctttaatgttaggcccattcaacttgaataagcttaagaaaattgatacaatccttaagtcaagcacctatcagctaatcccgtactggttgcgctatgagtactctattaatccgagatctttgtcatttctaatcactaaattacaacaatgccgaatttcatggtcagatatgatcacgatttctgaatttcgtaagaaatccaagcggcctatatttatcaaacgagtaatagggaatcaacagctaaaatcattctttaatgaaagctcaagtattgttttgactcgggctgaagttaaagtctgtataaagttcctcggtgcaatcatcaagttgaaataa

SEQ ID NO: 64 - protein, 2238 aa, artificial sequence
  L protein of ACO48302
MDIKQVDLIIQPEVHLDSPIILNKLALLWRLSGLPMPADLRQKSVVMHIPDHILEKSEYRIKHRLGKIKSDITHYCQYFNINLANLDPITHPKSLYWLSRLTIASAGTFRHMKDRILCTVGSEFGHKIQDLFSLLSHKLVGNGDLFNQSLSGTRLTASPLSPLCDQFVSDIKSAVTTPWSEARWSWLHIKQTMRYLIKQSCTTNSAHLTEIIKEEWGLVGITPDLVILFDRVNNSLTALTFEMVLMYSDVLESRDNIVLVGRLSTFLQPVVSRLEVLFDLVDSLAKILGDTIYEIIAVLESLSYGSVQLHDASHSHAGSFFSFNMNELDNTLSKRVDPKHKNTIMSIIRQCFSNLDVDQAAEMLCLMRLFGHPMLTAPDAAAKVRKAMCAPKLVEHDTILQTLSFFKGIIINGYRRSHSGLWPNVEPSSIYDDDLRQLYLESAEISHHFMLKNYKSLSMIEFKKSIDYDLHDDLSTFLKDRAICRPKSQWDVIFRKSLRRSHTQSQYLDEIKSNRLLIDFLDSAEFDPEKEFAYVTTMDYLHDNEFCASYSLKEKEIKTTGRIFAKMTRNMRSCQVILESLLSKHICKFFKENGVSMEQLSLTKSLLAMSQLSPKVSTLQDTASRHVGNSKSQIATSNPSRHHSTANQMSLSNRKTVVATFLTTDLEKYCLQWRYSTIKLFAQALNQLFGIDHGFEWIHLRLMNSTLFVGDPYSPPEDPTLEDIDKAPNDDIFIVSPRGGIEGLCQKMWTMISISAIHCVAEKIGARVAAMVQGDNQVIAITKELFRGEKACDVRDELDELGQVFFDEFKRHNYAIGHNLKLNETIQSQSFFVYSKRIFFEGRLLSQVLKNAAKLCMVADHLGENTVSSCSNLSSTIARLVENGFEKDTAFVLNLVYIMTQILFDEHYSIVCDHHSVKSLIGSKNHRNLLYSSLIPGQLGGFNFLNISRLFTRNIGDPVTCSLSDLKCFIAAGLLPPYVLKNVVLREPGPGTWLTLCSDPYTLNIPYTQLPTTYLKKHTQRSLLSRAVNPLLAGVQVPNQHEEEEVLARFLLDREYVMPRVAHVILETSVLGKRKQIQGLIDTTPTIIRTSLVNLPVSRKKCEKIINYSLNYIAECHDSLLSQVCFSDNKEYLWSTSLISVETCSVTIADYLRAVSWSNILGGRNISGVTTPDTIELIQGCLIGENSSCTLCESHDDAFTWMHLPGPLYIPEPSVTNSKMRVPYLGSKTEERKTASMAAIKGMSHHLRAVLRGTSVFIWAFGDTDINWDNALQIAQSRCNITLDQMRLLTPIPSSSNIQHRLDDGISTQKFTPASLARITSFVHICNDSQRLEKDGSSVDSNLIYQQIMLLGLSIFETMYSMDQKWVFNNHTLHLHTGHSCCPRELDISLVNPPRHQTPELTSTTTNPFLYDQLPLNQENLTTLEIKTFKFNELNIDGLDFGEGIQLLSRCTARLMAECILEEGIGSSVKNEAIVNFDNSVNWISECLMCDIRSLCVNLGQEILCSLAYQMYYLRIRGRRAILNYLDTTLQRIPVIQLANIALTISHPEIFRRIVNTGIHNQIKGPYVATTDFIAASRDIILSGAREYLSYLSSGQEDCYTFFNCQDGDLTPKMEQYLARRACLLTLLYNTGHQIPVIRSLTPIEKCKVLTEYNQQIEYADQEFSSVLKVVNALLQNPKIDALVSNLYFTTRRVLSNLRSCDKARSYIEYLYTEDFGEKEDTVQYDIMTTNDIILTHGLFTQIEISYQGNSLHKFLTPDNAPGSLIPFSISPNSLACDPLHHLLKSVGTSSTSWYKYAIAYAVSEKRSARLGGSLYIGEGSGSVMTLLEYLEPSVDIFYNSLFSNGMNPPQRNYGLMPLQFVNSVVYKNLTAKSECKLGFVQQFKPLWRDIDIETNVTDPSFVNFALNEIPMQSLKRVNCDVEFDRGMPIERVIQGYTHILLVATYGLQQDSILWVKVYRTSEKVFQFLLSAMIMIFGYVKIHRNGYMSTKDEEYILMSDCKEPVNYTAVPNILTRVSDLVSKNLSLIHPEDLRKVRCETDSLNLKCNHIYEKIIARKIPLQVSSTDSLLLQLGGVINSVGSTDPREVATLSSIECMDYVVSSIDLAILEANIVISESAGLDLALMLGPFNLNKLKKIDTILKSSTYQLIPYWLRYEYSINPRSLSFLITKLQQCRISWSDMITISEFRKKSKRPIFIKRVIGNQQLKSFFNESSSIVLTRAEVKVCIKFLGAIIKLK

SEQ ID NO: 65 - DNA, 1386 bp, artificial sequence
  NP gene of FJ215864
atgtcatctgtattcaatgaatatcaggcacttcaagaacaacttgtaaagccggctgtcaggagacctgatgttgcctcaacaggtttactcagggcggaaatacctgtctgtgttacattgtctcaagaccccggtgagagatggagccttgcttgccttaatatccgatggcttgtgagtgattcatcaaccacaccaatgaagcagggagcaatattgtcactgctgagtctacattcagacaatatgcgagctcacgcaacattagcagcaaggtctgcagatgcttcactcaccatacttgaggtagatgaagtagatattggcaactccctaatcaaattcaacgctagaagtggtgtatctgataaacgatcaaatcaattgcttgcaattgcggatgacatccccaaaagttgcagtaatgggcatccatttcttgacacagacattgagaccagagacccgctcgatctatcagagaccatagaccgcctgcagggtattgcagctcagatatgggtgtcagccataaagagcatgacagcgcctgacaccgcatcagagtcagaaagtaagaggctggccaaataccaacaacaaggccgactggttaagcaagtacttttgcattctgtagtcaggacagaatttatgagagttattcggggcagcttggtactgcgccagtttatggttagcgagtgcaagagggcttcagccatgggcggagacacatctaggtactatgctatggtgggtgacatcagtctgtacatcaagaatgcaggattgactgcatttttcctcaccctgaagttcggggttggtacccagtatccaaccttagcaatgagtgttttctccagtgaccttaaaagacttgctgcactcatcaggctgtacaaaaccaagggagacaatgcaccatacatggcattcctggaggactccgatatgggaaattttgctccagcaaattatagcacaatgtactcttatgccatgggcattgggacgattctggaagcatctgtatctcgataccagtatgctagagactttaccagtgagaattatttccgtcttggagttgagacagcccaaagccagcagggagcgtttgacgagagaacagcccgagagatgggcttgactgaggaatccaaacagcaggttagatcactgctaatgtcagtagacatgggtcccagttcagttcgcgagccatcccgccctgcattcatcagtcaagaagaaaataggcagcctgcccagaattcttcagatactcagggtcagaccaagccagtcccgaatcaacccgcaccaagggccgacccagatgacattgatccatacgagaacgggctagaatggtaa

SEQ ID NO: 66 - protein, 461 aa, artificial sequence
  NP protein of ACO48303
MSSVFNEYQALQEQLVKPAVRRPDVASTGLLRAEIPVCVTLSQDPGERWSLACLNIRWLVSDSSTTPMKQGAILSLLSLHSDNMRAHATLAARSADASLTILEVDEVDIGNSLIKFNARSGVSDKRSNQLLAIADDIPKSCSNGHPFLDTDIETRDPLDLSETIDRLQGIAAQIWVSAIKSMTAPDTASESESKRLAKYQQQGRLVKQVLLHSVVRTEFMRVIRGSLVLRQFMVSECKRASAMGGDTSRYYAMVGDISLYIKNAGLTAFFLTLKFGVGTQYPTLAMSVFSSDLKRLAALIRLYKTKGDNAPYMAFLEDSDMGNFAPANYSTMYSYAMGIGTILEASVSRYQYARDFTSENYFRLGVETAQSQQGAFDERTAREMGLTEESKQQVRSLLMSVDMGPSSVREPSRPAFISQEENRQPAQNSSDTQGQTKPVPNQPAPRADPDDIDPYENGLEW

SEQ ID NO: 67 - DNA, 1218 bp, artificial sequence
  P gene of FJ215864
atggacttcgccaatgatgaagaaattgcagaacttctgaacctcagcaccactgtaatcaaggagattcagaaatctgaactcaagcctccccaaaccactgggcgaccacctgtcagtcaagggaacacaagaaatctaactgatctatgggaaaaggagactgcaagtcagaacaagacatcggctcaatctccacaaaccacacaagttcagtctgatggaaatgaggaggaagaaatcaaatcagagtcaattgatggccacatcagtggaactgttaatcaattagagcaagtcccagaacaaaaccagagcagatcttcaccaggtgatgatctcgacagagctctcaacaagcttgaagggagaatcaactcaatcagctcaatggataaagaaattaaaaagggccctcgcatccagaatctccctgggtcccaagcagcaactcaacaggcgacccacccattggcaggggacaccccgaacatgcaagcacggacaaaacccctgaccaagccacatcaagaggcaatcaatcctggcaaccaggacacaggagagaatattcatttaccaccttccatggcaccaccagagtcattagttggtgcaatccgcaatgtaccccaattcgtgccagaccaatctatgacgaatgtagatgcggggagtgtccaactacatgcatcatgtgcagagatgataagtagaatgtttgtagaagttatatctaagcttgataaactcgagtcgagactgaatgatatagcaaaagttgtaaacaccaccccccttatcaggaatgatattaaccaacttaaggccacaactgcactgatgtccaaccaaattgcttccatacaaattcttgacccagggaatgcaggggtgaggtccctctctgaaatgagatctgtgacgaagaaagctgctgttgtaattgcaggatttggagacgacccaactcaaattattgaagaaggtatcatggccaaagatgctcttggaaaacctgtgcctccaacatctgttatcgcagccaaagctcagacttcttccggtgtgagtaagggtgaaatagaaggattgattgcattggtggaaacattagttgacaatgacaagaaggcagcgaaactgattaaaatgattgatcaagttaaatcccacgccgattacgcccgagtcaagcaggcaatatataatgcataa

SEQ ID NO: 68 - protein, 405 aa, artificial sequence
  P protein of ACO48304
MDFANDEEIAELLNLSTTVIKEIQKSELKPPQTTGRPPVSQGNTRNLTDLWEKETASQNKTSAQSPQTTQVQSDGNEEEEIKSESIDGHISGTVNQLEQVPEQNQSRSSPGDDLDRALNKLEGRINSISSMDKEIKKGPRIQNLPGSQAATQQATHPLAGDTPNMQARTKPLTKPHQEAINPGNQDTGENIHLPPSMAPPESLVGAIRNVPQFVPDQSMTNVDAGSVQLHASCAEMISRMFVEVISKLDKLESRLNDIAKVVNTTPLIRNDINQLKATTALMSNQIASIQILDPGNAGVRSLSEMRSVTKKAAVVIAGFGDDPTQIIEEGIMAKDALGKPVPPTSVIAAKAQTSSGVSKGEIEGLIALVETLVDNDKKAAKLIKMIDQVKSHADYARVKQAIYNA

SEQ ID NO: 69 - DNA, 1110 bp, artificial sequence
  M gene of FJ215864
atggcatatacaacactaaaactgtgggtggatgagggtgacatgtcgtcttcgcttctatcattcccgttggtactaaaagagacagacagaggcacaaagaagcttcaaccacaggtaagggtagattcaattggcgatgtgcagaatgccaaagagtcctcgatattcgtgactctatatggtttcatccaagcaattaaggagaattcagatcgatcgaaattcttccatccaaaagatgacttcaaacctgagacagtcactgcaggactggtagtagtgggtgcaatccgaatgatggctgatgtcaataccatctctaatgatgcactagcgctggagatcactgttaagaaatctgcaacttctcaagagaaaatgacggtgatgttccacaatagccccccttcattgagaactgcaataactatccgagcaggaggtttcatctcgaatgcagacgaaaatataaaatgtgccagcaagttgactgcaggagtgcagtacatattccgtccaatgtttgtttcaatcactaaattacacaatggcaaactatatagggtgcccaaaagtatccacagcatctcgtctaccctactgtatagtgtgatgttggaggtaggattcaaagtggacatcgggaaggatcatccccaggcaaaaatgctgaagagggtcacaattggcgatgcagacacatactggggatttgcatggttccacctgtgcaatttcaaaaagacatcctctaagggaaagccgagaacgctagacgaactgaagacaaaagtcaaaaatatggggttgaaattggagttacatgacctatggggtccgactattgtggtccaaatcactggcaagagcagcaaatatgctcaaggatttttttcttccaatggtacttgttgcctcccaatcagcagatctgcaccagagcttgggaagcttctgtggtcctgctcagcaactattggtgacgcaacagttgttatccaatcaagcgagaagggggaactcctaaggtctgatgatctcgagatacgaggtgctgtggcctccaagaaaggtagactgagctcatttcaccccttcaaaaaatga

SEQ ID NO: 70 - protein, 369 aa, artificial sequence
  M protein of ACO48305
MAYTTLKLWVDEGDMSSSLLSFPLVLKETDRGTKKLQPQVRVDSIGDVQNAKESSIFVTLYGFIQAIKENSDRSKFFHPKDDFKPETVTAGLVVVGAIRMMADVNTISNDALALEITVKKSATSQEKMTVMFHNSPPSLRTAITIRAGGFISNADENIKCASKLTAGVQYIFRPMFVSITKLHNGKLYRVPKSIHSISSTLLYSVMLEVGFKVDIGKDHPQAKMLKRVTIGDADTYWGFAWFHLCNFKKTSSKGKPRTLDELKTKVKNMGLKLELHDLWGPTIVVQITGKSSKYAQGFFSSNGTCCLPISRSAPELGKLLWSCSATIGDATVVIQSSEKGELLRSDDLEIRGAVASKKGRLSSFHPFKK

SEQ ID NO: 71 - DNA, 1632 bp, artificial sequence
  F gene of FJ215864
atgggtaaaatatcaatatatctaattaatagcgtgctattattgctggtatatcctgtgaattcgattgacaatacactcgttgccccaatcggagtcgccagcgcaaatgaatggcagcttgctgcatatacaacatcactttcagggacaattgccgtgcgattcctacctgtgctcccggataatatgactacctgtcttagagaaacaataactacatataataatactgtcaacaacatcttaggcccactcaaatccaatctggatgcactgctctcatctgagacttatccccagacaagattaattggggcagttataggttcaattgctcttggtgttgcaacatcggctcaaatcactgctgcagtcgctctcaagcaagcacaagataatgcaagaaacatactggcactcaaagaggcactgtccaaaactaatgaggcggtcaaggagcttagcagtggattgcaacaaacagctattgcacttggtaagatacagagctttgtgaatgaggaaattctgccatctatcaaccaactgagctgcgaggtgacagccaataaacttggggtgtatttatctctgtatctcacagaactgaccactatattcggtgcacagttgactaaccctgcattgacttcattatcatatcaagcgctgtacaacctgtgtggtggcaacatggcaatgcttactcagaagattggaattaaacagcaagacgttaattcgctatatgaagccggactaatcacaggacaagtcattggttatgactctcagtaccagctgctggtcatccaggtcaattatccaagcatttctgaggtaactggtgtgcgtgcgacagaattagtcactgttagtgtaacaacagacaagggtgaagggaaagcaattgtaccccaatttgtagctgaaagtcgggtgactattgaggagcttgatgtagcatcttgtaaattcagcagcacaaccctatactgcaggcaggtcaacacaagggcacttcccccgctagtggctagctgtctccgaggtaactatgatgattgtcaatataccacagagattggagcattatcatcccggtatataacactagatggaggggtcttagtcaattgtaagtcaattgtttgtaggtgccttaatccaagtaagatcatctctcaaaatacaaatgctgcagtaacatatgttgatgctacaatatgcaaaacaattcaattggatgacatacaactccagttggaagggtcactatcatcagtttatgcaaggaacatctcaattgagatcagtcaggtgactacctccggttctttggatatcagcagtgagatagggaacatcaataatacggtgaatcgtgtggaggatttaatccaccaatcggaggaatggctggcaaaagttaacccacacattgttaataatactacactaattgtactctgtgtgttaagtgcgcttgctgtgatctggctggcagtattaacggctattataatatacttgagaacaaagttgaagactatatcggcattggctgtaaccaatacaatacagtctaatccctatgttaaccaaacgaaacgtgaatctaagttttga

SEQ ID NO: 72 - protein, 543 aa, artificial sequence
  F protein of ACO48306
MGKISIYLINSVLLLLVYPVNSIDNTLVAPIGVASANEWQLAAYTTSLSGTIAVRFLPVLPDNMTTCLRETITTYNNTVNNILGPLKSNLDALLSSETYPQTRLIGAVIGSIALGVATSAQITAAVALKQAQDNARNILALKEALSKTNEAVKELSSGLQQTAIALGKIQSFVNEEILPSINQLSCEVTANKLGVYLSLYLTELTTIFGAQLTNPALTSLSYQALYNLCGGNMAMLTQKIGIKQQDVNSLYEAGLITGQVIGYDSQYQLLVIQVNYPSISEVTGVRATELVTVSVTTDKGEGKAIVPQFVAESRVTIEELDVASCKFSSTTLYCRQVNTRALPPLVASCLRGNYDDCQYTTEIGALSSRYITLDGGVLVNCKSIVCRCLNPSKIISQNTNAAVTYVDATICKTIQLDDIQLQLEGSLSSVYARNISIEISQVTTSGSLDISSEIGNINNTVNRVEDLIHQSEEWLAKVNPHIVNNTTLIVLCVLSALAVIWLAVLTAIIIYLRTKLKTISALAVTNTIQSNPYVNQTKRESKF

SEQ ID NO: 73 - DNA, 1734 bp, artificial sequence
  HN gene of FJ215864
atgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtatttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattga

SEQ ID NO: 74 - protein, 577 aa, artificial sequence
  HN protein of ACO48307
MSNIASSLENIVEQDSRKTTWRAIFRWSVLLITTGCLALSIVSIVQIGNLKIPSVGDLADEVVTPLKTTLSDTLRNPINQINDIFRIVALDIPLQVTSIQKDLASQFSMLIDSLNAIKLGNGTNLIIPTSDKEYAGGIGNPVFTVDAGGSIGFKQFSLIEHPSFIAGPTTTRGCTRIPTFHMSESHWCYSHNIIAAGCQDASASSMYISMGVLHVSSSGTPIFLTTASELIDDGVNRKSCSIVATQFGCDILCSIVIEKEGDDYWSDTPTPMRHGRFSFNGSFVETELPVSSMFSSFSANYPAVGSGEIVKDRILFPIYGGIKQTSPEFTELVKYGLFVSTPTTVCQSSWTYDQVKAAYRPDYISGRFWAQVILSCALDAVDLSSCIVKIMNSSTVMMAAEGRIIKIGIDYFYYQRSSSWWPLAFVTKLDPQELADTNSIWLTNSIPIPQSKFPRPSYSENYCTKPAVCPATCVTGVYSDIWPLTSSSSLPSIIWIGQYLDAPVGRTYPRFGIANQSHWYLQEDILPTSTASAYSTTTCFKNTARNRVFCVTIAEFADGLFGEYRITPQLYELVRNN

SEQ ID NO: 75 - DNA, 6717 bp, artificial sequence
  L gene of FJ215864
atggatgtaaaacaagttgacctaataatacaacccgaggttcatctcgattcacccatcatattgaataaactggcactattatggcgcttgagtggtttacccatgcctgcagacttacgacaaaaatccgtagtgatgcacatcccagaccacatcttagaaaaatcagaatatcggatcaagcaccgtctagggaaaatcaagagtgacatagcacattactgtcagtattttaatattaatttggcaaatcttgatccgataacccaccccaaaagtttgtattggttatccagactaacaatagctagtgctggaacctttagacatatgaaagatagaatcttatgtacagttggctccgaattcggacacaaaattcaagatttattttcactgctgagccataaattagtaggtaacggtgatttatttaatcaaagtctctcaggtacacgtttgactgcgagtccgttatcccctttatgcaatcaatttgtctctgacatcaagtctgcagtcacgacaccctggtcagaagctcgttggtcttggcttcatatcaaacaaacaatgagatacctgataaaacaatcacgcactacaaattcagctcatttaacagaaattataaaagaggaatggggtttagtaggtattactccagatcttgtcattctttttgacagagtcaataatagtctaactgcattaacatttgagatggttctaatgtattcagatgtattagaatcccgtgacaatattgtgctagtggggcgattatctacttttctgcagccagtagttagtagactggaggtgttgtttgatctagtagattcattggcaaaaaccttaggtgacacaatatacgaaattattgcggtgttagagagcttgtcttatgggtccgttcaactacatgatgcaagtcactctcatgcagggtctttcttttcatttaacatgaatgaacttgataacacactatcaaagagggtggatccgaaacacaagaacaccataatgagcattataagacaatgcttttctaatctagatgttgatcaagctgcagagatgctatgcctgatgagattatttggacacccaatgttaactgcaccggatgcagcagccaaagtaaggaaagcaatgtgtgctccaaaacttgttgaacatgacaccatcttgcagacattatccttcttcaagggaataattataaatgggtacagaagatcacactctggcctgtggcccaatgtagagccgtcttcaatctatgatgatgatctcagacagctgtacttagagtcagcagagatttcccatcatttcatgcttaaaaactacaagagtttgagcatgatagaattcaagaagagcatagactacgatcttcacgacgacttaagtactttcttaaaggatagagcaatttgccggccaaaatcccagtgggatgttatattccgtaagtctttacgcagatcccacacgcggtcccagtatatggacgaaattaagagcaaccgattgctaattgattttcttgattctgctgattttgaccctgaaaaggaatttgcatatgtaaccacaatggattatttgcacgataatgaattttgtgcttcatattctctaaaggaaaaggagatcaaaactaccgggaggatatttgcaaaaatgacacgcaatatgagaagttgccaagtgatacttgaatctctgttatcaaaacatatatgcaagttcttcaaagagaacggcgtttcgatggagcaattgtcattgaccaagagtctacttgcaatgtctcaactctcaccaaaagtctcgactctgcaggacactgcatcacgtcatgtaggcaactcaaaatctcagatcgcaaccagcaacccatctcggcatcactcaacaaccaatcagatgtcactctcaaatcggaaaacggttgtagcaactttcttaacaactgatttggaaaaatactgcctgcagtggcgatactcgactattaagttgtttgcacaagctctaaatcaactctttgggattgatcacggatttgaatggatacatttaagactcatgaacagcaccttatttgtcggtgatccttactcgcctcctgaagatccaacactagaggatatagataaagcaccaaatgacgatatcttcatagtttctccaaggggaggcatagagggtttatgtcagaagatgtggaccatgatatcaattagtgcgatacactgtgtagcagagaaaattggtgcacgagtggcagcaatggtgcagggtgataatcaagtaatagctatcaccaaagaactattcagaggagagaaagcctgtgatgtcagagatgagttagacgagctcggtcaggtgttttttgatgagttcaagaggcacaattatgcaattggacacaaccttaagctaaatgagacaatacaaagccaatccttttttgtatattccaaacgaatattctttgaagggcgattgcttagtcaagtcctcaaaaatgctgccaagttatgtatggttgctgaccatctaggtgaaaacacagtatcttcctgtagcaacctgagctctacaattgcccggttggtggaaaatgggtttgagaaggacactgcttttgtgttgaacctagtctacatcatgactcaaattctttttgatgagcattactcgattgtatgcgatcacaatagtgtcaaaagcttgatcggatcaaaaaactatcggaatctattgtactcatctctaataccaggtcagctcggtggtttcaacttcctcaatataagtcggttgttcactaggaatataggtgacccagtaacatgtagtctgtctgatctcaaatgcttcatagccgcaggtctccttccaccctatgtacttaaaaatgtggttctgcgtgagcctggtcctgggacatggttgacgttgtgctctgatccttacacccttaacataccatacacacagctaccaaccacatatctcaaaaagcacacccagcgatcgttgctttcacgtgcagtaaatcctttattagcaggtgtacaagtgccaaatcagcatgaggaagaagagatgttggctcgctttctccttgatcgtgaatatgtgatgccccgcgttgctcatgtaatactagaaacatcggtccttggcaaacggaaacaaatccaaggcttaattgatacaactccaactatcattagaacatctctagttaatctaccagtgtctaggaagaaatgcgaaaaaataatcaattattctctcaattatattgctgagtgtcatgactccttacttagtcagatctgcttcagtgataataaggaatacttgtggtccacctccttaatatcagttgagacctgtagtgtgacaattgcggactatttgagagctgtcagctggtctaatatattagggggaagaaacatatccggggtgactacacctgatactattgaattaattcaaggttgtttaataggtgaaaattccagttgtactctttgtgaatcgcatgacgacgcattcacatggatgcacttgcctggcccactttacatccctgaaccatcagttactaactctaaaatgcgtgtgccatatctgggttcaaaaacagaggagcgtaaaacagcttcaatggcagcaataaaaggaatgtcacatcacctgcgtgcagtcttaagaggtacatccgtatttatttgggcatttggggacacagatattaattgggataatgcattgcagattgcccaatcacggtgtaacatcacattggatcaaatgagattacttacaccaattcctagcagttcaaatatccaacatagactcgatgacggaatcagcacgcagaaatttactcctgcaagccttgctcgaatcacatcctttgttcacatctgtaatgacagccaaaggttagagaaggatggctcctctgtcgactcaaacttgatttaccagcaaattatgttacttggactcagcatctttgaaacaatgtactcaatggaccaaaagtgggtattcaataaccataccttacatttgcacactggacactcctgttgtccaagggaactagacataagtttagtgaacccgccaagacatcagaccccggagctgactagcacaacaaccaacccgttcctatatgatcagctcccactaaatcaggataatctgacaacacttgagattaagacattcaaatttaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagaagggccattcttaattacttggacacaactttgcaaaggatccctgtgatacaattagccaacattgcactcaccatttcacaccctgagatatttcgcagaattgtcaacaccgggatccataaccagattaagggcccatatgtggcaacaacagatttcatagctgcaagtagagatatcatattatcaggtgcaagggagtatctatcttatctaagcagtggacaggaagactgttacacattcttcaactgtcaagatggggatcttactccaaaaatggaacagtatcttgcaaggagggcatgccttttaacattactgtataatactgggcaccagatccccattatccgatcactgacaccaatagagaagtgcaaggtgctcacagaatacaatcaacaaattgagtatgcagatcaagagtttagctctgtattgaaagtggtcaatgcactactacaaaatcctaatatagatgcattggtttcaaatctctacttcaccaccagacgtgttttatcaaacctcagatcatgtgataaggctatatcatatattgaatatttgtacactgaggacttcggagaaaaagaagatacagtacaatatgacatcatgacaacaaacgatatcatacttactcatggtctattcacacagatcgaaatatcttaccaagggagtagtctccataaattcctaactccggataacgcgcctggatcattgatcccattctctatttcaccaaattcgcttgcatgtgatcctcttcaccacttactcaagtcggtcggtacatcaagcacaagctggtacaagtatgcaatcgcctatgcagtgtctgaaaagaggtcggctcgattaggagggagcttgtacattggtgaagggagcggaagtgtgatgactttgctagagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctaggatttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttacactcatatcttacttgttgctacttacggattgcagcaagattcaatactgtgggtgaaagtatataggacatctgaaaaagtatttcagttcttactgagtgccatgatcatgatctttggttatgtcaaaatccacaggaatggttatatgtcggcaaaggatgaggagtacatattgatgtctgactgcaaggaacctgtaaactatacagctgtccctaacattcttacacgtgtaagtgatttagtgtcgaagaatctgagtcttatccatccagaagacctcagaaaggtaaggtgtgaaacagattccctgaatttgaagtgcaatcatatttatgagaaaataattgctagaaaaattccattacaggtatcatcaactgattctttgctcctccagttaggcggtgtcatcaactcggtgggctcaactgatcctagagaggttgcaacattatcttccattgagtgtatggactatgttgtctcatcaattgatttggctatattagaggcaaatattgtgatctcagagagtgctggtcttgacctcgctttaatgttaggcccattcaacttgaataagcttaagaaaattgacacaatccttaagtcaagcacctatcagctaatcccgtattggttgcgctatgagtactctattaatccgagatctttgtcatttctaatcactaaattacaacaatgccgaatttcatggtcagatatgataacaatctctgaattttgcaagaaatccaagcggcctatatttattaaacgagtaatagggaatcaacagctgaaatcattctttaatgaaagctcaagtattgttttgacccgggctgaagtcaaagtctgtataaagttcctcggtgcaatcatcaagttgaaataa

SEQ ID NO: 76 - protein, 2238 aa, artificial sequence
  L protein of ACO48308
MDVKQVDLIIQPEVHLDSPIILNKLALLWRLSGLPMPADLRQKSVVMHIPDHILEKSEYRIKHRLGKIKSDIAHYCQYFNINLANLDPITHPKSLYWLSRLTIASAGTFRHMKDRILCTVGSEFGHKIQDLFSLLSHKLVGNGDLFNQSLSGTRLTASPLSPLCNQFVSDIKSAVTTPWSEARWSWLHIKQTMRYLIKQSRTTNSAHLTEIIKEEWGLVGITPDLVILFDRVNNSLTALTFEMVLMYSDVLESRDNIVLVGRLSTFLQPVVSRLEVLFDLVDSLAKTLGDTIYEIIAVLESLSYGSVQLHDASHSHAGSFFSFNMNELDNTLSKRVDPKHKNTIMSIIRQCFSNLDVDQAAEMLCLMRLFGHPMLTAPDAAAKVRKAMCAPKLVEHDTILQTLSFFKGIIINGYRRSHSGLWPNVEPSSIYDDDLRQLYLESAEISHHFMLKNYKSLSMIEFKKSIDYDLHDDLSTFLKDRAICRPKSQWDVIFRKSLRRSHTRSQYMDEIKSNRLLIDFLDSADFDPEKEFAYVTTMDYLHDNEFCASYSLKEKEIKTTGRIFAKMTRNMRSCQVILESLLSKHICKFFKENGVSMEQLSLTKSLLAMSQLSPKVSTLQDTASRHVGNSKSQIATSNPSRHHSTTNQMSLSNRKTVVATFLTTDLEKYCLQWRYSTIKLFAQALNQLFGIDHGFEWIHLRLMNSTLFVGDPYSPPEDPTLEDIDKAPNDDIFIVSPRGGIEGLCQKMWTMISISAIHCVAEKIGARVAAMVQGDNQVIAITKELFRGEKACDVRDELDELGQVFFDEFKRHNYAIGHNLKLNETIQSQSFFVYSKRIFFEGRLLSQVLKNAAKLCMVADHLGENTVSSCSNLSSTIARLVENGFEKDTAFVLNLVYIMTQILFDEHYSIVCDHNSVKSLIGSKNYRNLLYSSLIPGQLGGFNFLNISRLFTRNIGDPVTCSLSDLKCFIAAGLLPPYVLKNVVLREPGPGTWLTLCSDPYTLNIPYTQLPTTYLKKHTQRSLLSRAVNPLLAGVQVPNQHEEEEMLARFLLDREYVMPRVAHVILETSVLGKRKQIQGLIDTTPTIIRTSLVNLPVSRKKCEKIINYSLNYIAECHDSLLSQICFSDNKEYLWSTSLISVETCSVTIADYLRAVSWSNILGGRNISGVTTPDTIELIQGCLIGENSSCTLCESHDDAFTWMHLPGPLYIPEPSVTNSKMRVPYLGSKTEERKTASMAAIKGMSHHLRAVLRGTSVFIWAFGDTDINWDNALQIAQSRCNITLDQMRLLTPIPSSSNIQHRLDDGISTQKFTPASLARITSFVHICNDSQRLEKDGSSVDSNLIYQQIMLLGLSIFETMYSMDQKWVFNNHTLHLHTGHSCCPRELDISLVNPPRHQTPELTSTTTNPFLYDQLPLNQDNLTTLEIKTFKFNELNIDGLDFGEGIQLLSRCTARLMAECILEEGIGSSVKNEAIVNFDNSVNWISECLMCDIRSLCVNLGQEILCSLAYQMYYLRIRGRRAILNYLDTTLQRIPVIQLANIALTISHPEIFRRIVNTGIHNQIKGPYVATTDFIAASRDIILSGAREYLSYLSSGQEDCYTFFNCQDGDLTPKMEQYLARRACLLTLLYNTGHQIPIIRSLTPIEKCKVLTEYNQQIEYADQEFSSVLKVVNALLQNPNIDALVSNLYFTTRRVLSNLRSCDKAISYIEYLYTEDFGEKEDTVQYDIMTTNDIILTHGLFTQIEISYQGSSLHKFLTPDNAPGSLIPFSISPNSLACDPLHHLLKSVGTSSTSWYKYAIAYAVSEKRSARLGGSLYIGEGSGSVMTLLEYLEPSVDIFYNSLFSNGMNPPQRNYGLMPLQFVNSVVYKNLTAKSECKLGFVQQFKPLWRDIDIETNVTDPSFVNFALNEIPMQSLKRVNCDVEFDRGMPIERVIQGYTHILLVATYGLQQDSILWVKVYRTSEKVFQFLLSAMIMIFGYVKIHRNGYMSAKDEEYILMSDCKEPVNYTAVPNILTRVSDLVSKNLSLIHPEDLRKVRCETDSLNLKCNHIYEKIIARKIPLQVSSTDSLLLQLGGVINSVGSTDPREVATLSSIECMDYVVSSIDLAILEANIVISESAGLDLALMLGPFNLNKLKKIDTILKSSTYQLIPYWLRYEYSINPRSLSFLITKLQQCRISWSDMITISEFCKKSKRPIFIKRVIGNQQLKSFFNESSSIVLTRAEVKVCIKFLGAIIKLK

SEQ ID NO: 77 - DNA, 1570 bp, artificial sequence
  NP gene of FJ619036
gggggcgaagcaagtggatctcgggctcgaggccgaaacactggatttcgctggaggttttgaataggtcgctataagactcaatatgtcatctgtattcaatgaatatcaggcacttcaagaacaacttgtaaagccggctgtcaggagacctgatgttgcctcaacaggtttactcagggcggaaatacctgtctgtgttacattgtctcaagaccccggtgagagatggagccttgcttgccttaatatccgatggcttgtgagtgattcatcaaccacaccaatgaagcagggagcaatattgtcactgctgagtctacattcagacaatatgcgagctcacgcaacattagcagcaaggtctgcagatgcttcactcaccatacttgaggtagatgaagtagatattggcaactccctaatcaaattcaacgctagaagtggtgtatctgataaacgatcaaatcaattgcttgcaattgcggatgacatccccaaaagttgcagtaatgggcatccatttcttgacacagacattgagaccagagacccgctcgatctatcagagaccatagaccgcctgcagggtattgcagctcagatatgggtgtcagccataaagagcatgacagcgcctgacaccgcatcagagtcagaaagtaagaggctggccaaataccaacaacaaggccgactggttaagcaagtacttttgcattctgtagtcaggacagaatttatgagagttattcggggcagcttggtactgcgccagtttatggttagcgagtgcaagagggcttcagccatgggcggagacacatctaggtactatgctatggtgggtgacatcagtctgtacatcaagaatgcaggattgactgcatttttcctcaccctgaagttcggggttggtacccagtatccaaccttagcaatgagtgttttctccagtgaccttaaaagacttgctgcactcatcaggctgtacaaaaccaagggagacaatgcaccatacatggcattcctggaggactccgatatgggaaattttgctccagcaaattatagcacaatgtactcttatgccatgggcattgggacgattctggaagcatctgtatctcgataccagtatgctagagactttaccagtgagaattatttccgtcttggagttgagacagcccaaagccagcagggagcgtttgacgagagaacagcccgagagatgggcttgactgaggaatccaaacagcaggttagatcactgctaatgtcagtagacatgggtcccagttcagttcgcgagccatcccgccctgcattcatcagtcaagaagaaaataggcagcctgcccagaattcttcagatactcagggtcagaccaagccagtcccgaatcaacccgcaccaagggccgacccagatgacattgatccatacgagaacgggctagaatggtaattcaatcacctcgacacatccacctatacaccaattctgtgacatattaacctaatcaaacatttcataaactatagtagtcattgatttaagaaaaaa

SEQ ID NO: 78 - protein, 461 aa, artificial sequence; identical to SEQ ID NO: 66
  NP protein of ACN88139

SEQ ID NO: 79 - DNA, 1374 bp, artificial sequence
  P gene of FJ619036
gggggcgacctcaactgtgaaacacgccagatctgtccacaacaccactcaacaacccacacaagatggacttcgccaatgatgaagaaattgcagaacttctgaacctcagcaccactgtaatcaaggagattcagaaatctgaactcaagcctccccaaaccactgggcgaccacctgtcagtcaagggaacacaagaaatctaactgatctatgggaaaaggagactgcaagtcagaacaagacatcggctcaatctccacaaaccacacaagttcagtctgatggaaatgaggaggaagaaatcaaatcagagtcaattgatggccacatcagtggaactgttaatcaattagagcaagtcccagaacaaaaccagagcagatcttcaccaggtgatgatctcgacagagctctcaacaagcttgaagggagaatcaactcaatcagctcaatggataaagaaattaaaaagggccctcgcatccagaatctccctgggtcccaagcagcaactcaacaggcgacccacccattggcaggggacaccccgaacatgcaggcacggacaaaacccctgaccaagccacatcaagaggcaatcaatcctggcaaccaggacacaggagagaatattcatttaccaccttccatggcaccaccagagtcattagttggtgcaatccgcaatgtaccccaattcgtgccagaccaatctatgacgaatgtagatgcggggagtgtccaactacatgcatcatgtgcagagatgataagtagaatgcttgtagaagttatatctaagcttgataaactcgagtcgagactgaatgatatagcaaaagttgtaaacaccaccccccttatcaggaatgatattaaccaacttaaggccacaactgcactgatgtccaaccaaattgcttccatacaaattcttgacccagggaatgcaggggtgaggtccctctctgaaatgagatctgtgacgaagaaagctgctgttgtaattgcaggatttggagacgacccaactcaaattattgaagaaggtatcatggccaaagatgctcttggaaaacctgtgcctccaacatctgttatcgcagccaaagctcagacttcttccggtgtgagtaagggtgaaatagaaggattgattgcattggtggaaacattagttgacaatgacaagaaggcagcgaaactgattaaaatgattgatcaagttaaatcccacgccgattacgcccgagtcaagcaggcaatatataatgcataatattgtaattatacaaacaatcaatactgctgtcggttgcacccaccttagcaaatcaataatcttttaaaattgattgattaagaaaaaa

SEQ ID NO: 80 - protein, 405 aa, artificial sequence
  P protein of ACN88140
MDFANDEEIAELLNLSTTVIKEIQKSELKPPQTTGRPPVSQGNTRNLTDLWEKETASQNKTSAQSPQTTQVQSDGNEEEEIKSESIDGHISGTVNQLEQVPEQNQSRSSPGDDLDRALNKLEGRINSISSMDKEIKKGPRIQNLPGSQAATQQATHPLAGDTPNMQARTKPLTKPHQEAINPGNQDTGENIHLPPSMAPPESLVGAIRNVPQFVPDQSMTNVDAGSVQLHASCAEMISRMLVEVISKLDKLESRLNDIAKVVNTTPLIRNDINQLKATTALMSNQIASIQILDPGNAGVRSLSEMRSVTKKAAVVIAGFGDDPTQIIEEGIMAKDALGKPVPPTSVIAAKAQTSSGVSKGEIEGLIALVETLVDNDKKAAKLIKMIDQVKSHADYARVKQAIYNA

SEQ ID NO: 81 - DNA, 1110 bp, artificial sequence
  M gene of FJ619036
atggcatatacaacactaaaactgtgggtggatgagggtgacatgtcgtcttcgcttctatcattcccgttggtactaaaagagacagacagaggcacaaagaagcttcaaccacaggtaagggtagattcaattggcgatgtgcagaatgccaaagagtcctcgatattcgtgactctatatggtttcatccaagcaattaaggagaattcagatcgatcgaaattcttccatccaaaagatgacttcaaacctgagacagtcactgcaggactggtagtagtgggtgcaatccgaatgatggctgatgtcaataccatctctaatgatgcactagcgctggagatcactgttaagaaatctgcaacttctcaagagaaaatgacggtgatgttccacaatagccccccttcattgagaactgcaataactatccgagcaggaggtttcatctcgaatgcagacgaaaatataaaatgtgccagcaagttgactgcaggagtgcagtacatattccgtccaatgtttgtttcaatcactaaattacacaatggcaaactatatagggtgcccaaaagtatccacagcatctcgtctaccctactgtatagtgtgatgttggaggtaggattcaaagtggacatcgggaaggatcatccccaggcaaaaatgctgaagagggtcacaattggcgatgcagacacatactggggatttgcatggttccacctgtgcaatttcaaaaagacatcctctaagggaaagccgagaacgctagacgaactgaggacaaaagtcaaaaatatggggttgaaattggagttacatgacctatggggtccgactattgtggtccaaatcactggcaagagcagcaaatatgctcaaggatttttttcttccaatggtacttgttgcctcccaatcagcagatctgcaccagagcttgggaagcttctgtggtcctgctcagcaactattggtgacgcaacagttgttatccaatcaagcgagaagggggaactcctaaggtctgatgatctcgagatacgaggtgctgtggcctccaagaaaggtagactgagctcatttcaccccttcaaaaaatga

SEQ ID NO: 82 - protein, 369 aa, artificial sequence
  M protein of ACN88143
MAYTTLKLWVDEGDMSSSLLSFPLVLKETDRGTKKLQPQVRVDSIGDVQNAKESSIFVTLYGFIQAIKENSDRSKFFHPKDDFKPETVTAGLVVVGAIRMMADVNTISNDALALEITVKKSATSQEKMTVMFHNSPPSLRTAITIRAGGFISNADENIKCASKLTAGVQYIFRPMFVSITKLHNGKLYRVPKSIHSISSTLLYSVMLEVGFKVDIGKDHPQAKMLKRVTIGDADTYWGFAWFHLCNFKKTSSKGKPRTLDELRTKVKNMGLKLELHDLWGPTIVVQITGKSSKYAQGFFSSNGTCCLPISRSAPELGKLLWSCSATIGDATVVIQSSEKGELLRSDDLEIRGAVASKKGRLSSFHPFKK

SEQ ID NO: 83 - DNA, 1632 bp, artificial sequence; identical to SEQ ID NO: 71
  F gene of FJ619036

SEQ ID NO: 84 - protein, 543 aa, artificial sequence; identical to SEQ ID NO: 72
  F protein of ACN88144

SEQ ID NO: 85 - DNA, 1734 bp, artificial sequence
  HN gene of FJ619036
atgagtaacattgcatccagtttagaaaatattgtggagcaggatagtcgaaaaacaacttggagggccatctttagatggtccgttcttcttattacaacaggatgcttagccttatccattgttagcatagttcaaattgggaatttgaaaattccttctgtaggggatctggcggacgaggtggtaacacctttgaaaaccactctgtctgatacactcaggaatccaattaaccagataaatgacatattcaggattgttgcccttgatattccattgcaagtaactagtatccaaaaagacctcgcaagtcaatttagcatgttgatagatagtttaaatgctatcaaattgggcaacgggaccaaccttatcatacctacatcagataaggagtatgcaggaggaattggaaaccctgtctttactgtcgatgctggaggttctataggattcaagcaatttagcttaatagaacatccgagctttattgctggacctacaacgacccgaggctgtacaagaatacccacttttcacatgtcagaaagtcattggtgctactcacacaacatcatcgctgctggctgtcaagatgccagtgcatctagtatgtatatctcaatgggggttctccatgtgtcttcatctggcactcctatctttcttactactgcaagtgaactgatagacgatggagttaatcgtaagtcatgcagtattgtagcaacccaattcggctgtgacattttgtgcagtattgtcatagagaaggagggagatgattattggtctgatactccgactccaatgcgccacggccgtttttcattcaatgggagttttgtagaaaccgaactacccgtgtccagtatgttctcgtcattctctgccaactaccctgctgtgggatcaggcgaaattgtaaaagatagaatattattcccaatttacggaggtataaagcagacttcaccagagtttaccgaattagtgaaatatggactctttgtgtcaacacctacaactgtatgtcagagtagctggacttatgaccaggtaaaagcagcgtataggccagattacatatcaggccggttctgggcacaagtgatactcagctgcgctcttgatgcagtcgacttatcaagttgtattgtaaagattatgaatagcagcacagtgatgatggcagcagaaggaaggataataaagatagggattgattacttttactatcagcggtcatcttcttggtggccattggcatttgttacaaaactagacccgcaagagttagcagacacaaactcgatatggctgaccaattccataccaatcccacaatcaaagttccctcggccttcatattcagaaaattattgcacaaagccagcagtttgccctgctacttgtgtcactggtgtatactctgatatttggcccttgacctcatcttcatcactcccgagcataatttggatcggccagtaccttgatgcccctgttggaaggacttatcccagatttggaattgcaaatcaatcacactggtaccttcaagaagatattctacccacctccactgcaagtgcgtattcaaccactacatgttttaagaatactgccaggaatagagtgttctgcgtcaccattgctgaatttgcagatgggttgtttggagagtacaggataacacctcagttgtatgaattagtgagaaataattga

SEQ ID NO: 86 - protein, 577 aa, artificial sequence; identical to SEQ ID NO: 74
  HN protein of ACN88145

SEQ ID NO: 87 - DNA, 6717 bp, artificial sequence
  L gene of FJ619036
atggatgtaaaacaagttgacctaataatacaacccgaggttcatctcgattcacccatcatattgaataaactggcactattatggcgcttgagtggtttacccatgcctgcagacttacgacaaaaatccgtagtgatgcacatcccagaccacatcttagaaaaatcagaatatcggatcaagcaccgtctagggaaaatcaagagtgacatagcacattactgtcagtattttaatattaatttggcaaatcttgatccgataacccaccccaaaagtttgtattggttatccagactaacaatagctagtgctggaacctttagacatatgaaagatagaatcttatgtacagttggctccgaattcggacacaaaattcaagatttattttcactgctgagccataaattagtaggtaacggtgatttatttaatcaaagtctctcaggtacacgtttgactgcgagtccgttatcccctttatgcaatcaatttgtctctgacatcaagtctgcagtcacgacaccctggtcagaagctcgttggtcttggcttcatatcaaacaaacaatgagatacctgataaaacaatcacgcactacaaattcagctcatttaacagaaattataaaagaggaatggggtttagtaggtattactccagatcttgtcattctttttgacagagtcaataatagtctaactgcattaacatttgagatggttctaatgtattcagatgtattagaatcccgtgacaatattgtgctagtggggcgattatctacttttctgcagccagtagttagtagactggaggtgttgtttgatctagtagattcattggcaaaaaccttaggtgacacaatatacgaaattattgcggtgttagagagcttgtcttatgggtccgttcaactacatgatgcaagtcactctcatgcagggtctttcttttcatttaacatgaatgaacttgataacacactatcaaagagggtggatccgaaacacaagaacaccataatgagcattataagacaatgcttttctaatctagatgttgatcaagctgcagagatgctatgcctgatgagattatttggacacccaatgttaactgcaccggatgcagcagccaaagtaaggaaagcaatgtgtgctccaaaacttgttgaacatgacaccatcttgcagacattatccttcttcaagggaataattataaatgggtacagaagatcacactctggcctgtggcccaatgtagagccgtcttcaatctatgatgatgatctcagacagctgtacttagagtcagcagagatttcccatcatttcatgcttaaaaactacaagagtttgagcatgatagaattcaagaagagcatagactacgatcttcacgacgacttaagtactttcttaaaggatagagcaatttgccggccaaaatcccagtgggatgttatattccgtaagtctttacgcagatcccacacgcggtcccagtatatggacgaaattaagagcaaccgattgctaattgattttcttgattctgctgattttgaccctgaaaaggaatttgcatatgtaaccacaatggattatttgcacgataatgaattttgtgcttcatattctctaaaggaaaaggagatcaaaactaccgggaggatatttgcaaaaatgacacgcaatatgagaagttgccaagtgatacttgaatctctgttatcaaaacatatatgcaagttcttcaaagagaacggcgtttcgatggagcaattgtcattgaccaagagtctacttgcaatgtctcaactctcaccaaaagtctcgactctgcaggacactgcatcacgtcatgtaggcaactcaaaatctcagatcgcaaccagcaacccatctcggcatcactcaacaaccaatcagatgtcactctcaaatcggaaaacggttgtagcaactttcttaacaactgatttggaaaaatactgcctgcagtggcgatactcgactattaagttgtttgcacaagctctaaatcaactctttgggattgatcacggatttgaatggatacatttaagactcatgaacagcaccttatttgtcggtgatccttactcgcctcctgaagatccaacactagaggatatagataaagcaccaaatgacgatatcttcatagtttctccaaggggaggcatagagggtttatgtcagaagatgtggaccatgatatcaattagtgcgatacactgtgtagcagagaaaattggtgcacgagtggcagcaatggtgcagggtgataatcaagtaatagctatcaccaaagaactattcagaggagagaaagcctgtgatgtcagagatgagttagacgagctcggtcaggtgttttttgatgagttcaagaggcacaattatgcaattggacacaaccttaagctaaatgagacaatacaaagccaatccttttttgtatattccaaacgaatattctttgaagggcgattgcttagtcaagtcctcaaaaatgctgccaagttatgtatggttgctgaccatctaggtgaaaacacagtatcttcctgtagcaacctgagctctacaattgcccggttggtggaaaatgggtttgagaaggacactgcttttgtgttgaacctagtctacatcatgactcaaattctttttgatgagcattactcgattgtatgcgatcacaatagtgtcaaaagcttgatcggatcaaaaaactatcggaatctattgtactcatctctaataccaggtcagctcggtggtttcaacttcctcaatataagtcggttgttcactaggaatataggtgacccagtaacatgtagtctgtctgatctcaaatgcttcatagccgcaggtctccttccaccctatgtacttaaaaatgtggttctgcgtgagcctggtcctgggacatggttgacgttgtgctctgatccttacacccttaacataccatacacacagctaccaaccacatatctcaaaaagcacacccagcgatcgttgctttcacgtgcagtaaatcctttattagcaggtgtacaagtgccaaatcagcatgaggaagaagagatgttggctcgctttctccttgatcgtgaatatgtgatgccccgcgttgctcatgtaacactagaaacatcggtccttggcaaacggaaacaaatccaaggcttaattgatacaactccaactatcattagaacatctctagtcaatctaccagtgtctaggaagaaatgcgaaaaaataatcaattattctctcaattatattgctgagtgtcatgactccttacttagtcagatctgcttcagtgataataaggaatacttgtggtccacctccttaatatcagttgagacctgtagtgtgacaattgcggactatttgagagctgtcagctggtctaatatattagggggaagaagcatatccggggtgactacacctgatactattgaattaattcaaggttgtttaataggtgaaaattccagttgtactctttgtgaatcgcatgacgacgcattcacatggatgcacttgcctggcccactttacatccctgaaccatcagttactaactctaaaatgcgtgtgccatatctgggttcaaaaacagaggagcgtaaaacagcttcaatggcagcaataaaaggaatgtcacatcacctgcgtgcagtcttaagaggtacatccgtatttatttgggcatctggggacacagatattaattgggataatgcattgcagattgcccaatcacggtgtaacatcacattggatcaaatgagattacttacaccaattcctagcagttcaaatatccaacgtagactcgatgacggaatcagcacgcagaaatttactcctgcaagccttgctcgaatcacatcctctgttcacatctgtaatgacagccaaaggttagagaaggatggctcctctgtcgactcaaacttgatttaccagcaaattatgttacttggactcagcatctttgaaacaatgtactcaatggaccaaaagtgggtattcaataaccataccttacatttgcacactggacactcctgttgtccaagggaactagacataagtttagtgaacccgccaagacatcagaccccggagctgactagcacaacaaccaacccgttcctatatgatcagctcccactaaatcaggataatctgacaacacttgagattaagacattcaaatttaatgagctcaacattgatggtttagattttggtgaaggaatacaattattgagtcgttgtactgcaagattaatggcagaatgtattctagaggagggaataggctcgtcagttaaaaatgaagcaattgtcaattttgataattcagtcaattggatttcagagtgcctaatgtgtgatattcgctcactttgtgttaatttaggtcaagagatactatgtagcctggcataccaaatgtattacttgcgaatcaggggtagaagggccattcttaattacttggacacaactttgcaaaggatccctgtgatacagttagccaacattgcactcaccatttcacaccctgagatatttcgcagaattgtcaacaccgggatccataaccagattaagggcccatatgtggcaacaacagatttcatagctgcaagtagagatatcatattatcaggtgcaagggagtatctatcttatctaagcagtggacaggaagactgttacacattcttcaactgtcaagatggggatcttactccaaaaatggaacagtatcttgcaaggagggcatgccttttaacattactgtataatactgggcaccagatccccattatccgatcactgacaccaatagagaagtgcaaggtgctcacagaatacaatcaacaaattgagtatgcagatcaagagtttagctctgtattgaaagtggtcaatgcactactacaaaatcctaatatagatgcattggtttcaaatctctacttcaccaccagacgtgttttatcaaacctcagatcatgtgataaggctatatcatatattgaatatttgtacactgaggacttcggagaaaaagaagatacagtacaatatgacatcatgacaacaaacgatatcatacttactcatggtctattcacacagatcgaaatatcttaccaagggagtagtctccataaattcctaactccggataacgcgcctggatcattgatcccattctctatttcaccaaattcgcttgcatgtgatcctcttcaccacttactcaagtcggtcggtacatcaagcacaagctggtacaagtatgcaatcgcctatgcagtgtctgaaaagaggtcggctcgattaggagggagcttgtacattggtgaagggagcggaagtgtgatgactttgctagagtatcttgagccatctgttgacatattttacaattcactcttctcaaatggtatgaacccaccacaacgaaattatgggcttatgccactacaatttgtgaattcggtggtttataagaacttaacggctaaatcagaatgtaagctaggatttgtccagcaatttaaaccgttgtggagagacatagacattgagactaatgttacagatccatcatttgtcaattttgcattgaatgaaatcccaatgcaatcattaaaacgagtaaattgtgatgtggaatttgaccgtggtatgccgattgaacgggttattcagggttacactcatatcttacttgttgctacttacggattgcagcaagattcaatactgtgggtgaaagtatataggacatctgaaaaagtatttcagttcttactgagtgccatgatcatgatctttggttatgtcaaaatccacaggaatggttatatgtcggcaaaggatgaggagtacatattgatgtctgactgcaaggaacctgtaaactatacagctgtccctaacattcttacacgtgtaagtgatttagtgtcgaagaatctgagtcttatccatccagaagacctcagaaaggtaaggtgtgaaacagattccctgaatttgaagtgcaatcatatttatgagaaaataattgctagaaaaattccattacaggtgtcatcaactgattctttgctcctccagttaggcggtgtcatcaactcggtgggctcaactgatcctagagaggttgcaacgttatcttccattgagtgtatggactatgttgtctcatcaattgatttggctatattagaggcaaatattgtgatctcagagagtgctgatcttgacctcgctttaatgttaggcccattcaacttgaataagcttaagaaaattgacacaatccttaagtcaagcacctatcagctaatcccgtattggttgcgctatgagtactctattaatccgagatctttgtcatttctaatcactaaattacaacaatgccgaatttcatggtcagatatgataacaatctctgaattttgcaagaaatccaagcggcctatatttattaaacgagtaatagggaatcaacggctgaaatcattctttaatgaaagctcaagtattgttttgacccgggctgaagtcaaagtctgtataaagttcctcggtgcgatcatcaagttgaaataa

SEQ ID NO: 88 - protein, 2238 aa, artificial sequence
  L protein of ACN88146
MDVKQVDLIIQPEVHLDSPIILNKLALLWRLSGLPMPADLRQKSVVMHIPDHILEKSEYRIKHRLGKIKSDIAHYCQYFNINLANLDPITHPKSLYWLSRLTIASAGTFRHMKDRILCTVGSEFGHKIQDLFSLLSHKLVGNGDLFNQSLSGTRLTASPLSPLCNQFVSDIKSAVTTPWSEARWSWLHIKQTMRYLIKQSRTTNSAHLTEIIKEEWGLVGITPDLVILFDRVNNSLTALTFEMVLMYSDVLESRDNIVLVGRLSTFLQPVVSRLEVLFDLVDSLAKTLGDTIYEIIAVLESLSYGSVQLHDASHSHAGSFFSFNMNELDNTLSKRVDPKHKNTIMSIIRQCFSNLDVDQAAEMLCLMRLFGHPMLTAPDAAAKVRKAMCAPKLVEHDTILQTLSFFKGIIINGYRRSHSGLWPNVEPSSIYDDDLRQLYLESAEISHHFMLKNYKSLSMIEFKKSIDYDLHDDLSTFLKDRAICRPKSQWDVIFRKSLRRSHTRSQYMDEIKSNRLLIDFLDSADFDPEKEFAYVTTMDYLHDNEFCASYSLKEKEIKTTGRIFAKMTRNMRSCQVILESLLSKHICKFFKENGVSMEQLSLTKSLLAMSQLSPKVSTLQDTASRHVGNSKSQIATSNPSRHHSTTNQMSLSNRKTVVATFLTTDLEKYCLQWRYSTIKLFAQALNQLFGIDHGFEWIHLRLMNSTLFVGDPYSPPEDPTLEDIDKAPNDDIFIVSPRGGIEGLCQKMWTMISISAIHCVAEKIGARVAAMVQGDNQVIAITKELFRGEKACDVRDELDELGQVFFDEFKRHNYAIGHNLKLNETIQSQSFFVYSKRIFFEGRLLSQVLKNAAKLCMVADHLGENTVSSCSNLSSTIARLVENGFEKDTAFVLNLVYIMTQILFDEHYSIVCDHNSVKSLIGSKNYRNLLYSSLIPGQLGGFNFLNISRLFTRNIGDPVTCSLSDLKCFIAAGLLPPYVLKNVVLREPGPGTWLTLCSDPYTLNIPYTQLPTTYLKKHTQRSLLSRAVNPLLAGVQVPNQHEEEEMLARFLLDREYVMPRVAHVTLETSVLGKRKQIQGLIDTTPTIIRTSLVNLPVSRKKCEKIINYSLNYIAECHDSLLSQICFSDNKEYLWSTSLISVETCSVTIADYLRAVSWSNILGGRSISGVTTPDTIELIQGCLIGENSSCTLCESHDDAFTWMHLPGPLYIPEPSVTNSKMRVPYLGSKTEERKTASMAAIKGMSHHLRAVLRGTSVFIWASGDTDINWDNALQIAQSRCNITLDQMRLLTPIPSSSNIQRRLDDGISTQKFTPASLARITSSVHICNDSQRLEKDGSSVDSNLIYQQIMLLGLSIFETMYSMDQKWVFNNHTLHLHTGHSCCPRELDISLVNPPRHQTPELTSTTTNPFLYDQLPLNQDNLTTLEIKTFKFNELNIDGLDFGEGIQLLSRCTARLMAECILEEGIGSSVKNEAIVNFDNSVNWISECLMCDIRSLCVNLGQEILCSLAYQMYYLRIRGRRAILNYLDTTLQRIPVIQLANIALTISHPEIFRRIVNTGIHNQIKGPYVATTDFIAASRDIILSGAREYLSYLSSGQEDCYTFFNCQDGDLTPKMEQYLARRACLLTLLYNTGHQIPIIRSLTPIEKCKVLTEYNQQIEYADQEFSSVLKVVNALLQNPNIDALVSNLYFTTRRVLSNLRSCDKAISYIEYLYTEDFGEKEDTVQYDIMTTNDIILTHGLFTQIEISYQGSSLHKFLTPDNAPGSLIPFSISPNSLACDPLHHLLKSVGTSSTSWYKYAIAYAVSEKRSARLGGSLYIGEGSGSVMTLLEYLEPSVDIFYNSLFSNGMNPPQRNYGLMPLQFVNSVVYKNLTAKSECKLGFVQQFKPLWRDIDIETNVTDPSFVNFALNEIPMQSLKRVNCDVEFDRGMPIERVIQGYTHILLVATYGLQQDSILWVKVYRTSEKVFQFLLSAMIMIFGYVKIHRNGYMSAKDEEYILMSDCKEPVNYTAVPNILTRVSDLVSKNLSLIHPEDLRKVRCETDSLNLKCNHIYEKIIARKIPLQVSSTDSLLLQLGGVINSVGSTDPREVATLSSIECMDYVVSSIDLAILEANIVISESADLDLALMLGPFNLNKLKKIDTILKSSTYQLIPYWLRYEYSINPRSLSFLITKLQQCRISWSDMITISEFCKKSKRPIFIKRVIGNQRLKSFFNESSSIVLTRAEVKVCIKFLGAIIKLK